(19) United States
(12) Patent Application Publication
(10) Pub. No.: US 2026/0223533 A1
(43) Pub. Date: Jul. 30, 2026

(54) ORGANIC COMPOUND AND ORGANIC ELECTROLUMINESCENT DEVICE USING THE SAME

(71) Applicant: SOLUS ADVANCED MATERIALS CO., LTD., Iksan-si, Jeollabuk-do (KR)

(72) Inventors: Sangeun SIM, Seongnam-si (KR); Uigeon LEE, Seongnam-si (KR); Hyosuk SON, Seongnam-si (KR); Jaeyi SIM, Seongnam-si (KR)

(73) Assignee: SOLUS ADVANCED MATERIALS CO., LTD., Iksan-si, Jeollabuk-do (KR)

(21) Appl. No.: 19/143,677

(22) PCT Filed: Dec. 27, 2023
(86) PCT No.: PCT/KR2023/021673

§ 371 Date: Jun. 26, 2025

(30) Foreign Application Priority Data
Dec. 27, 2022 (KR) 10-2022-0186335
Dec. 26, 2023 (KR) 10-2023-0191531

(51) Int. Cl. H10K 50/16; C07D 401/14; C09K 11/06; H10K 50/19; H10K 85/60

(52) U.S. Cl. CPC H10K 50/16; C07D 401/14; C09K 11/06; H10K 50/19; H10K 85/622; H10K 85/626; H10K 85/633; H10K 85/654; H10K 85/6572; C09K 2211/1044

(57) ABSTRACT

A novel organic compound and an organic electroluminescent device using the novel organic compound are disclosed. The novel organic compound has excellent electron injection and transport capabilities. The organic electroluminescent device containing the compound in one or more organic layers thereof, is improved not only in terms of characteristics such as luminous efficiency, driving voltage, and lifespan, but also in terms of progressive driving voltage.

### TECHNICAL FIELD

The present disclosure relates to a novel organic compound and an organic electroluminescent device including the same, and more specifically, to a compound having excellent electron capability, and an organic electroluminescent device which includes the compound in one or more organic layers, thereby improving not only characteristics such as luminous efficiency, driving voltage, lifetime, but also progressive driving voltage.

### BACKGROUND ART

In organic electroluminescent devices (hereinafter, referred to as “organic EL devices”), upon application of voltage across two electrodes, holes are injected from an anode to an organic layer and electrons are injected from a cathode into the organic layer. Injected holes and electrons recombine to form excitons, and light may be emitted when these excitons decay to the ground state. In such cases, materials used for the organic layer may be classified, for example, as light emitting materials, hole injection materials, hole transport materials, electron transport materials and electron injection materials, depending on their function.

Light emitting materials in an organic EL device may be classified into blue-, green- and red-light emitting materials depending on their emission colors. Additionally, yellow and orange light emitting materials may be employed as a light emitting material to achieve natural color reproduction. In addition, a host/dopant system may also be used in the light emitting material to enhance color purity and luminescence efficiency through energy transfer. Dopant materials may be classified into fluorescent dopants, which use organic materials, and phosphorescent dopants, which use metal complex compounds containing heavy atoms such as iridium (Ir) and platinum (Pt). The phosphorescent materials may theoretically achieve up to four times higher luminescence efficiency than the fluorescent materials, so attention has been directed toward phosphorescent dopants as well as phosphorescent host materials.

To date, NPB, BCP and Alq₃, whose chemical formulas are shown below, have been widely recognized as materials used in the hole injection layer, hole transport layer, hole blocking layer and electron transport layer, and anthracene derivatives have also been reported as fluorescent dopant/host materials for light emitting materials. In particular, Ir-based metal complexes such as FIrpic, Ir(ppy)₃, and Ir(btp)₂ (acac) are known as phosphorescent dopant materials for improving efficiency among light emitting materials, and are used as blue, green and red dopant materials. Among phosphorescent host materials group, CBP has demonstrated excellent performance to date.

However, despite exhibiting good luminescence properties, conventional materials have shown low glass transition temperatures and poor thermal stability, and are accordingly inadequate in terms of operational lifetime characteristics for organic EL devices. Accordingly, there is a growing demand for the development of organic layer materials with enhanced performance.

### DESCRIPTION OF THE INVENTION

### Technical Objectives

The present disclosure is directed to a novel compound that exhibits excellent electron injection and transport capabilities, electrochemical stability, and thermal stability, and can thus be used as a material for the organic layer in an organic EL device, more specifically as a material for an electron transport layer or an N-type charge generation layer.

The present disclosure is also directed to an organic EL device including the aforementioned compound, thereby having reduced driving voltage, improved luminous efficiency, and enhanced lifetime characteristics.

### Technical Means

In order to achieve the above objective, the present disclosure provides a compound represented by the following Chemical Formula 1:

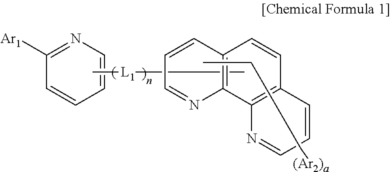

(wherein,
Ar₁ is a C9-C40 heteroaryl group containing one nitrogen (N) atom,
n is an integer in a range of 1 to 3,
L₁ is a C6-C40 arylene group,
a is an integer in a range of 0 to 7,
Ar₂ is selected from the group consisting of: hydrogen, deuterium (D), halogen group, cyano group, nitro group, amino group, C1-C40 alkyl group, C2-C40 alkenyl group, C2-C40 alkynyl group, C3-C40 cycloalkyl group, heterocycloalkyl group having 3 to 40 nuclear atoms, C6-C60 aryl group, heteroaryl group having 5 to 60 nuclear atoms, C1-C40 alkyloxy group, C6-C60 aryloxy group, C1-C40 alkylsilyl group, C6-C60 arylsilyl group, C1-C40 alkylboron group, C6-C60 arylboron group, C6-C60 arylphosphine group, C6-C60 arylphosphine oxide group, C6-C60 arylamine group, (C6-C60 aryl) (heteroaryl having 5 to 60 nuclear atoms) amine group, and heteroarylamine group having 5 to 60 nuclear atoms, or fused with an adjacent group to form a fused ring;
the heteroaryl group of Ar₁, the arylene group of L₁, and the alkyl group, alkenyl group, alkynyl group, cycloalkyl group, heterocycloalkyl group, aryl group, heteroaryl group, alkyloxy group, aryloxy group, alkylsilyl group, arylsilyl group, alkylboron group, arylboron group, arylphosphine group, arylphosphine oxide, arylamine group, (aryl) (heteroaryl)amine, and heteroarylamine group of Ar₂ are each independently substituted or unsubstituted with one or more substituents selected from the group consisting of: deuterium (D), halogen group, cyano group, nitro group, amino group, C1-C40 alkyl group, C2-C40 alkenyl group, C2-C40 alkynyl group, C3-C40 cycloalkyl group, heterocycloalkyl group having 3 to 40 nuclear atoms, C6-C60 aryl group, heteroaryl group having 5 to 60 nuclear atoms, C1-C40 alkyloxy group, C6-C60 aryloxy group, C1-C40 alkylsilyl group, C6-C60 arylsilyl group, C1-C40 alkylboron group, C6-C60 arylboron group, C6-C60 arylphosphine group, C6-C60 arylphosphine oxide group, C6-C60 arylamine group, (C6-C60 aryl) (heteroaryl having 5 to 60 nuclear atoms) amine group, and heteroarylamine group having 5 to 60 nuclear atoms, and when a plurality of the substituents are present, they are the same as or different from each other).

Also, the present disclosure provides an organic EL device including: an anode; a cathode; and one or more organic layers disposed between the anode and the cathode, wherein at least one of the one or more organic layers comprises the aforementioned compound.

Furthermore, the present disclosure provides an organic EL device including: an anode and a cathode that are arranged at a distance; a plurality of lighting units interposed between the anode and the cathode; and an N-type charge generation layer and a P-type charge generation layer, interposed between the adjacent lighting units, wherein each of the lighting units comprises a hole transport layer, a light-emitting layer, and an electron transport layer, and the N-type charge generation layer includes the compound.

### Effect of the Invention

The compound according to the present disclosure exhibits excellent electron transport ability, luminescent ability, electrochemical stability, and thermal stability, and accordingly may be used as a material for an organic layer of an organic EL device. In particular, when used as material for at least one of an electron transport layer and an N-type charge generation layer, the compound of the present disclosure allows for the fabrication of organic EL with superior luminous performance, low driving voltage, high efficiency, and long lifespan compared to conventional materials, and furthermore, it is possible to produce full color display panels with improved performance and durability.

### BRIEF DESCRIPTION OF THE DRAWINGS

FIG. 1 is a schematic cross-sectional view of an organic EL device according to the first embodiment of the present disclosure.

FIG. 2 is a schematic cross-sectional view of an organic EL device according to the second embodiment of the present disclosure.

FIG. 3 is a schematic cross-sectional view of an organic EL device according to the third embodiment of the present disclosure.

FIG. 4 is a schematic cross-sectional view of an organic EL device according to the fourth embodiment of the present disclosure.

### REFERENCE NUMERAL

100: anode, 200: cathode,
300: organic layer, 310: hole injection layer,
320: hole transport layer, 330: light-emitting layer,
340: electron transport layer, 350: electron
injection layer,
360: electron transport auxiliary layer, 400: 1^{st} light-emitting unit,
410: 1^{st} hole transport layer, 420: 1^{st} light-emitting layer,
430: 1^{st} electron transport layer, 440: hole injection layer,

500: 2^{nd} light-emitting unit, 510: 2^{nd} hole transport layer,
520: 2^{nd} light-emitting layer, 530: electron transport,
600: charge generation layer, 610: N-type charge generation layer,
620: P-type charge generation layer

### MODES FOR IMPLEMENTING THE INVENTION

Hereinafter, the present disclosure will be described.

### <Novel Compound>

The compound according to the present disclosure has a structure including: a pyridine moiety substituted with an N-containing heteroaryl group; and a phenanthroline moiety linked to the pyridine moiety via a linker, and is represented by Chemical Formula 1. Such a compound has excellent electron injection and transport capability, electrochemical stability, and thermal stability, and thus can be used as an organic layer material of an organic electroluminescent device. Especially, the use of the compound of the present disclosure as any one of an electron transport layer material and an N-type charge generation layer material of an organic electroluminescent device enables the production of electroluminescent devices with excellent luminous performance, low deriving voltage, high efficiency, and long lifespan characteristics compared with conventional materials, and furthermore, enables the production of full color display panels with improved performance and lifespan.

Specifically, in the compound represented by Chemical Formula 1, the pyridine moiety and phenanthroline moiety are electron withdrawing groups with strong electron withdrawing. Due to this, the compound of Chemical Formula 1 has a structure where both sides of the molecule are electron withdrawing groups (EWG), respectively, and these two electron withdrawing groups (EWG) are linked via a linker group (e.g., a phenylene group, a naphthalene group, etc.). Therefore, the compound of the present disclosure has excellent electron injection and transport capability, electrochemical stability, and thermal stability.

In addition, one hydrogen of the pyridine moiety may be substituted with a nitrogen (N)-containing heteroaryl group. Particularly, the N-containing heteroaryl group is introduced at the ortho-position with respect to the nitrogen (N) atom of the pyridine moiety, so that the pyridine moiety is capable of metal binding. Meanwhile, the phenanthroline moiety also has a structure in which two adjacent nitrogen atoms are adjacent to each other, and thus, the phenanthroline moiety can form a covalent bond with a neighboring hydrogen atom (H) or a coordination bond with an alkali metal or alkaline earth metal, such as Li or Yb. That is, the compound of the present disclosure contains two moieties capable of metal binding. Therefore, the compound of the present disclosure can form a gap state by binding to a metal, such as an alkali metal or an alkali earth metal (e.g., Li, Yb, etc.), which is a dopant in the N-type charge generation layer, and thus, the application of the compound of the present disclosure as an N-type charge generation layer material can exhibit improved electron transport characteristics to the electron transport layer. Specifically, even though used as a host material alone without being mixed with a different host material, the compound of the present disclosure can smoothly transport electrons from the N-type charge generation layer to the electron transport layer due to the gap state. Additionally, the use of the compound of the present disclosure as an N-type charge generation layer material allows the pyridine moiety and phenanthroline moiety to be bound to an alkali metal or an alkali earth metal in the N-type charge generation layer, thereby preventing the diffusion of the alkali metal or the alkali earth metal into a P-type charge generation layer. Therefore, the use of the compound of the present disclosure as an N-type charge generation layer material enables a low driving voltage, improved luminous efficiency, and high lifespan for the organic electroluminescent device.

As described above, the compound of the present disclosure exhibits excellent electron injection and transport capability. Therefore, the compound of the present disclosure can be used as an organic layer material, specifically, an electron transport layer material for an organic electroluminescent device. In addition, the compound of the present disclosure may be also used as an N-type charge generation layer material for a tandem organic electroluminescent device. Therefore, when applied as an electron transport layer material or an N-type charge generation layer material for an organic electroluminescent device, the compound represented by Chemical Formula 1 of the present disclosure can improve characteristics of the device, such as driving voltage, luminous efficiency, and lifespan, as well as prevent the increase of a progressive driving voltage and, furthermore, maximize the performance of a full-color organic light-emitting panel employing the organic electroluminescent device.

In the compound represented by the above Chemical Formula 1, Ar₁ may be a C₉-C₄₀ heteroaryl group containing one nitrogen (N) atom, and specifically, a C₉-C₁₈ fused polycyclic heteroaryl group containing one nitrogen (N) atom. The compound of the present disclosure contains such a heteroaryl group containing one nitrogen atom, and thus exhibits an enhanced binding strength with a metal, leading to an enhancement in electron transport capability, thereby achieving a low driving voltage, high luminous efficiency, and high lifespan for the organic electroluminescent device.

Here, the heteroaryl group of Ar₁ may each independently be substituted or unsubstituted with one or more substituents selected from the group consisting of: deuterium (D), halogen group (e.g., —F, —Cl, —Br, —I, etc.), cyano group (—CN), nitro group (—NO₂), amino group (—NH₂), C1-C40 alkyl group, C2-C40 alkenyl group, C2-C40 alkynyl group, C3-C40 cycloalkyl group, heterocycloalkyl group having 3 to 40 nuclear atoms, C6-C60 aryl group, heteroaryl group having 5 to 60 nuclear atoms, C1-C40 alkyloxy group, C6-C60 aryloxy group, C1-C40 alkylsilyl group, C6-C60 arylsilyl group, C1-C40 alkylboron group, C6-C60 arylboron group, C6-C60 arylphosphine group, C6-C60 arylphosphine oxide group, C6-C60 arylamine group, (C6-C60 aryl) (heteroaryl having 5 to 60 nuclear atoms) amine group. In such a case, when a plurality of substituents are present, they may be the same as or different from each other.

In an example, Ar₁ may be a substituent represented by the following Chemical Formula S1-1 or S1-2, but the present disclosure is not limited thereto.

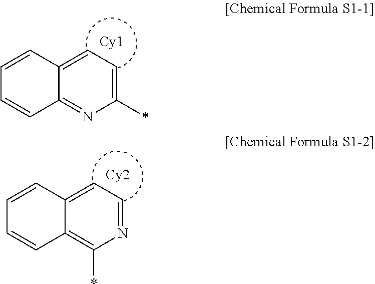

In the above Chemical Formulas S1-1 and S1-2,
may represent a site bonded to Chemical Formula 1,
Cy1 and Cy2 may each independently be a C6-C30 fused aromatic ring, and specifically, may each independently be a C6-C18 fused aromatic ring,
the fused aromatic ring of Cy1 and Cy2 may each independently be substituted or unsubstituted with one or more substituents selected from the group consisting of: deuterium (D), halogen group, cyano group, nitro group, amino group, C1-C40 alkyl group, C2-C40 alkenyl group, C2-C40 alkynyl group, C3-C40 cycloalkyl group, heterocycloalkyl group having 3 to 40 nuclear atoms, C6-C60 aryl group, heteroaryl group having 5 to 60 nuclear atoms, C1-C40 alkyloxy group, C6-C60 aryloxy group, C1-C40 alkylsilyl group, C6-C60 arylsilyl group, C1-C40 alkylboron group, C6-C60 arylboron group, C6-C60 arylphosphine group, C6-C60 arylphosphine oxide group, C6-C60 arylamine group, (C6-C60 aryl) (heteroaryl having 5 to 60 nuclear atoms) amine group, and heteroarylamine group having 5 to 60 nuclear atoms, and when a plurality of the substituents are present, they may be the same as or different from each other.

In another example, Ar₁ may be selected from the group consisting of substituents represented by the following Chemical Formulas S2-1 to S2-9, but the present disclosure is not limited thereto.

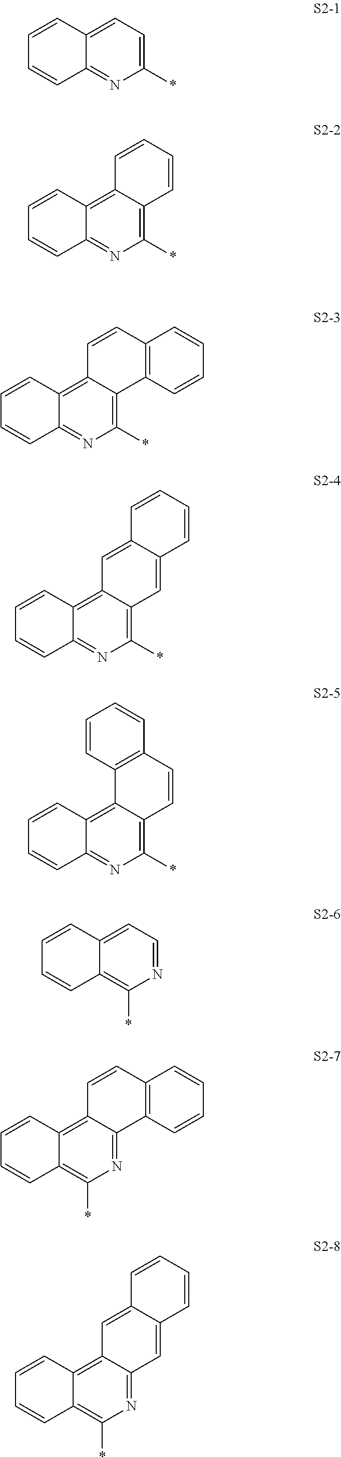

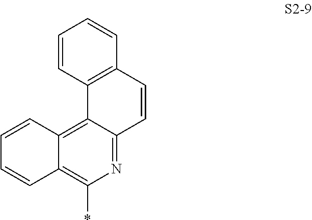 [SMILES: *c1nc2ccc3ccccc3c2c2ccccc12]

In the above Chemical Formulas S2-1 to S2-9,
* may represent a site bonded to Chemical Formula 1.

Hydrogens of the above substituents S2-1 to S2-9 may each independently be substituted or unsubstituted with one or more substituents selected from the group consisting of: deuterium (D), halogen group (e.g., —F, —Cl, —Br, —I, and the like), cyano group (—CN), nitro group (—NO₂), amino group (—NH₂), C1-C12 alkyl group (e.g., methyl group, ethyl group, propyl group, butyl group, and the like), C6-C10 aryl group (e. g., phenyl group, biphenyl group, terphenyl group, naphthyl group, and the like), and heteroaryl group having 5 to 10 ring atoms (e.g., a monovalent pyrazine group, a monovalent pyrimidine group, a monovalent pyridazine group, a monovalent triazine group, and the like).

In the compound represented by the above Chemical Formula 1, n may be an integer in a range of 1 to 3.

When n may be an integer in a range of 1 to 3, L₁ may be divalent linkers, which may be a C6-C40 arylene group, and specifically, may be a C6-C18 arylene group. Herein, the plurality of L₁ may be the same as or different from each other.

In such a case, the arylene group of L₁ may each independently be substituted or unsubstituted with one or more substituents selected from the group consisting of: deuterium (D), halogen group (e.g., —F, —Cl, —Br, —I, etc.), cyano group (—CN), nitro group (—NO₂), amino group (—NH₂), C1-C40 alkyl group, C2-C40 alkenyl group, C2-C40 alkynyl group, C3-C40 cycloalkyl group, heterocycloalkyl group having 3 to 40 nuclear atoms, C6-C60 aryl group, heteroaryl group having 5 to 60 nuclear atoms, C1-C40 alkyloxy group, C6-C60 aryloxy group, C1-C40 alkylsilyl group, C6-C60 arylsilyl group, C1-C40 alkylboron group, C6-C60 arylboron group, C6-C60 arylphosphine group, C6-C60 arylphosphine oxide group, C6-C60 arylamine group, (C6-C60 aryl) (heteroaryl having 5 to 60 nuclear atoms) amine group, and heteroarylamine group having 5 to 60 nuclear atoms. Herein, when a plurality of the substituents are present, they may be the same as or different from each other.

In an example, one or more L₁ may be the same as or different from each other and each independently be selected from the group consisting of: phenylene group, biphenylene group, terphenylene group, naphthylene group, phenanthrylene group, anthrylene group, triphenylene group, fluorenylene group, and combinations thereof. Here, hydrogens of phenylene group, biphenylene group, terphenylene group, naphthylene group, phenanthrylene group, anthrylene group, triphenylene group, fluorenylene group may each independently be substituted or unsubstituted with one or more substituents selected from the group consisting of: deuterium (D), halogen group (e.g., —F, —Cl, —Br, —I, and the like), cyano group (—CN), nitro group (—NO₂), amino group (—NH₂), C1 to C12 alkyl group, C6 to C10 aryl group, and heteroaryl group having 5 to 10 nuclear atoms.

In another example, one or more L₁ may be the same as or different from each other and each independently be selected from the group consisting of: the following linker groups L₁-1 to L₁-3, but the present disclosure is not limited thereto.

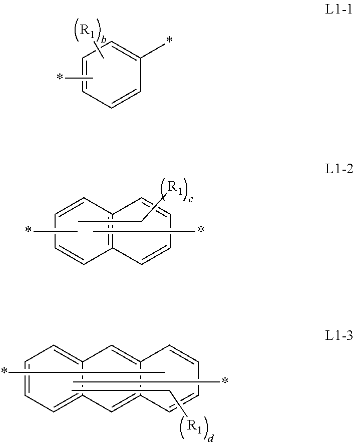

In the linker groups L₁-1 to L₁-3,
b may be an integer in a range of 0 to 4,
c may be an integer in a range of 0 to 6,
d may be an integer in a range of 0 to 8,
R₁ may be selected from the group consisting of: hydrogen, deuterium (D), halogen group (e.g., —F, —Cl, —Br, —I, and the like), cyano group (—CN), nitro group (—NO₂), amino group (—NH₂), C1-C40 alkyl group, C2-C40 alkenyl group, C2-C40 alkynyl group, C3-C40 cycloalkyl group, heterocycloalkyl group having 3 to 40 nuclear atoms, C6-C60 aryl group, heteroaryl group having 5 to 60 nuclear atoms, C1-C40 alkyloxy group, C6-C60 aryloxy group, C1-C40 alkylsilyl group, C6-C60 arylsilyl group, C1-C40 alkylboron group, C6-C60 arylboron group, C6-C60 arylphosphine group, C6-C60 arylphosphine oxide group, C6-C60 arylamine group, (C6-C60 aryl) (heteroaryl having 5 to 60 nuclear atoms) amine group, and heteroarylamine group having 5 to 60 nuclear atoms, and specifically, may be selected from the group consisting of: hydrogen, deuterium (D), cyano group (—CN), C1-C40 alkyl group, C3-C40 cycloalkyl group, heterocycloalkyl group having 3 to 40 nuclear atoms, C6-C60 aryl group, heteroaryl group having 5 to 60 nuclear atoms, and C6-C60 arylamine group.

In another example, one or more L₁ may be the same as or different from each other and each independently be selected from the group consisting of: the following linker groups L2-1 to L2-22, but the present disclosure is not limited thereto.

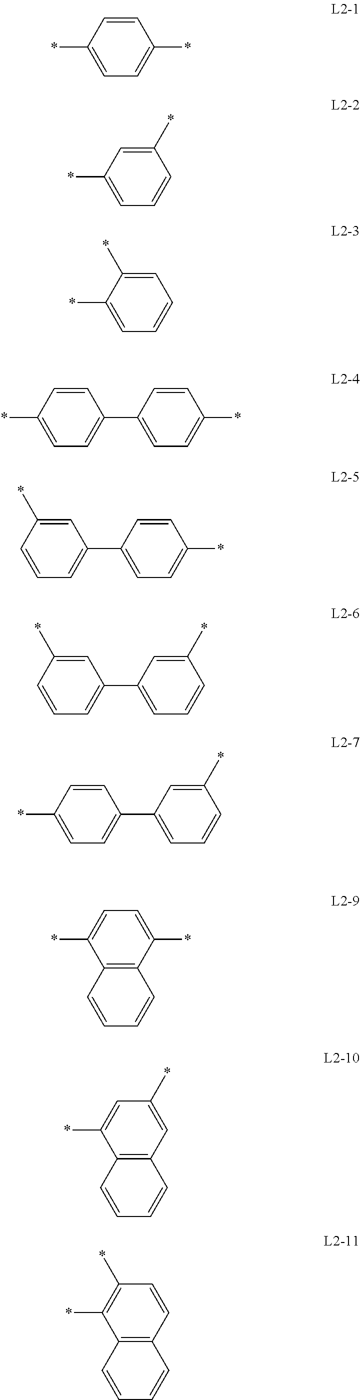

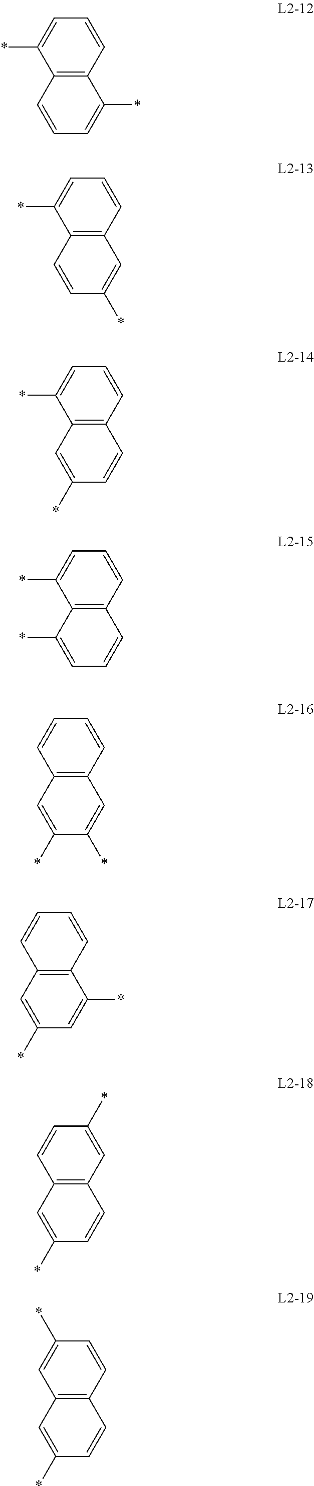

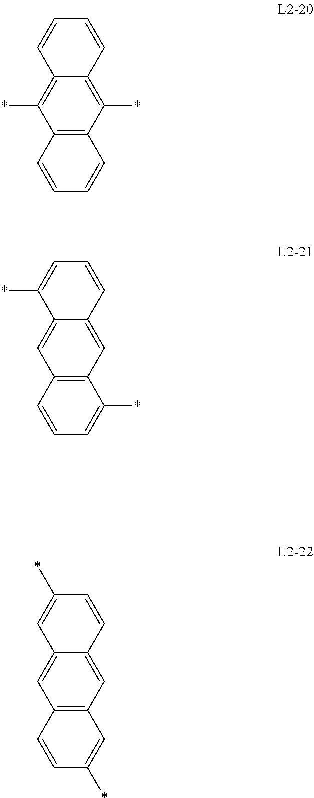 [SMILES: *c1c2ccccc2c(*)c2ccccc12 ; *c1cccc2cc3c(*)cccc3cc12 ; *c1ccc2cc3cc(*)ccc3cc2c1]

Hydrogens of the above linker groups L2-1 to L2-22 may each independently be substituted or unsubstituted with one or more substituents selected from the group consisting of: deuterium (D), halogen group (e.g., —F, —Cl, —Br, —I, and the like), cyano group (—CN), nitro group (—NO₂), amino group (—NH₂), C1-C12 alkyl group (e.g., methyl group, ethyl group, propyl group, butyl group, and the like), C6-C10 aryl group (e.g., phenyl group, biphenyl group, terphenyl group, naphthyl group, and the like), and heteroaryl group having 5 to 10 ring atoms (e.g., a monovalent pyrazine group, a monovalent pyrimidine group, a monovalent pyridazine group, a monovalent triazine group, and the like).

In the compound represented by the above Chemical Formula 1, a may be an integer in a range of 0 to 4, specifically, a may be an integer in a range of 0 to 3, and more specifically, a may be 0 or 1.

Herein, when a is 0, it means that hydrogen is not substituted with substituents Ar₂. On the other hand, when a is an integer in a range of 1 to 7, it means that hydrogen is substituted with the substituent Ar₂. In such a case, the plurality of Ar₂ may be the same as or different from each other.

Ar₂ may be selected from the group consisting of: hydrogen, deuterium (D), halogen group (e.g., —F, —Cl, —Br, —I, etc.), cyano group (—CN), nitro group (—NO₂), amino group (—NH₂), C1-C40 alkyl group, C2-C40 alkenyl group, C2-C40 alkynyl group, C3-C40 cycloalkyl group, heterocycloalkyl group having 3 to 40 nuclear atoms, C6-C60 aryl group, heteroaryl group having 5 to 60 nuclear atoms, C1-C40 alkyloxy group, C6-C60 aryloxy group, C1-C40 alkylsilyl group, C6-C60 arylsilyl group, C1-C40 alkylboron group, C6-C60 arylboron group, C6-C60 arylphosphine group, C6-C60 arylphosphine oxide group, C6-C60 arylamine group, (C6-C60 aryl) (heteroaryl having 5 to 60 nuclear atoms) amine group, and heteroarylamine group having 5 to 60 nuclear atoms, or fused with an adjacent group (e. g., Ar₂-Ar₂ and Ar₂-L₁) to form a fused ring, and specifically, may be selected from the group consisting of: hydrogen, deuterium (D), cyano group (—CN), C1-C40 alkyl group, C3-C40 cycloalkyl group, heterocycloalkyl group having 3 to 40 nuclear atoms, C6-C60 aryl group, heteroaryl group having 5 to 60 nuclear atoms, and C6-C60 arylamine group.

In such a case, the alkyl group, alkenyl group, alkynyl group, cycloalkyl group, heterocycloalkyl group, aryl group, heteroaryl alkyloxy group, group, aryloxy group, alkylsilyl group, arylsilyl group, alkylboron group, arylboron group, arylphosphine group, arylphosphine oxide, arylamine group, (aryl) (heteroaryl)amine, and heteroarylamine group of Ar₂ may each independently be substituted or unsubstituted with one or more substituents selected from the group consisting of: deuterium (D), halogen group, cyano group, nitro group, amino group, C1-C40 alkyl group, C2-C40 alkenyl group, C2-C40 alkynyl group, C3-C40 cycloalkyl group, heterocycloalkyl group having 3 to 40 nuclear atoms, C6-C60 aryl group, heteroaryl group having 5 to 60 nuclear atoms, C1-C40 alkyloxy group, C6-C60 aryloxy group, C1-C40 alkylsilyl group, C6-C60 arylsilyl group, C1-C40 alkylboron group, C6-C60 arylboron group, C6-C60 arylphosphine group, C6-C60 arylphosphine oxide group, C6-C60 arylamine group, (C6-C60 aryl) (heteroaryl having 5 to 60 nuclear atoms) amine group, and heteroarylamine group having 5 to 60 nuclear atoms. Herein, when a plurality of the substituents are present, they may be the same as or different from each other.

Depending on the type of the above Ar₁ and the introduction position of L₁, the compound represented by the above Chemical Formula 1 may be a compound represented by any one of the following Chemical Formulas 2 to 7. However, the present disclosure is not limited thereto.

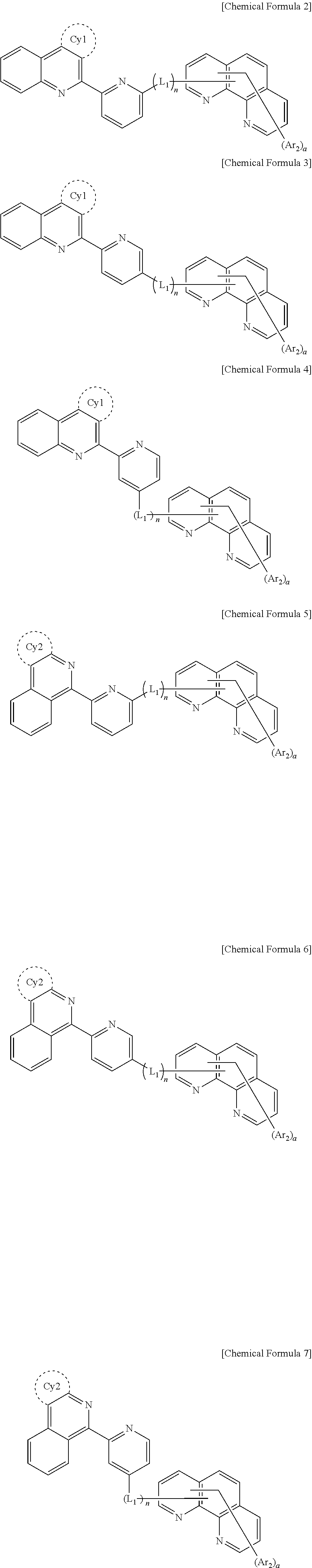

In the above Chemical Formulas 2 to 7,
n, L₁, a, and Ar₂ may be the same as defined in Chemical Formula 1, respectively,
Cy1 and Cy2 may each independently be a C6-C30 fused aromatic ring, and specifically may each independently be a C6-C18 fused aromatic ring,
the fused aromatic ring of Cy1 and Cy2 may each independently be substituted or unsubstituted with one or more substituents selected from the group consisting of: deuterium (D), halogen group, cyano group, nitro group, amino group, C1-C40 alkyl group, C2-C40 alkenyl group, C2-C40 alkynyl group, C3-C40 cycloalkyl group, heterocycloalkyl group having 3 to 40 nuclear atoms, C6-C60 aryl group, heteroaryl group having 5 to 60 nuclear atoms, C1-C40 alkyloxy group, C6-C60 aryloxy group, C1-C40 alkylsilyl group, C6-C60 arylsilyl group, C1-C40 alkylboron group, C6-C60 arylboron group, C6-C60 arylphosphine group, C6-C60 arylphosphine oxide group, C6-C60 arylamine group, (C6-C60 aryl) (heteroaryl having 5 to 60 nuclear atoms) amine group, and heteroarylamine group having 5 to 60 nuclear atoms, and when a plurality of the substituents are present, they are the same as or different from each other.

The compound represented by the above Chemical Formula 1 according to the present disclosure may be embodied as any one of Compounds 001 to 146 illustrated below, but the present disclosure is not limited thereto.

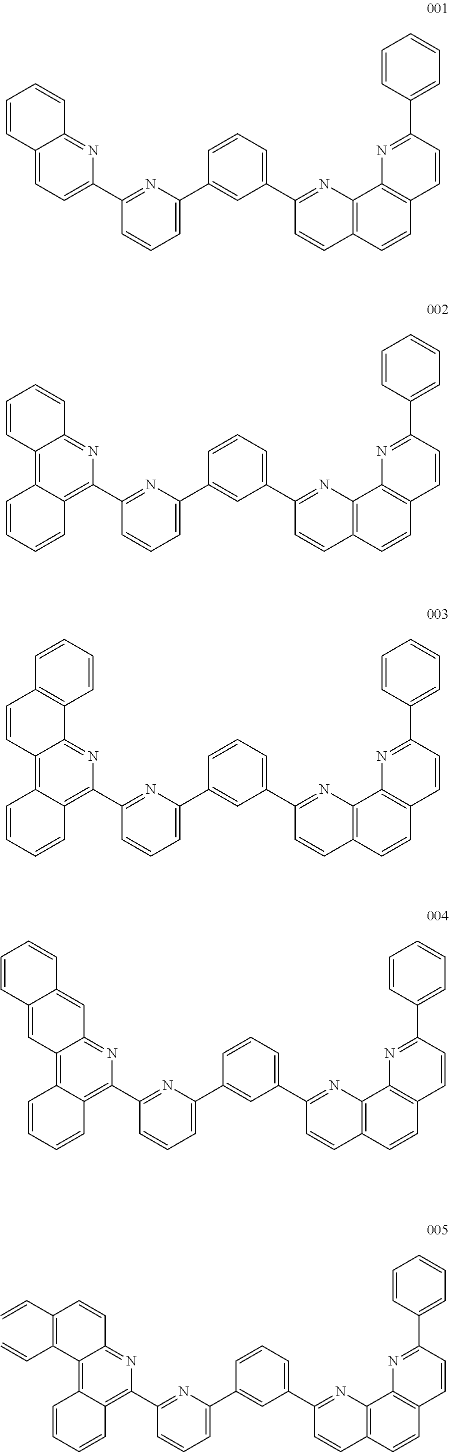 [SMILES: c1ccc(-c2ccc3ccc4ccc(-c5cccc(-c6cccc(-c7ccc8ccccc8n7)n6)c5)nc4c3n2)cc1 ; c1ccc(-c2ccc3ccc4ccc(-c5cccc(-c6cccc(-c7nc8ccccc8c8ccccc78)n6)c5)nc4c3n2)cc1 ; c1ccc(-c2ccc3ccc4ccc(-c5cccc(-c6cccc(-c7nc8c9ccccc9ccc8c8ccccc78)n6)c5)nc4c3n2)cc1 ; c1ccc(-c2ccc3ccc4ccc(-c5cccc(-c6cccc(-c7nc8cc9ccccc9cc8c8ccccc78)n6)c5)nc4c3n2)cc1 ; C=Cc1ccc2nc(-c3cccc(-c4cccc(-c5ccc6ccc7ccc(-c8ccccc8)nc7c6n5)c4)n3)c3ccccc3c2c1C=C]

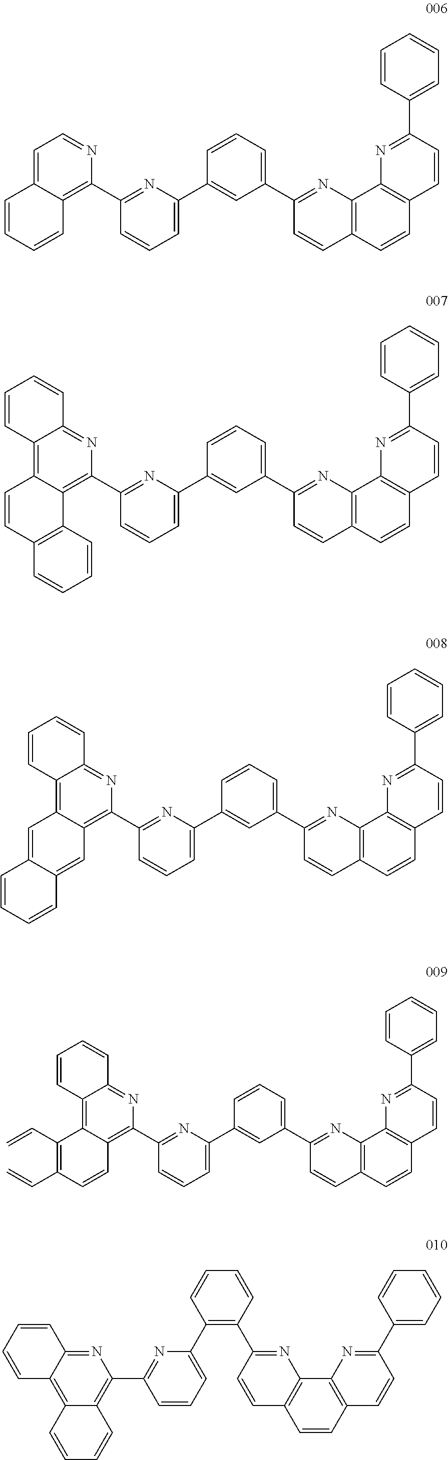 [SMILES: c1ccc(-c2ccc3ccc4ccc(-c5cccc(-c6cccc(-c7nccc8ccccc78)n6)c5)nc4c3n2)cc1 ; c1ccc(-c2ccc3ccc4ccc(-c5cccc(-c6cccc(-c7nc8ccccc8c8ccc9ccccc9c78)n6)c5)nc4c3n2)cc1 ; c1ccc(-c2ccc3ccc4ccc(-c5cccc(-c6cccc(-c7nc8ccccc8c8cc9ccccc9cc78)n6)c5)nc4c3n2)cc1 ; C=Cc1ccc2c(-c3cccc(-c4cccc(-c5ccc6ccc7ccc(-c8ccccc8)nc7c6n5)c4)n3)nc3ccccc3c2c1C=C ; c1ccc(-c2ccc3ccc4ccc(-c5ccccc5-c5cccc(-c6nc7ccccc7c7ccccc67)n5)nc4c3n2)cc1]

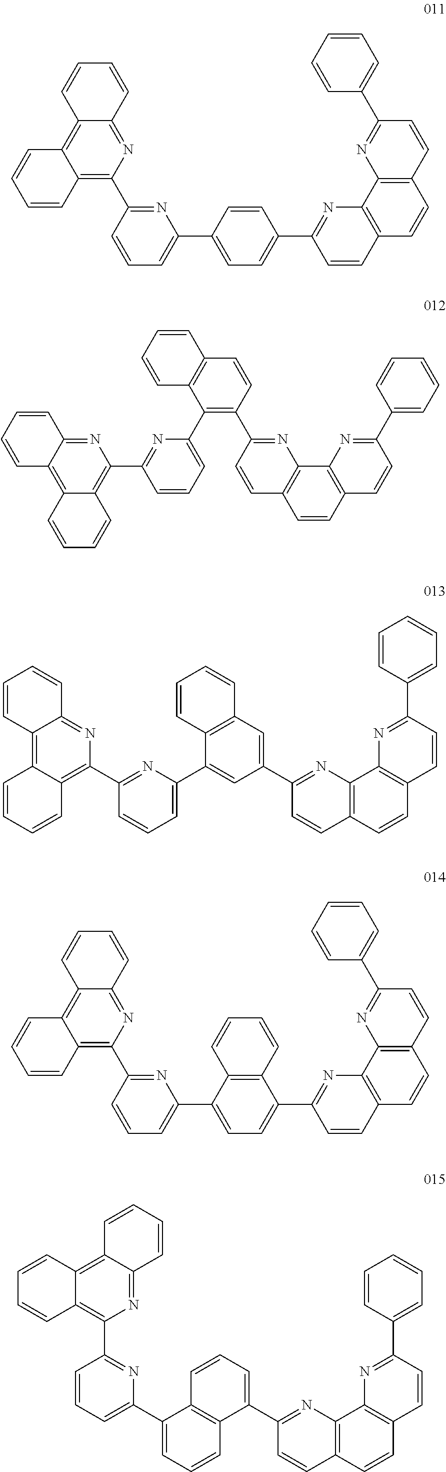 [SMILES: c1ccc(-c2ccc3ccc4ccc(-c5ccc(-c6cccc(-c7nc8ccccc8c8ccccc78)n6)cc5)nc4c3n2)cc1 ; c1ccc(-c2ccc3ccc4ccc(-c5ccc6ccccc6c5-c5cccc(-c6nc7ccccc7c7ccccc67)n5)nc4c3n2)cc1 ; c1ccc(-c2ccc3ccc4ccc(-c5cc(-c6cccc(-c7nc8ccccc8c8ccccc78)n6)c6ccccc6c5)nc4c3n2)cc1 ; c1ccc(-c2ccc3ccc4ccc(-c5ccc(-c6cccc(-c7nc8ccccc8c8ccccc78)n6)c6ccccc56)nc4c3n2)cc1 ; c1ccc(-c2ccc3ccc4ccc(-c5cccc6c(-c7cccc(-c8nc9ccccc9c9ccccc89)n7)cccc56)nc4c3n2)cc1]

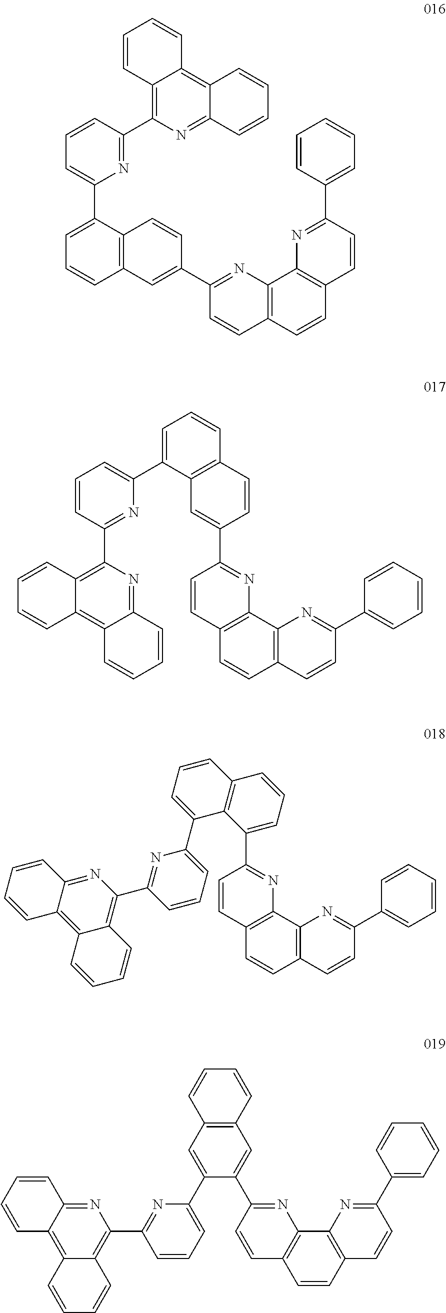 [SMILES: c1ccc(-c2ccc3ccc4ccc(-c5ccc6c(-c7cccc(-c8nc9ccccc9c9ccccc89)n7)cccc6c5)nc4c3n2)cc1 ; c1ccc(-c2ccc3ccc4ccc(-c5ccc6cccc(-c7cccc(-c8nc9ccccc9c9ccccc89)n7)c6c5)nc4c3n2)cc1 ; c1ccc(-c2ccc3ccc4ccc(-c5cccc6cccc(-c7cccc(-c8nc9ccccc9c9ccccc89)n7)c56)nc4c3n2)cc1 ; c1ccc(-c2ccc3ccc4ccc(-c5cc6ccccc6cc5-c5cccc(-c6nc7ccccc7c7ccccc67)n5)nc4c3n2)cc1]

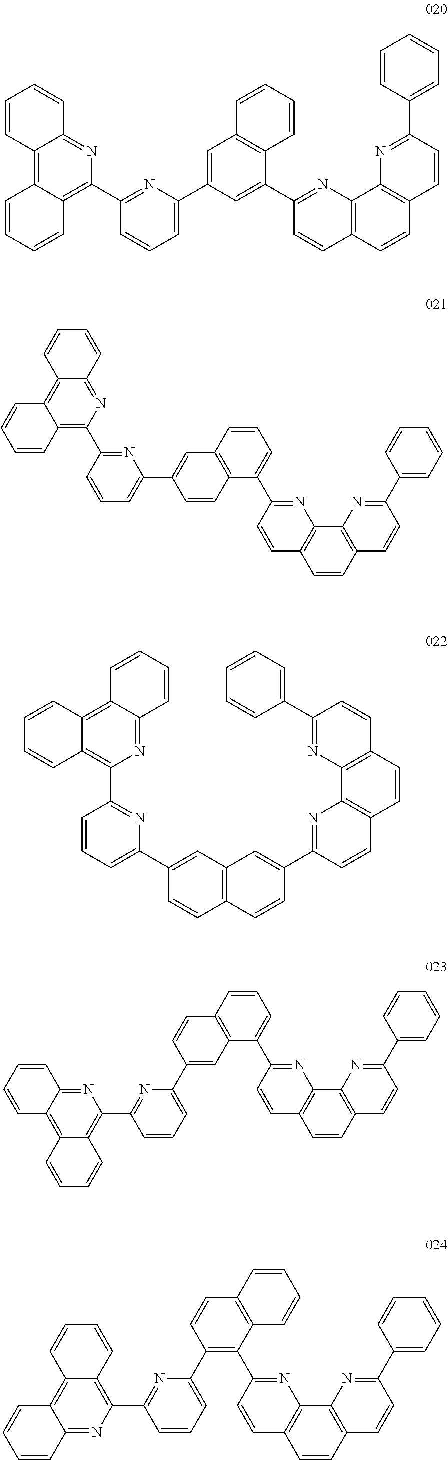 [SMILES: c1ccc(-c2ccc3ccc4ccc(-c5cc(-c6cccc(-c7nc8ccccc8c8ccccc78)n6)cc6ccccc56)nc4c3n2)cc1 ; c1ccc(-c2ccc3ccc4ccc(-c5cccc6cc(-c7cccc(-c8nc9ccccc9c9ccccc89)n7)ccc56)nc4c3n2)cc1 ; c1ccc(-c2ccc3ccc4ccc(-c5ccc6ccc(-c7cccc(-c8nc9ccccc9c9ccccc89)n7)cc6c5)nc4c3n2)cc1 ; c1ccc(-c2ccc3ccc4ccc(-c5cccc6ccc(-c7cccc(-c8nc9ccccc9c9ccccc89)n7)cc56)nc4c3n2)cc1 ; c1ccc(-c2ccc3ccc4ccc(-c5c(-c6cccc(-c7nc8ccccc8c8ccccc78)n6)ccc6ccccc56)nc4c3n2)cc1]

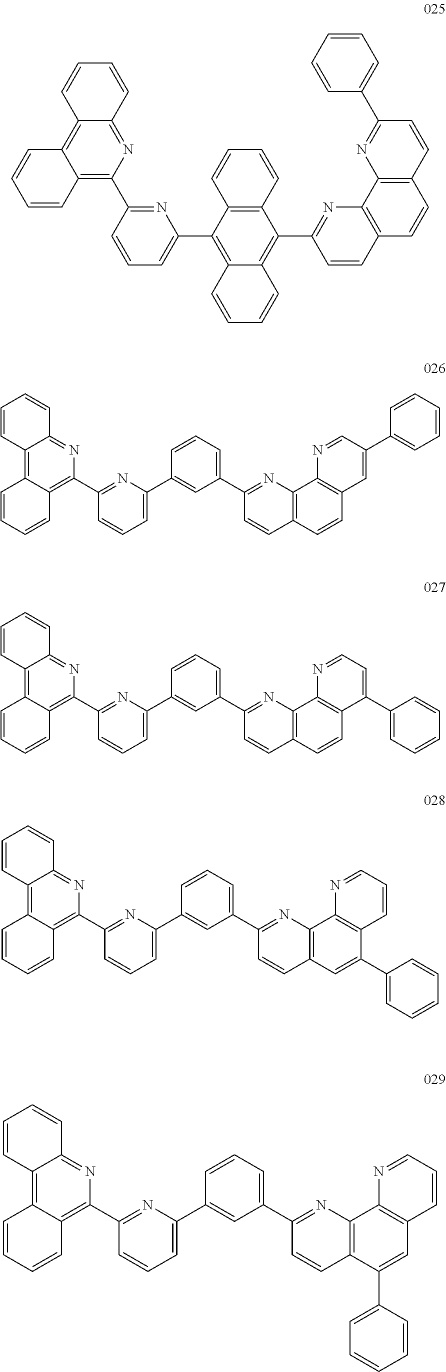 [SMILES: c1ccc(-c2ccc3ccc4ccc(-c5c6ccccc6c(-c6cccc(-c7nc8ccccc8c8ccccc78)n6)c6ccccc56)nc4c3n2)cc1 ; c1ccc(-c2cnc3c(ccc4ccc(-c5cccc(-c6cccc(-c7nc8ccccc8c8ccccc78)n6)c5)nc43)c2)cc1 ; c1ccc(-c2ccnc3c2ccc2ccc(-c4cccc(-c5cccc(-c6nc7ccccc7c7ccccc67)n5)c4)nc23)cc1 ; c1ccc(-c2cc3ccc(-c4cccc(-c5cccc(-c6nc7ccccc7c7ccccc67)n5)c4)nc3c3ncccc23)cc1 ; c1ccc(-c2cc3cccnc3c3nc(-c4cccc(-c5cccc(-c6nc7ccccc7c7ccccc67)n5)c4)ccc23)cc1]

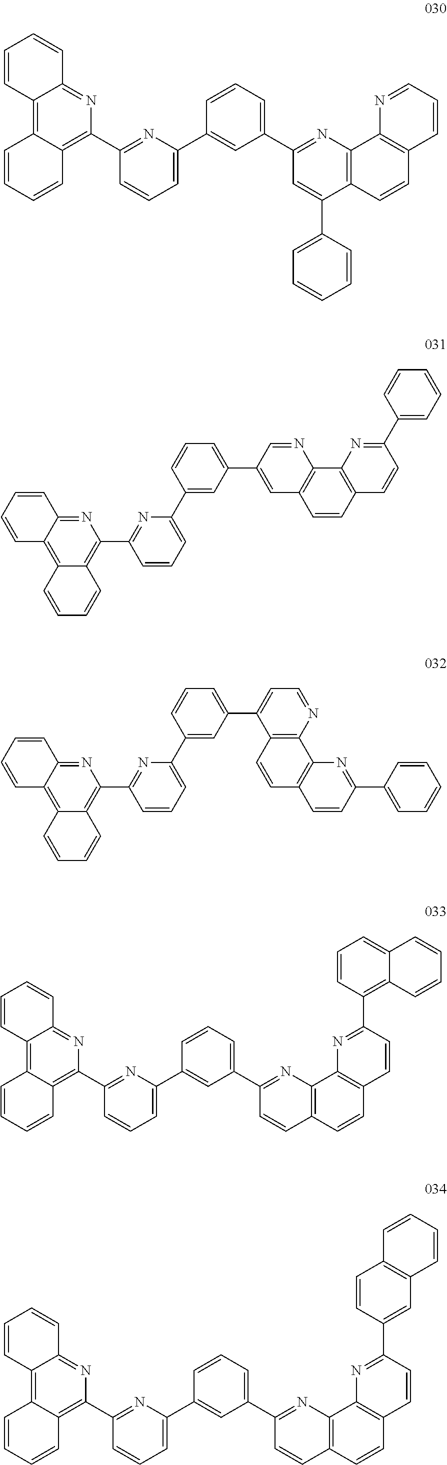 [SMILES: c1ccc(-c2cc(-c3cccc(-c4cccc(-c5nc6ccccc6c6ccccc56)n4)c3)nc3c2ccc2cccnc23)cc1 ; c1ccc(-c2ccc3ccc4cc(-c5cccc(-c6cccc(-c7nc8ccccc8c8ccccc78)n6)c5)cnc4c3n2)cc1 ; c1ccc(-c2ccc3ccc4c(-c5cccc(-c6cccc(-c7nc8ccccc8c8ccccc78)n6)c5)ccnc4c3n2)cc1 ; c1cc(-c2cccc(-c3nc4ccccc4c4ccccc34)n2)cc(-c2ccc3ccc4ccc(-c5cccc6ccccc56)nc4c3n2)c1 ; c1cc(-c2cccc(-c3nc4ccccc4c4ccccc34)n2)cc(-c2ccc3ccc4ccc(-c5ccc6ccccc6c5)nc4c3n2)c1]

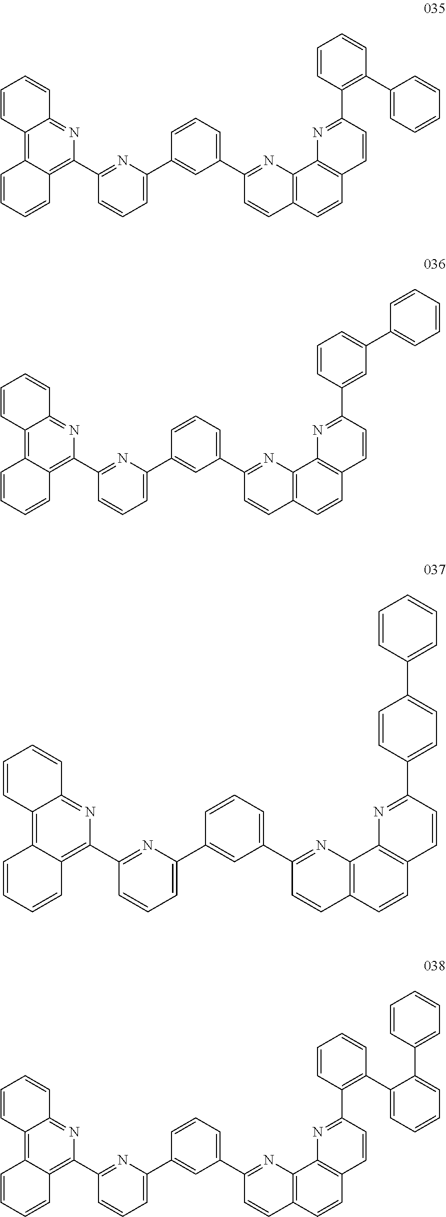 [SMILES: c1ccc(-c2ccccc2-c2ccc3ccc4ccc(-c5cccc(-c6cccc(-c7nc8ccccc8c8ccccc78)n6)c5)nc4c3n2)cc1 ; c1ccc(-c2cccc(-c3ccc4ccc5ccc(-c6cccc(-c7cccc(-c8nc9ccccc9c9ccccc89)n7)c6)nc5c4n3)c2)cc1 ; c1ccc(-c2ccc(-c3ccc4ccc5ccc(-c6cccc(-c7cccc(-c8nc9ccccc9c9ccccc89)n7)c6)nc5c4n3)cc2)cc1 ; c1ccc(-c2ccccc2-c2ccccc2-c2ccc3ccc4ccc(-c5cccc(-c6cccc(-c7nc8ccccc8c8ccccc78)n6)c5)nc4c3n2)cc1]

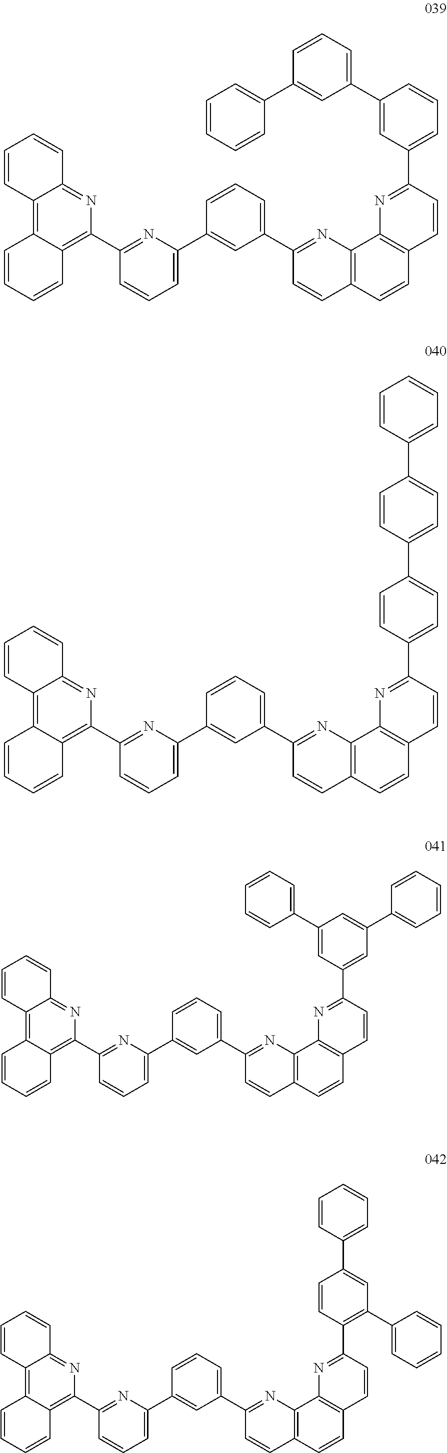 [SMILES: c1ccc(-c2cccc(-c3cccc(-c4ccc5ccc6ccc(-c7cccc(-c8cccc(-c9nc%10ccccc%10c%10ccccc9%10)n8)c7)nc6c5n4)c3)c2)cc1 ; c1ccc(-c2ccc(-c3ccc(-c4ccc5ccc6ccc(-c7cccc(-c8cccc(-c9nc%10ccccc%10c%10ccccc9%10)n8)c7)nc6c5n4)cc3)cc2)cc1 ; c1ccc(-c2cc(-c3ccccc3)cc(-c3ccc4ccc5ccc(-c6cccc(-c7cccc(-c8nc9ccccc9c9ccccc89)n7)c6)nc5c4n3)c2)cc1 ; c1ccc(-c2ccc(-c3ccc4ccc5ccc(-c6cccc(-c7cccc(-c8nc9ccccc9c9ccccc89)n7)c6)nc5c4n3)c(-c3ccccc3)c2)cc1]

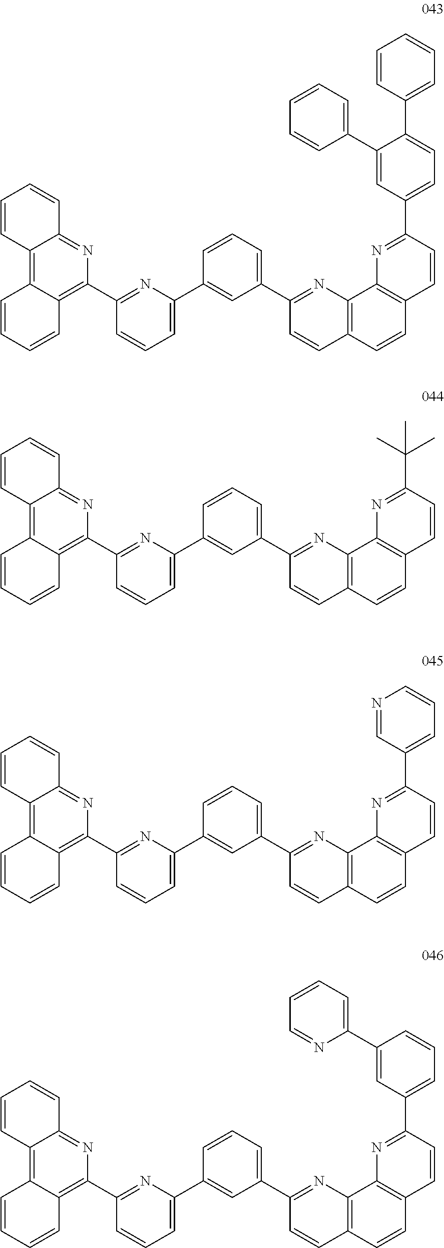 [SMILES: c1ccc(-c2ccc(-c3ccc4ccc5ccc(-c6cccc(-c7cccc(-c8nc9ccccc9c9ccccc89)n7)c6)nc5c4n3)cc2-c2ccccc2)cc1 ; CC(C)(C)c1ccc2ccc3ccc(-c4cccc(-c5cccc(-c6nc7ccccc7c7ccccc67)n5)c4)nc3c2n1 ; c1cncc(-c2ccc3ccc4ccc(-c5cccc(-c6cccc(-c7nc8ccccc8c8ccccc78)n6)c5)nc4c3n2)c1 ; c1ccc(-c2cccc(-c3ccc4ccc5ccc(-c6cccc(-c7cccc(-c8nc9ccccc9c9ccccc89)n7)c6)nc5c4n3)c2)nc1]

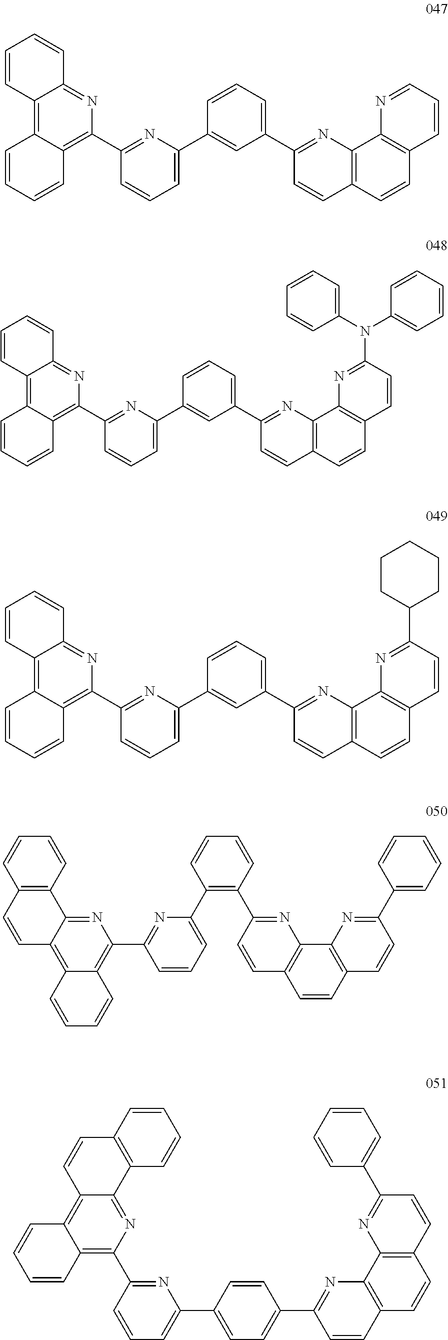 [SMILES: c1cc(-c2cccc(-c3nc4ccccc4c4ccccc34)n2)cc(-c2ccc3ccc4cccnc4c3n2)c1 ; c1ccc(N(c2ccccc2)c2ccc3ccc4ccc(-c5cccc(-c6cccc(-c7nc8ccccc8c8ccccc78)n6)c5)nc4c3n2)cc1 ; c1cc(-c2cccc(-c3nc4ccccc4c4ccccc34)n2)cc(-c2ccc3ccc4ccc(C5CCCCC5)nc4c3n2)c1 ; c1ccc(-c2ccc3ccc4ccc(-c5ccccc5-c5cccc(-c6nc7c8ccccc8ccc7c7ccccc67)n5)nc4c3n2)cc1 ; c1ccc(-c2ccc3ccc4ccc(-c5ccc(-c6cccc(-c7nc8c9ccccc9ccc8c8ccccc78)n6)cc5)nc4c3n2)cc1]

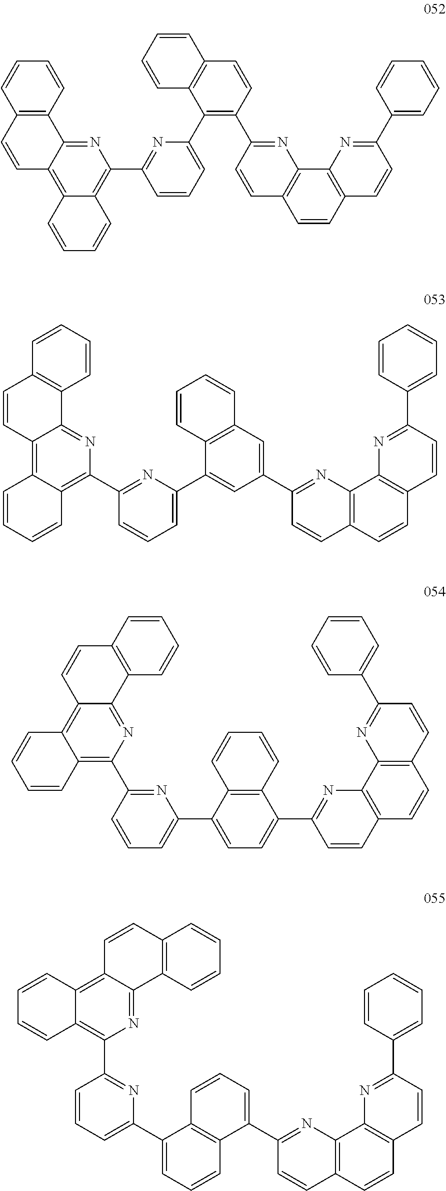 [SMILES: c1ccc(-c2ccc3ccc4ccc(-c5ccc6ccccc6c5-c5cccc(-c6nc7c8ccccc8ccc7c7ccccc67)n5)nc4c3n2)cc1 ; c1ccc(-c2ccc3ccc4ccc(-c5cc(-c6cccc(-c7nc8c9ccccc9ccc8c8ccccc78)n6)c6ccccc6c5)nc4c3n2)cc1 ; c1ccc(-c2ccc3ccc4ccc(-c5ccc(-c6cccc(-c7nc8c9ccccc9ccc8c8ccccc78)n6)c6ccccc56)nc4c3n2)cc1 ; c1ccc(-c2ccc3ccc4ccc(-c5cccc6c(-c7cccc(-c8nc9c%10ccccc%10ccc9c9ccccc89)n7)cccc56)nc4c3n2)cc1]

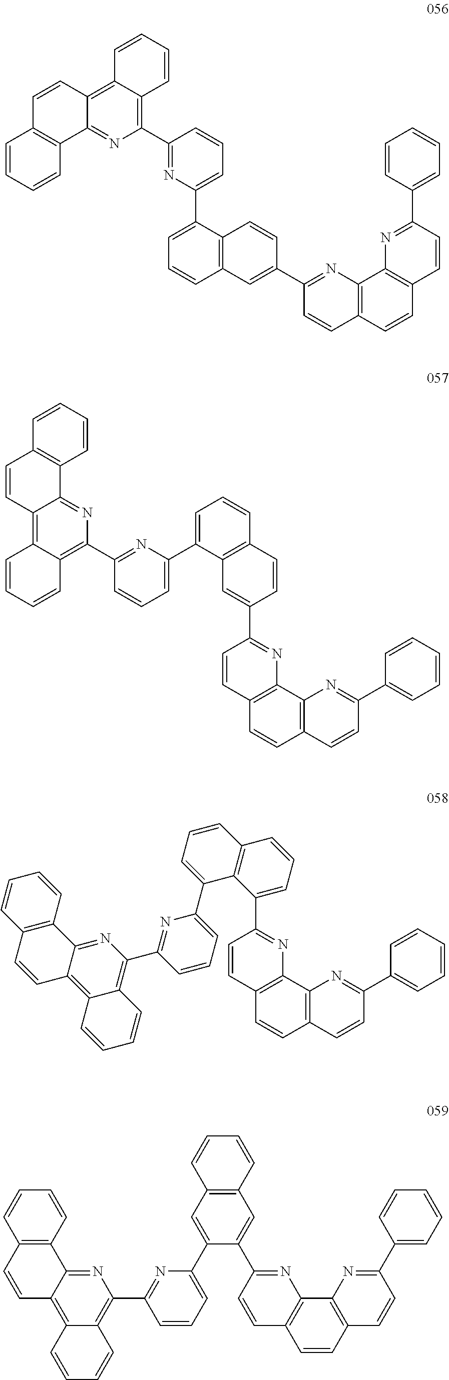 [SMILES: c1ccc(-c2ccc3ccc4ccc(-c5ccc6c(-c7cccc(-c8nc9c%10ccccc%10ccc9c9ccccc89)n7)cccc6c5)nc4c3n2)cc1 ; c1ccc(-c2ccc3ccc4ccc(-c5ccc6cccc(-c7cccc(-c8nc9c%10ccccc%10ccc9c9ccccc89)n7)c6c5)nc4c3n2)cc1 ; c1ccc(-c2ccc3ccc4ccc(-c5cccc6cccc(-c7cccc(-c8nc9c%10ccccc%10ccc9c9ccccc89)n7)c56)nc4c3n2)cc1 ; c1ccc(-c2ccc3ccc4ccc(-c5cc6ccccc6cc5-c5cccc(-c6nc7c8ccccc8ccc7c7ccccc67)n5)nc4c3n2)cc1]

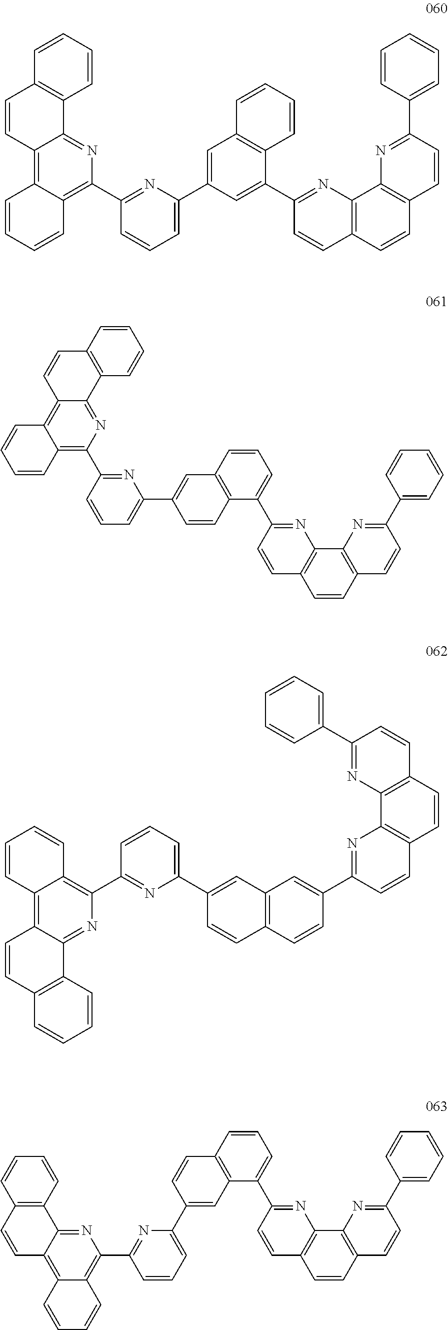 [SMILES: c1ccc(-c2ccc3ccc4ccc(-c5cc(-c6cccc(-c7nc8c9ccccc9ccc8c8ccccc78)n6)cc6ccccc56)nc4c3n2)cc1 ; c1ccc(-c2ccc3ccc4ccc(-c5cccc6cc(-c7cccc(-c8nc9c%10ccccc%10ccc9c9ccccc89)n7)ccc56)nc4c3n2)cc1 ; c1ccc(-c2ccc3ccc4ccc(-c5ccc6ccc(-c7cccc(-c8nc9c%10ccccc%10ccc9c9ccccc89)n7)cc6c5)nc4c3n2)cc1 ; c1ccc(-c2ccc3ccc4ccc(-c5cccc6ccc(-c7cccc(-c8nc9c%10ccccc%10ccc9c9ccccc89)n7)cc56)nc4c3n2)cc1]

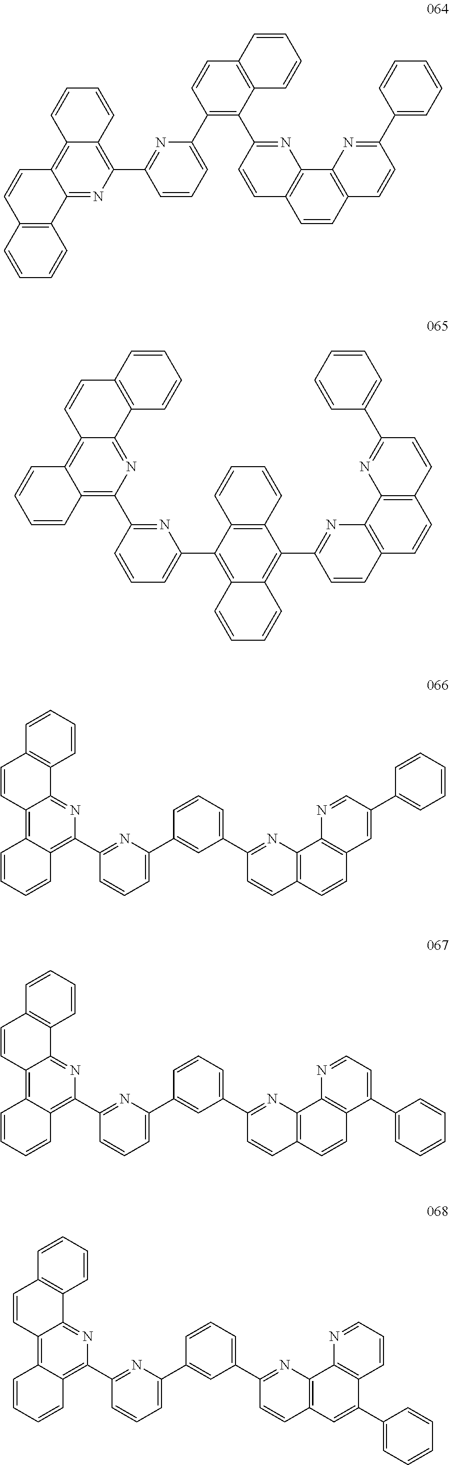 [SMILES: c1ccc(-c2ccc3ccc4ccc(-c5c(-c6cccc(-c7nc8c9ccccc9ccc8c8ccccc78)n6)ccc6ccccc56)nc4c3n2)cc1 ; c1ccc(-c2ccc3ccc4ccc(-c5c6ccccc6c(-c6cccc(-c7nc8c9ccccc9ccc8c8ccccc78)n6)c6ccccc56)nc4c3n2)cc1 ; c1ccc(-c2cnc3c(ccc4ccc(-c5cccc(-c6cccc(-c7nc8c9ccccc9ccc8c8ccccc78)n6)c5)nc43)c2)cc1 ; c1ccc(-c2ccnc3c2ccc2ccc(-c4cccc(-c5cccc(-c6nc7c8ccccc8ccc7c7ccccc67)n5)c4)nc23)cc1 ; c1ccc(-c2cc3ccc(-c4cccc(-c5cccc(-c6nc7c8ccccc8ccc7c7ccccc67)n5)c4)nc3c3ncccc23)cc1]

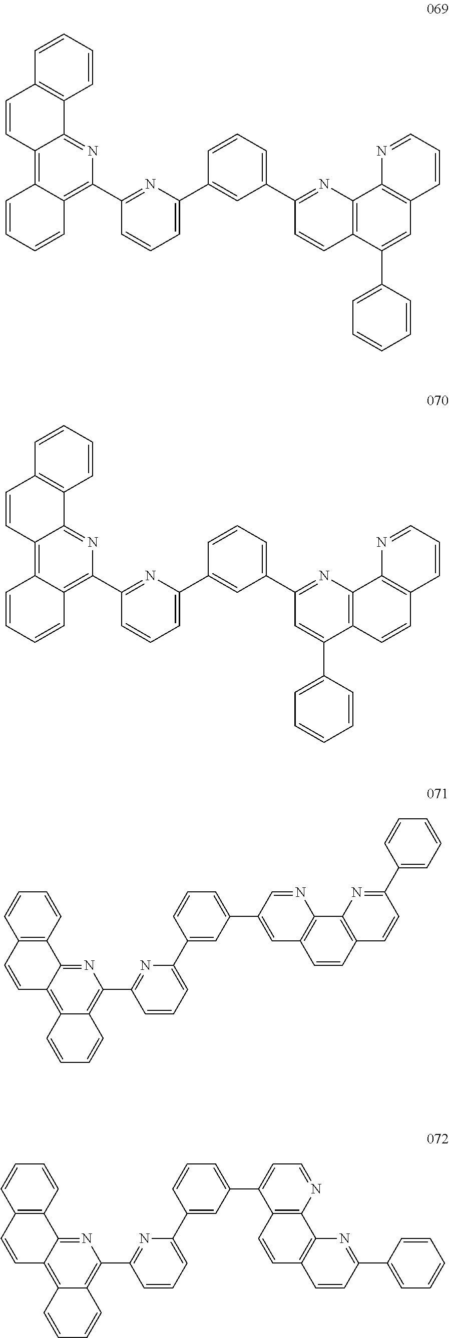 [SMILES: c1ccc(-c2cc3cccnc3c3nc(-c4cccc(-c5cccc(-c6nc7c8ccccc8ccc7c7ccccc67)n5)c4)ccc23)cc1 ; c1ccc(-c2cc(-c3cccc(-c4cccc(-c5nc6c7ccccc7ccc6c6ccccc56)n4)c3)nc3c2ccc2cccnc23)cc1 ; c1ccc(-c2ccc3ccc4cc(-c5cccc(-c6cccc(-c7nc8c9ccccc9ccc8c8ccccc78)n6)c5)cnc4c3n2)cc1 ; c1ccc(-c2ccc3ccc4c(-c5cccc(-c6cccc(-c7nc8c9ccccc9ccc8c8ccccc78)n6)c5)ccnc4c3n2)cc1]

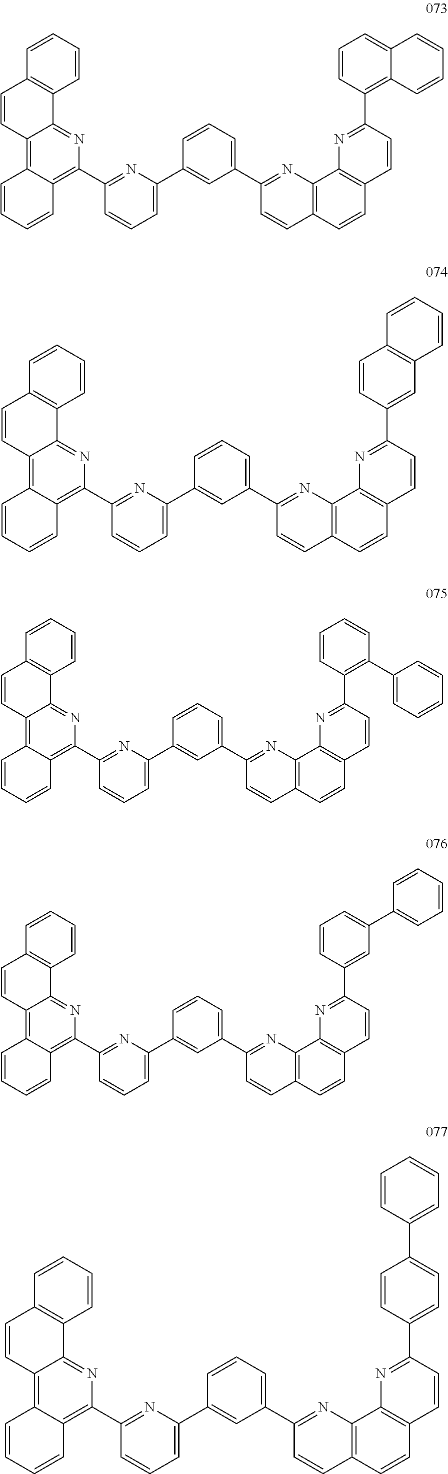 [SMILES: c1cc(-c2cccc(-c3nc4c5ccccc5ccc4c4ccccc34)n2)cc(-c2ccc3ccc4ccc(-c5cccc6ccccc56)nc4c3n2)c1 ; c1cc(-c2cccc(-c3nc4c5ccccc5ccc4c4ccccc34)n2)cc(-c2ccc3ccc4ccc(-c5ccc6ccccc6c5)nc4c3n2)c1 ; c1ccc(-c2ccccc2-c2ccc3ccc4ccc(-c5cccc(-c6cccc(-c7nc8c9ccccc9ccc8c8ccccc78)n6)c5)nc4c3n2)cc1 ; c1ccc(-c2cccc(-c3ccc4ccc5ccc(-c6cccc(-c7cccc(-c8nc9c%10ccccc%10ccc9c9ccccc89)n7)c6)nc5c4n3)c2)cc1 ; c1ccc(-c2ccc(-c3ccc4ccc5ccc(-c6cccc(-c7cccc(-c8nc9c%10ccccc%10ccc9c9ccccc89)n7)c6)nc5c4n3)cc2)cc1]

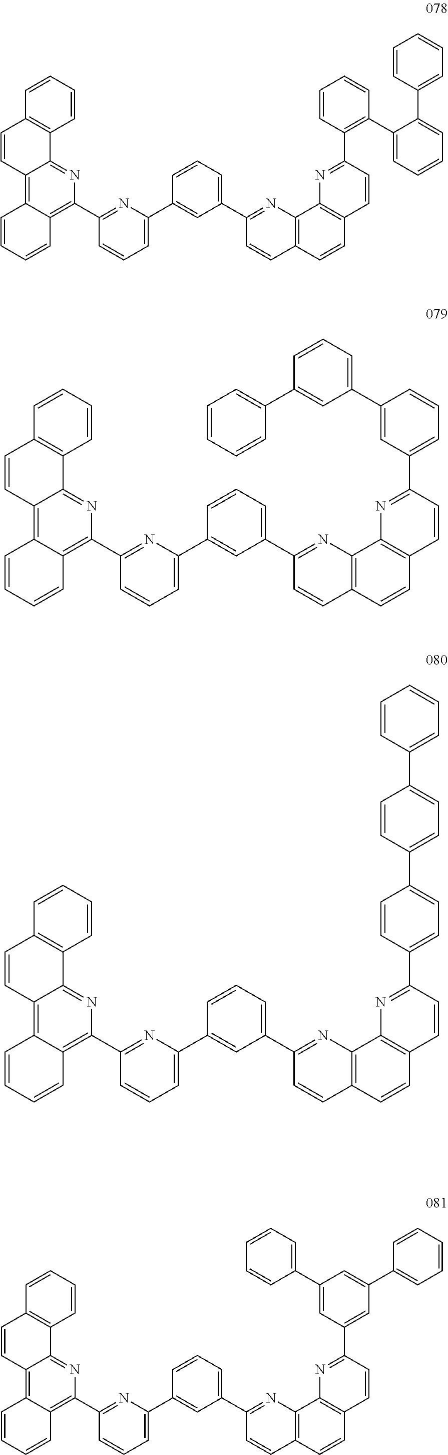 [SMILES: c1ccc(-c2ccccc2-c2ccccc2-c2ccc3ccc4ccc(-c5cccc(-c6cccc(-c7nc8c9ccccc9ccc8c8ccccc78)n6)c5)nc4c3n2)cc1 ; c1ccc(-c2cccc(-c3cccc(-c4ccc5ccc6ccc(-c7cccc(-c8cccc(-c9nc%10c%11ccccc%11ccc%10c%10ccccc9%10)n8)c7)nc6c5n4)c3)c2)cc1 ; c1ccc(-c2ccc(-c3ccc(-c4ccc5ccc6ccc(-c7cccc(-c8cccc(-c9nc%10c%11ccccc%11ccc%10c%10ccccc9%10)n8)c7)nc6c5n4)cc3)cc2)cc1 ; c1ccc(-c2cc(-c3ccccc3)cc(-c3ccc4ccc5ccc(-c6cccc(-c7cccc(-c8nc9c%10ccccc%10ccc9c9ccccc89)n7)c6)nc5c4n3)c2)cc1]

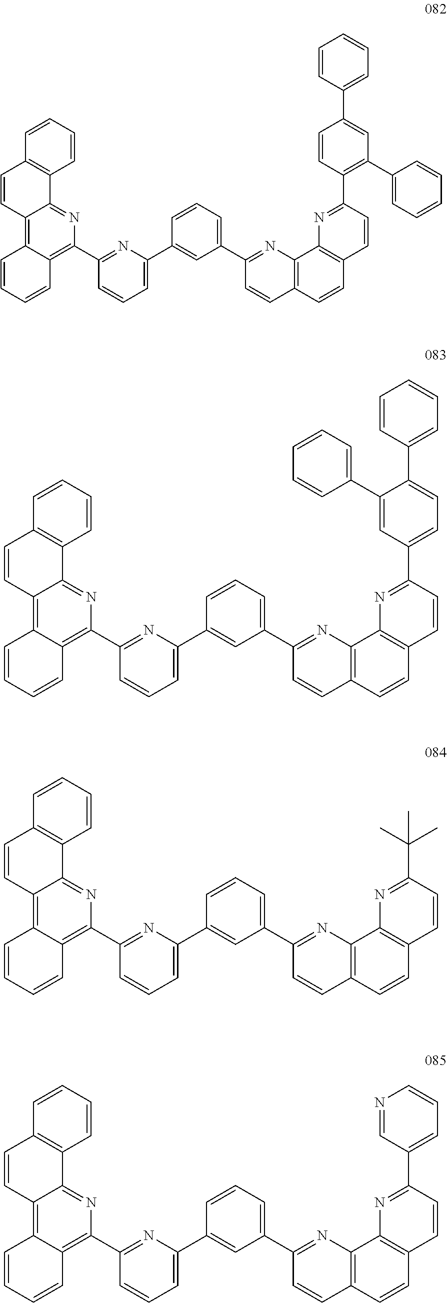 [SMILES: c1ccc(-c2ccc(-c3ccc4ccc5ccc(-c6cccc(-c7cccc(-c8nc9c%10ccccc%10ccc9c9ccccc89)n7)c6)nc5c4n3)c(-c3ccccc3)c2)cc1 ; c1ccc(-c2ccc(-c3ccc4ccc5ccc(-c6cccc(-c7cccc(-c8nc9c%10ccccc%10ccc9c9ccccc89)n7)c6)nc5c4n3)cc2-c2ccccc2)cc1 ; CC(C)(C)c1ccc2ccc3ccc(-c4cccc(-c5cccc(-c6nc7c8ccccc8ccc7c7ccccc67)n5)c4)nc3c2n1 ; c1cncc(-c2ccc3ccc4ccc(-c5cccc(-c6cccc(-c7nc8c9ccccc9ccc8c8ccccc78)n6)c5)nc4c3n2)c1]

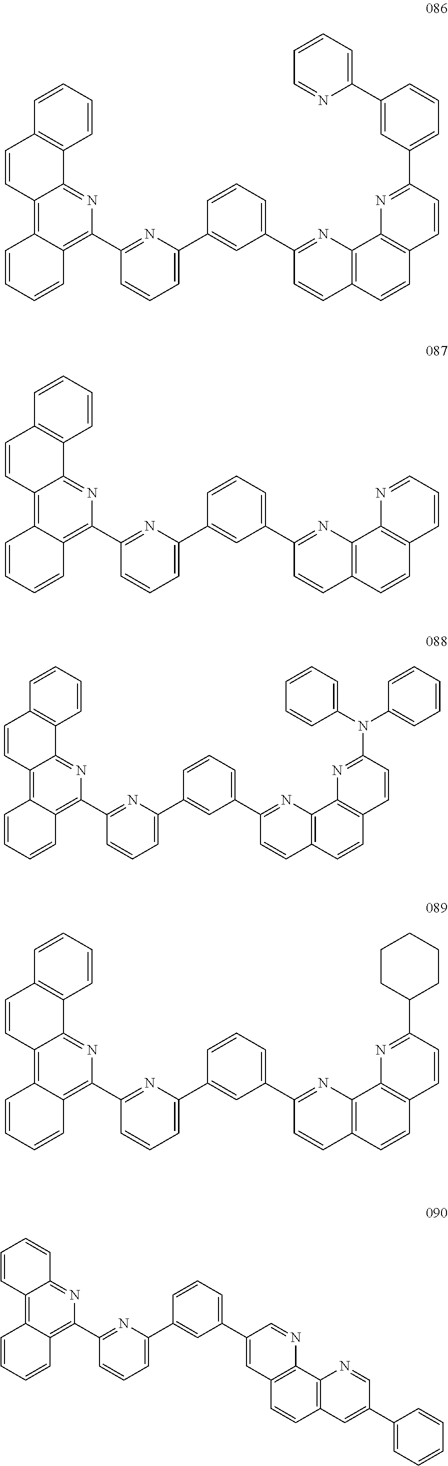 [SMILES: c1ccc(-c2cccc(-c3ccc4ccc5ccc(-c6cccc(-c7cccc(-c8nc9c%10ccccc%10ccc9c9ccccc89)n7)c6)nc5c4n3)c2)nc1 ; c1cc(-c2cccc(-c3nc4c5ccccc5ccc4c4ccccc34)n2)cc(-c2ccc3ccc4cccnc4c3n2)c1 ; c1ccc(N(c2ccccc2)c2ccc3ccc4ccc(-c5cccc(-c6cccc(-c7nc8c9ccccc9ccc8c8ccccc78)n6)c5)nc4c3n2)cc1 ; c1cc(-c2cccc(-c3nc4c5ccccc5ccc4c4ccccc34)n2)cc(-c2ccc3ccc4ccc(C5CCCCC5)nc4c3n2)c1 ; c1ccc(-c2cnc3c(ccc4cc(-c5cccc(-c6cccc(-c7nc8ccccc8c8ccccc78)n6)c5)cnc43)c2)cc1]

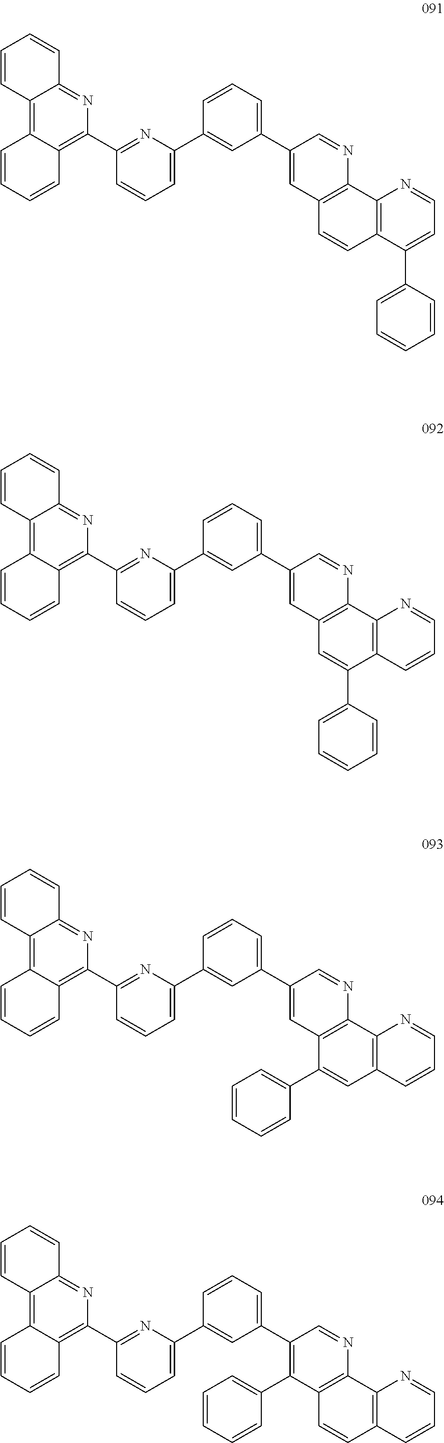 [SMILES: c1ccc(-c2ccnc3c2ccc2cc(-c4cccc(-c5cccc(-c6nc7ccccc7c7ccccc67)n5)c4)cnc23)cc1 ; c1ccc(-c2cc3cc(-c4cccc(-c5cccc(-c6nc7ccccc7c7ccccc67)n5)c4)cnc3c3ncccc23)cc1 ; c1ccc(-c2cc3cccnc3c3ncc(-c4cccc(-c5cccc(-c6nc7ccccc7c7ccccc67)n5)c4)cc23)cc1 ; c1ccc(-c2c(-c3cccc(-c4cccc(-c5nc6ccccc6c6ccccc56)n4)c3)cnc3c2ccc2cccnc23)cc1]

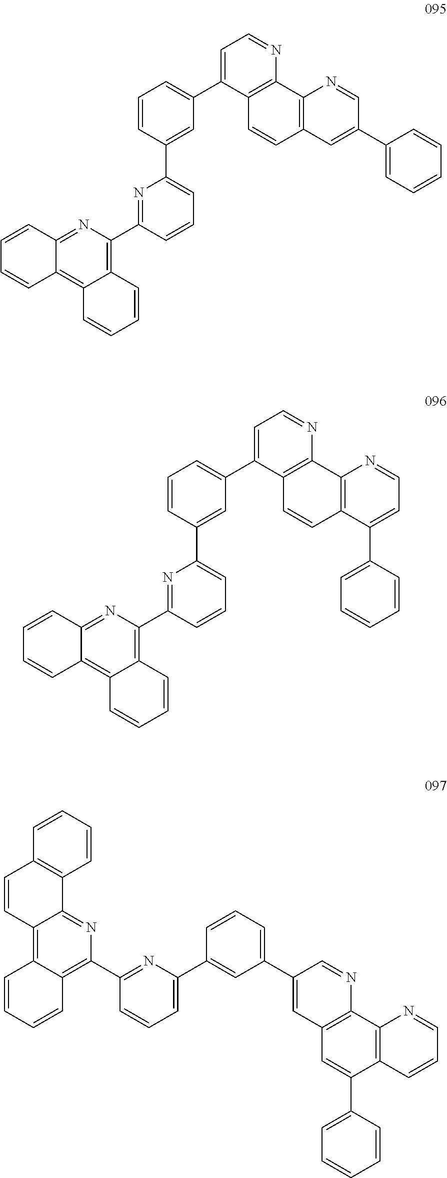 [SMILES: c1ccc(-c2cnc3c(ccc4c(-c5cccc(-c6cccc(-c7nc8ccccc8c8ccccc78)n6)c5)ccnc43)c2)cc1 ; c1ccc(-c2ccnc3c2ccc2c(-c4cccc(-c5cccc(-c6nc7ccccc7c7ccccc67)n5)c4)ccnc23)cc1 ; c1ccc(-c2cc3cc(-c4cccc(-c5cccc(-c6nc7c8ccccc8ccc7c7ccccc67)n5)c4)cnc3c3ncccc23)cc1]

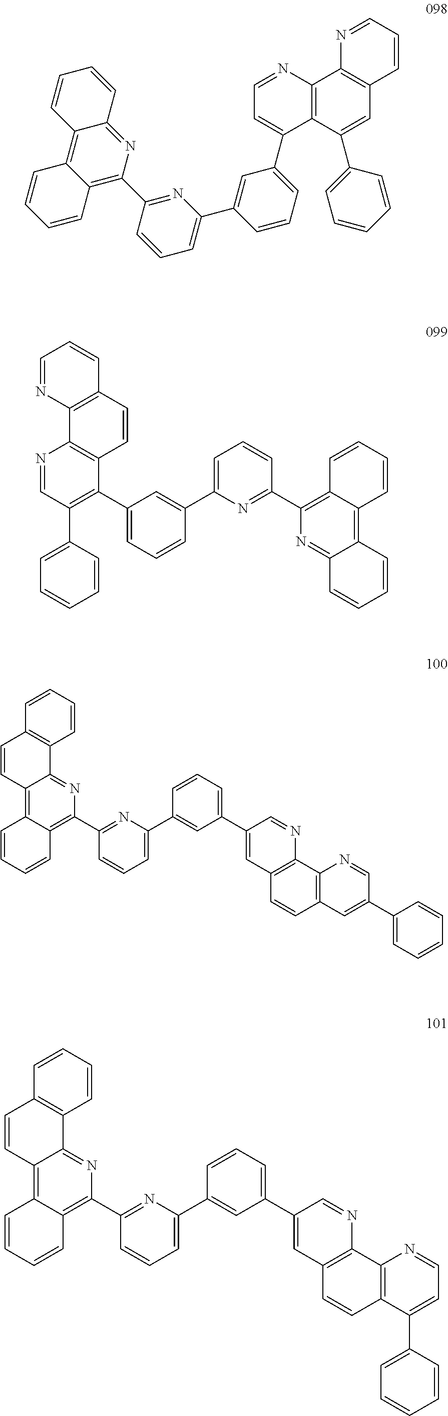 [SMILES: c1ccc(-c2cc3cccnc3c3nccc(-c4cccc(-c5cccc(-c6nc7ccccc7c7ccccc67)n5)c4)c23)cc1 ; c1ccc(-c2cnc3c(ccc4cccnc43)c2-c2cccc(-c3cccc(-c4nc5ccccc5c5ccccc45)n3)c2)cc1 ; c1ccc(-c2cnc3c(ccc4cc(-c5cccc(-c6cccc(-c7nc8c9ccccc9ccc8c8ccccc78)n6)c5)cnc43)c2)cc1 ; c1ccc(-c2ccnc3c2ccc2cc(-c4cccc(-c5cccc(-c6nc7c8ccccc8ccc7c7ccccc67)n5)c4)cnc23)cc1]

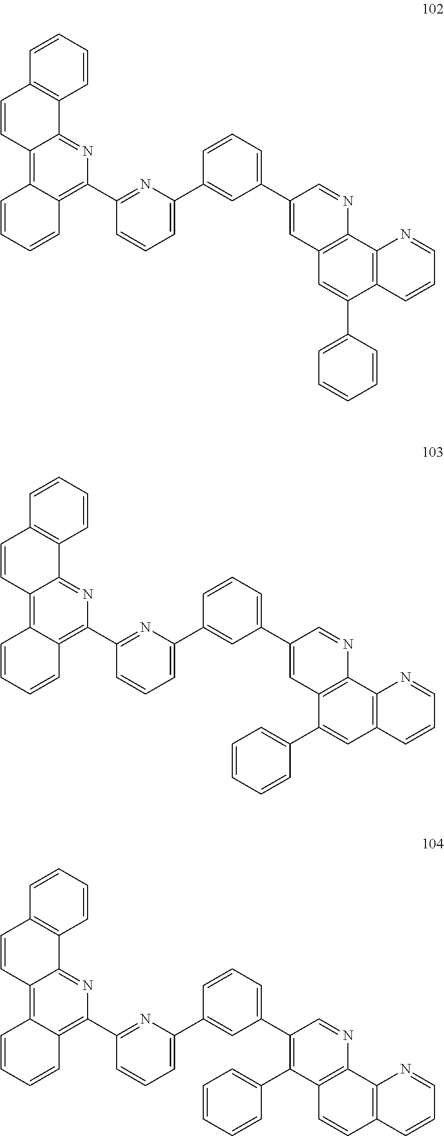 [SMILES: c1ccc(-c2cc3cc(-c4cccc(-c5cccc(-c6nc7c8ccccc8ccc7c7ccccc67)n5)c4)cnc3c3ncccc23)cc1 ; c1ccc(-c2cc3cccnc3c3ncc(-c4cccc(-c5cccc(-c6nc7c8ccccc8ccc7c7ccccc67)n5)c4)cc23)cc1 ; c1ccc(-c2c(-c3cccc(-c4cccc(-c5nc6c7ccccc7ccc6c6ccccc56)n4)c3)cnc3c2ccc2cccnc23)cc1]

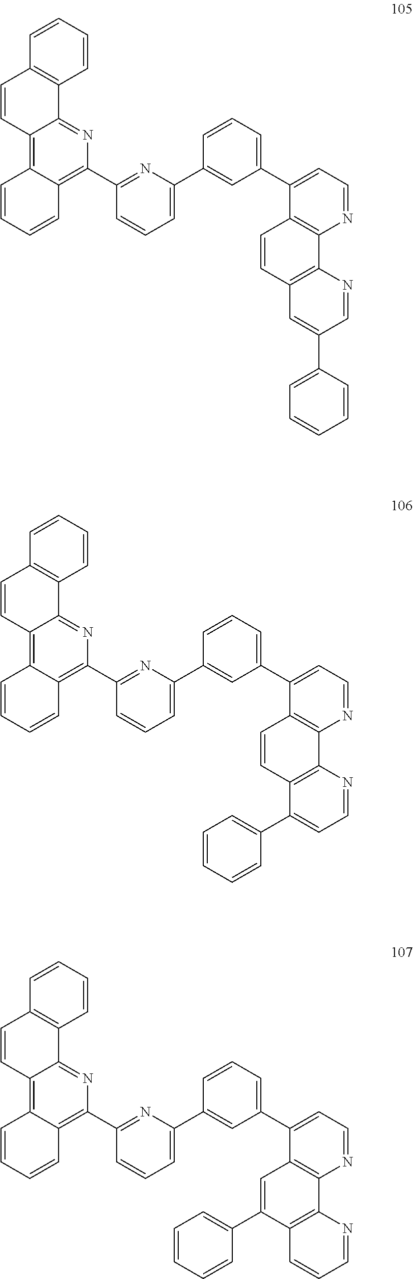 [SMILES: c1ccc(-c2cnc3c(ccc4c(-c5cccc(-c6cccc(-c7nc8c9ccccc9ccc8c8ccccc78)n6)c5)ccnc43)c2)cc1 ; c1ccc(-c2ccnc3c2ccc2c(-c4cccc(-c5cccc(-c6nc7c8ccccc8ccc7c7ccccc67)n5)c4)ccnc23)cc1 ; c1ccc(-c2cc3c(-c4cccc(-c5cccc(-c6nc7c8ccccc8ccc7c7ccccc67)n5)c4)ccnc3c3ncccc23)cc1]

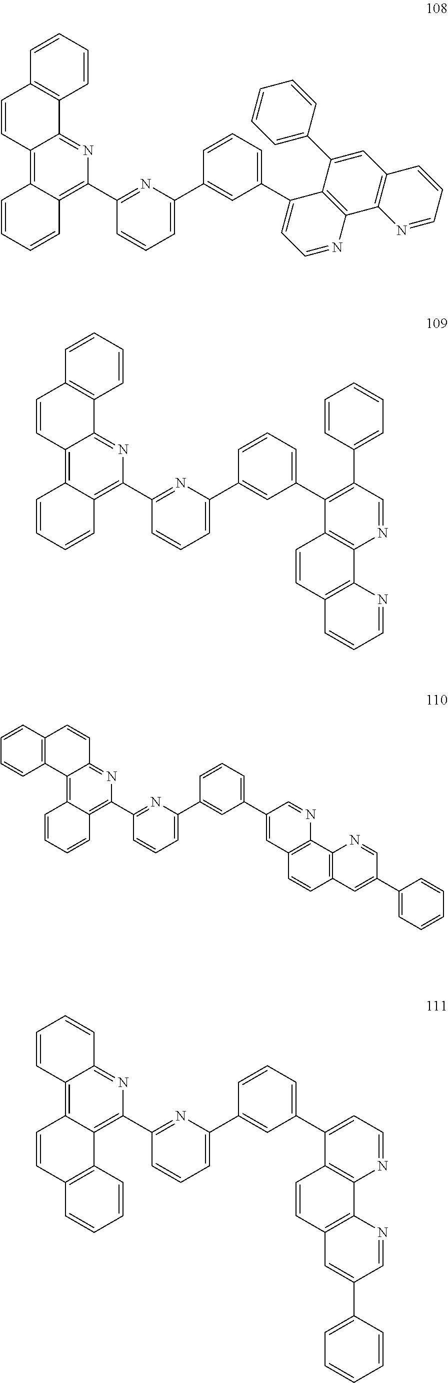 [SMILES: c1ccc(-c2cc3cccnc3c3nccc(-c4cccc(-c5cccc(-c6nc7c8ccccc8ccc7c7ccccc67)n5)c4)c23)cc1 ; c1ccc(-c2cnc3c(ccc4cccnc43)c2-c2cccc(-c3cccc(-c4nc5c6ccccc6ccc5c5ccccc45)n3)c2)cc1 ; c1ccc(-c2cnc3c(ccc4cc(-c5cccc(-c6cccc(-c7nc8ccc9ccccc9c8c8ccccc78)n6)c5)cnc43)c2)cc1 ; c1ccc(-c2cnc3c(ccc4c(-c5cccc(-c6cccc(-c7nc8ccccc8c8ccc9ccccc9c78)n6)c5)ccnc43)c2)cc1]

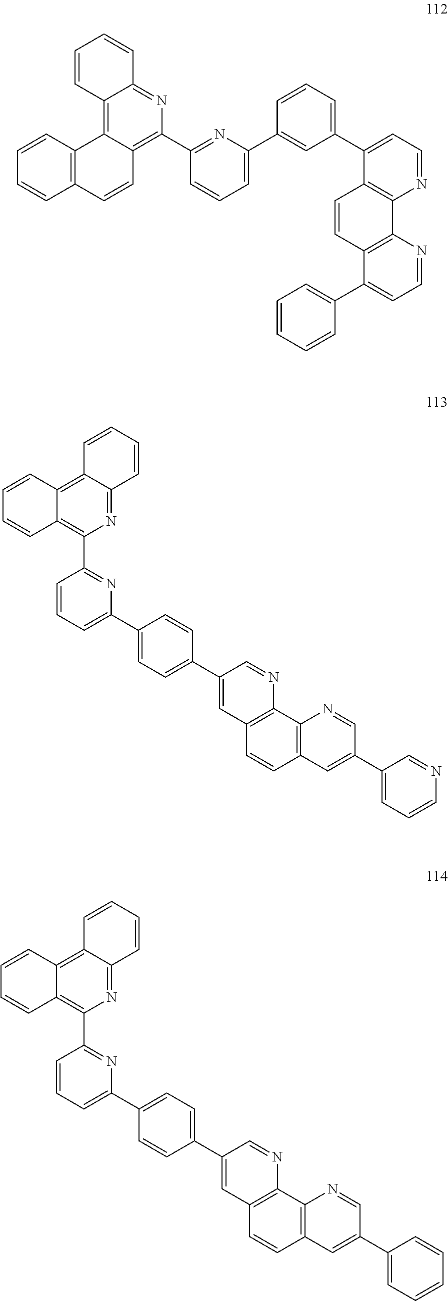 [SMILES: c1ccc(-c2ccnc3c2ccc2c(-c4cccc(-c5cccc(-c6nc7ccccc7c7c6ccc6ccccc67)n5)c4)ccnc23)cc1 ; c1cncc(-c2cnc3c(ccc4cc(-c5ccc(-c6cccc(-c7nc8ccccc8c8ccccc78)n6)cc5)cnc43)c2)c1 ; c1ccc(-c2cnc3c(ccc4cc(-c5ccc(-c6cccc(-c7nc8ccccc8c8ccccc78)n6)cc5)cnc43)c2)cc1]

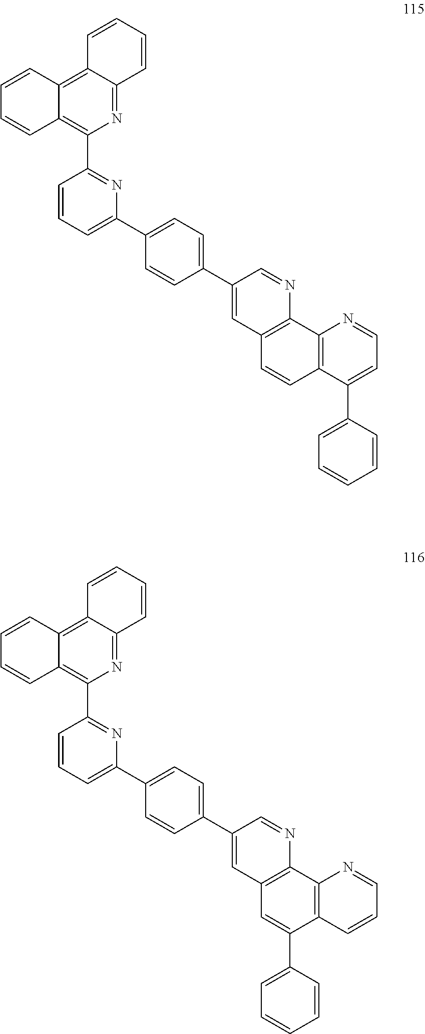 [SMILES: c1ccc(-c2ccnc3c2ccc2cc(-c4ccc(-c5cccc(-c6nc7ccccc7c7ccccc67)n5)cc4)cnc23)cc1 ; c1ccc(-c2cc3cc(-c4ccc(-c5cccc(-c6nc7ccccc7c7ccccc67)n5)cc4)cnc3c3ncccc23)cc1]

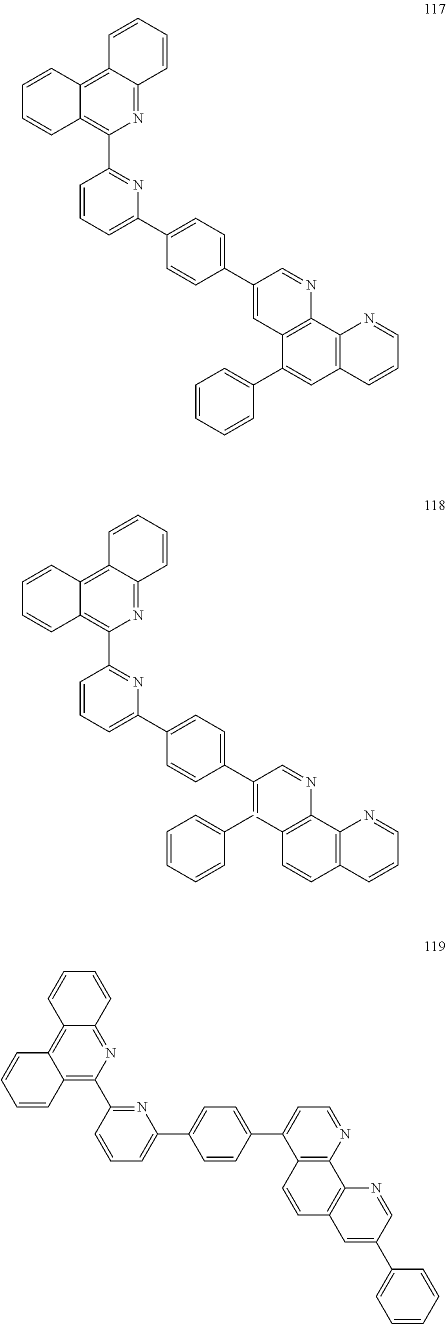

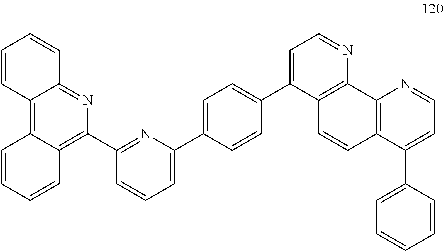 [SMILES: c1ccc(-c2ccnc3c2ccc2c(-c4ccc(-c5cccc(-c6nc7ccccc7c7ccccc67)n5)cc4)ccnc23)cc1]

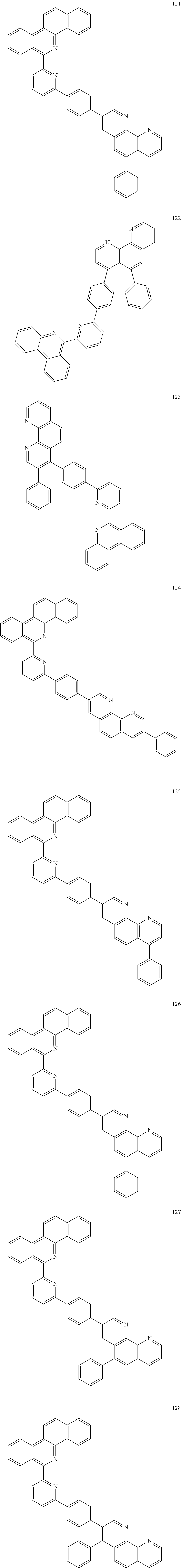

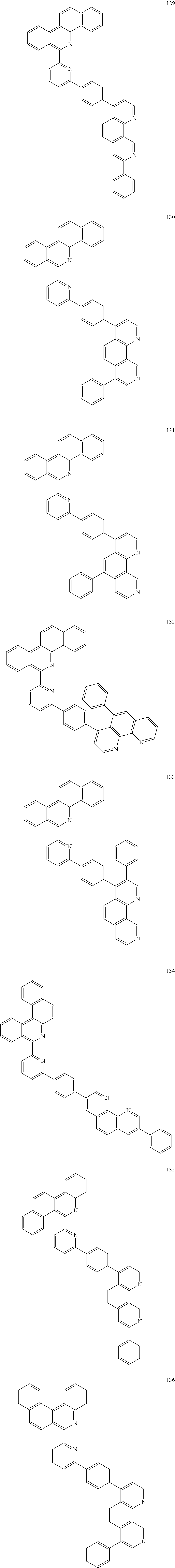

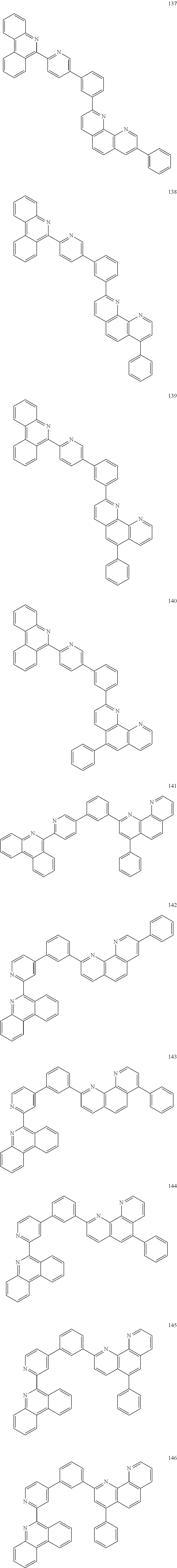

As used herein, “alkyl” may refer to a monovalent substituent derived from a linear or branched chain saturated hydrocarbon having 1 to 40 carbon atoms. Examples of such alkyl may include, but are not limited to, methyl, ethyl, propyl, isobutyl, sec-butyl, pentyl, iso-amyl, hexyl or the like.

As used herein, “alkenyl” may refer to a monovalent substituent derived from a linear or branched chain unsaturated hydrocarbon having 2 to 40 carbon atoms, having at least one carbon-carbon double bond. Examples of such alkenyl may include, but are not limited to, vinyl, allyl, isopropenyl, 2-butenyl or the like.

As used herein, “alkynyl” may refer to a monovalent substituent derived from a linear or branched chain unsaturated hydrocarbon having 2 to 40 carbon atoms, having at least one carbon-carbon triple bond. Examples of such alkynyl may include, but are not limited to, ethynyl, 2-propynyl or the like.

As used herein, “cycloalkyl” may refer to a monovalent substituent derived from a monocyclic or polycyclic non-aromatic hydrocarbon having 3 to 40 carbon atoms. Examples of such cycloalkyl may include, but are not limited to, cyclopropyl, cyclopentyl, cyclohexyl, norbornyl, adamantyl or the like.

As used herein, “heterocycloalkyl” may refer to a monovalent substituent derived from a non-aromatic hydrocarbon having 3 to 40 nuclear atoms, where one or more carbons in the ring, preferably one to three carbons, are substituted with a heteroatom such as N, O, S or Se. Examples of such heterocycloalkyl may include, but are not limited to, morpholine group, piperazine group or the like.

As used herein, “aryl” may refer to a monovalent substituent derived from an aromatic hydrocarbon having 6 to 60 carbon atoms which is in a structure with a single ring or two or more rings combined with each other. In addition, a form in which two or more rings are pendant to or fused with each other may also be included. Examples of such aryl may include, but are not limited to, phenyl, naphthyl, phenanthryl, anthryl or the like.

As used herein, “heteroaryl” may refer to a monovalent substituent derived from a monoheterocyclic or polyheterocyclic aromatic hydrocarbon having 5 to 60 nuclear atoms. In such a case, one or more carbons in the ring, preferably one to three carbons, are substituted with a heteroatom such as N, O, S or Se. In addition, a form in which two or more rings are pendant to or fused with each other may be included, and a form fused with an aryl group may be included. Examples of such heteroaryl may include, but are not limited to, a 6-membered monocyclic ring including, for example, pyridyl, pyrazinyl, pyrimidinyl, pyridazinyl and triazinyl; a polycyclic ring including, for example, phenoxathienyl, indolinzinyl, indolyl, purinyl, quinolyl, benzothiazolyl, and carbazolyl; 2-furanyl; N-imidazolyl; 2-isoxazolyl; 2-pyridinyl; 2-pyrimidinyl or the like.

As used herein, “alkyloxy” refers to a monovalent substituent represented by R′O—, where R′ refers to an C1 to C40 alkyl, and such alkyloxy may include a linear, branched or cyclic structure. Examples of such alkyloxy may include, but are not limited to, methoxy, ethoxy, n-propoxy, 1-propoxy, t-butoxy, n-butoxy, pentoxy or the like.

As used herein, “aryloxy” refers to a monovalent substituent represented by RO—, where R refers to a C5 to C40 aryl. Examples of such aryloxy may include, but are not limited to, phenyloxy, naphthyloxy, diphenyloxy or the like.

As used herein, “alkylsilyl” refers to a silyl substituted with a C1 to C40 alkyl and includes mono-, di- and tri-alkylsilyl. In addition, “arylsilyl” refers to a silyl substituted with a C5 to C60 aryl, and includes mono-, as well as polyarylsilyl such as di- and tri-arylsilyl.

As used herein, “alkylboron group” refers to a boron group substituted with a C1 to C40 alkyl, and “arylboron group” refers to a boron group substituted with a C6 to C60 aryl.

As used herein, “alkylphosphinyl group” refers to a phosphine group substituted with an C1 to C40 alkyl, and includes mono- and di-alkylphosphinyl group. In addition, as used herein, “arylphosphinyl group” refers to a phosphine group substituted with a C1 to C40 monoaryl or diaryl, and includes mono- and di-arylphosphinyl group.

As used herein, “arylamine group” refers to an amine substituted with a C6 to C60 aryl, and includes mono- and di-arylamine group.

As used herein, “heteroarylamine group” refers to an amine substituted with a heteroaryl having 5 to 60 nuclear atoms and includes mono- and di-heteroarylamine groups.

As used herein, “(aryl) (heteroaryl)amine group” refers to an amine group substituted with a C6-C60 aryl and a heteroaryl having 5 to 60 nuclear atoms.

As used herein, “fused ring” refers to a C3-C40 fused aliphatic ring, a C6-C60 fused aromatic ring, a fused heteroaliphatic ring having 3 to 60 nuclear atoms, a fused heteroaromatic ring with 5 to 60 nuclear atoms, or combinations thereof.

### <Organic EL Device>

The present disclosure provides an organic electroluminescence device (hereinafter referred to as “organic EL device”) comprising the compound represented by the above Chemical Formula 1.

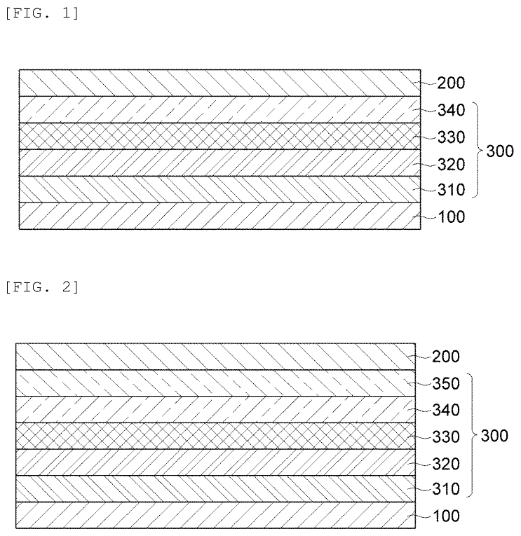

FIGS. 1 to 4 are schematic cross-sectional views of organic EL devices according to the first to fourth embodiments of the present disclosure.

Hereinafter, the organic EL devices according to the first to third embodiments of the present disclosure will be explained in detail with reference to FIGS. 1 to 3.

As shown in FIGS. 1 to 3, the organic EL device according to the present disclosure includes an anode (100), a cathode (200), and one or more organic layers (300) interposed between the anode and cathode, wherein at least one of the one or more organic layers includes the compound represented by Chemical Formula 1. In this regard, the compound can be used alone or in combination with another compound.

The one or more organic layers (300) may include any one or more of a hole injection layer (310), a hole transport layer (320), a light-emitting layer (330), an electron transport auxiliary layer (360), an electron transport layer (340), and an electron injection layer (350), wherein at least one organic layer (300) contains the compound represented by Chemical Formula 1. Specifically, the organic layer containing the compound represented by Chemical Formula 1 may be an electron transport layer (340). That is, the compound represented by Chemical Formula 1 is included as an electron transport layer material in the organic EL device. In such an organic EL device, electrons are easily injected from the cathode or electron injection layer into the electron transport layer due to the compound of Chemical Formula 1 and can also quickly move from the electron transport layer to the light-emitting layer, resulting in a high binding force of holes and electrons in the light-emitting layer. Therefore, the organic EL device of the present disclosure exhibits excellent luminous efficiency, power efficiency, and brightness. Moreover, the compound of Chemical Formula 1 has excellent thermal and electrochemical stability, thereby improving the performance of the organic EL device.

The compound of Chemical Formula 1 can be used alone or in combination with an electron transport layer material known in the art.

The electron transport layer materials that can be mixed with the compound of Chemical Formula 1 include commonly known electron transport materials in the art. Non-limiting examples of usable electron transport materials include oxazole compounds, isoxazole compounds, triazole compounds, isothiazole compounds, oxadiazole compounds, thiadiazole compounds, perylene compounds, aluminum complexes (e.g., Alq₃, tris(8-quinolinolato)aluminum), gallium complexes (e.g., Gaq′2OPiv, Gaq′ 2OAc, 2(Gaq′2)), and others. These may be used alone or in combination.

In the present disclosure, when mixing the compound of Chemical Formula 1 with electron transport layer materials, the mixing ratio is not particularly limited and can be appropriately adjusted within the known range in the art.

The structure of the organic EL device of the present disclosure is not particularly limited, but for example, the anode (100), one or more organic layers (300), and the cathode (200) may be sequentially laminated on a substrate (see FIGS. 1 to 3). Furthermore, although not shown, an insulating layer or an adhesive layer may be inserted at the interface between the electrode and the organic layer.

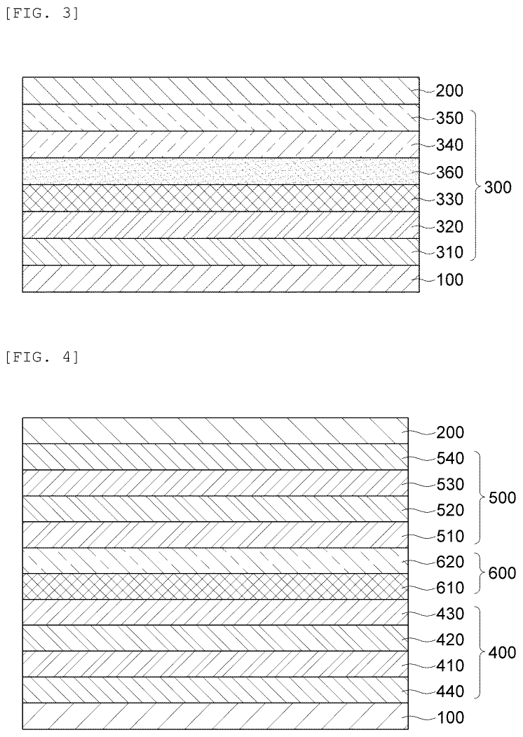

In an example, as shown in FIG. 1, the organic EL device may have a structure in which the anode (100), a hole injection layer (310), a hole transport layer (320), a light-emitting layer (330), an electron transport layer (340), and the cathode (200) are sequentially laminated on a substrate. Optionally, as shown in FIG. 2, an electron injection layer (350) may be located between the electron transport layer (340) and the cathode (200). Additionally, an electron transport auxiliary layer (360) may be arranged between the light-emitting layer (330) and the electron transport layer (340) (see FIG. 3).

The organic EL device of the present disclosure may be manufactured by forming the organic layer and electrode using known materials and methods in the art, except that at least one of the organic layers (e.g., the electron transport layer (340)) includes the compound represented by Chemical Formula 1.

The organic layer may be formed by a vacuum deposition method or a solution coating method. Examples of the solution coating method include spin coating, dip coating, doctor blading, inkjet printing, or thermal transfer, but are not limited thereto.

The substrate usable in the present disclosure is not particularly limited and may include, for example, silicon wafers, quartz, glass plates, metal plates, plastic films, and sheets.

Examples of anode materials include metals such as vanadium, chromium, copper, zinc, and gold, or alloys thereof; metal oxides such as zinc oxide, indium oxide, indium tin oxide (ITO), and indium zinc oxide (IZO); combinations of metals and oxides such as ZnO:Al or SnO₂:Sb; conductive polymers such as polythiophene, poly(3-methylthiophene), poly[3, 4-(ethylene-1, 2-dioxy)thiophene] (PEDT), polypyrrole, or polyaniline; and carbon black, but are not limited thereto.

Examples of cathode materials include metals such as magnesium, calcium, sodium, potassium, titanium, indium, yttrium, lithium, gadolinium, aluminum, silver (Ag), tin, lead, and alloys thereof; and multilayer structures such as LiF/Al or LiO₂/Al, but are not limited thereto.

Furthermore, the hole injection layer, hole transport layer, light-emitting layer, and electron injection layer are not particularly limited and may employ conventional materials known in the art.

Hereinafter, the organic EL device according to the fourth embodiment of the present disclosure will be described with reference to FIG. 4.

As shown in FIG. 4, the organic electroluminescent device according to the fourth embodiment of the present disclosure is a tandem device, which includes: an anode 100 and a cathode 200 facing each other on a substrate (not shown); a plurality of light-emitting units 400 and 500 interposed between the anode 100 and the cathode 200; and a charge generation layer 600 interposed between the adjacent light-emitting units 400 and 500 and including an N-type charge generation layer 610 and a P-type charge generation layer 620. Particularly, the N-type charge generation layer 610 contains the above-described compound represented by Chemical Formula 1.

Such a tandem organic electroluminescent device includes at least two light-emitting units, with a charge generation layer interposed between adjacent light-emitting units, thereby increasing the number of light-emitting units.

In an example, the plurality of light-emitting units may include a first light-emitting unit 400, a second light-emitting unit 500, . . . , and an m-th light-emitting unit (m=an integer of 3 or greater, specifically 3-4). Particularly, a charge generation layer 600 is disposed between the adjacent light-emitting units, and comprises an N-type charge generation layer 610 and a P-type charge generation layer 620, wherein the N-type charge generation layer 610 contains the compound represented by Chemical Formula 1.

Specifically, the organic electroluminescent device according to the present disclosure includes: an anode 100 and a cathode 200 facing each other; a first light-emitting unit 400 disposed on the anode 100; a second light-emitting unit 500 disposed above the first light-emitting unit 400; a charge generation layer 600, disposed between the first and the second light-emitting unit 400 and 500 and including an N-type charge generation layer 610 and a P-type charge generation layer 620. Particularly, the N-type charge generation layer 610 contains the above-described compound represented by Chemical Formula 1.

The light-emitting units 400 and 500 include hole transport layers 410 and 510, light-emitting layers 420 and 520, and electron transport layers 430 and 530, respectively. Specifically, the first light-emitting unit 400 may include a first hole transport layer 410, a first light-emitting layer 420, and a first electron transport layer 430, and the second light-emitting unit 500 may include a hole transport layer 510, a light-emitting layer 520, and an electron transport layer 530. Optionally, the first light-emitting unit 400 may further include a hole injection layer 440.

The hole transport layers 410 and 510, the light-emitting layers 420 and 520, the electron transport layers 430 and 530, and the hole injection layer 440 may employ, but are not particularly limited to, commonly used materials known in the art.

The charge generation layer (CGL, 600) is disposed between the adjacent light-emitting units 400 and 500, and thus can control the charges between the light-emitting units 400 and 500 to make a charge balance.

The charge generation layer 600 includes: an N-type charge generation layer 610 disposed adjacent to the first light-emitting unit 400 to supply electrons to the first light-emitting unit 400; and a P-type charge generation layer 620 disposed adjacent to the second light-emitting unit 500 to supply holes to the second light-emitting unit 500.

The N-type charge generation layer 610 contains the above-described compound represented by Chemical Formula 1. The compound of Chemical Formula 1 exhibits excellent electron injection and transport capability due to excellent electron mobility. Therefore, the application of the compound of Chemical Formula 1 as an N-type charge generation layer material to an organic electroluminescent device can prevent the increase in progressive driving voltage and the decrease in lifespan for the device.

In an example, the N-type charge generation layer 610 contains one host having electron transport characteristics, wherein the one host is the compound represented by Chemical Formula 1. Unlike the N-type charge generation layer containing two hosts, the N-type charge generation layer 610 of the present disclosure can be formed through co-deposition, leading to an improvement in process efficiency.

The N-type charge generation layer 610 may further contain an N-type dopant.

The N-type dopant usable in the present disclosure is not particularly limited as long as the N-type dopant is a material that is generally used for an N-type charge generation layer. Examples of the material include: alkali metals, such as Li, Na, K, Rb, Cs, and Fr; alkali earth metals, such as Be, Mg, Ca, Sr, Ba, and Ra; group 15 metals, such as Bi (bismuth) and Sb (antimony); lanthanide metals, such as La (lanthanum), Ce (cerium), Pr (praseodyminum), Nd (neodymium), Pm (promethium), Sm (samarium), europium (europium), Gd (gadolinium), Tb (terbium), Dy (dysprosium), Ho (holmium), Er (erbium), Tm (thulium), Yb (ytterbium), and Lu (lutetium); and compounds of at least one of the metals. Alternatively, the N-type dopant may be an organic N-type dopant that has electron donor characteristics and can donor at least some of electric charges to an organic host (e.g., the compound of Chemical Formula 1) to form a charge-transfer complex with the organic host, and examples thereof may be include bis(ethylenedithio)tetrathiafulvalene (BEDT-TTF), tetrathiafulvalene (TTF), and the like.

The thickness of the N-type charge generation layer 610 is not particularly limited and may be in the range of, for example, about 5 to 30 nm.

The P-type charge generation layer 620 may be composed of a metal or a P-type doped organic material. Especially, the metal may be exemplified by Al, Cu, Fe, Pb, Zn, Au, Pt, W, In, Mo, Ni, and Ti, which may be used alone or as an alloy of two or more thereof. In addition, the P-type dopant and host material used in the P-type doped organic material are not particularly limited as long as the P-type dopant and host material are commonly used. Examples of the P-type dopant may include F₄-TCNQ (2, 3, 5, 6-tetrafluoro-7, 7, 8, 8-tetracyano-quinodimethane), iodide, FeCl₃, FeF₃, SbCl₅, and the like, which may be used alone or as a mixture of two or more thereof. Non-limiting examples of the host include NPB (N,N′-bis(naphthaen-1-yl)-N,N′-bis(phenyl)-benzidine), TPD (N,N′-bis(3-methylphenyl) N,N′-bis(phenyl)-benzidine), and TNB (N, N,N′, N′-tetra-naphthalenyl-benzidine), which may be used alone or as a mixture of two or more thereof.

The descriptions of the anode 100 and the cathode 200 are the same as those in the above-described first to third embodiments and are thus omitted.

Hereinafter, the present disclosure will be described in detail with reference to the following examples. However, the following examples are merely to illustrate the disclosure, and the scope of the present disclosure is not limited thereto.

### [Preparation Example 1] Synthesis of 2-(6-chloropyridin-2-yl)quinoline (A-1)

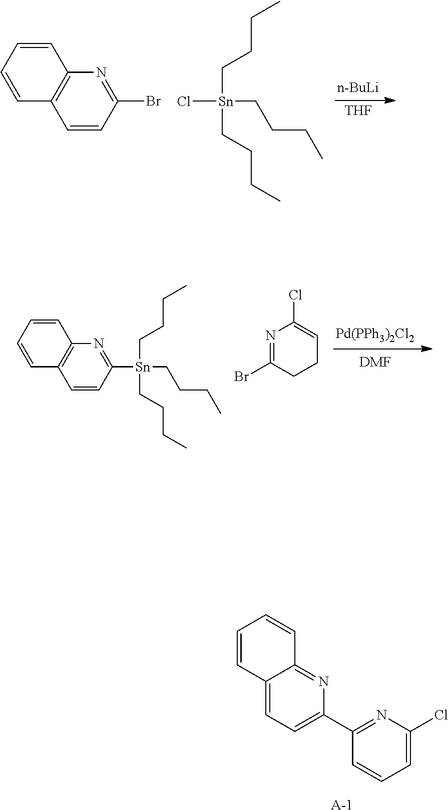

### Step 1. Synthesis of 2-(tributylstannyl)quinoline

2-Bromoquinoline (10.0 g, 48.1 mmol) was dissolved in 150 ml of dry THE, and then cooled at −78° C. in a nitrogen atmosphere, and subsequently, n-BuLi (30.0 mL of 1.6 M in hexane, 48.1 mmol) was slowly added dropwise. After stirring at −78° C. for 1 hour, tributyltin chloride (15.6 g, 48.1 mmol) was added, followed by reaction for 3 hours, and the reaction was completed by a NH₄Cl solution at room temperature. The mixture that had been reacted was subjected to extraction with EtOAc, followed by washing with Brine. The organic layer that had been washed was concentrated to give 2-(tributylstannyl)quinoline (18.3 g, 43.8 mmol, yield: 91%).

Mass: [(M+H)⁺]: 419

### Step 2. Synthesis of 2-(6-chloropyridin-2-yl)quinoline

After (tributylstannyl)quinoline (18.3 g, 43.8 mmol) synthesized in Step 1 of Preparation Example 1 and 2-bromo-6-chloropyridine (8.4 g, 43.8 mmol) were placed in dry DMF, Pd(PPh₃)₂Cl₂ (3.1 g, 4.4 mmol) was added, followed by heating and stirring at 110° C. for 18 hours. After completion of the reaction, the resultant product was diluted with EtOAc and washed with water, and then the organic layer was dried over magnesium sulfate, concentrated, and then purified by column chromatography to give 2-(6-chloropyridin-2-yl)quinoline (4.2 g, 17.5 mmol, yield: 40%).

Mass: [(M+H)⁺]: 242

### [Preparation Example 2] Synthesis of 6-(6-chloropyridin-2-yl)phenanthridine (A-2)

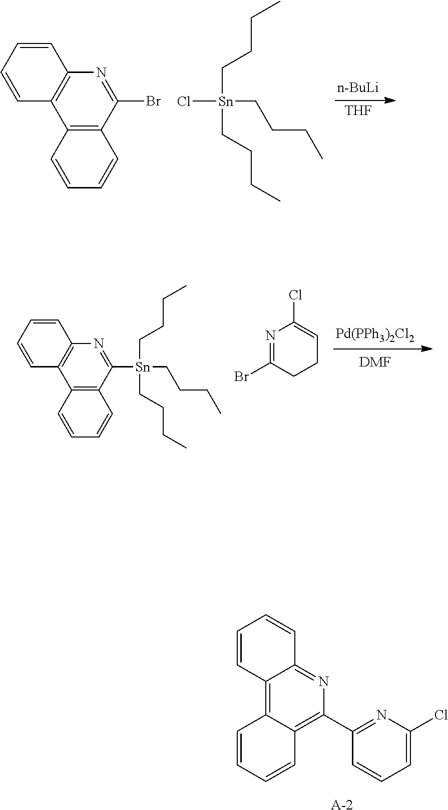

### Step 1. Synthesis of 6-(tributylstannyl)phenanthridine

6-bromophenanthridine (10.0 g, 38.7 mmol) was dissolved in 150 ml of dry THE, and then cooled at −78° C. in a nitrogen atmosphere, and subsequently, n-BuLi (24.2 mL of 1.6 M in hexane, 38.7 mmol) was slowly added dropwise. After stirring at −78° C. for 1 hour, tributyltin chloride (12.6 g, 38.7 mmol) was added, followed by reaction for 3 hours, and the reaction was completed by a NH₄Cl solution at room temperature. The mixture that had been reacted was subjected to extraction with EtOAc, followed by washing with Brine. The organic layer that had been washed was concentrated to give 6-(tributylstannyl) phenanthridine (16.1 g, 34.5 mmol, yield: 89%).

Mass: [(M+H)⁺]: 469

### Step 2. Synthesis of 6-(6-chloropyridin-2-yl)phenanthridine

After 6-(tributylstannyl)phenanthridine (16.1 g, 34.5 mmol) synthesized in Step 1 of Preparation Example 2 and 2-bromo-6-chloropyridine (6.6 g, 34.5 mmol) were placed in dry DMF, Pd(PPh₃)₂Cl₂ (2.4 g, 3.4 mmol) was added, followed by heating and stirring at 110° C. for 18 hours. After completion of the reaction, the resultant product was diluted with EtOAc and washed with water, and then the organic layer was dried over magnesium sulfate, concentrated, and then purified by column chromatography to give 6-(6-chloropyridin-2-yl)phenanthridine (4.2 g, 14.4 mmol, yield: 42%).

Mass: [(M+H)⁺]: 292

### [Preparation Example 3] Synthesis of 6-(6-chloropyridin-2-yl)benzo[c]phenanthridine (A-3)

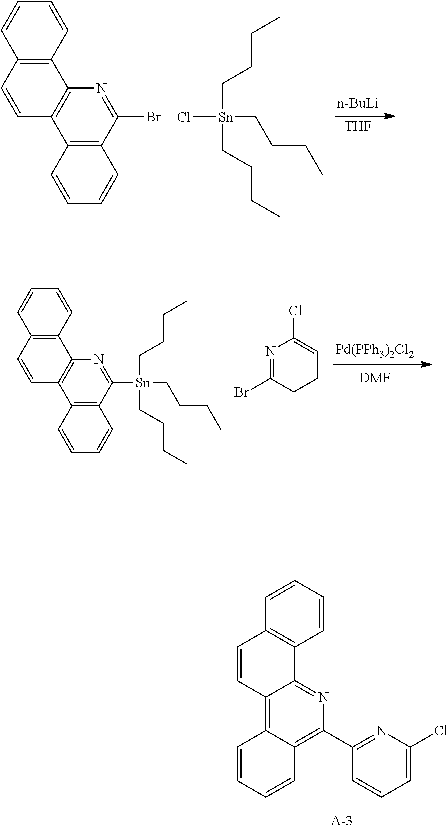

### Step 1. Synthesis of 6-(tributylstannyl)benzo[c]phenanthridine

6-bromobenzo[c]-phenanthridine (10.0 g, 32.4 mmol) was dissolved in 150 ml of dry THE, and then cooled at −78° C. in a nitrogen atmosphere, and subsequently, n-BuLi (20.3 mL of 1.6 M in hexane, 32.4 mmol) was slowly added dropwise. After stirring at −78° C. for 1 hour, tributyltin chloride (10.6 g, 32.4 mmol) was added, followed by reaction for 3 hours, and the reaction was completed by a NH₄Cl solution at room temperature. The mixture that had been reacted was subjected to extraction with EtOAc, followed by washing with Brine. The organic layer that had been washed was concentrated to give 6-(tributylstannyl)benzo[c]phenanthridine (15.0 g, 28.9 mmol, yield: 89%).

Mass: [(M+H)⁺]: 519

### Step 2. Synthesis of 6-(6-chloropyridin-2-yl)benzo[c]phenanthridine

After 6-(tributylstannyl)benzo[c]phenanthridine (15.0 g, 28.9 mmol) synthesized in Step 1 of Preparation Example 3 and 2-bromo-6-chloropyridine (5.6 g, 28.9 mmol) were placed in dry DMF, Pd(PPh₃)₂Cl₂ (2.0 g, 2.9 mmol) was added, followed by heating and stirring at 110° C. for 18 hours. After completion of the reaction, the resultant product was diluted with EtOAc and washed with water, and then the organic layer was dried over magnesium sulfate, concentrated, and then purified by column chromatography to give 6-(6-chloropyridin-2-yl)benzo[c]phenanthridine (3.9 g, 11.6 mmol, yield: 40%).

Mass: [(M+H)⁺]: 342

### [Preparation Example 4] Synthesis of 2-(6-(3-chlorophenyl)pyridin-2-yl)quinoline (B-1)

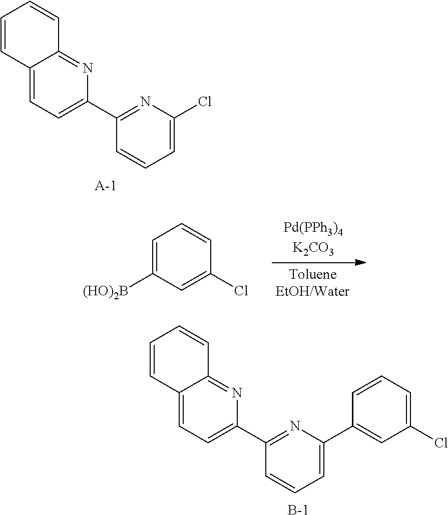

Compound A-1 (10.0 g, 41.5 mmol) synthesized by the method in Preparation Example 1, (3-chlorophenyl) boronic acid (6.5 g, 41.5 mmol), Pd(PPh₃)₄ (1.4 g, 1.2 mmol), and K₂CO₃ (17.2 g, 124.6 mmol) were placed in 100 ml of toluene, 15 ml of EtOH, and 15 ml of water, and then the mixture was heated to reflux with stirring for 2 hours. After completion of the reaction, the organic layer was extracted by methylene chloride, and the extracted organic layer was dried over magnesium sulfate, concentrated, and then crystallized by acetone and MeOH to give 2-(6-(3-chlorophenyl)pyridin-2-yl)quinoline (11.2 g, 35.3 mmol, yield: 85%).

Mass: [(M+H)⁺]: 318

### [Preparation Example 5] Synthesis of 6-(6-(3-chlorophenyl)pyridin-2-yl)phenanthridine (B-2)

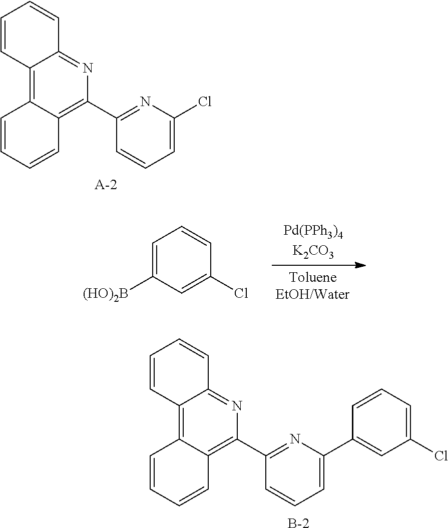

Compound A-2 (10 g, 34.4 mmol) synthesized by the method in Preparation Example 2, (3-chlorophenyl) boronic acid (5.4 g, 34.4 mmol), Pd(PPh₃)₄ (1.2 g, 1.0 mmol), and K₂CO₃ (14.3 g, 103.2 mmol) were placed in 100 ml of toluene, 15 ml of EtOH, and 15 ml of water, and then the mixture was heated to reflux with stirring for 2 hours. After completion of the reaction, the organic layer was extracted by methylene chloride, and the extracted organic layer was dried over magnesium sulfate, concentrated, and then crystallized by acetone and MeOH to give 6-16-(3-chlorophenyl)pyridin-2-yl)phenanthridine (11.0 g, 29.9 mmol, yield: 87%).

Mass: [(M+H)⁺]: 368

### [Preparation Example 6] Synthesis of 6-(6-(3-chlorophenyl)pyridin-2-yl)benzo[c]phenanthridine (B-3)

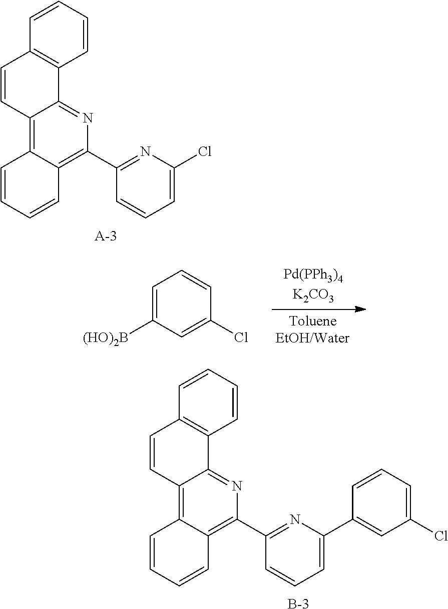

Compound A-3 (10.0 g, 29.3 mmol) synthesized by the method in Preparation Example 3, (3-chlorophenyl) boronic acid (4.6 g, 29.3 mmol), Pd(PPh₃)₄ (1.0 g, 0.9 mmol), and K₂CO₃ (12.2 g, 88.0 mmol) were placed in 100 ml of toluene, 15 ml of EtOH, and 15 ml of water, and then the mixture was heated to reflux with stirring for 2 hours. After completion of the reaction, the organic layer was extracted by methylene chloride, and the extracted organic layer was dried over magnesium sulfate, concentrated, and then crystallized by acetone and MeOH to give 6-(6-(3-chlorophenyl)pyridin-2-yl)benzo[c]phenanthridine (10.3 g, 24.6 mmol, yield: 84%).

Mass: [(M+H)⁺]: 418

### [Preparation Example 7] Synthesis of 6-(6-(4-chlorophenyl)pyridin-2-yl)phenanthridine (B-4)

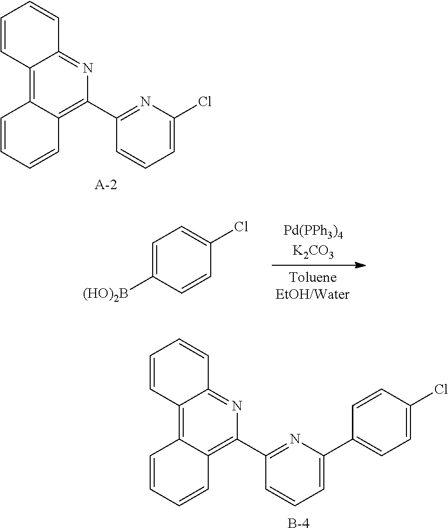

Compound A-2 (10.0 g, 34.4 mmol) synthesized by the method in Preparation Example 2, (4-chlorophenyl) boronic acid (5.4 g, 34.4 mmol), Pd(PPh₃)₄ (1.2 g, 1.0 mmol), and K₂CO₃ (14.3 g, 103.2 mmol) were placed in 100 ml of toluene, 15 ml of EtOH, and 15 ml of water, and then the mixture was heated to reflux with stirring for 2 hours. After completion of the reaction, the organic layer was extracted by methylene chloride, and the extracted organic layer was dried over magnesium sulfate, concentrated, and then crystallized by acetone and MeOH to give 6-(6-(4-chlorophenyl)pyridin-2-yl)phenanthridine (10.5 g, 28.5 mmol, yield: 83%).

Mass: [(M+H)⁺]: 368

### [Preparation Example 8] Synthesis of 6-(6-(3-chloronaphthalen-1-yl)pyridin-2-yl)phenanthridine (B-5)

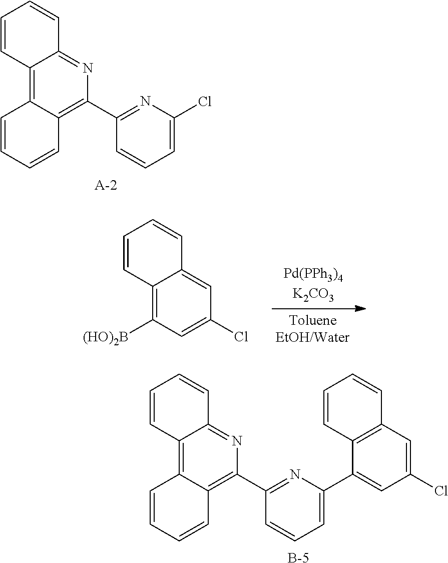

Compound A-2 (10.0 g, 34.4 mmol) synthesized by the method in Preparation Example 2, (3-chloronaphthalen-1-yl) boronic acid (7.1 g, 34.4 mmol), Pd(PPh₃)₄ (1.2 g, 1.0 mmol), and K₂CO₃ (14.3 g, 103.2 mmol) were placed in 100 ml of toluene, 15 ml of EtOH, and 15 ml of water, and then the mixture was heated to reflux with stirring for 2 hours. After completion of the reaction, the organic layer was extracted by methylene chloride, and the extracted organic layer was dried over magnesium sulfate, concentrated, and then crystallized by acetone and MeOH to give 6-(6-(3-chloronaphthalen-1-yl)pyridin-2-yl)phenanthridine (11.9 g, 28.5 mmol, yield: 83%).

Mass: [(M+H)⁺]: 418

### [Preparation Example 9] Synthesis of 6-(6-(7-chloronaphthalen-2-yl)pyridin-2-yl)phenanthridine (B-6)

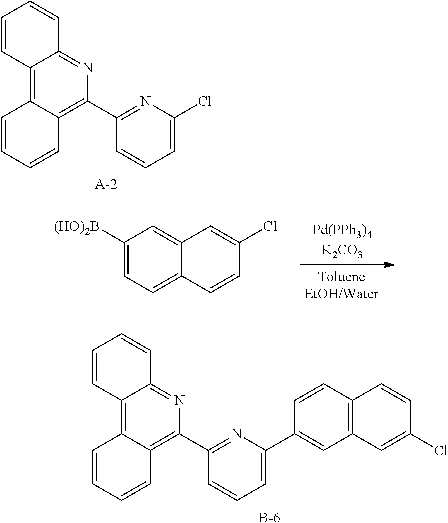

Compound A-2 (10.0 g, 34.4 mmol) synthesized by the method in Preparation Example 2, (7-chloronaphthalen-2-yl) boronic acid (7.1 g, 34.4 mmol), Pd(PPh₃)₄ (1.2 g, 1.0 mmol), and K₂CO₃ (14.3 g, 103.2 mmol) were placed in 100 ml of toluene, 15 ml of EtOH, and 15 ml of water, and then the mixture was heated to reflux with stirring for 2 hours. After completion of the reaction, the organic layer was extracted by methylene chloride, and the extracted organic layer was dried over magnesium sulfate, concentrated, and then crystallized by acetone and MeOH to give 6-(6-(7-chloronaphthalen-2-yl)pyridin-2-yl)phenanthridine (11.6 g, 27.9 mmol, yield: 81%).

Mass: [(M+H)⁺]: 418

### [Preparation Example 10] Synthesis of 6-(6-(7-chloronaphthalen-2-yl)pyridin-2-yl)benzo[c]phenanthridine (B-7)

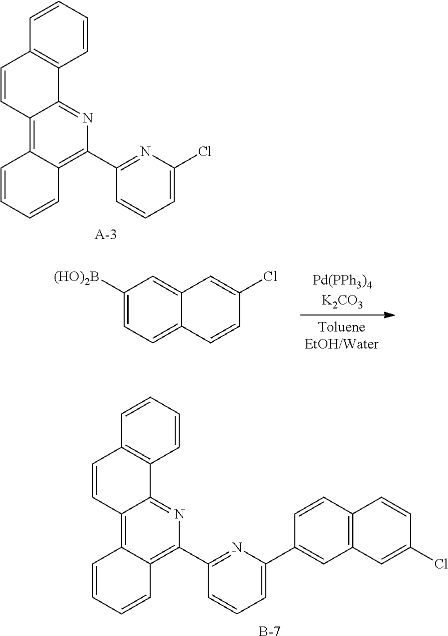

Compound A-3 (10.0 g, 29.3 mmol) synthesized by the method in Preparation Example 3, (7-chloronaphthalen-2-yl) boronic acid (6.1 g, 29.3 mmol), Pd(PPh₃)₄ (1.0 g, 0.9 mmol), and K₂CO₃ (12.2 g, mmol) were placed in 100 ml of toluene, 15 ml of EtOH, and 15 ml of water, and then the mixture was heated to reflux with stirring for 2 hours. After completion of the reaction, the organic layer was extracted by methylene chloride, and the extracted organic layer was dried over magnesium sulfate, concentrated, and then crystallized by acetone and MeOH to give 6-(6-(7-chloronaphthalen-2-yl)pyridin-2-yl)benzo[c]phenanthridine (11.8 g, 25.2 mmol, yield: 86%).

Mass: [(M+H)⁺]: 468

### [Preparation Example 11] Synthesis of 2-(6-(3-(4, 4, 5,5-tetramethyl-1, 3, 2-dioxaborolan-2-yl)phenyl)pyridine-2-yl)quinoline (C-1)

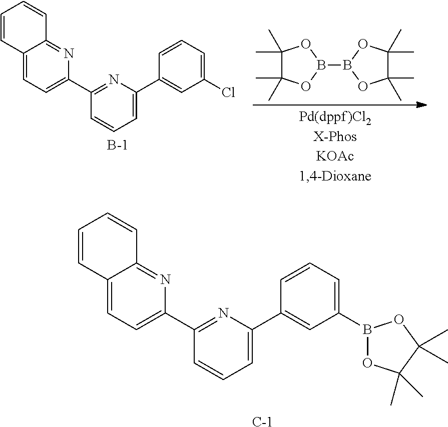

Compound B-1 (10 g, 31.6 mmol) synthesized by the method in Preparation Example 4, bis(pinacolato)diboron (10.4 g, 41.0 mmol), Pd(dppf)Cl₂ (0.7 g, 0.9 mmol), X-Phos (0.9 g, 1.9 mmol), and KOAc (6.2 g, 63.1 mmol) were placed in 100 ml of 1, 4-Dioxane, and then the mixture was heated to reflux with stirring for 6 hours. After completion of the reaction, KOAc was removed by filtration, and the organic layer was concentrated. And then, crystallized by acetone and MeOH to give 2-(6-(3-(4, 4,5,5-tetramethyl-1, 3, 2-dioxaborolan-2-yl)phenyl)pyridin-2-yl)quinoline (11.6 g, 28.4 mmol, yield: 90%).

Mass: [(M+H)⁺]: 409

### [Preparation Example 12] Synthesis of 6-(6-(3-(4, 4, 5,5-tetramethyl-1,3, 2-dioxaborolan-2-yl)phenyl)pyridin-2-yl)phenanthridine (C-2)

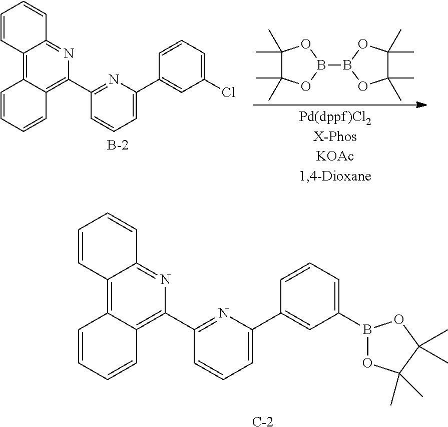

Compound B-2 (10 g, 27.3 mmol) synthesized by the method in Preparation Example 5, bis(pinacolato)diboron (9.0 g, 35.4 mmol), Pd(dppf)Cl₂ (0.6 g, 0.8 mmol), X-Phos (0.8 g, 1.6 mmol), and KOAc (5.4 g, 54.5 mmol) were placed in 100 ml of 1, 4-Dioxane, and then the mixture was heated to reflux with stirring for 6 hours. After completion of the reaction, KOAc was removed by filtration, and the organic layer was concentrated. And then, crystallized by acetone and MeOH to give 6-(6-(3-(4, 4,5,5-tetramethyl-1, 3, 2-dioxaborolan-2-yl)phenyl)pyridin-2-yl)phenanthridine (11.4 g, 24.8 mmol, yield: 91%).

Mass: [(M+H)⁺]: 459

### [Preparation Example 13] Synthesis of 6-(6-(3-(4, 4, 5,5-tetramethyl-1, 3, 2-dioxaborolan-2-yl)phenyl)pyridin-2-yl)benzo[c]phenanthridine (C-3)

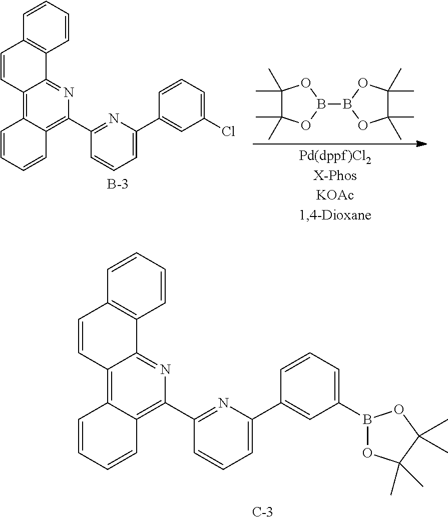

Compound B-3 (10 g, 24.0 mmol) synthesized by the method in Preparation Example 6, bis(pinacolato)diboron (7.9 g, 31.2 mmol), Pd(dppf)Cl₂ (0.5 g, 0.7 mmol), X-Phos (0.7 g, 1.4 mmol), and KOAc (4.7 g, 48.0 mmol) were placed in 100 ml of 1, 4-Dioxane, and then the mixture was heated to reflux with stirring for 6 hours. After completion of the reaction, KOAc was removed by filtration, and the organic layer was concentrated. And then, crystallized by acetone and MeOH to give 6-(6-(3-(4, 4, 5,5-tetramethyl-1, 3, 2-dioxaborolan-2-yl)phenyl)pyridin-2-yl)benzo[c]phenanthridine (10.9 g, 21.3 mmol, yield: 89%).

Mass: [(M+H)⁺]: 509

### [Preparation Example 14] Synthesis of 6-(6-(4-(4, 4, 5, 5-tetramethyl-1, 3, 2-dioxaborolan-2-yl)phenyl)pyridin-2-yl)phenanthridine (C-4)

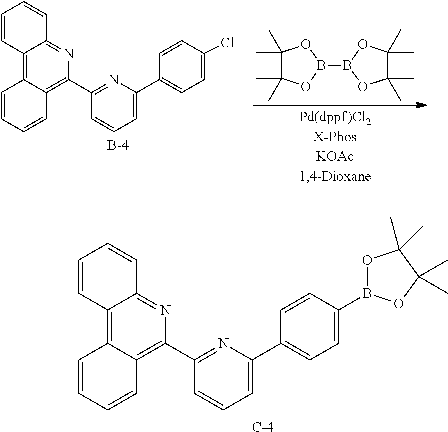

Compound B-4 (10 g, 27.3 mmol) synthesized by the method in Preparation Example 7, bis(pinacolato)diboron (9.0 g, 35.4 mmol), Pd(dppf)Cl₂ (0.6 g, 0.8 mmol), X-Phos (0.8 g, 1.6 mmol), and KOAc (5.4 g, 54.5 mmol) were placed in 100 ml of 1, 4-Dioxane, and then the mixture was heated to reflux with stirring for 6 hours. After completion of the reaction, KOAc was removed by filtration, and the organic layer was concentrated. And then, crystallized by acetone and MeOH to give 6-(6-(4-(4, 4, 5, 5-tetramethyl-1, 3, 2-dioxaborolan-2-yl)phenyl)pyridin-2-yl)phenanthridine (11.1 g, 24.3 mmol, yield: 89%).

Mass: [(M+H)⁺]: 459

### [Preparation Example 15] Synthesis of 6-(6-(3-(4, 4, 5,5-tetramethyl-1, 3,2-dioxaborolan-2-yl) naphthalen-1-yl)pyridin-2-yl)phenanthridine (C-5)

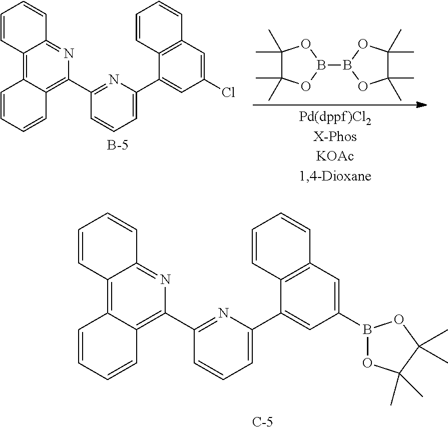

Compound B-5 (10 g, 24.0 mmol) synthesized by the method in Preparation Example 8, bis(pinacolato)diboron (7.9 g, 31.2 mmol), Pd(dppf)Cl₂ (0.5 g, 0.7 mmol), X-Phos (0.7 g, 1.4 mmol), and KOAc (4.7 g, 48.0 mmol) were placed in 100 ml of 1, 4-Dioxane, and then the mixture was heated to reflux with stirring for 6 hours. After completion of the reaction, KOAc was removed by filtration, and the organic layer was concentrated. And then, crystallized by acetone and MeOH to give 6-(6-(3-(4, 4, 5,5-tetramethyl-1, 3, 2-dioxaborolan-2-yl) naphthalen-1-yl)pyridin-2-yl)phenanthridine (11.0 g, 21.6 mmol, yield: 90%).

Mass: [(M+H)⁺]: 509

### [Preparation Example 16] Synthesis of 6-(6-(7-(4, 4, 5, 5-tetramethyl-1, 3, 2-dioxaborolan-2-yl) naphthalen-2-yl)pyridin-2-yl)phenanthridine (C-6)

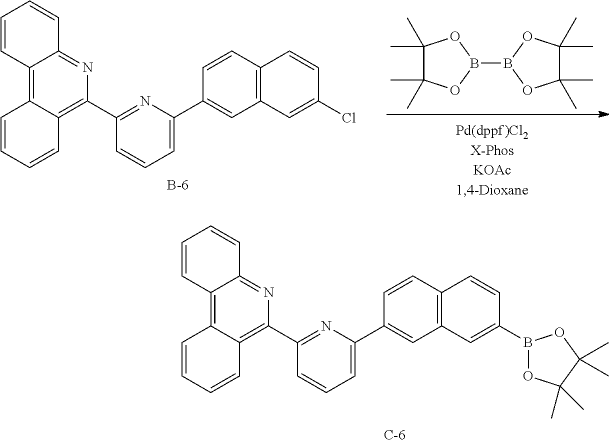

Compound B-6 (10 g, 24.0 mmol) synthesized by the method in Preparation Example 9, bis(pinacolato)diboron (7.9 g, 31.2 mmol), Pd (dppf) C12 (0.5 g, 0.7 mmol), X-Phos (0.7 g, 1.4 mmol), and KOAc (4.7 g, 48.0 mmol) were placed in 100 ml of 1, 4-Dioxane, and then the mixture was heated to reflux with stirring for 6 hours. After completion of the reaction, KOAc was removed by filtration, and the organic layer was concentrated. And then, crystallized by acetone and MeOH to give 6-(6-(7-(4, 4, 5,5-tetramethyl-1, 3, 2-dioxaborolan-2-yl) naphthalen-2-yl)pyridin-2-yl)phenanthridine (11.2 g, 22.1 mmol, yield: 92%).

Mass: [(M+H)⁺]: 509

### [Preparation Example 17] Synthesis of 6-(6-(7-(4, 4, 5,5-tetramethyl-1, 3, 2-dioxaborolan-2-yl) naphthalen-2-yl)pyridin-2-yl)benzo[c]phenanthridine (C-7)

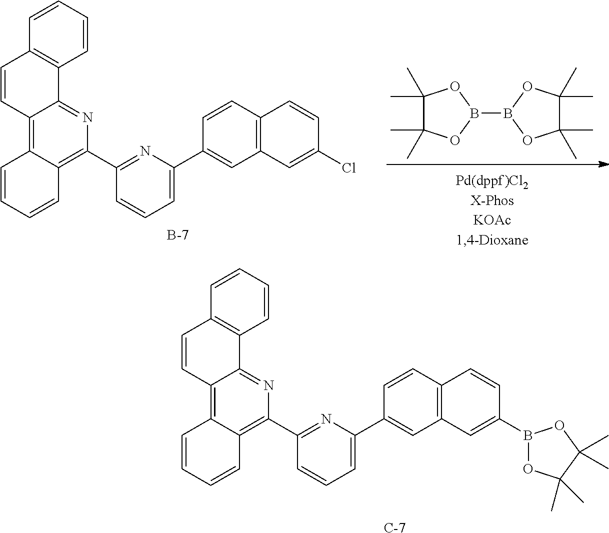

Compound B-7 (10 g, 21.4 mmol) synthesized by the method in Preparation Example 10, bis(pinacolato)diboron (7.1 g, 27.8 mmol), Pd(dppf)Cl₂ (0.5 g, 0.6 mmol), X-Phos (0.6 g, 1.3 mmol), and KOAc (4.2 g, 42.8 mmol) were placed in 100 ml of 1, 4-Dioxane, and then the mixture was heated to reflux with stirring for 6 hours. After completion of the reaction, KOAc was removed by filtration, and the organic layer was concentrated. And then, crystallized by acetone and MeOH to give 6-(6-(7-(4, 4, 5,5-tetramethyl-1, 3, 2-dioxaborolan-2-yl) naphthalen-2-yl)pyridin-2-yl)benzo[c]phenanthridine (10.2 g, 18.2 mmol, yield: 85%).

Mass: [(M+H)⁺]: 560

### [Preparation Example 18] Synthesis of 2-chloro-9-phenyl-1,10-phenanthroline (S-1)

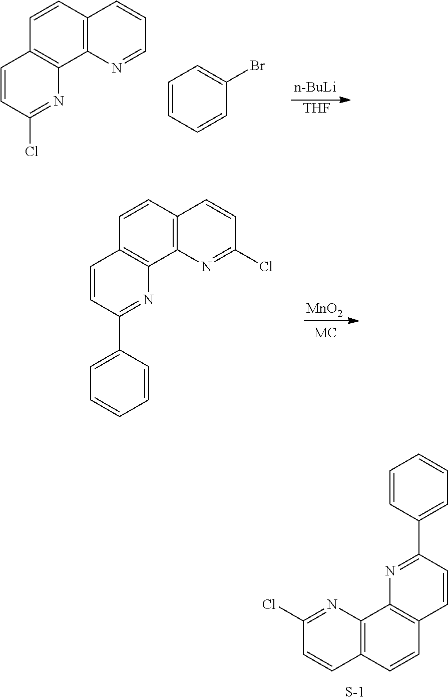

Bromobenzene (7.3 g, 46.6 mmol) was dissolved in 150 ml of dry THE, and then cooled at −78° C. in a nitrogen atmosphere, and subsequently, n-BuLi (29.1 mL of 1.6 M in hexane, 46.6 mmol) was slowly added dropwise. After stirring at −78° C. for 1 hour, 2-chloro-1, 10-phenanthroline (10.0 g, 46.6 mmol) added, followed by reaction for 3 hours, and the reaction was completed by a NH₄Cl solution at room temperature. The mixture that had been reacted was subjected to extraction with EtOAc, followed by washing with Brine. The organic layer that has been washed was concentrated and then dissolved in 150 ml of methylene chloride, and subsequently, MnO₂ (40.5 g, 465.8 mmol) was added, and then the mixture was heated to reflux with stirring for 12 hours. After completion of the reaction, MnO₂ was removed by filtration, and the organic layer was washed with water, concentrated, and then crystallized by acetone and MeOH to give 2-chloro-9-phenyl-1,10-phenanthroline (12.3 g, 42.4 mmol, yield: 91%).

Mass: [(M+H)⁺]: 292

### [Preparation Example 19] Synthesis of 7-chloro-2-phenyl-1, 10-phenanthroline (S-2)

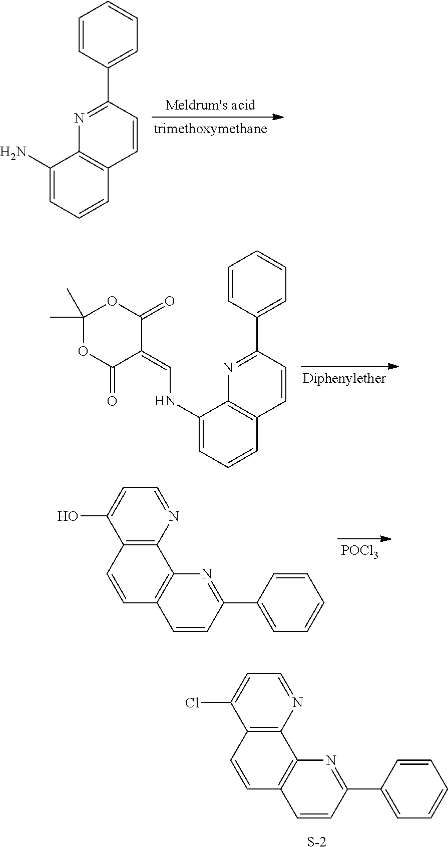

### Step 1. Synthesis of 2, 2-dimethyl-5-(((2-phenylquinolin-8-yl)amino)methylene)-1, 3-dioxane-4, 6-dione

Meldrum's acid (7.4 g, 51.2 mmol) was placed in trimethoxymethane (81.6 g, 768.5 mmol), and then the mixture was heated to reflux with stirring for 2 hours, followed by cooling. Thereafter, 2-phenylquinolin-8-amine (10.0 g, 46.6 mmol) was added, and then the solution was heated to reflux with stirring for 1.5 hours. Upon completion of the reaction, the resultant product was cooled at room temperature, and then the reaction was terminated by a NH₄Cl solution. The mixture that had been reacted was subjected to extraction with EtOAc, followed by washing with Brine. The organic layer that had been washed was dried over magnesium sulfate, concentrated, and then recrystallized with EtOH, to give 2,2-dimethyl-5-(((2-phenylquinolin-8-yl)amino)methylene)-1, 3-dioxane-4, 6-dione (12.2 g, 32.6 mmol, yield: 70%).

Mass: [(M+H)⁺]: 375

### Step 2. Synthesis of 9-phenyl-1,10-phenanthrolin-4-ol

2, 2-Dimethyl-5-(((2-phenylquinolin-8-yl)amino)methylene)-1, 3-dioxane-4, 6-dione (12.2 g, 32.6 mmol) synthesized in Step 1 of Preparation Example 19 was placed in 122 ml of diphenylether, and then the mixture was heated to flux with stirring for 1 hour. Upon completion of the reaction, the resultant product was cooled, and crystallized by addition of petroleum ether, and then the generated crystals were filtered to give 9-phenyl-1,10-phenanthrolin-4-ol (6.3 g, 23.1 mmol, yield: 71%).

Mass: [(M+H)⁺]: 273

### Step 3. Synthesis of 7-chloro-2-phenyl-1,10-phenanthroline

After 9-phenyl-1,10-phenanthrolin-4-ol (6.3 g, 23.1 mmol) synthesized in Step 2 of Preparation Example 19 was added to POCl₃ (71.0 g, 463.0 mmol), the mixture was heated to reflux with stirring for 1 hour. After the solution was cooled, 200 ml of water was slowly added, and then the adjustment to pH 13 was conducted by a 20% NaOH solution. Thereafter, the resultant solution was subjected to extraction with methylene chloride, and subsequently, the extract was dried over magnesium sulfate and then concentrated to give 7-chloro-2-phenyl-1,10-phenanthroline (6.4 g, 22.0 mmol, yield: 95%).

Mass: [(M+H)⁺]: 292

### [Preparation Example 20] Synthesis of 2-chloro-9-(naphthalen-2-yl)-1,10-phenanthroline (S-3)

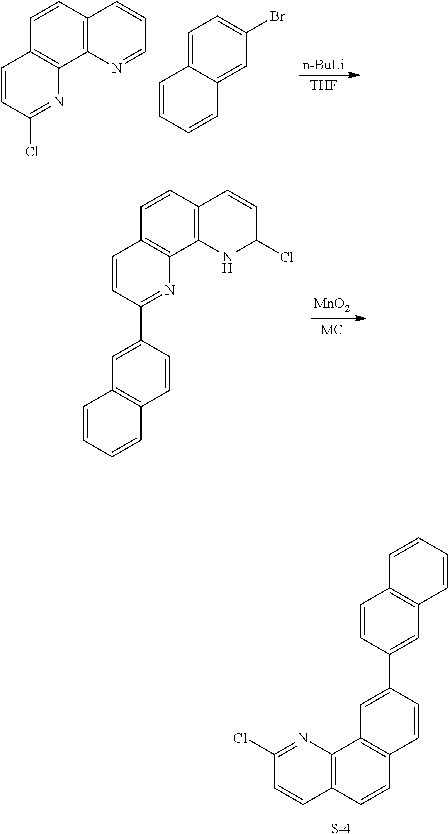

2-bromonaphthalene (9.6 g, 46.6 mmol) was dissolved in 150 ml of dry THE, and then cooled at −78° C. in a nitrogen atmosphere, and subsequently, n-BuLi (29.1 mL of 1.6 M in hexane, 46.6 mmol) was slowly added dropwise. After stirring at −78° C. for 1 hour, 2-chloro-1, 10-phenanthroline (10.0 g, 46.6 mmol) was added, followed by reaction for 3 hours, and the reaction was completed by a NH₄Cl solution at room temperature. The mixture that had been reacted was subjected to extraction with EtOAc, followed by washing with Brine. The organic layer that has been washed was concentrated and then dissolved in 150 ml of methylene chloride, and subsequently, MnO₂ (40.5 g, 465.8 mmol) was added, and then the mixture was heated to reflux with stirring for 12 hours. After completion of the reaction, MnO₂ was removed by filtration, and the organic layer was washed with water, concentrated, and then crystallized by acetone and MeOH to give 2-chloro-9-(naphthalen-2-yl)-1, 10-phenanthroline (14.0 g, 41.0 mmol, yield: 88%).

Mass: [(M+H)⁺]: 342

### [Preparation Example 21] Synthesis of 2-([1,1′: 2′, 1″-terphenyl]-4′-yl)-9-chloro-1, 10-phenanthroline (S-4)

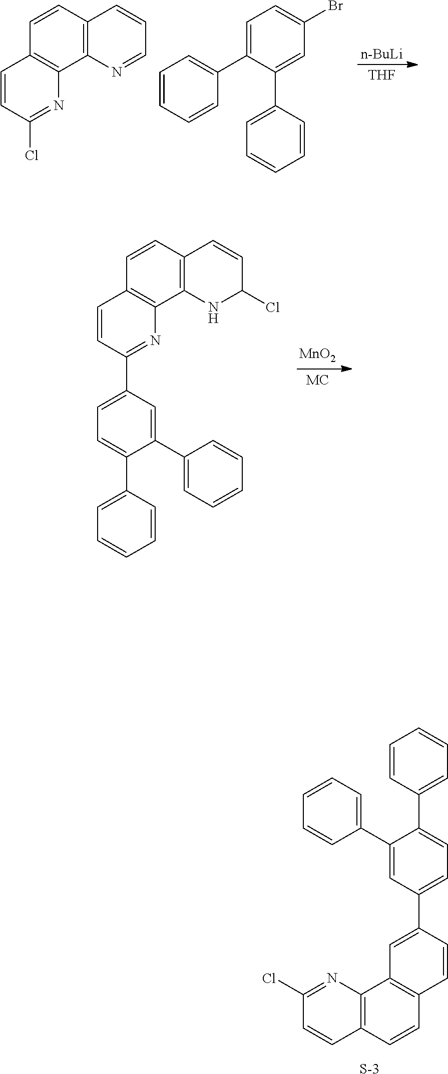

4′-bromo-1,1′: 2′, 1″-terphenyl (14.4 g, 46.6 mmol) was dissolved in 150 ml of dry THE, and then cooled at −78° C. in a nitrogen atmosphere, and subsequently, n-BuLi (29.1 mL of 1.6 M in hexane, 46.6 mmol) was slowly added dropwise. After stirring at −78° C. for 1 hour, 2-chloro-1, 10-phenanthroline (10.0 g, 46.6 mmol) was added, followed by reaction for 3 hours, and the reaction was completed by a NH₄Cl solution at room temperature. The mixture that had been reacted was subjected to extraction with EtOAc, followed by washing with Brine. The organic layer that has been washed was concentrated and then dissolved in 150 ml of methylene chloride, and subsequently, MnO₂ (40.5 g, 465.8 mmol) was added, and then the mixture was heated to reflux with stirring for 2 hours. After completion of the reaction, MnO₂ was removed by filtration, and the organic layer was washed with water, concentrated, and then crystallized by acetone and MeOH to give 2-([1,1′: 2′, 1″-terphenyl]-4′-yl)-9-chloro-1, 10-phenanthroline (18.8 g, 42.4 mmol, yield: 91%).

Mass: [(M+H)⁺]: 444

### [Preparation Example 22] Synthesis of 4-chloro-7-phenyl-1,10-phenanthroline (S-5)

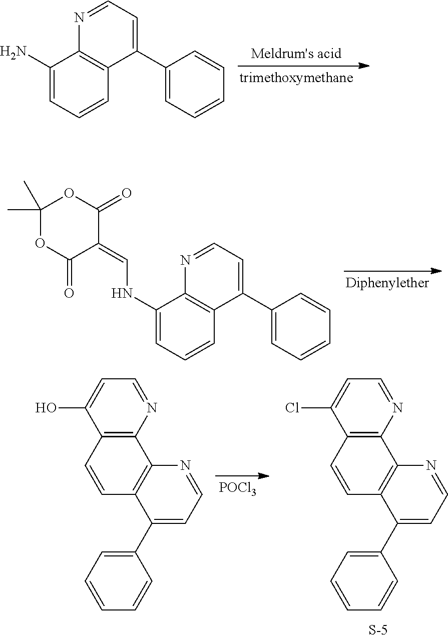

### Step 1. Synthesis of 2, 2-dimethyl-5-(((4-phenylquinolin-8-yl)amino)methylene)-1, 3-dioxane-4, 6-dione

Meldrum's acid (7.4 g, 51.2 mmol) was placed in trimethoxymethane (81.6 g, 768.5 mmol), and then the mixture was heated to reflux with stirring for 2 hours, followed by cooling. Thereafter, 4-phenylquinolin-8-amine (10.0 g, 46.6 mmol) was added, and then the solution was heated to reflux with stirring for 1.5 hours. Upon completion of the reaction, the resultant product was cooled at room temperature, and then the reaction was terminated by a NH₄Cl solution. The mixture that had been reacted was subjected to extraction with EtOAc, followed by washing with Brine. The organic layer that had been washed was dried over magnesium sulfate, concentrated, and then recrystallized with EtOH, to give 2,2-dimethyl-5-(((4-phenylquinolin-8-yl)amino)methylene)-1, 3-dioxane-4, 6-dione (12.7 g, 34.0 mmol, yield: 73%).

Mass: [(M+H)⁺]: 375

### Step 2. Synthesis of 7-phenyl-1,10-phenanthrolin-4-ol

2, 2-Dimethyl-5-(((4-phenylquinolin-8-yl)amino)methylene)-1, 3-dioxane-4, 6-dione (12.7 g, 34.0 mmol) synthesized in Step 1 of Preparation Example 22 was placed in 128 ml of diphenylether, and then the mixture was heated to flux with stirring for 1 hour. Upon completion of the reaction, the resultant product was cooled, and crystallized by addition of petroleum ether, and then the generated crystals were filtered to give 7-phenyl-1,10-phenanthrolin-4-ol (6.7 g, 24.5 mmol, yield: 72%).

Mass: [(M+H)⁺]: 273

### Step 3. Synthesis of 4-chloro-7-phenyl-1,10-phenanthroline

After 7-phenyl-1,10-phenanthrolin-4-ol (6.7 g, 24.5 mmol) synthesized in Step 2 of Preparation Example 22 was added to POCl₃ (75.1 g, 489.6 mmol), the mixture was heated to reflux with stirring for 1 hour. After the solution was cooled, 200 ml of water was slowly added, and then the adjustment to pH 13 was conducted by a 20% NaOH solution. Thereafter, the resultant solution was subjected to extraction with methylene chloride, and subsequently, the extract was dried over magnesium sulfate and then concentrated to give 4-chloro-7-phenyl-1,10-phenanthroline (6.5 g, 22.5 mmol, yield: 92%).

Mass: [(M+H)⁺]: 292

### [Synthesis Example 1] Synthesis of 2-phenyl-9-(3-(6-(quinolin-2-yl)pyridin-2-yl)phenyl)-1,10-phenanthroline (001)

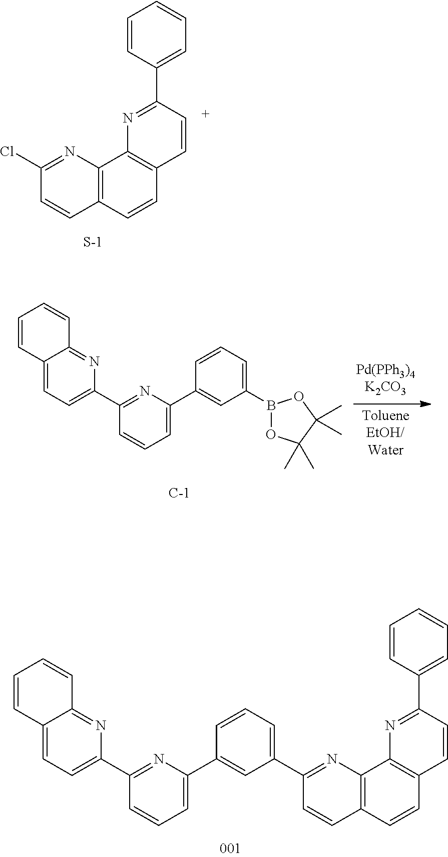

Compound S-1 (10.0 g, 34.4 mmol) synthesized by the method in Preparation Example 18, Compound C-1 (14.0 g, 34.4 mmol) synthesized by the method in Preparation Example 11, Pd(PPh₃)₄ (1.2 g, 1.0 mmol), and K₂CO₃ (14.3 g, 103.2 mmol) were placed in 150 ml of toluene, 30 ml of EtOH, and 30 ml of water, and then the mixture was heated to reflux with stirring for 2 hours. After completion of the reaction, the organic layer was extracted by methylene chloride, and the extracted organic layer was dried over magnesium sulfate, concentrated, and then purified by column chromatography to give 2-phenyl-9-(3-(6-(quinolin-2-yl)pyridin-2-yl)phenyl)-1, 10-phenanthroline (16.6 g, 30.9 mmol, yield: 90%).

Mass: [(M+H)⁺]: 538

### [Synthesis Example 2] Synthesis of 2-(3-(6-(phenanthridin-6-yl)pyridin-2-yl)phenyl)-9-phenyl-1,10-phenanthroline (002)

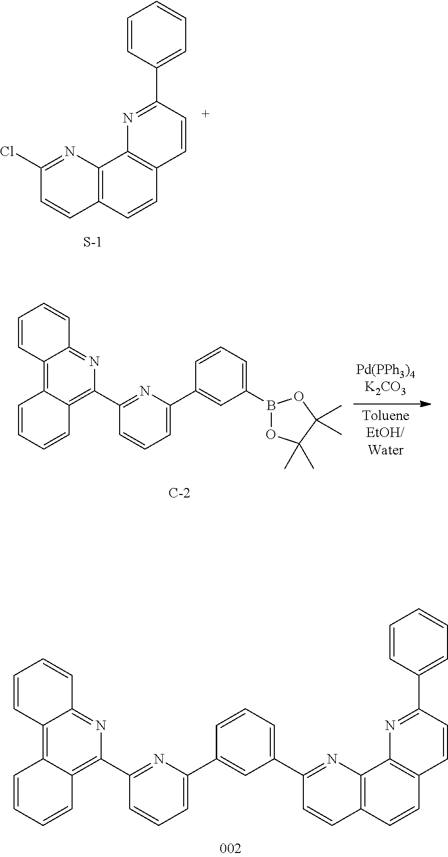

Compound S-1 (10.0 g, 34.4 mmol) synthesized by the method in Preparation Example 18, Compound C-2 (15.8 g, 34.4 mmol) synthesized by the method in Preparation Example 12, Pd(PPh₃)₄ (1.2 g, 1.0 mmol), and K₂CO₃ (14.3 g, 103.2 mmol) were placed in 150 ml of toluene, 30 ml of EtOH, and 30 ml of water, and then the mixture was heated to reflux with stirring for 2 hours. After completion of the reaction, the organic layer was extracted by methylene chloride, and the extracted organic layer was dried over magnesium sulfate, concentrated, and then purified by column chromatography to give 2-(3-(6-(phenanthridin-6-yl)pyridin-2-yl)phenyl)-9-phenyl-1,10-phenanthroline (17.8 g, 30.3 mmol, yield: 88%).

Mass: [(M+H)⁺]: 588

### [Synthesis Example 3] Synthesis of 6-(6-(3-(9-phenyl-1,10-phenanthrolin-2-yl)phenyl)pyridin-2-yl)benzo[c]phenanthridine (003)

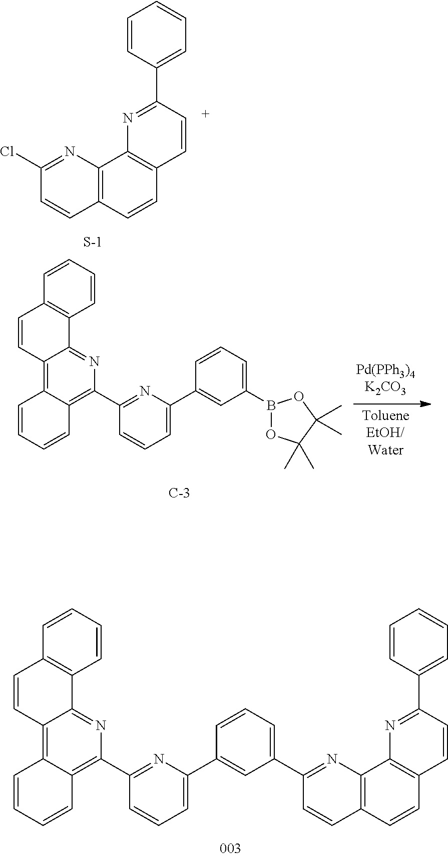

Compound S-1 (10.0 g, 34.4 mmol) synthesized by the method in Preparation Example 18, Compound C-3 (17.5 g, 34.4 mmol) synthesized by the method in Preparation Example 13, Pd(PPh₃)₄ (1.2 g, 1.0 mmol), and K₂CO₃ (14.3 g, 103.2 mmol) were placed in 150 ml of toluene, 30 ml of EtOH, and 30 ml of water, and then the mixture was heated to reflux with stirring for 2 hours. After completion of the reaction, the organic layer was extracted by methylene chloride, and the extracted organic layer was dried over magnesium sulfate, concentrated, and then purified by column chromatography to give Jun. 16, 2013-(9-phenyl-1,10-phenanthrolin-2-yl)phenyl)pyridin-2-yl)benzo[c]phenanthridine (19.1 g, 29.9 mmol, yield: 87%).

Mass: [(M+H)⁺]: 638

### [Synthesis Example 4] Synthesis of 2-(4-(6-(phenanthridin-6-yl)pyridin-2-yl)phenyl)-9-phenyl-1,10-phenanthroline (011)

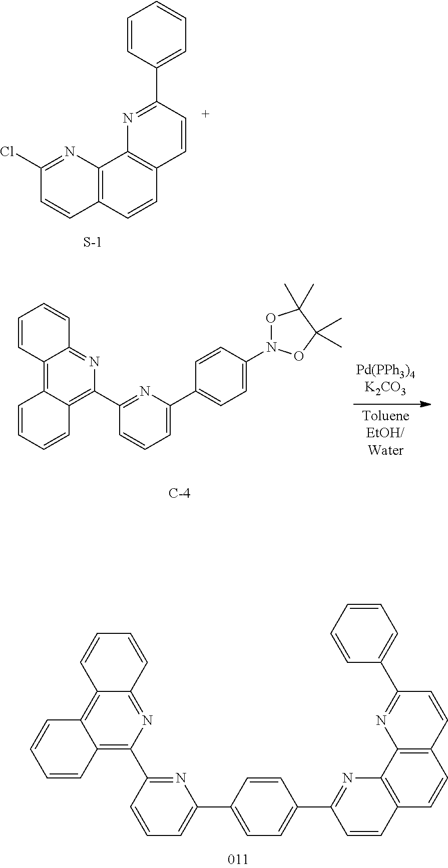

Compound S-1 (10.0 g, 34.4 mmol) synthesized by the method in Preparation Example 18, Compound C-4 (15.8 g, 34.4 mmol) synthesized by the method in Preparation Example 14, Pd(PPh₃)₄ (1.2 g, 1.0 mmol), and K₂CO₃ (14.3 g, 103.2 mmol) were placed in 150 ml of toluene, 30 ml of EtOH, and 30 ml of water, and then the mixture was heated to reflux with stirring for 2 hours. After completion of the reaction, the organic layer was extracted by methylene chloride, and the extracted organic layer was dried over magnesium sulfate, concentrated, and then purified by column chromatography to give 2-(4-(6-(phenanthridin-6-yl)pyridin-2-yl)phenyl)-9-phenyl-1,10-phenanthroline (17.8 g, 30.3 mmol, yield: 88%).

Mass: [(M+H)⁺]: 588

### [Synthesis Example 5] Synthesis of 2-(4-(6-(phenanthridin-6-yl)pyridin-2-yl) naphthalen-2-yl)-9-phenyl-1,10-phenanthroline (013)

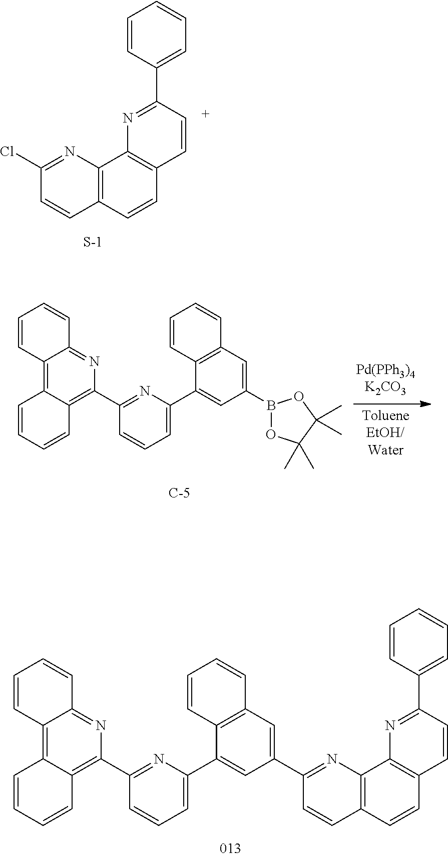

Compound S-1 (10.0 g, 34.4 mmol) synthesized by the method in Preparation Example 18, Compound C-5 (17.5 g, 34.4 mmol) synthesized by the method in Preparation Example 15, Pd(PPh₃)₄ (1.2 g, 1.0 mmol), and K₂CO₃ (14.3 g, 103.2 mmol) were placed in 150 ml of toluene, 30 ml of EtOH, and 30 ml of water, and then the mixture was heated to reflux with stirring for 2 hours. After completion of the reaction, the organic layer was extracted by methylene chloride, and the extracted organic layer was dried over magnesium sulfate, concentrated, and then purified by column chromatography to give 2-(4-(6-(phenanthridin-6-yl)pyridin-2-yl) naphthalen-2-yl)-9-phenyl-1,10-phenanthroline (19.7 g, 30.9 mmol, yield: 90%).

Mass: [(M+H)⁺]: 638

### [Synthesis Example 6] Synthesis of 2-(7-(6-(phenanthridin-6-yl)pyridin-2-yl) naphthalen-2-yl)-9-phenyl-1,10-phenanthroline (022)

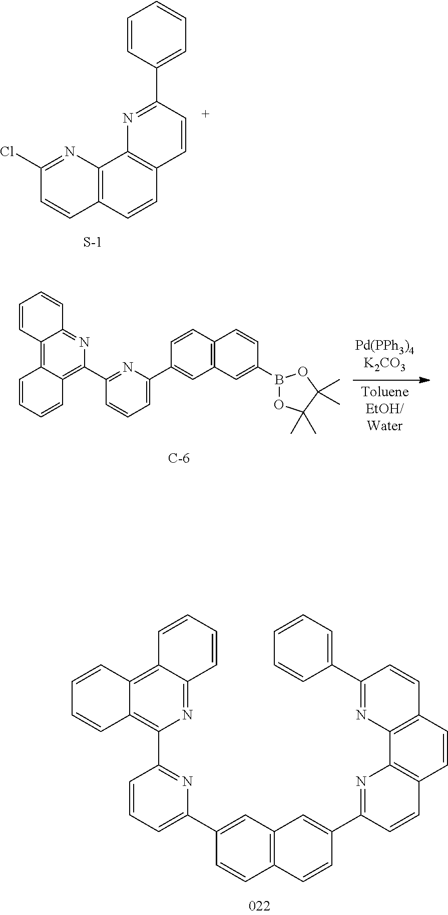

Compound S-1 (10.0 g, 34.4 mmol) synthesized by the method in Preparation Example 18, Compound C-6 (17.5 g, 34.4 mmol) synthesized by the method in Preparation Example 16, Pd(PPh₃)₄ (1.2 g, 1.0 mmol), and K₂CO₃ (14.3 g, 103.2 mmol) were placed in 150 ml of toluene, 30 ml of EtOH, and 30 ml of water, and then the mixture was heated to reflux with stirring for 2 hours. After completion of the reaction, the organic layer was extracted by methylene chloride, and the extracted organic layer was dried over magnesium sulfate, concentrated, and then purified by column chromatography to give 2-(7-(6-(phenanthridin-6-yl)pyridin-2-yl) naphthalen-2-yl)-9-phenyl-1,10-phenanthroline (18.6 g, 29.2 mmol, yield: 85%).

Mass: [(M+H)⁺]: 638

### [Synthesis Example 7] Synthesis of 6-(6-(7-(9-phenyl-1,10-phenanthrolin-2-yl) naphthalen-2-yl)pyridin-2-yl)benzo[c]phenanthridine (062)

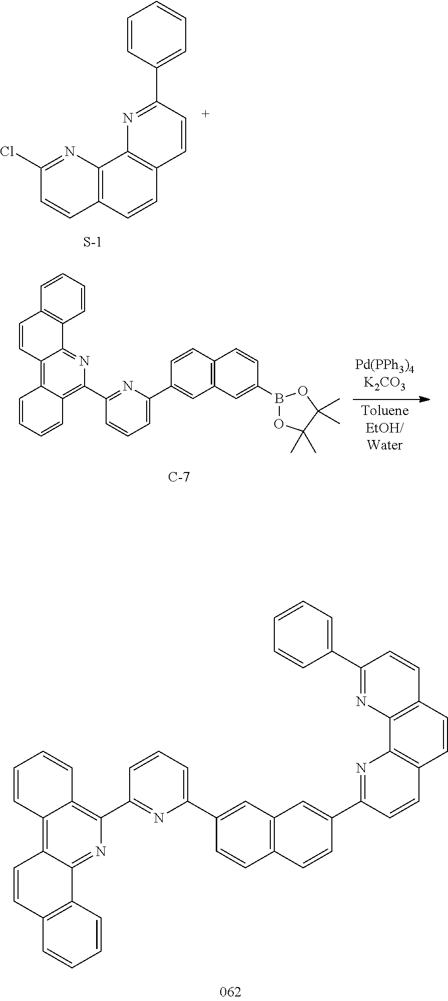

Compound S-1 (10.0 g, 34.4 mmol) synthesized by the method in Preparation Example 18, Compound C-7 (19.2 g, 34.4 mmol) synthesized by the method in Preparation Example 17, Pd(PPh₃)₄ (1.2 g, 1.0 mmol), and K₂CO₃ (14.3 g, 103.2 mmol) were placed in 150 ml of toluene, 30 ml of EtOH, and 30 ml of water, and then the mixture was heated to reflux with stirring for 2 hours. After completion of the reaction, the organic layer was extracted by methylene chloride, and the extracted organic layer was dried over magnesium sulfate, concentrated, and then purified by column chromatography to give 6-(6-(7-(9-phenyl-1,10-phenanthrolin-2-yl) naphthalen-2-yl)pyridin-2-yl)benzo[c]phenanthridine (20.1 g, 29.2 mmol, yield: 85%).

Mass: [(M+H)⁺]: 688

### [Synthesis Example 8] Synthesis of 6-(6-(3-(9-phenyl-1, 10-phenanthrolin-4-yl)phenyl)pyridin-2-yl)benzo[c]phenanthridine (072)

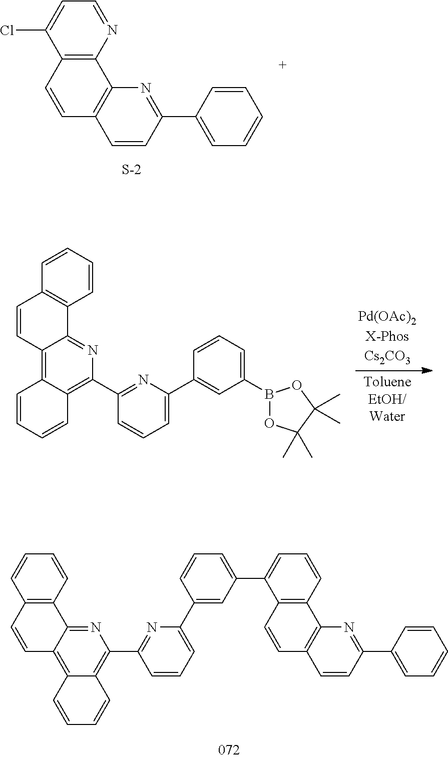

Compound S-2 (10.0 g, 34.4 mmol) synthesized by the method in Preparation Example 19, Compound C-3 (17.5 g, 34.4 mmol) synthesized by the method in Preparation Example 13, and Cs₂CO₃ (33.6 g, 103.2 mmol) were placed in 150 ml of toluene, 30 ml of EtOH, and 30 ml of water, and then Pd(OAc) 2 (0.2 g, 1.0 mmol) and X-Phos (1.0 g, 2.1 mmol) were added. Thereafter, the mixture was heated to reflux with stirring for 4 hours. After completion of the reaction, the organic layer was extracted by methylene chloride, and the extracted organic layer was dried over magnesium sulfate, concentrated, and then purified by column chromatography to give 6-(6-(3-(9-phenyl-1,10-phenanthrolin-4-yl)phenyl)pyridin-2-yl)benzo[c]phenanthridine (18.8 g, 29.6 mmol, yield: 86%).

Mass: [(M+H)⁺]: 638

### [Synthesis Example 9] Synthesis of 6-(6-(3-(9-([1,1′: 2′, 1″-terphenyl]-4′-yl)-1, 10-phenanthrolin-2-yl)phenyl)pyridin-2-yl)benzo[c]phenanthridine (074)

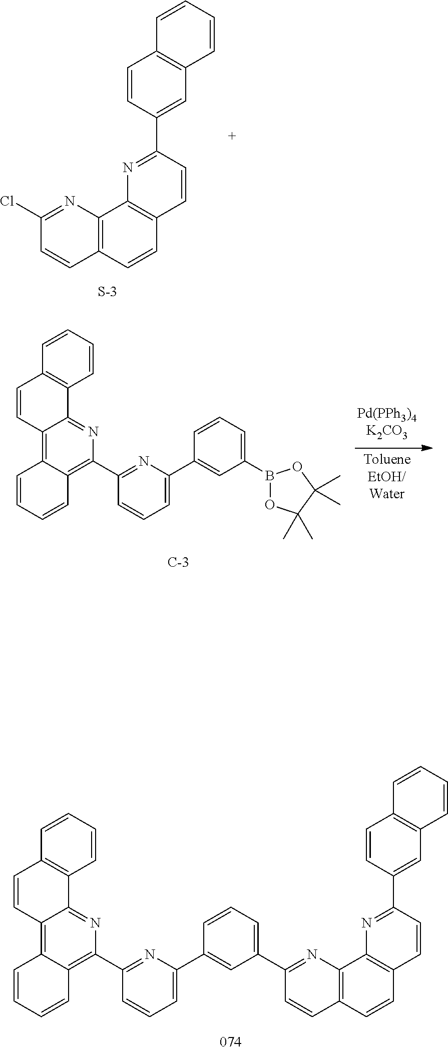

Compound S-3 (10.0 g, 22.6 mmol) synthesized by the method in Preparation Example 20, Compound C-3 (11.5 g, 22.6 mmol) synthesized by the method in Preparation Example 13, Pd(PPh₃)₄ (0.8 g, 0.7 mmol), and K₂CO₃ (9.4 g, 67.7 mmol) were placed in 150 ml of toluene, 30 ml of EtOH, and 30 ml of water, and then the mixture was heated to reflux with stirring for 2 hours. After completion of the reaction, the organic layer was extracted by methylene chloride, and the extracted organic layer was dried over magnesium sulfate, concentrated, and then purified by column chromatography to give 6-(6-(3-(9-([1,1′: 2′, 1″-terphenyl]-4′-yl)-1, 10-phenanthrolin-2-yl)phenyl)pyridin-2-yl)benzo[c]phenanthridine (15.0 g, 19.0 mmol, yield: 84%).

Mass: [(M+H)⁺]: 790

### [Synthesis Example 10] Synthesis of 6-(6-(3-(9-(naphthalen-2-yl)-1, 10-phenanthrolin-2-yl)phenyl)pyridin-2-yl)benzo[c]phenanthridine (083)

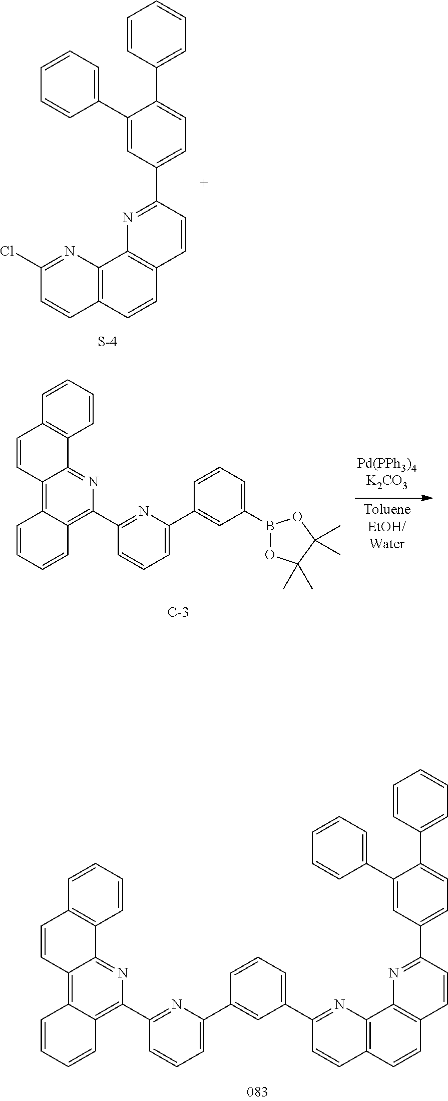

Compound S-4 (10.0 g, 29.3 mmol) synthesized by the method in Preparation Example 21, Compound C-3 (14.9 g, 29.3 mmol) synthesized by the method in Preparation Example 13, Pd(PPh₃)₄ (1.0 g, 0.9 mmol), and K₂CO₃ (12.2 g, 88.0 mmol) were placed in 150 ml of toluene, 30 ml of EtOH, and 30 ml of water, and then the mixture was heated to reflux with stirring for 2 hours. After completion of the reaction, the organic layer was extracted by methylene chloride, and the extracted organic layer was dried over magnesium sulfate, concentrated, and then purified by column chromatography to give 6-(6-(3-(9-(naphthalen-2-yl)-1, 10-phenanthrolin-2-yl)phenyl)pyridin-2-yl)benzo[c]phenanthridine (17.1 g, 24.9 mmol, yield: 85%).

Mass: [(M+H)⁺]: 688

### [Synthesis Example 11] Synthesis of 4-(3-(6-(phenanthridin-6-yl)pyridin-2-yl)phenyl)-7-phenyl-1,10-phenanthroline (096)

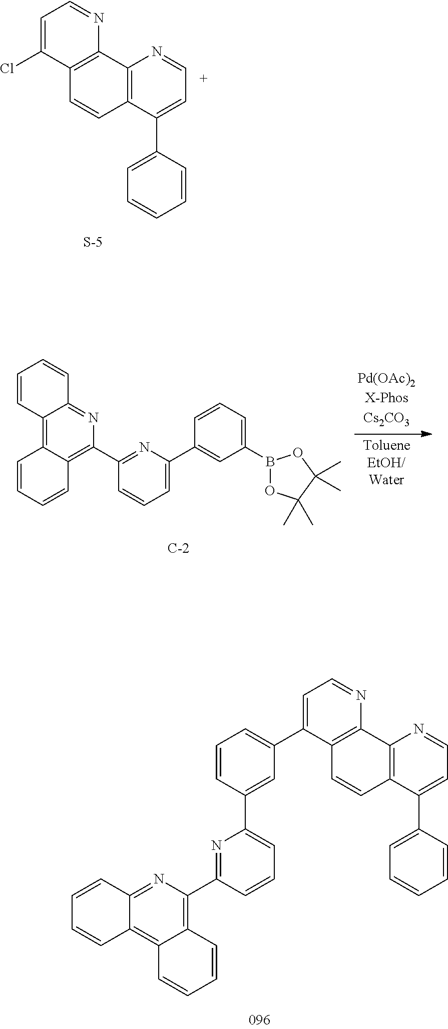

Compound S-5 (10.0 g, 34.4 mmol) synthesized by the method in Preparation Example 22, Compound C-2 (15.8 g, 34.4 mmol) synthesized by the method in Preparation Example 12, and Cs₂CO₃ (33.6 g, 103.2 mmol) were placed in 150 ml of toluene, 30 ml of EtOH, and 30 ml of water, and then Pd(OAc) 2 (0.2 g, 1.0 mmol) and X-Phos (1.0 g, 2.1 mmol) were added. Thereafter, the mixture was heated to reflux with stirring for 4 hours. After completion of the reaction, the organic layer was extracted by methylene chloride, and the extracted organic layer was dried over magnesium sulfate, concentrated, and then purified by column chromatography to give 4-(3-(6-(phenanthridin-6-yl)pyridin-2-yl)phenyl)-7-phenyl-1,10-phenanthroline (16.7 g, 28.5 mmol, yield: 83%).

Mass: [(M+H)⁺]: 588

### [Embodiment 1] Manufacture of Blue Organic Electroluminescent Device

Compound 001 synthesized in Synthesis Example 1 was subjected to high-purity sublimation purification by a commonly known method, and then a blue organic electroluminescent device was manufactured as follows.

First a glass substrate coated with a 1200 Å-thick indium tin oxide (ITO) thin film was ultrasonically washed with distilled water. After completion of washing with distilled water, the glass substrate was ultrasonically washed with a solvent, such as isopropyl alcohol, acetone, or methanol, dried, and then transferred to a UV Ozone cleaner (Power sonic 405, Hwashin Tech). Subsequently, the substrate was cleaned for 5 minutes by using UV and transferred to a vacuum deposition system.

On the ITO transparent electrode prepared as above, Compound 1 and Compound 2 were co-deposited at a weight ratio of 98:2 to form a hole injection layer with a thickness of 100 Å. Thereafter, Compound 1 was deposited on the hole injection layer to a form a hole transport layer with a thickness of 1400 Å, and then Compound 3 was deposited to a thickness of 50 Å on the hole transport layer to form a hole transport auxiliary layer. Thereafter, Compound 4 and Compound 5 were co-deposited at a weight ratio of 98:2 to form a light-emitting layer with a thickness of 200 Å. Thereafter, Compound 6 was deposited on the light-emitting layer to form an electron transport auxiliary layer with a thickness of 50 Å, and then Compound 001 and Compound 7 were co-deposited at a weight ratio of 1:1 to form an electron transport layer with a thickness of 300 Å. Thereafter, LiF was deposited on the electron transport layer to form an electron injection layer with a thickness of 10 Å, and then Al was deposited on the electron injection layer to form a cathode with a thickness of 1000 Å, thereby manufacturing a blue organic electroluminescent device. The structures of Compounds 1 to 7 used are as follows.

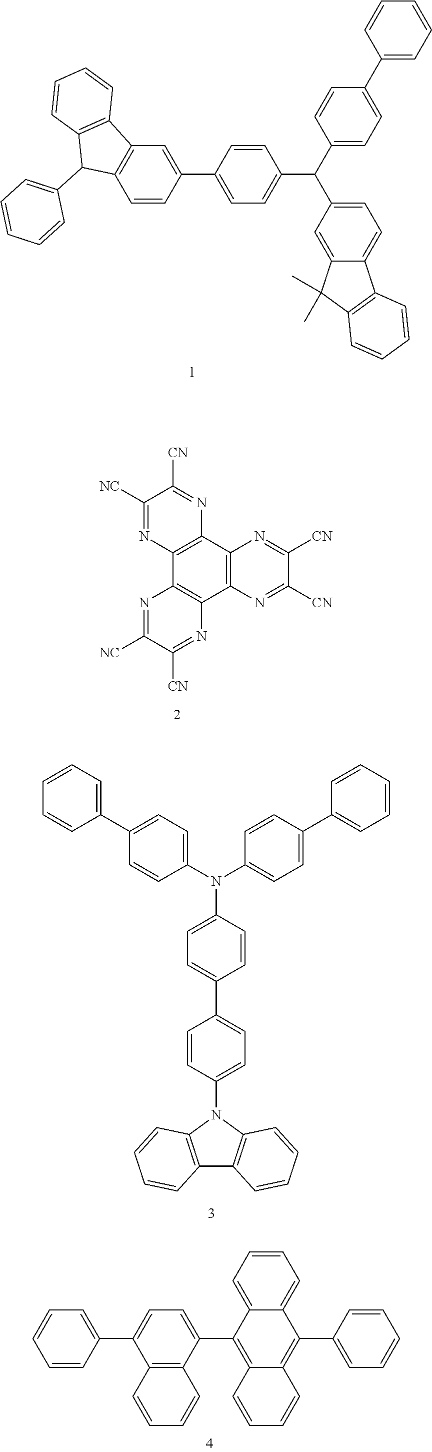

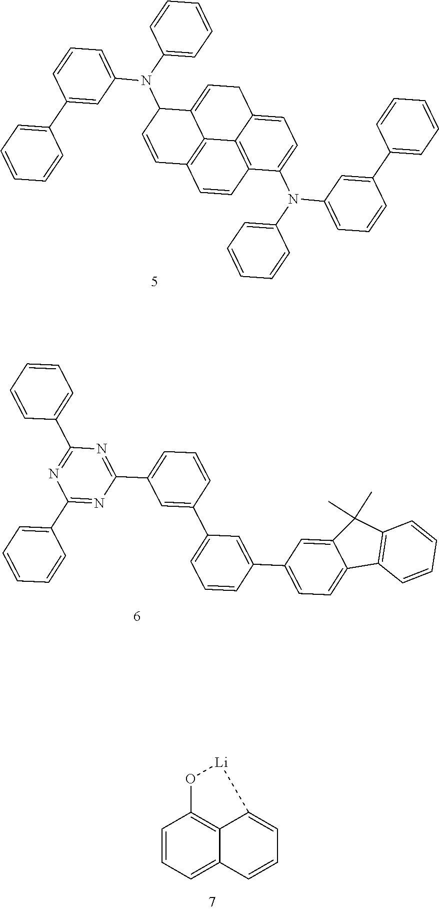

### [Embodiments 2 to 11] Fabrication of Blue Organic EL Devices

Blue organic EL devices were fabricated in the same manner as in Embodiment 1, except that the compounds shown in Table 1 were respectively used instead of Compound 001 which was used as the electron transport layer material in Embodiment 1.

### [Comparative Examples 1 to 5] Fabrication of Blue Organic EL Device

Blue organic EL devices were fabricated in the same manner as in Embodiment 1, except that the following compounds A to E were respectively used instead of Compound 001 which was used as the electron transport layer material in Embodiment 1.

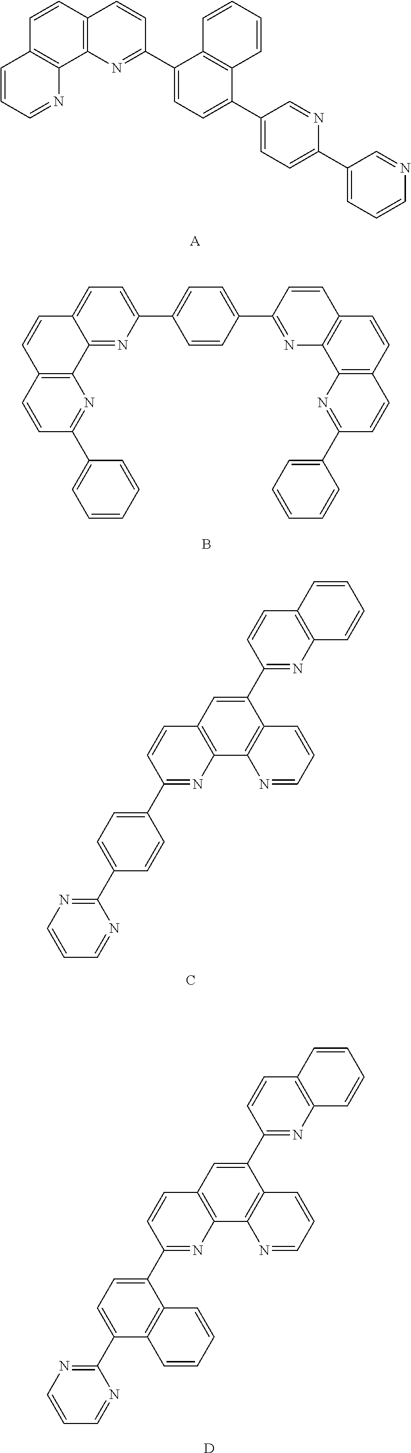

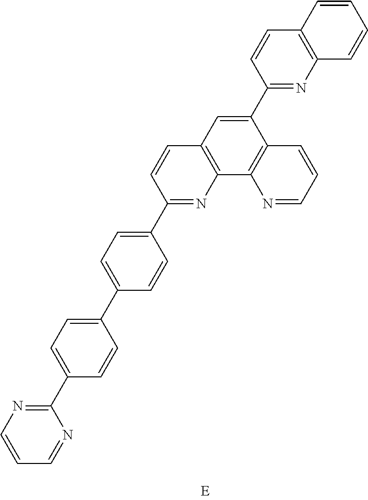 [SMILES: c1cnc(-c2ccc(-c3ccc(-c4ccc5cc(-c6ccc7ccccc7n6)c6cccnc6c5n4)cc3)cc2)nc1]

### Evaluation Example 1

For each of the organic EL devices fabricated in Embodiments 1 to 11 and Comparative Examples 1 to 5, a driving voltage, an emission wavelength, current efficiency, and lifespan at a current density of 10 mA/cm² were measured, and the results are shown in Table 1 below.

TABLE 1

| | electron | Driving | Emission | Current | |
|---|---|---|---|---|---|
| | transport | voltage | peak | efficiency | lifespan |
| Sample | layer | (V) | (nm) | (cd/A) | (h) |
| Embodiment | Compound | 3.8 | 457 | 7.9 | 328 |
| 1 | 001 | | | | |
| Embodiment | Compound | 4.0 | 456 | 8.1 | 320 |
| 2 | 002 | | | | |
| Embodiment | Compound | 3.9 | 456 | 8.0 | 317 |
| 3 | 003 | | | | |
| Embodiment | Compound | 4.0 | 457 | 8.1 | 298 |
| 4 | 011 | | | | |
| Embodiment | Compound | 4.1 | 456 | 8.3 | 285 |
| 5 | 013 | | | | |
| Embodiment | Compound | 3.7 | 458 | 8.2 | 312 |
| 6 | 022 | | | | |
| Embodiment | Compound | 3.8 | 458 | 8.4 | 326 |
| 7 | 062 | | | | |
| Embodiment | Compound | 3.9 | 454 | 7.9 | 250 |
| 8 | 072 | | | | |
| Embodiment | Compound | 4.0 | 457 | 8.2 | 380 |
| 9 | 074 | | | | |
| Embodiment | Compound | 4.0 | 458 | 8.3 | 376 |
| 10 | 083 | | | | |
| Embodiment | Compound | 3.8 | 454 | 8.5 | 298 |
| 11 | 096 | | | | |
| Comp. Ex. | Compound | 4.5 | 456 | 6.7 | 210 |
| 1 | A | | | | |
| Comp. Ex. | Compound | 4.5 | 458 | 6.8 | 220 |
| 2 | B | | | | |
| Comp. Ex. | Compound | 4.7 | 459 | 6.7 | 180 |
| 3 | C | | | | |
| Comp. Ex. | Compound | 4.9 | 459 | 6.8 | 165 |
| 4 | D | | | | |
| Comp. Ex. | Compound | 4.7 | 460 | 6.2 | 150 |
| 5 | E | | | | |

As shown in Table 1 above, the compound according to the present disclosure has a structure in which a pyridine moiety substituted with an N-containing heteroaryl group having 10 to 40 ring members is linked to a phenanthroline moiety via a linker. The blue organic electroluminescent devices of Embodiments 1 to 11, in which the compounds of the present disclosure were used in an electron transport layer, exhibited superior performance in terms of driving voltage, light emission peaks, current efficiency, and device lifespan, compared to the organic electroluminescent devices of Comparative Examples 1 to 5, in which compounds lacking a pyridine moiety substituted with an N-containing heteroaryl group having 10 to 40 ring members were in an electron transport layer.

### [Embodiment 12] Manufacture of Organic Electroluminescent Device

Compound 001 synthesized in Synthesis Example 1 was subjected to high-purity sublimation purification by a commonly known method, and then a blue organic electroluminescent device was manufactured as follows.

First a glass substrate coated with a 1500 Å-thick indium tin oxide (ITO) thin film was ultrasonically washed with distilled water. After completion of washing with distilled water, the glass substrate was ultrasonically washed with a solvent, such as isopropyl alcohol, acetone, or methanol, dried, and then transferred to a UV Ozone cleaner (Power sonic 405, Hwashin Tech). Subsequently, the substrate was cleaned for 5 minutes by using UV and transferred to a vacuum deposition system.

On the ITO transparent electrode prepared as above, Compound 1 and Compound 2 were co-deposited at a weight ratio of 98:2 to form a hole injection layer with a thickness of 100 Å. Thereafter, Compound 1 was deposited on the hole injection layer to a form a hole transport layer with a thickness of 200 Å, and then Compound 3 was deposited to a thickness of 50 Å on the hole transport layer to form a hole transport auxiliary layer. Thereafter, Compound 4 and Compound 5 were co-deposited at a weight ratio of 98:2 to form a light-emitting layer with a thickness of 200 Å. Thereafter, Compound 7 was deposited to a thickness of 150 Å on the light-emitting layer to form an electron transport region, and then Compound 001 was deposited to a thickness of 80 Å on the electron transport region to form an N-type charge generation layer. Thereafter, Compounds 1 and 2 were co-deposited at a weight ratio of 98:2 on the N-type charge generation layer to form a P-type charge generation layer with a thickness of 100 Å. Then, Compound 1 was deposited on the P-type charge generation layer to form a hole transport layer with a thickness of 350 Å, and Compound 3 was subsequently deposited to a thickness of 50 Å on the hole transport layer to form a hole transport auxiliary layer. And then, Compounds 4 and 5 were co-deposited at a weight ratio of 98:2 to form a light-emitting layer with a thickness of 200 Å. Thereafter, Compound 6 was deposited on the light-emitting layer to form an electron transport auxiliary layer with a thickness of 50 Å, and then Compounds 7 and 8 were co-deposited at a weight ratio of 1:1 to form an electron transport layer with a thickness of 300 Å. Thereafter, LiF was deposited on the electron transport layer to form an electron injection layer with a thickness of 10 Å, and then Al was deposited on the electron injection layer to form a cathode with a thickness of 1000 Å, thereby manufacturing an organic electroluminescent device. The structures of Compounds 1 to 7 used herein are the same as those described in Embodiment 1, and the structure of Compound 8 is as follows.

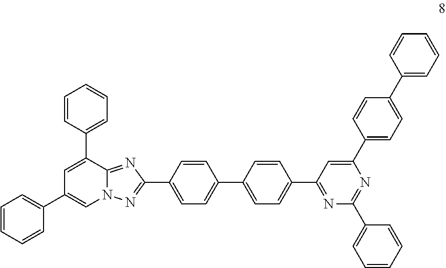 [SMILES: c1ccc(-c2ccc(-c3cc(-c4ccc(-c5ccc(-c6nc7c(-c8ccccc8)cc(-c8ccccc8)cn7n6)cc5)cc4)nc(-c4ccccc4)n3)cc2)cc1]

### [Embodiments 13 to 22] Fabrication of Blue Organic EL Devices

Organic EL devices were fabricated in the same manner as in Embodiment 12, except that the compounds shown in Table 2 were respectively used instead of Compound 001 which was used as the charge generation layer material in Embodiment 12.

### [Comparative Examples 6 to 10] Fabrication of Organic EL Device

Organic EL devices were fabricated in the same manner as in Embodiment 12, except that Compounds A to E were respectively used instead of Compound 001 which was used as the charge generation layer material in Embodiment 12. The structures of Compounds A to E used herein are the same as those described in Comparative Examples 1 to 5.

### Evaluation Example 2

For each of the organic EL devices fabricated in Embodiments 12 to 22 and Comparative Examples 6 to 10, a driving voltage, current efficiency, and an emission wavelength at a current density of 10 mA/cm² were measured, and the results are shown in Table 2 below.

TABLE 2

| | N-type charge | Driving | Current | |
|---|---|---|---|---|
| | generation | voltage | efficiency | lifespan |
| Sample | layer | (V) | (cd/A) | (h) |
| Embodiment | Compound 001 | 7.8 | 15.9 | 320 |
| 12 | | | | |
| Embodiment | Compound 002 | 8.0 | 15.8 | 350 |
| 13 | | | | |
| Embodiment | Compound 003 | 8.0 | 15.5 | 351 |
| 14 | | | | |
| Embodiment | Compound 011 | 7.9 | 15.5 | 351 |
| 15 | | | | |
| Embodiment | Compound 013 | 7.9 | 15.6 | 349 |
| 16 | | | | |
| Embodiment | Compound 022 | 7.9 | 15.6 | 340 |
| 17 | | | | |
| Embodiment | Compound 062 | 7.7 | 15.6 | 340 |
| 18 | | | | |
| Embodiment | Compound 072 | 8.0 | 15.7 | 352 |
| 19 | | | | |
| Embodiment | Compound 074 | 8.0 | 15.4 | 351 |
| 20 | | | | |
| Embodiment | Compound 083 | 7.9 | 15.5 | 355 |
| 21 | | | | |
| Embodiment | Compound 096 | 7.9 | 15.6 | 333 |
| 22 | | | | |
| Comp. Ex. | Compound A | 8.9 | 14.1 | 150 |
| 6 | | | | |
| Comp. Ex. | Compound B | 8.7 | 14.2 | 160 |
| 7 | | | | |
| Comp. Ex. | Compound C | 8.8 | 14.0 | 160 |
| 8 | | | | |
| Comp. Ex. | Compound D | 8.9 | 14.0 | 162 |
| 9 | | | | |
| Comp. Ex. | Compound E | 9.0 | 12.0 | 172 |
| 10 | | | | |

As shown in Table 2 above, the compound according to the present disclosure has a structure in which a pyridine moiety substituted with an N-containing heteroaryl group having 10 to 40 ring members is linked to a phenanthroline moiety via a linker. The organic electroluminescent devices of Embodiments 12 to 22, in which the compounds of the present disclosure were used in a N-type charge generation layer, exhibited superior performance in terms of driving voltage, light emission peaks, current efficiency, and device lifespan, compared to the organic electroluminescent devices of Comparative Examples 6 to 10, in which compounds lacking a pyridine moiety substituted with an N-containing heteroaryl group having 10 to 40 ring members were in a N-type charge generation layer.

1. A compound represented by the following Chemical Formula 1: 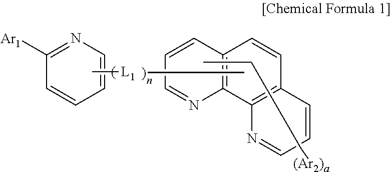
(wherein,
Ar₁ is a C9-C40 heteroaryl group containing one nitrogen (N) atom,
n is an integer in a range of 1 to 3,
L₁ is a C6-C40 arylene group,
a is an integer in a range of 0 to 7,
Ar₂ is selected from the group consisting of: hydrogen, deuterium (D), halogen group, cyano group, nitro group, amino group, C1-C40 alkyl group, C2-C40 alkenyl group, C2-C40 alkynyl group, C3-C40 cycloalkyl group, heterocycloalkyl group having 3 to 40 nuclear atoms, C6-C60 aryl group, heteroaryl group having 5 to 60 nuclear atoms, C1-C40 alkyloxy group, C6-C60 aryloxy group, C1-C40 alkylsilyl group, C6-C60 arylsilyl group, C1-C40 alkylboron group, C6-C60 arylboron group, C6-C60 arylphosphine group, C6-C60 arylphosphine oxide group, C6-C60 arylamine group, (C6-C60 aryl) (heteroaryl having 5 to 60 nuclear atoms) amine group, and heteroarylamine group having 5 to 60 nuclear atoms, or fused with an adjacent group to form a fused ring;
the heteroaryl group of Ar₁, the arylene group of L₁, and the alkyl group, alkenyl group, alkynyl group, cycloalkyl group, heterocycloalkyl group, aryl group, heteroaryl group, alkyloxy group, aryloxy group, alkylsilyl group, arylsilyl group, alkylboron group, arylboron group, arylphosphine group, arylphosphine oxide, arylamine group, (aryl) (heteroaryl)amine, and heteroarylamine group of Ar₂ are each independently substituted or unsubstituted with one or more substituents selected from the group consisting of: deuterium (D), halogen group, cyano group, nitro group, amino group, C1-C40 alkyl group, C2-C40 alkenyl group, C2-C40 alkynyl group, C3-C40 cycloalkyl group, heterocycloalkyl group having 3 to 40 nuclear atoms, C6-C60 aryl group, heteroaryl group having 5 to 60 nuclear atoms, C1-C40 alkyloxy group, C6-C60 aryloxy group, C1-C40 alkylsilyl group, C6-C60 arylsilyl group, C1-C40 alkylboron group, C6-C60 arylboron group, C6-C60 arylphosphine group, C6-C60 arylphosphine oxide group, C6-C60 arylamine group, (C6-C60 aryl) (heteroaryl having 5 to 60 nuclear atoms) amine group, and heteroarylamine group having 5 to 60 nuclear atoms, and when a plurality of the substituents are present, they are the same as or different from each other).

2. The compound of claim 1,
wherein Ar₁ is a substituent represented by the following Chemical Formula S1-1 or S1-2: 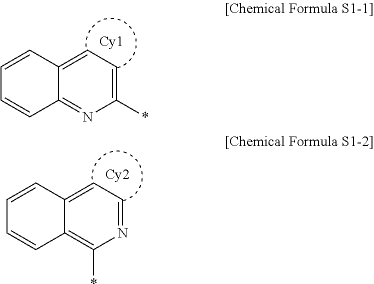
(wherein in the above Chemical Formulas S1-1 and S1-2,
Cy1 and Cy2 are each independently a C6-C30 fused aromatic ring,
the fused aromatic ring of Cy1 and Cy2 are each independently substituted or unsubstituted with one or more substituents selected from the group consisting of: deuterium (D), halogen group, cyano group, nitro group, amino group, C1-C40 alkyl group, C2-C40 alkenyl group, C2-C40 alkynyl group, C3-C40 cycloalkyl group, heterocycloalkyl group having 3 to 40 nuclear atoms, C6-C60 aryl group, heteroaryl group having 5 to 60 nuclear atoms, C1-C40 alkyloxy group, C6-C60 aryloxy group, C1-C40 alkylsilyl group, C6-C60 arylsilyl group, C1-C40 alkylboron group, C6-C60 arylboron group, C6-C60 arylphosphine group, C6-C60 arylphosphine oxide group, C6-C60 arylamine group, (C6-C60 aryl) (heteroaryl having 5 to 60 nuclear atoms) amine group, and heteroarylamine group having 5 to 60 nuclear atoms, and when a plurality of the substituents are present, they are the same as or different from each other).

3. The compound of claim 1,
wherein Ar₁ is selected from the group consisting of substituents represented by the following Chemical Formulas S2-1 to S2-9:

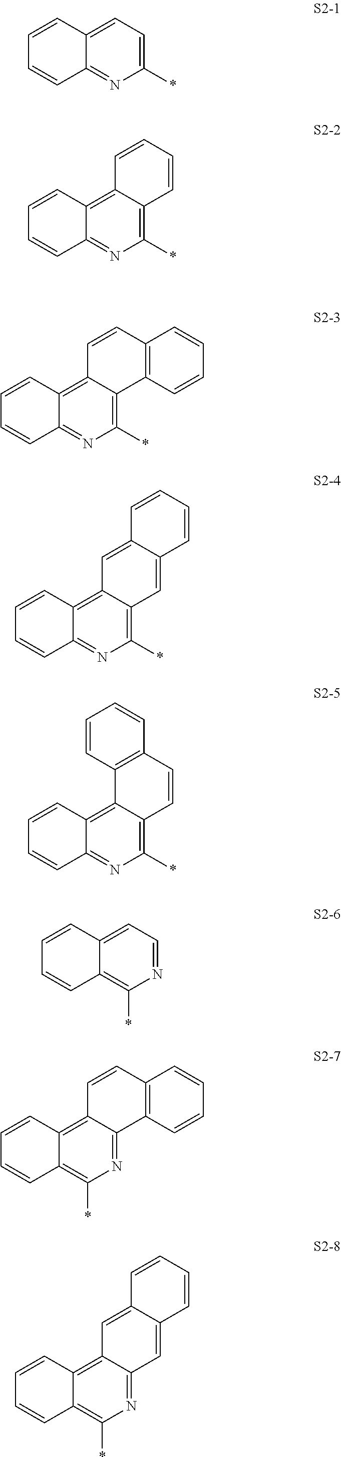

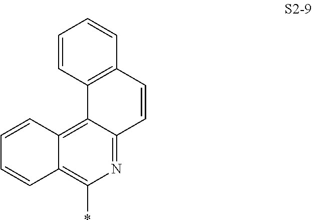 [SMILES: *c1nc2ccc3ccccc3c2c2ccccc12]

4. The compound of claim 1,
wherein L₁ is selected from the group consisting of the following linker groups L1-1 to L1-3: 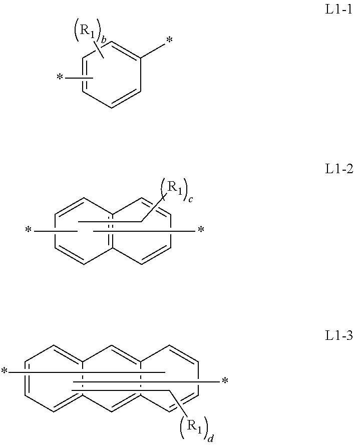
(wherein the linker groups L1-1 to L1-3,
b is an integer in a range of 0 to 4,
c is an integer in a range of 0 to 6,
d is an integer in a range of 0 to 8,
R₁ is selected from the group consisting of: hydrogen, deuterium (D), halogen group, cyano group, nitro group, amino group, C1-C40 alkyl group, C2-C40 alkenyl group, C2-C40 alkynyl group, C3-C40 cycloalkyl group, heterocycloalkyl group having 3 to 40 nuclear atoms, C6-C60 aryl group, heteroaryl group having 5 to 60 nuclear atoms, C1-C40 alkyloxy group, C6-C60 aryloxy group, C1-C40 alkylsilyl group, C6-C60 arylsilyl group, C1-C40 alkylboron group, C6-C60 arylboron group, C6-C60 arylphosphine group, C6-C60 arylphosphine oxide group, C6-C60 arylamine group, (C6-C60 aryl) (heteroaryl having 5 to 60 nuclear atoms) amine group, and heteroarylamine group having 5 to 60 nuclear atoms).

5. The compound of claim 1,
wherein the compound represented by the above Chemical Formula 1 is represented by any one of Chemical Formulas 2 to 7: 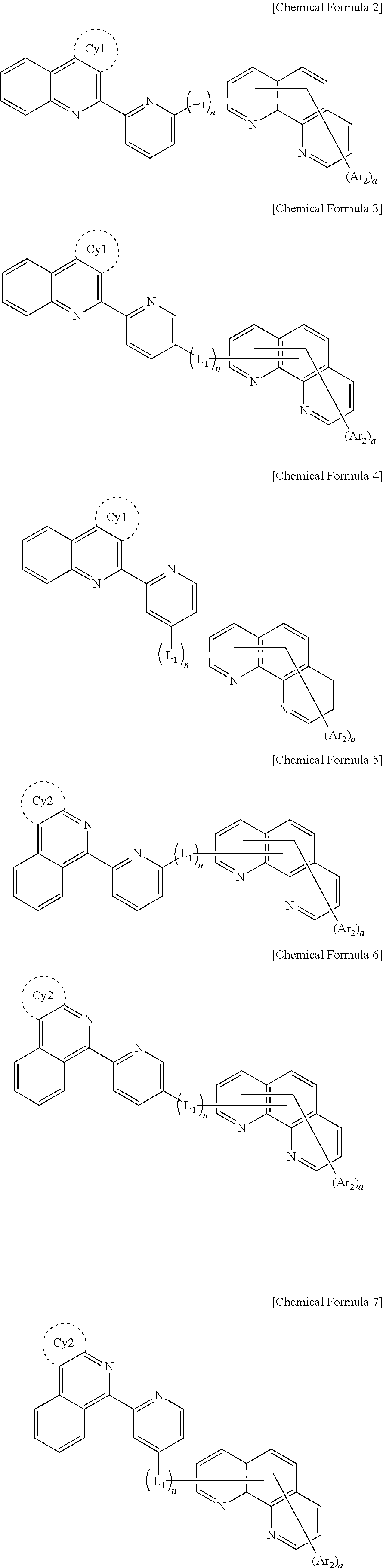
(wherein in the above Chemical Formulas 2 to 7,
n, L₁, a, and Ar₂ are the same as defined in claim 1, respectively,
Cy1 and Cy2 are each independently a C6-C30 fused aromatic ring,
the fused aromatic ring of Cy1 and Cy2 are each independently substituted or unsubstituted with one or more substituents selected from the group consisting of: deuterium (D), halogen group, cyano group, nitro group, amino group, C1-C40 alkyl group, C2-C40 alkenyl group, C2-C40 alkynyl group, C3-C40 cycloalkyl group, heterocycloalkyl group having 3 to 40 nuclear atoms, C6-C60 aryl group, heteroaryl group having 5 to 60 nuclear atoms, C1-C40 C40 alkylboron group, C6-C60 arylboron group, C6-C60 arylphosphine group, C6-C60 arylphosphine oxide group, C6-C60 arylamine group, (C6-C60 aryl) (heteroaryl having 5 to 60 nuclear atoms) amine group, and heteroarylamine group having 5 to 60 nuclear atoms, and when a plurality of the substituents are present, they are the same as or different from each other).

6. The compound of claim 1,
wherein the compound represented by the above Chemical Formula 1 is selected from the group consisting of the following Compounds 001 to 146:

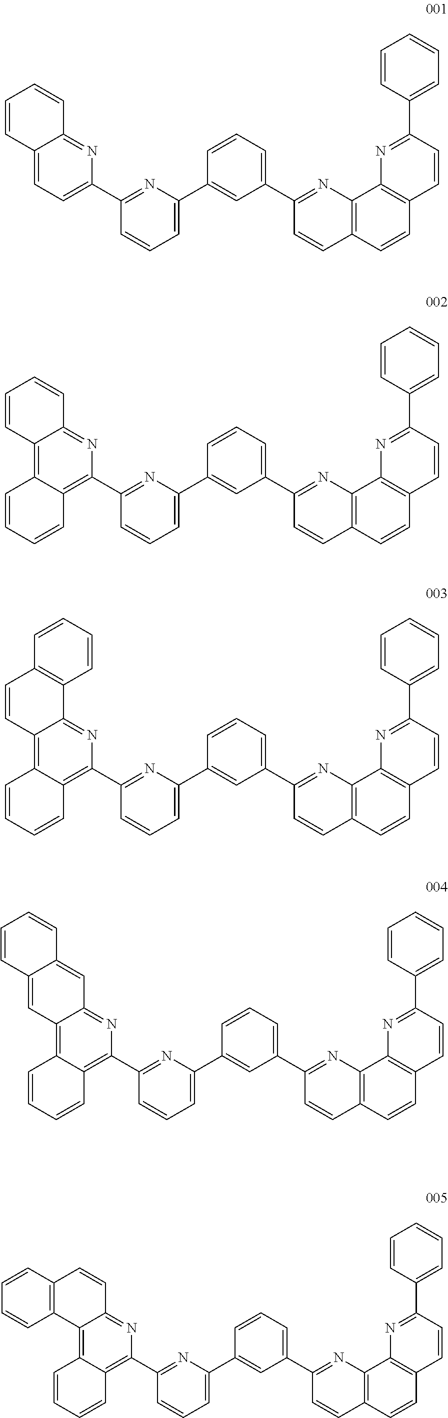 [SMILES: c1ccc(-c2ccc3ccc4ccc(-c5cccc(-c6cccc(-c7ccc8ccccc8n7)n6)c5)nc4c3n2)cc1 ; c1ccc(-c2ccc3ccc4ccc(-c5cccc(-c6cccc(-c7nc8ccccc8c8ccccc78)n6)c5)nc4c3n2)cc1 ; c1ccc(-c2ccc3ccc4ccc(-c5cccc(-c6cccc(-c7nc8c9ccccc9ccc8c8ccccc78)n6)c5)nc4c3n2)cc1 ; c1ccc(-c2ccc3ccc4ccc(-c5cccc(-c6cccc(-c7nc8cc9ccccc9cc8c8ccccc78)n6)c5)nc4c3n2)cc1 ; c1ccc(-c2ccc3ccc4ccc(-c5cccc(-c6cccc(-c7nc8ccc9ccccc9c8c8ccccc78)n6)c5)nc4c3n2)cc1]

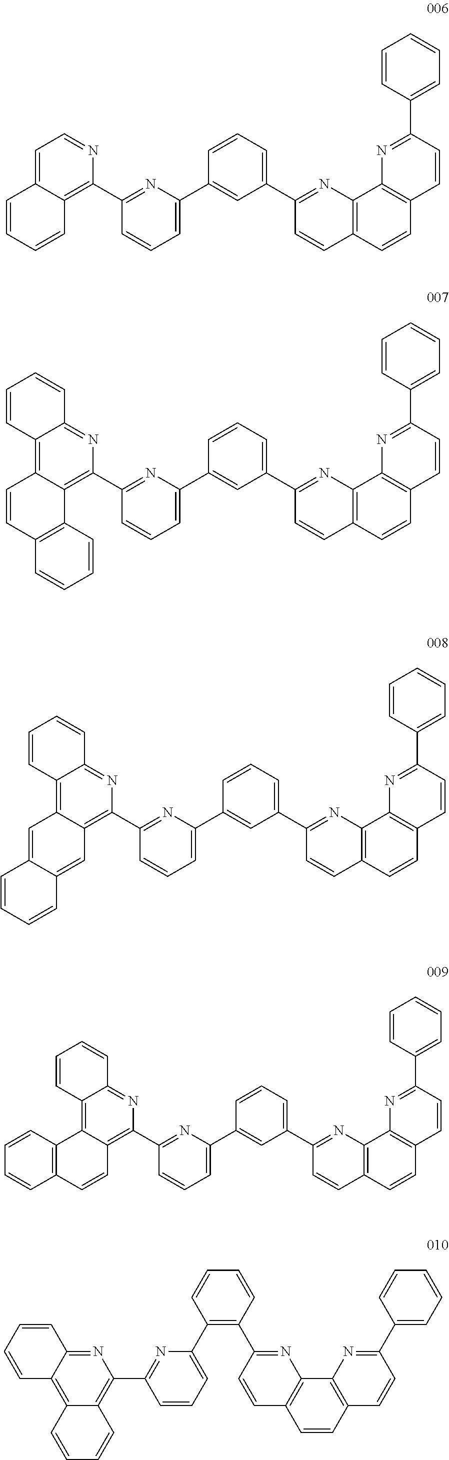 [SMILES: c1ccc(-c2ccc3ccc4ccc(-c5cccc(-c6cccc(-c7nccc8ccccc78)n6)c5)nc4c3n2)cc1 ; c1ccc(-c2ccc3ccc4ccc(-c5cccc(-c6cccc(-c7nc8ccccc8c8ccc9ccccc9c78)n6)c5)nc4c3n2)cc1 ; c1ccc(-c2ccc3ccc4ccc(-c5cccc(-c6cccc(-c7nc8ccccc8c8cc9ccccc9cc78)n6)c5)nc4c3n2)cc1 ; c1ccc(-c2ccc3ccc4ccc(-c5cccc(-c6cccc(-c7nc8ccccc8c8c7ccc7ccccc78)n6)c5)nc4c3n2)cc1 ; c1ccc(-c2ccc3ccc4ccc(-c5ccccc5-c5cccc(-c6nc7ccccc7c7ccccc67)n5)nc4c3n2)cc1]

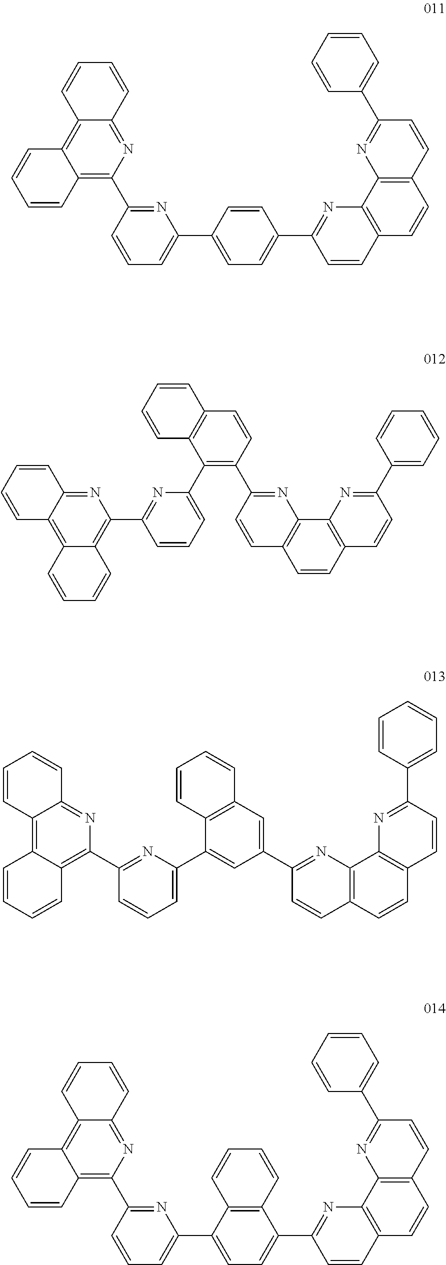 [SMILES: c1ccc(-c2ccc3ccc4ccc(-c5ccc(-c6cccc(-c7nc8ccccc8c8ccccc78)n6)cc5)nc4c3n2)cc1 ; c1ccc(-c2ccc3ccc4ccc(-c5ccc6ccccc6c5-c5cccc(-c6nc7ccccc7c7ccccc67)n5)nc4c3n2)cc1 ; c1ccc(-c2ccc3ccc4ccc(-c5cc(-c6cccc(-c7nc8ccccc8c8ccccc78)n6)c6ccccc6c5)nc4c3n2)cc1 ; c1ccc(-c2ccc3ccc4ccc(-c5ccc(-c6cccc(-c7nc8ccccc8c8ccccc78)n6)c6ccccc56)nc4c3n2)cc1]

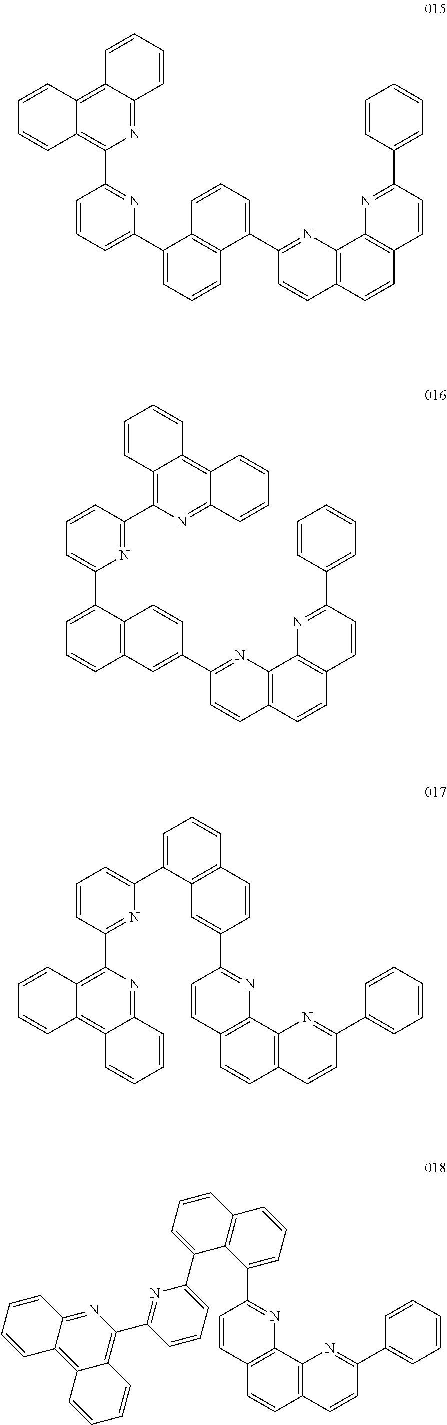 [SMILES: c1ccc(-c2ccc3ccc4ccc(-c5cccc6c(-c7cccc(-c8nc9ccccc9c9ccccc89)n7)cccc56)nc4c3n2)cc1 ; c1ccc(-c2ccc3ccc4ccc(-c5ccc6c(-c7cccc(-c8nc9ccccc9c9ccccc89)n7)cccc6c5)nc4c3n2)cc1 ; c1ccc(-c2ccc3ccc4ccc(-c5ccc6cccc(-c7cccc(-c8nc9ccccc9c9ccccc89)n7)c6c5)nc4c3n2)cc1 ; c1ccc(-c2ccc3ccc4ccc(-c5cccc6cccc(-c7cccc(-c8nc9ccccc9c9ccccc89)n7)c56)nc4c3n2)cc1]

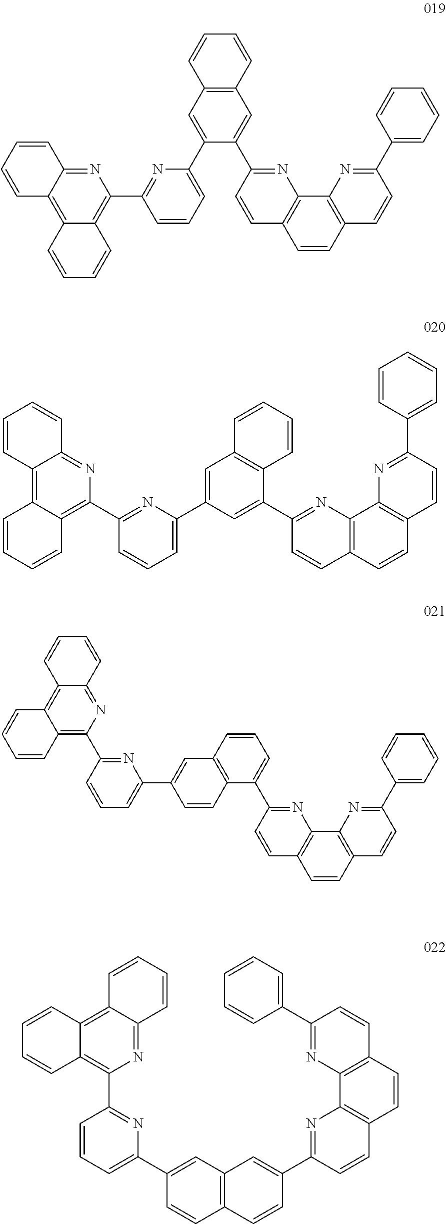 [SMILES: c1ccc(-c2ccc3ccc4ccc(-c5cc6ccccc6cc5-c5cccc(-c6nc7ccccc7c7ccccc67)n5)nc4c3n2)cc1 ; c1ccc(-c2ccc3ccc4ccc(-c5cc(-c6cccc(-c7nc8ccccc8c8ccccc78)n6)cc6ccccc56)nc4c3n2)cc1 ; c1ccc(-c2ccc3ccc4ccc(-c5cccc6cc(-c7cccc(-c8nc9ccccc9c9ccccc89)n7)ccc56)nc4c3n2)cc1 ; c1ccc(-c2ccc3ccc4ccc(-c5ccc6ccc(-c7cccc(-c8nc9ccccc9c9ccccc89)n7)cc6c5)nc4c3n2)cc1]

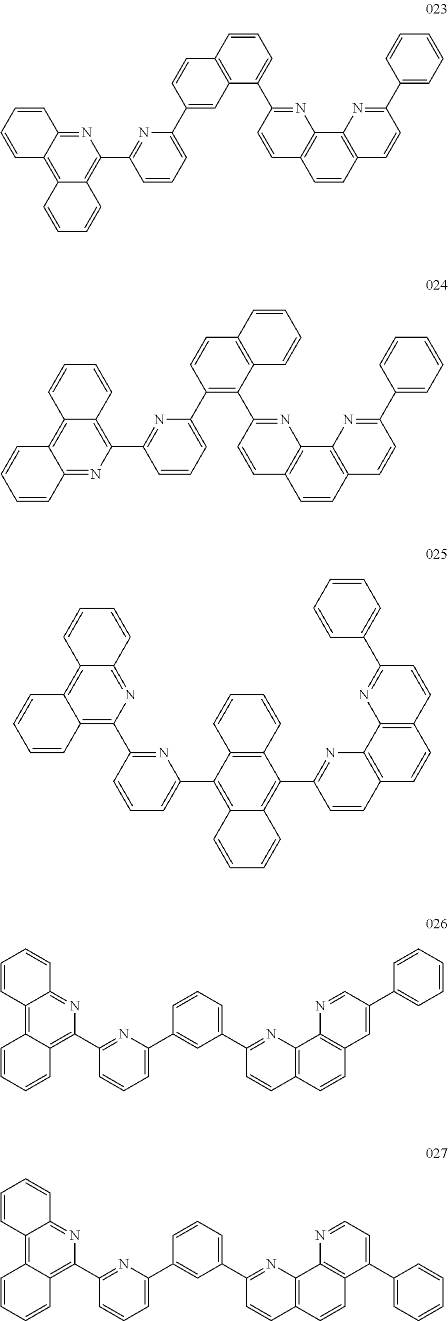 [SMILES: c1ccc(-c2ccc3ccc4ccc(-c5cccc6ccc(-c7cccc(-c8nc9ccccc9c9ccccc89)n7)cc56)nc4c3n2)cc1 ; c1ccc(-c2ccc3ccc4ccc(-c5c(-c6cccc(-c7nc8ccccc8c8ccccc78)n6)ccc6ccccc56)nc4c3n2)cc1 ; c1ccc(-c2ccc3ccc4ccc(-c5c6ccccc6c(-c6cccc(-c7nc8ccccc8c8ccccc78)n6)c6ccccc56)nc4c3n2)cc1 ; c1ccc(-c2cnc3c(ccc4ccc(-c5cccc(-c6cccc(-c7nc8ccccc8c8ccccc78)n6)c5)nc43)c2)cc1 ; c1ccc(-c2ccnc3c2ccc2ccc(-c4cccc(-c5cccc(-c6nc7ccccc7c7ccccc67)n5)c4)nc23)cc1]

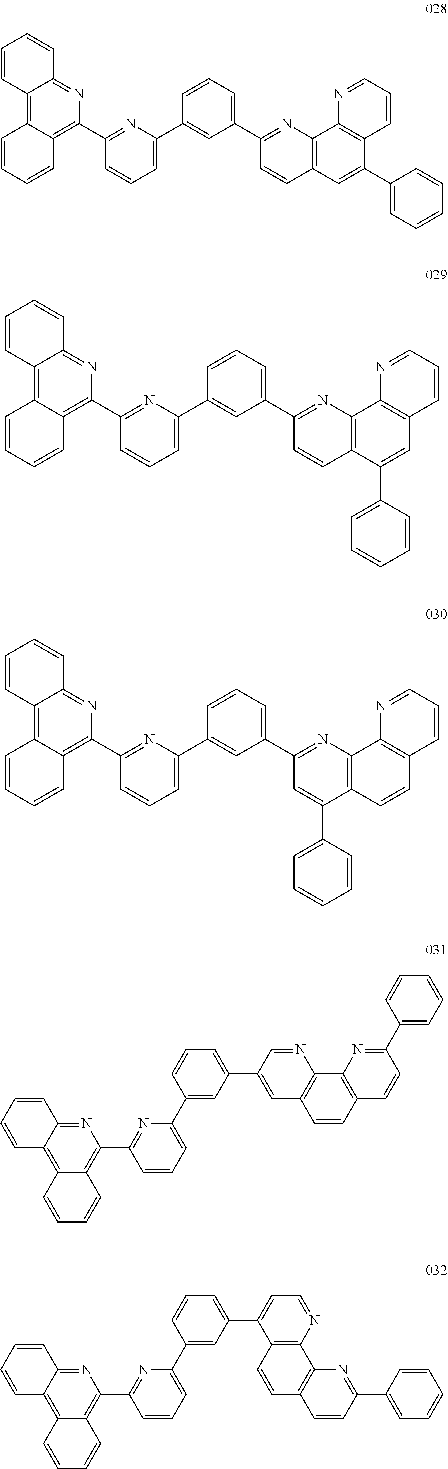 [SMILES: c1ccc(-c2cc3ccc(-c4cccc(-c5cccc(-c6nc7ccccc7c7ccccc67)n5)c4)nc3c3ncccc23)cc1 ; c1ccc(-c2cc3cccnc3c3nc(-c4cccc(-c5cccc(-c6nc7ccccc7c7ccccc67)n5)c4)ccc23)cc1 ; c1ccc(-c2cc(-c3cccc(-c4cccc(-c5nc6ccccc6c6ccccc56)n4)c3)nc3c2ccc2cccnc23)cc1 ; c1ccc(-c2ccc3ccc4cc(-c5cccc(-c6cccc(-c7nc8ccccc8c8ccccc78)n6)c5)cnc4c3n2)cc1 ; c1ccc(-c2ccc3ccc4c(-c5cccc(-c6cccc(-c7nc8ccccc8c8ccccc78)n6)c5)ccnc4c3n2)cc1]

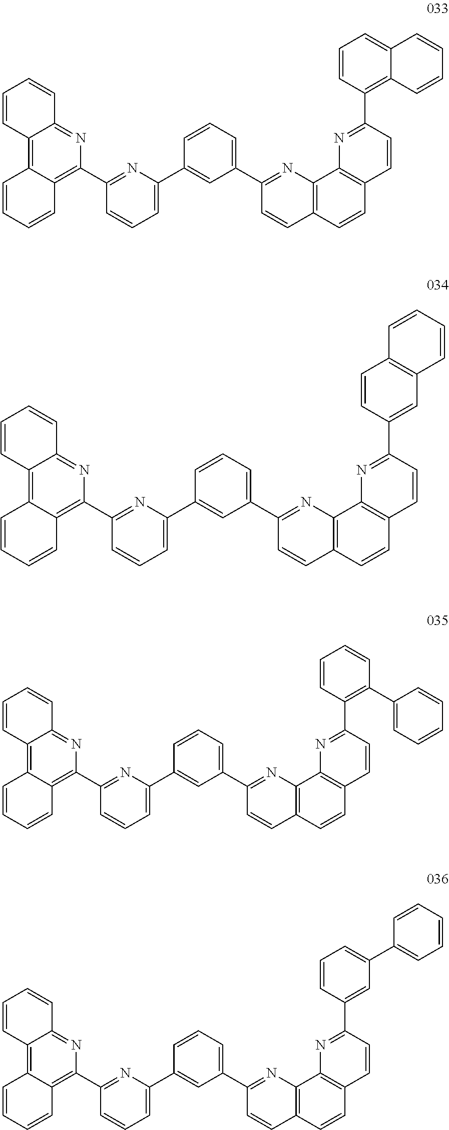 [SMILES: c1cc(-c2cccc(-c3nc4ccccc4c4ccccc34)n2)cc(-c2ccc3ccc4ccc(-c5cccc6ccccc56)nc4c3n2)c1 ; c1cc(-c2cccc(-c3nc4ccccc4c4ccccc34)n2)cc(-c2ccc3ccc4ccc(-c5ccc6ccccc6c5)nc4c3n2)c1 ; c1ccc(-c2ccccc2-c2ccc3ccc4ccc(-c5cccc(-c6cccc(-c7nc8ccccc8c8ccccc78)n6)c5)nc4c3n2)cc1 ; c1ccc(-c2cccc(-c3ccc4ccc5ccc(-c6cccc(-c7cccc(-c8nc9ccccc9c9ccccc89)n7)c6)nc5c4n3)c2)cc1]

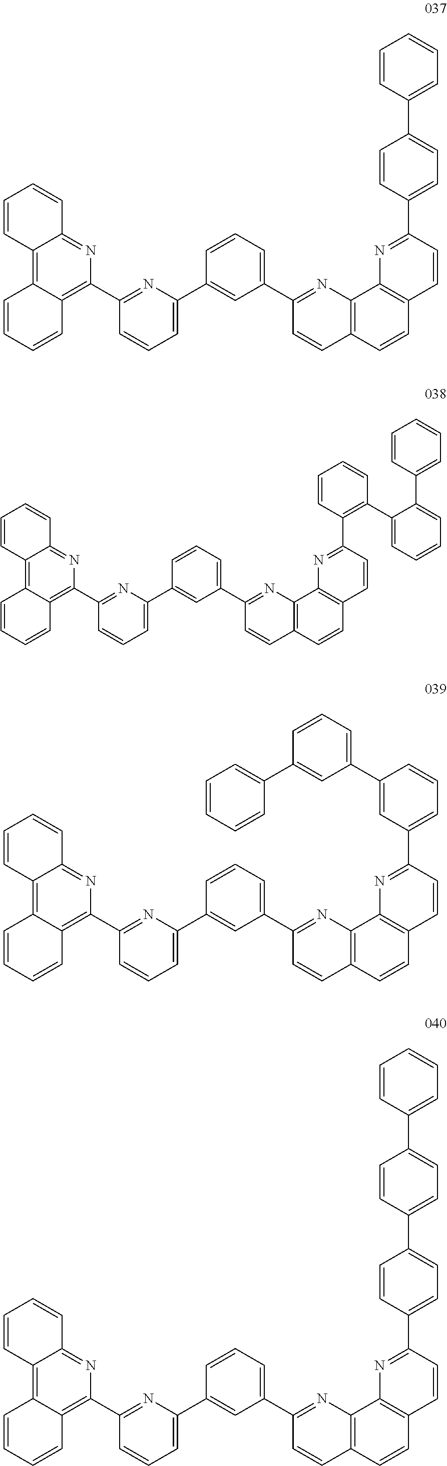 [SMILES: c1ccc(-c2ccc(-c3ccc4ccc5ccc(-c6cccc(-c7cccc(-c8nc9ccccc9c9ccccc89)n7)c6)nc5c4n3)cc2)cc1 ; c1ccc(-c2ccccc2-c2ccccc2-c2ccc3ccc4ccc(-c5cccc(-c6cccc(-c7nc8ccccc8c8ccccc78)n6)c5)nc4c3n2)cc1 ; c1ccc(-c2cccc(-c3cccc(-c4ccc5ccc6ccc(-c7cccc(-c8cccc(-c9nc%10ccccc%10c%10ccccc9%10)n8)c7)nc6c5n4)c3)c2)cc1 ; c1ccc(-c2ccc(-c3ccc(-c4ccc5ccc6ccc(-c7cccc(-c8cccc(-c9nc%10ccccc%10c%10ccccc9%10)n8)c7)nc6c5n4)cc3)cc2)cc1]

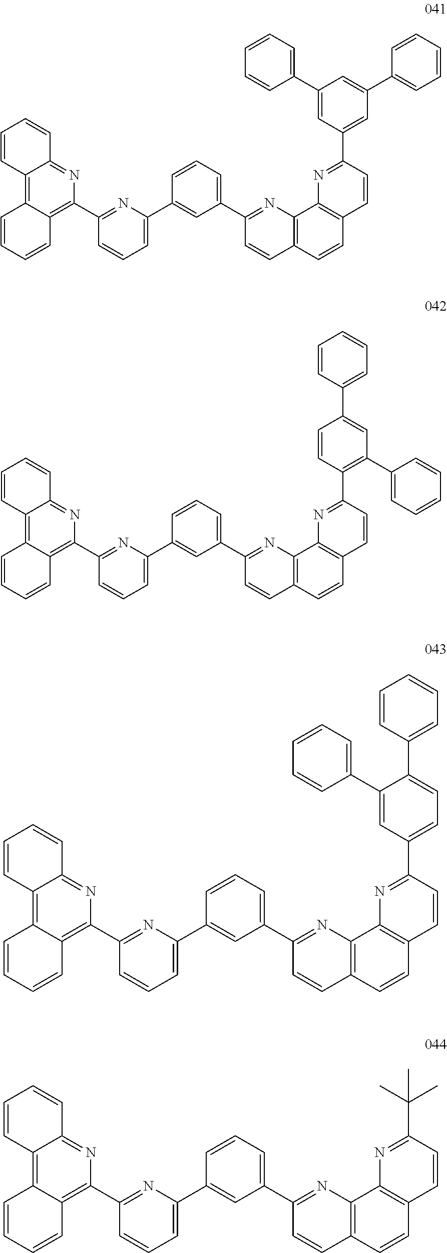 [SMILES: c1ccc(-c2cc(-c3ccccc3)cc(-c3ccc4ccc5ccc(-c6cccc(-c7cccc(-c8nc9ccccc9c9ccccc89)n7)c6)nc5c4n3)c2)cc1 ; c1ccc(-c2ccc(-c3ccc4ccc5ccc(-c6cccc(-c7cccc(-c8nc9ccccc9c9ccccc89)n7)c6)nc5c4n3)c(-c3ccccc3)c2)cc1 ; c1ccc(-c2ccc(-c3ccc4ccc5ccc(-c6cccc(-c7cccc(-c8nc9ccccc9c9ccccc89)n7)c6)nc5c4n3)cc2-c2ccccc2)cc1 ; CC(C)(C)c1ccc2ccc3ccc(-c4cccc(-c5cccc(-c6nc7ccccc7c7ccccc67)n5)c4)nc3c2n1]

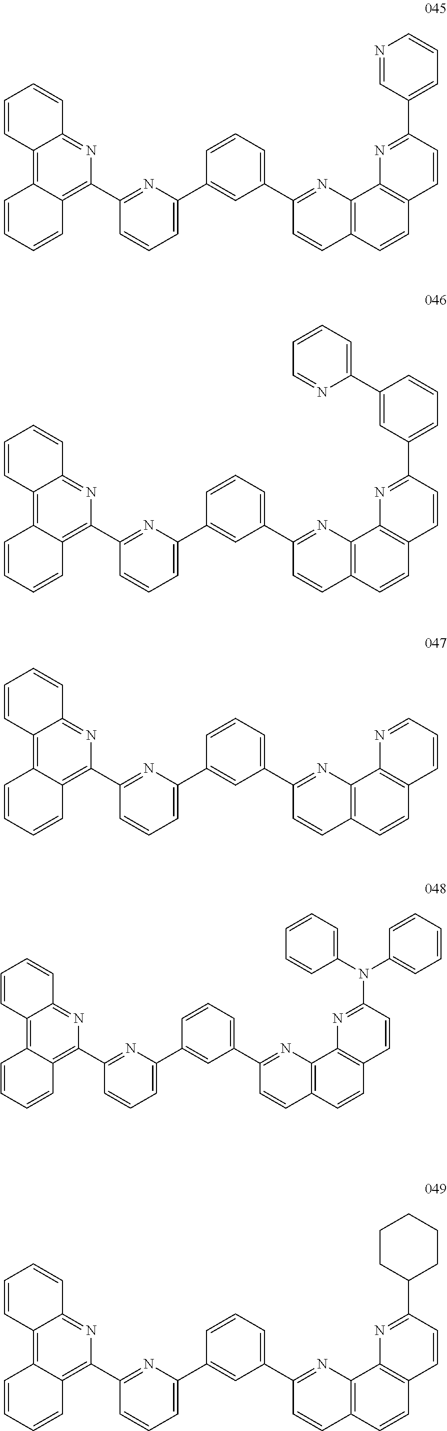 [SMILES: c1cncc(-c2ccc3ccc4ccc(-c5cccc(-c6cccc(-c7nc8ccccc8c8ccccc78)n6)c5)nc4c3n2)c1 ; c1ccc(-c2cccc(-c3ccc4ccc5ccc(-c6cccc(-c7cccc(-c8nc9ccccc9c9ccccc89)n7)c6)nc5c4n3)c2)nc1 ; c1cc(-c2cccc(-c3nc4ccccc4c4ccccc34)n2)cc(-c2ccc3ccc4cccnc4c3n2)c1 ; c1ccc(N(c2ccccc2)c2ccc3ccc4ccc(-c5cccc(-c6cccc(-c7nc8ccccc8c8ccccc78)n6)c5)nc4c3n2)cc1 ; c1cc(-c2cccc(-c3nc4ccccc4c4ccccc34)n2)cc(-c2ccc3ccc4ccc(C5CCCCC5)nc4c3n2)c1]

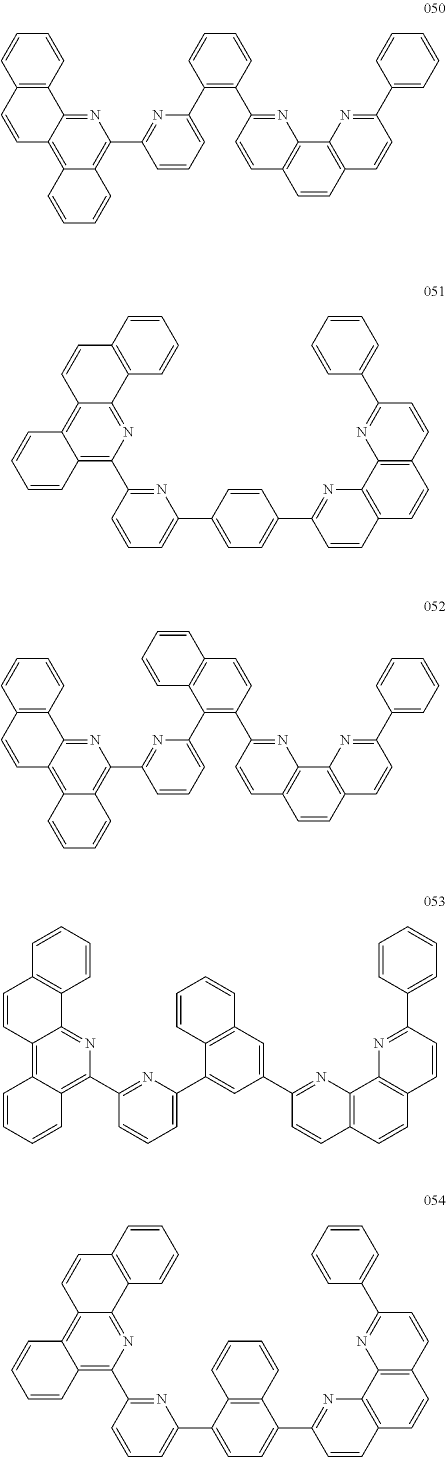 [SMILES: c1ccc(-c2ccc3ccc4ccc(-c5ccccc5-c5cccc(-c6nc7c8ccccc8ccc7c7ccccc67)n5)nc4c3n2)cc1 ; c1ccc(-c2ccc3ccc4ccc(-c5ccc(-c6cccc(-c7nc8c9ccccc9ccc8c8ccccc78)n6)cc5)nc4c3n2)cc1 ; c1ccc(-c2ccc3ccc4ccc(-c5ccc6ccccc6c5-c5cccc(-c6nc7c8ccccc8ccc7c7ccccc67)n5)nc4c3n2)cc1 ; c1ccc(-c2ccc3ccc4ccc(-c5cc(-c6cccc(-c7nc8c9ccccc9ccc8c8ccccc78)n6)c6ccccc6c5)nc4c3n2)cc1 ; c1ccc(-c2ccc3ccc4ccc(-c5ccc(-c6cccc(-c7nc8c9ccccc9ccc8c8ccccc78)n6)c6ccccc56)nc4c3n2)cc1]

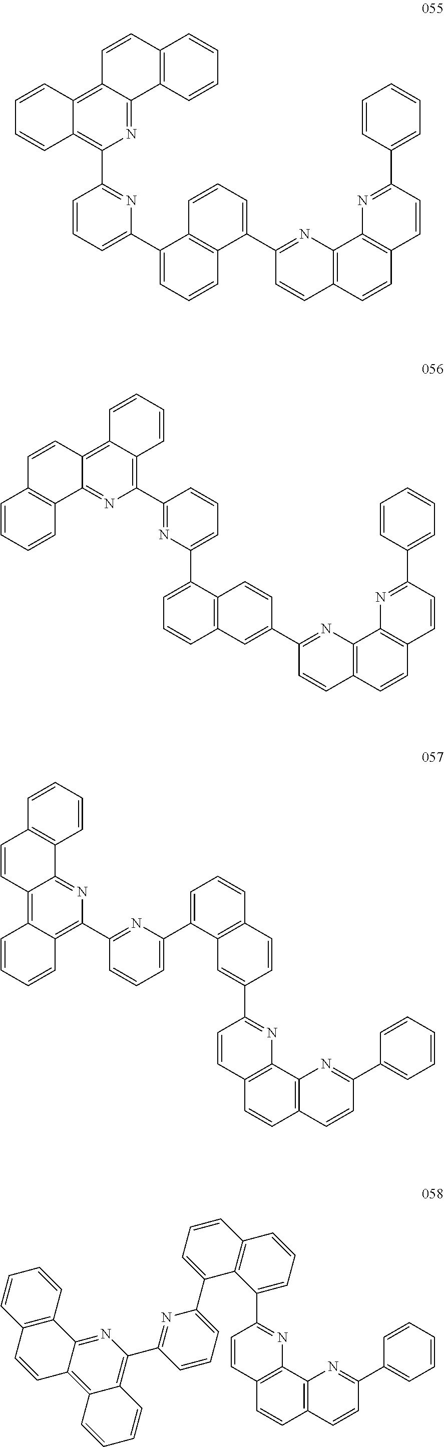 [SMILES: c1ccc(-c2ccc3ccc4ccc(-c5cccc6c(-c7cccc(-c8nc9c%10ccccc%10ccc9c9ccccc89)n7)cccc56)nc4c3n2)cc1 ; c1ccc(-c2ccc3ccc4ccc(-c5ccc6c(-c7cccc(-c8nc9c%10ccccc%10ccc9c9ccccc89)n7)cccc6c5)nc4c3n2)cc1 ; c1ccc(-c2ccc3ccc4ccc(-c5ccc6cccc(-c7cccc(-c8nc9c%10ccccc%10ccc9c9ccccc89)n7)c6c5)nc4c3n2)cc1 ; c1ccc(-c2ccc3ccc4ccc(-c5cccc6cccc(-c7cccc(-c8nc9c%10ccccc%10ccc9c9ccccc89)n7)c56)nc4c3n2)cc1]

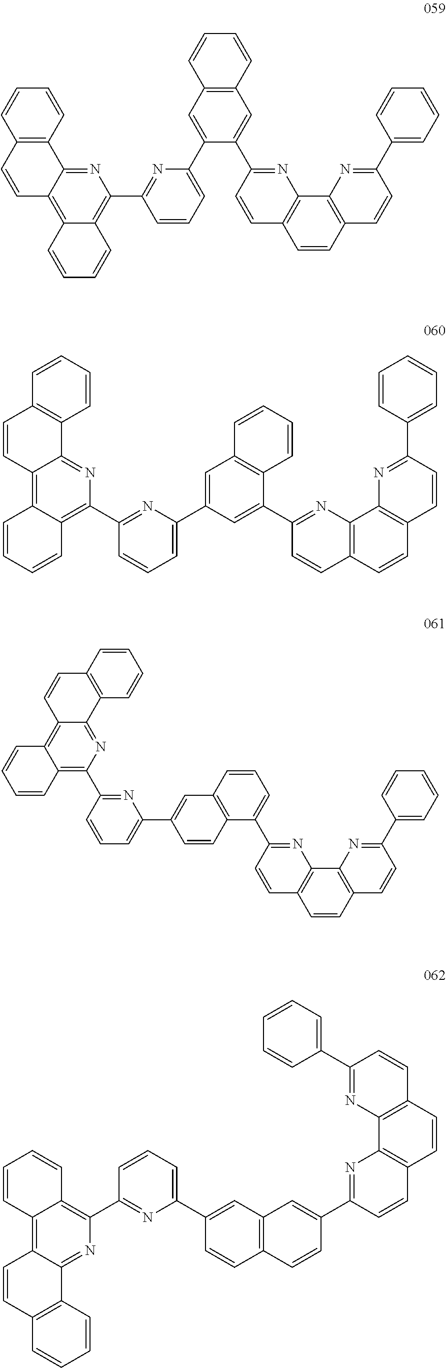 [SMILES: c1ccc(-c2ccc3ccc4ccc(-c5cc6ccccc6cc5-c5cccc(-c6nc7c8ccccc8ccc7c7ccccc67)n5)nc4c3n2)cc1 ; c1ccc(-c2ccc3ccc4ccc(-c5cc(-c6cccc(-c7nc8c9ccccc9ccc8c8ccccc78)n6)cc6ccccc56)nc4c3n2)cc1 ; c1ccc(-c2ccc3ccc4ccc(-c5cccc6cc(-c7cccc(-c8nc9c%10ccccc%10ccc9c9ccccc89)n7)ccc56)nc4c3n2)cc1 ; c1ccc(-c2ccc3ccc4ccc(-c5ccc6ccc(-c7cccc(-c8nc9c%10ccccc%10ccc9c9ccccc89)n7)cc6c5)nc4c3n2)cc1]

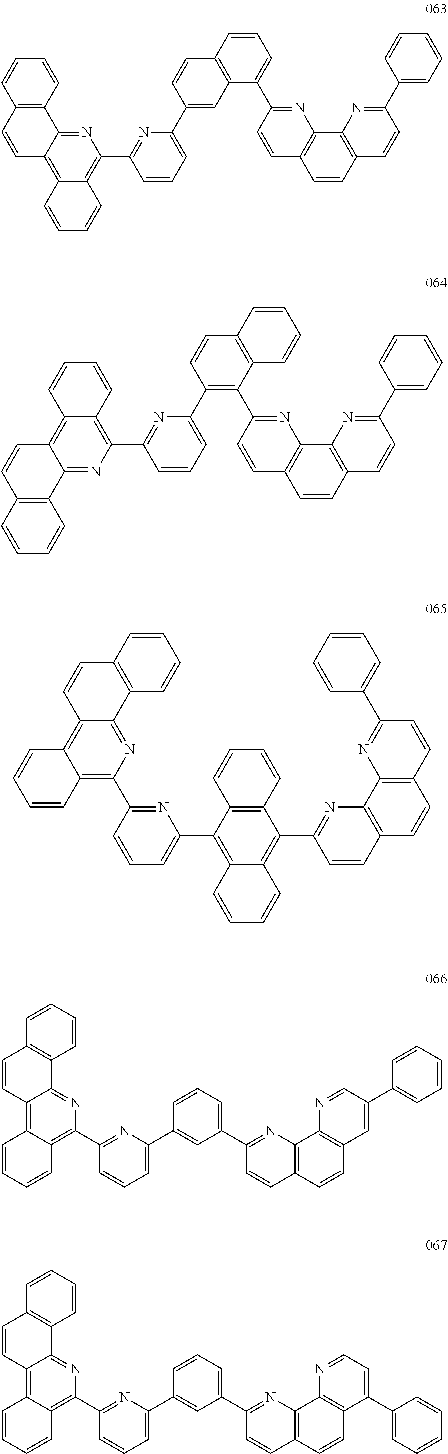 [SMILES: c1ccc(-c2ccc3ccc4ccc(-c5cccc6ccc(-c7cccc(-c8nc9c%10ccccc%10ccc9c9ccccc89)n7)cc56)nc4c3n2)cc1 ; c1ccc(-c2ccc3ccc4ccc(-c5c(-c6cccc(-c7nc8c9ccccc9ccc8c8ccccc78)n6)ccc6ccccc56)nc4c3n2)cc1 ; c1ccc(-c2ccc3ccc4ccc(-c5c6ccccc6c(-c6cccc(-c7nc8c9ccccc9ccc8c8ccccc78)n6)c6ccccc56)nc4c3n2)cc1 ; c1ccc(-c2cnc3c(ccc4ccc(-c5cccc(-c6cccc(-c7nc8c9ccccc9ccc8c8ccccc78)n6)c5)nc43)c2)cc1 ; c1ccc(-c2ccnc3c2ccc2ccc(-c4cccc(-c5cccc(-c6nc7c8ccccc8ccc7c7ccccc67)n5)c4)nc23)cc1]

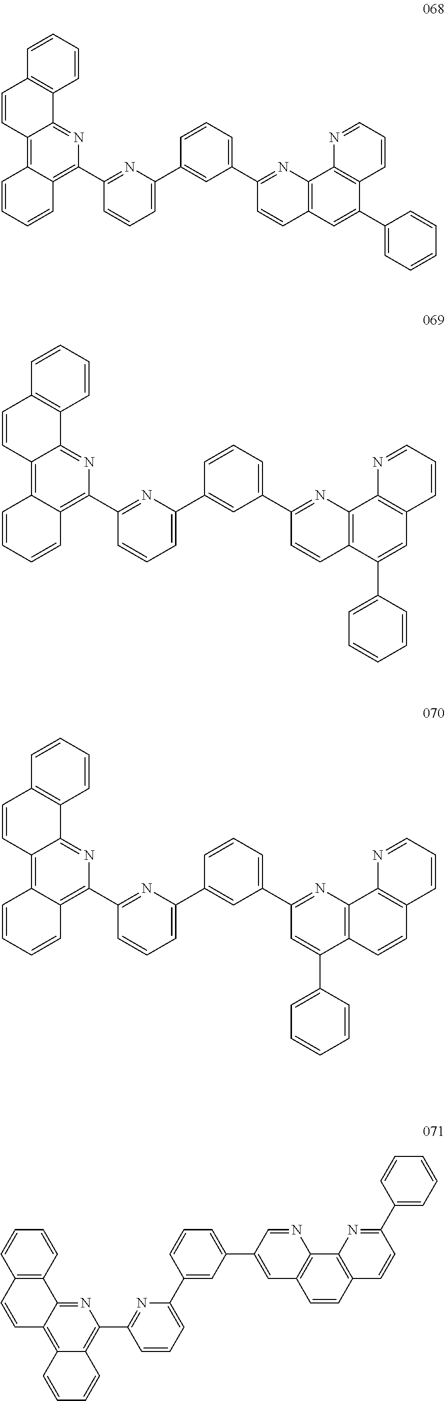 [SMILES: c1ccc(-c2cc3ccc(-c4cccc(-c5cccc(-c6nc7c8ccccc8ccc7c7ccccc67)n5)c4)nc3c3ncccc23)cc1 ; c1ccc(-c2cc3cccnc3c3nc(-c4cccc(-c5cccc(-c6nc7c8ccccc8ccc7c7ccccc67)n5)c4)ccc23)cc1 ; c1ccc(-c2cc(-c3cccc(-c4cccc(-c5nc6c7ccccc7ccc6c6ccccc56)n4)c3)nc3c2ccc2cccnc23)cc1 ; c1ccc(-c2ccc3ccc4cc(-c5cccc(-c6cccc(-c7nc8c9ccccc9ccc8c8ccccc78)n6)c5)cnc4c3n2)cc1]

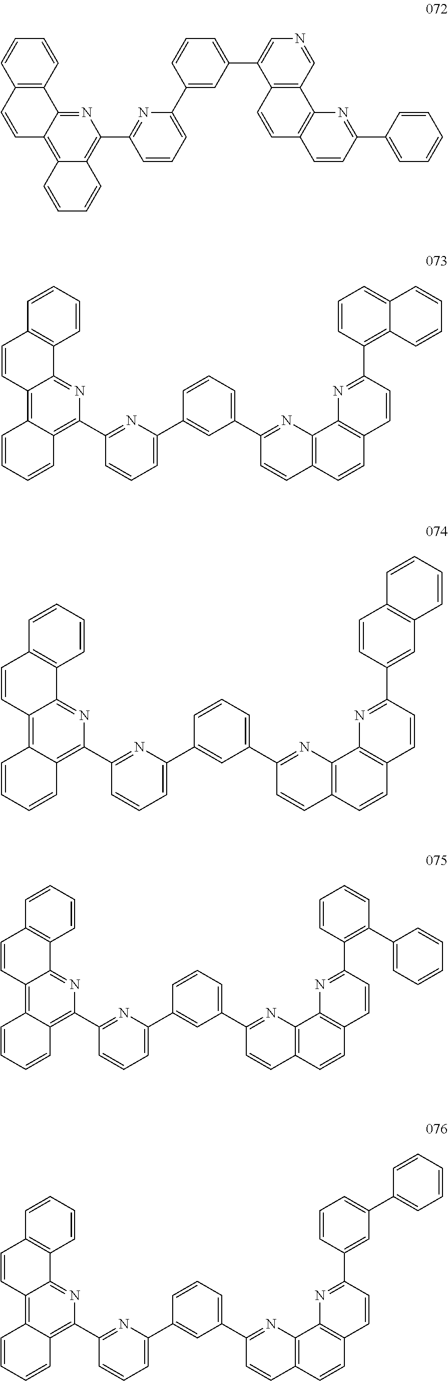 [SMILES: c1ccc(-c2ccc3ccc4c(-c5cccc(-c6cccc(-c7nc8c9ccccc9ccc8c8ccccc78)n6)c5)cncc4c3n2)cc1 ; c1cc(-c2cccc(-c3nc4c5ccccc5ccc4c4ccccc34)n2)cc(-c2ccc3ccc4ccc(-c5cccc6ccccc56)nc4c3n2)c1 ; c1cc(-c2cccc(-c3nc4c5ccccc5ccc4c4ccccc34)n2)cc(-c2ccc3ccc4ccc(-c5ccc6ccccc6c5)nc4c3n2)c1 ; c1ccc(-c2ccccc2-c2ccc3ccc4ccc(-c5cccc(-c6cccc(-c7nc8c9ccccc9ccc8c8ccccc78)n6)c5)nc4c3n2)cc1 ; c1ccc(-c2cccc(-c3ccc4ccc5ccc(-c6cccc(-c7cccc(-c8nc9c%10ccccc%10ccc9c9ccccc89)n7)c6)nc5c4n3)c2)cc1]

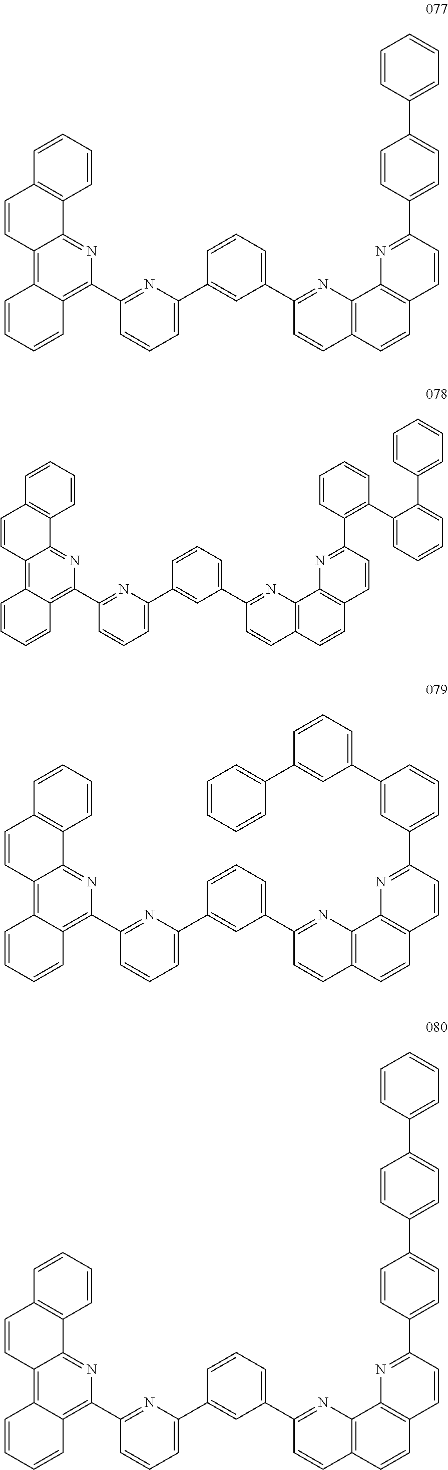 [SMILES: c1ccc(-c2ccc(-c3ccc4ccc5ccc(-c6cccc(-c7cccc(-c8nc9c%10ccccc%10ccc9c9ccccc89)n7)c6)nc5c4n3)cc2)cc1 ; c1ccc(-c2ccccc2-c2ccccc2-c2ccc3ccc4ccc(-c5cccc(-c6cccc(-c7nc8c9ccccc9ccc8c8ccccc78)n6)c5)nc4c3n2)cc1 ; c1ccc(-c2cccc(-c3cccc(-c4ccc5ccc6ccc(-c7cccc(-c8cccc(-c9nc%10c%11ccccc%11ccc%10c%10ccccc9%10)n8)c7)nc6c5n4)c3)c2)cc1 ; c1ccc(-c2ccc(-c3ccc(-c4ccc5ccc6ccc(-c7cccc(-c8cccc(-c9nc%10c%11ccccc%11ccc%10c%10ccccc9%10)n8)c7)nc6c5n4)cc3)cc2)cc1]

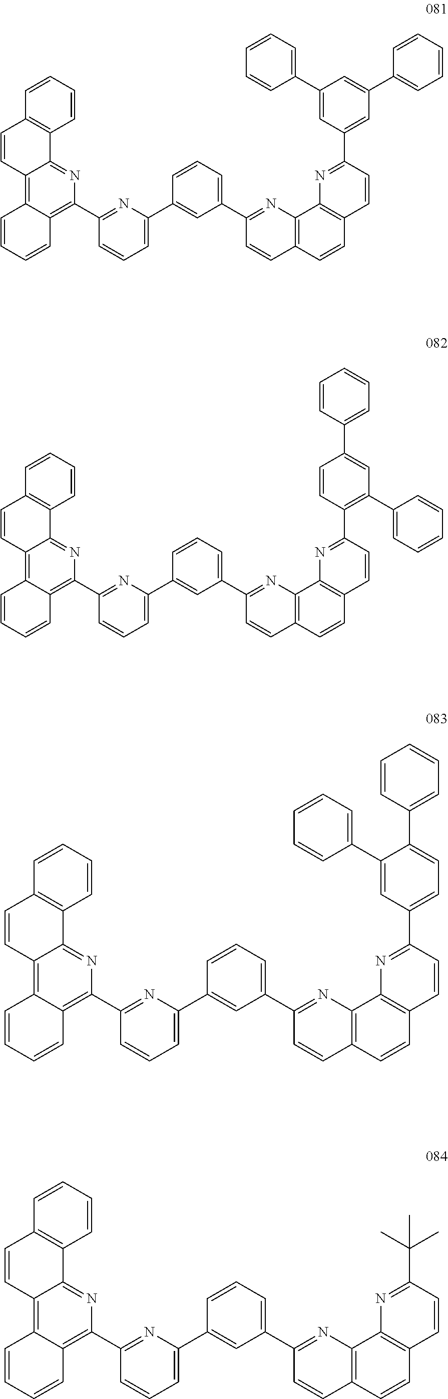 [SMILES: c1ccc(-c2cc(-c3ccccc3)cc(-c3ccc4ccc5ccc(-c6cccc(-c7cccc(-c8nc9c%10ccccc%10ccc9c9ccccc89)n7)c6)nc5c4n3)c2)cc1 ; c1ccc(-c2ccc(-c3ccc4ccc5ccc(-c6cccc(-c7cccc(-c8nc9c%10ccccc%10ccc9c9ccccc89)n7)c6)nc5c4n3)c(-c3ccccc3)c2)cc1 ; c1ccc(-c2ccc(-c3ccc4ccc5ccc(-c6cccc(-c7cccc(-c8nc9c%10ccccc%10ccc9c9ccccc89)n7)c6)nc5c4n3)cc2-c2ccccc2)cc1 ; CC(C)(C)c1ccc2ccc3ccc(-c4cccc(-c5cccc(-c6nc7c8ccccc8ccc7c7ccccc67)n5)c4)nc3c2n1]

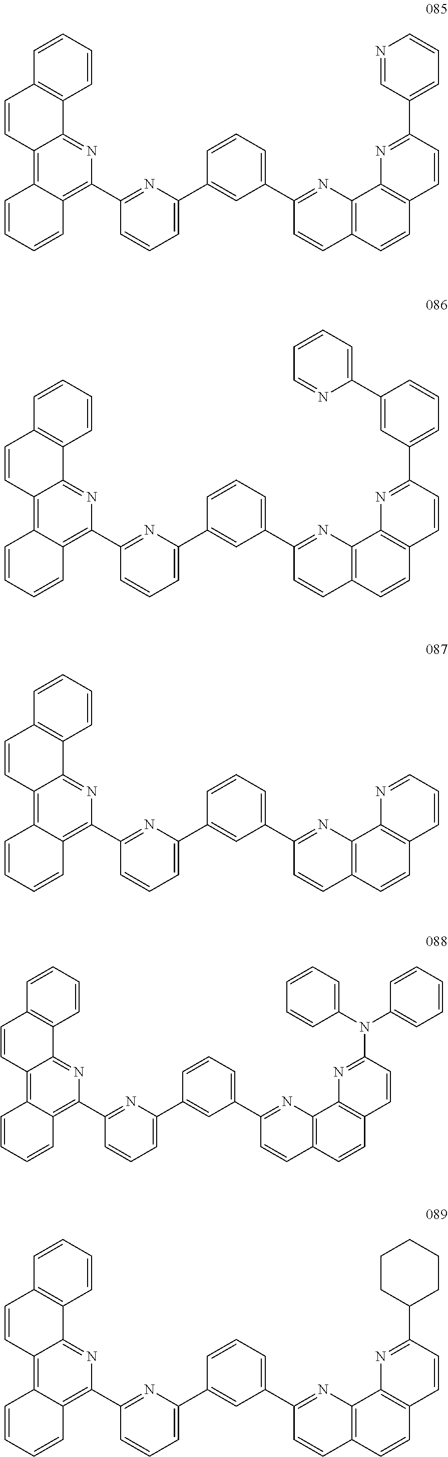 [SMILES: c1cncc(-c2ccc3ccc4ccc(-c5cccc(-c6cccc(-c7nc8c9ccccc9ccc8c8ccccc78)n6)c5)nc4c3n2)c1 ; c1ccc(-c2cccc(-c3ccc4ccc5ccc(-c6cccc(-c7cccc(-c8nc9c%10ccccc%10ccc9c9ccccc89)n7)c6)nc5c4n3)c2)nc1 ; c1cc(-c2cccc(-c3nc4c5ccccc5ccc4c4ccccc34)n2)cc(-c2ccc3ccc4cccnc4c3n2)c1 ; c1ccc(N(c2ccccc2)c2ccc3ccc4ccc(-c5cccc(-c6cccc(-c7nc8c9ccccc9ccc8c8ccccc78)n6)c5)nc4c3n2)cc1 ; c1cc(-c2cccc(-c3nc4c5ccccc5ccc4c4ccccc34)n2)cc(-c2ccc3ccc4ccc(C5CCCCC5)nc4c3n2)c1]

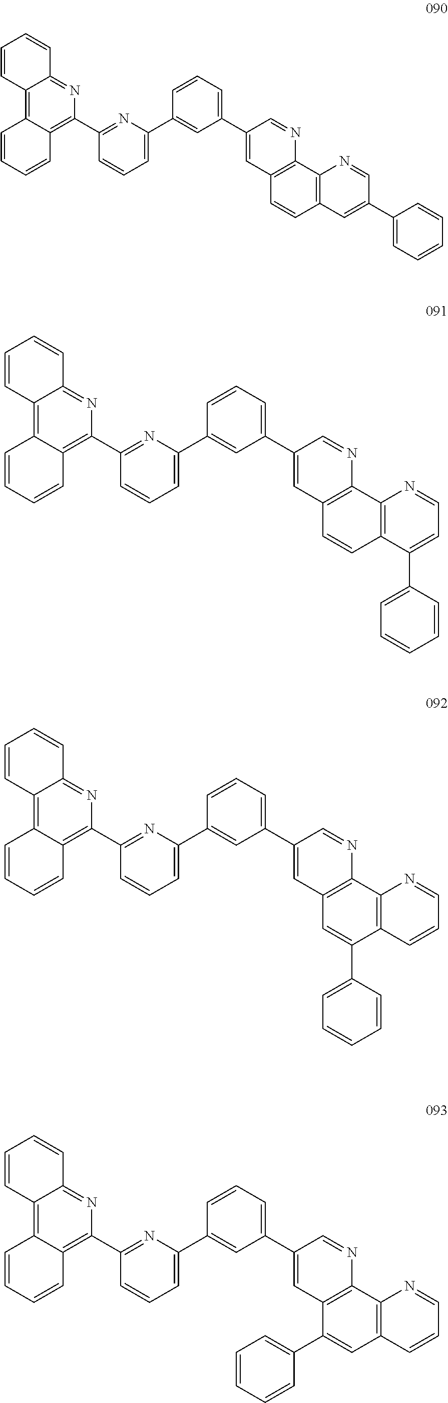 [SMILES: c1ccc(-c2cnc3c(ccc4cc(-c5cccc(-c6cccc(-c7nc8ccccc8c8ccccc78)n6)c5)cnc43)c2)cc1 ; c1ccc(-c2ccnc3c2ccc2cc(-c4cccc(-c5cccc(-c6nc7ccccc7c7ccccc67)n5)c4)cnc23)cc1 ; c1ccc(-c2cc3cc(-c4cccc(-c5cccc(-c6nc7ccccc7c7ccccc67)n5)c4)cnc3c3ncccc23)cc1 ; c1ccc(-c2cc3cccnc3c3ncc(-c4cccc(-c5cccc(-c6nc7ccccc7c7ccccc67)n5)c4)cc23)cc1]

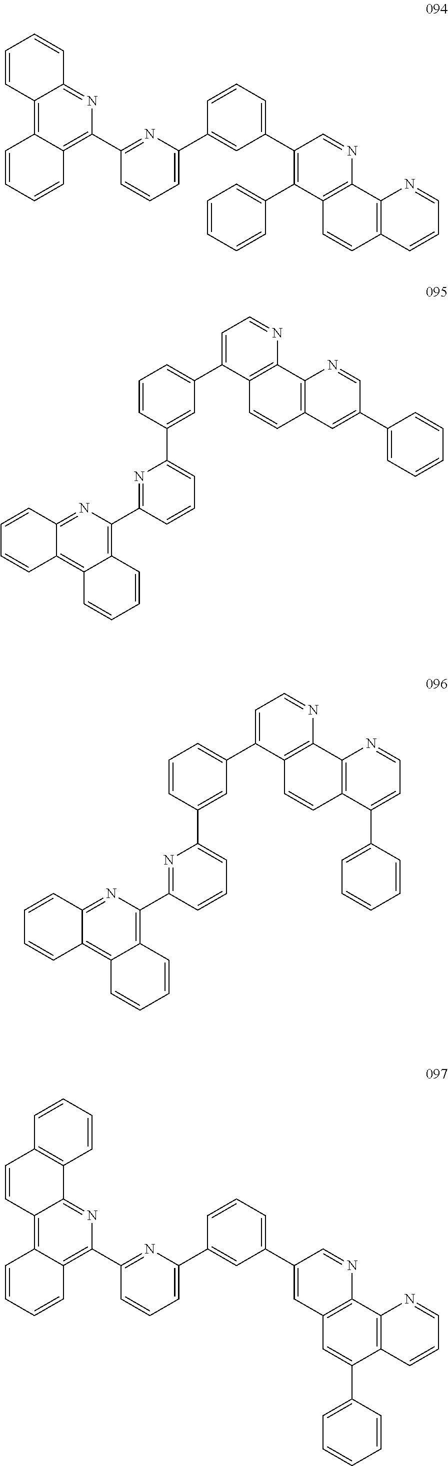 [SMILES: c1ccc(-c2c(-c3cccc(-c4cccc(-c5nc6ccccc6c6ccccc56)n4)c3)cnc3c2ccc2cccnc23)cc1 ; c1ccc(-c2cnc3c(ccc4c(-c5cccc(-c6cccc(-c7nc8ccccc8c8ccccc78)n6)c5)ccnc43)c2)cc1 ; c1ccc(-c2ccnc3c2ccc2c(-c4cccc(-c5cccc(-c6nc7ccccc7c7ccccc67)n5)c4)ccnc23)cc1 ; c1ccc(-c2cc3cc(-c4cccc(-c5cccc(-c6nc7c8ccccc8ccc7c7ccccc67)n5)c4)cnc3c3ncccc23)cc1]

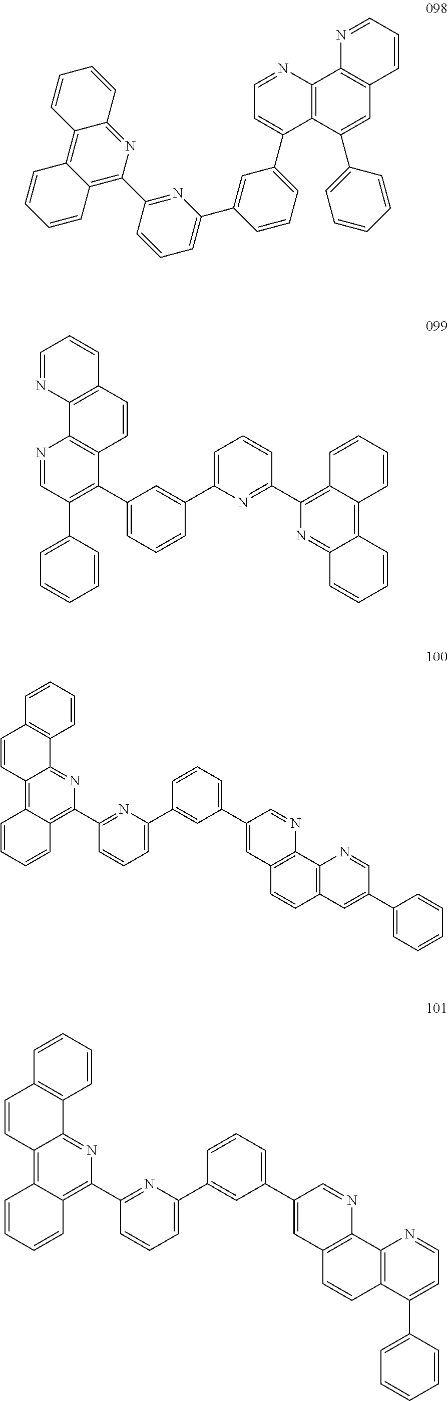 [SMILES: c1ccc(-c2cc3cccnc3c3nccc(-c4cccc(-c5cccc(-c6nc7ccccc7c7ccccc67)n5)c4)c23)cc1 ; c1ccc(-c2cnc3c(ccc4cccnc43)c2-c2cccc(-c3cccc(-c4nc5ccccc5c5ccccc45)n3)c2)cc1 ; c1ccc(-c2cnc3c(ccc4cc(-c5cccc(-c6cccc(-c7nc8c9ccccc9ccc8c8ccccc78)n6)c5)cnc43)c2)cc1 ; c1ccc(-c2ccnc3c2ccc2cc(-c4cccc(-c5cccc(-c6nc7c8ccccc8ccc7c7ccccc67)n5)c4)cnc23)cc1]

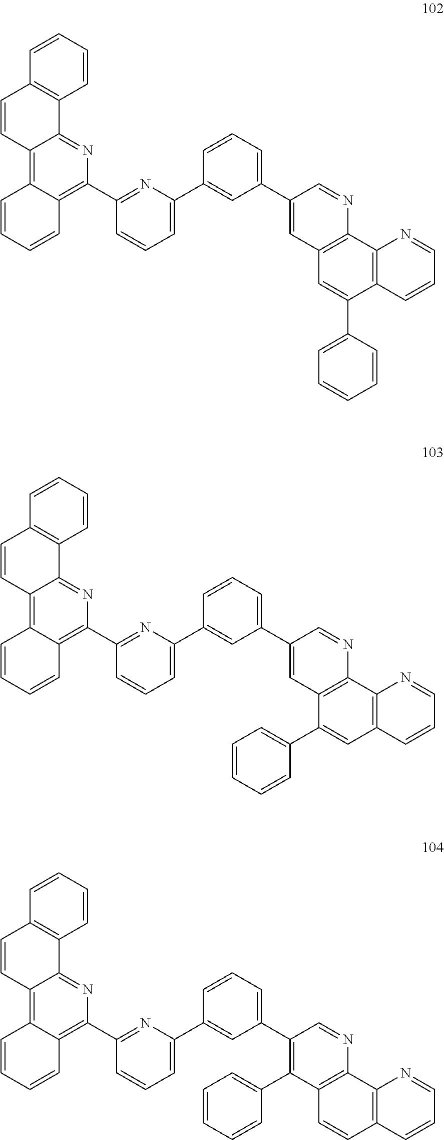 [SMILES: c1ccc(-c2cc3cc(-c4cccc(-c5cccc(-c6nc7c8ccccc8ccc7c7ccccc67)n5)c4)cnc3c3ncccc23)cc1 ; c1ccc(-c2cc3cccnc3c3ncc(-c4cccc(-c5cccc(-c6nc7c8ccccc8ccc7c7ccccc67)n5)c4)cc23)cc1 ; c1ccc(-c2c(-c3cccc(-c4cccc(-c5nc6c7ccccc7ccc6c6ccccc56)n4)c3)cnc3c2ccc2cccnc23)cc1]

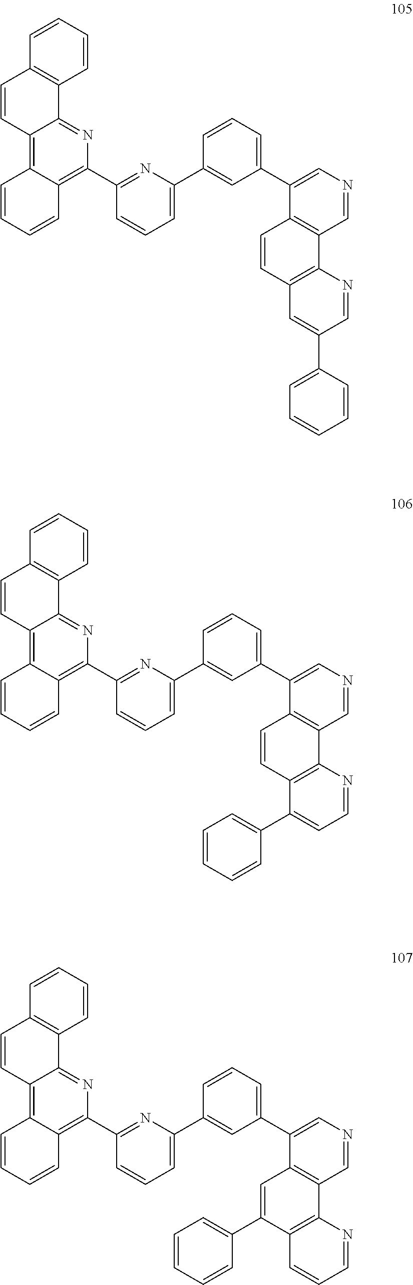 [SMILES: c1ccc(-c2cnc3c(ccc4c(-c5cccc(-c6cccc(-c7nc8c9ccccc9ccc8c8ccccc78)n6)c5)cncc43)c2)cc1 ; c1ccc(-c2ccnc3c2ccc2c(-c4cccc(-c5cccc(-c6nc7c8ccccc8ccc7c7ccccc67)n5)c4)cncc23)cc1 ; c1ccc(-c2cc3c(-c4cccc(-c5cccc(-c6nc7c8ccccc8ccc7c7ccccc67)n5)c4)cncc3c3ncccc23)cc1]

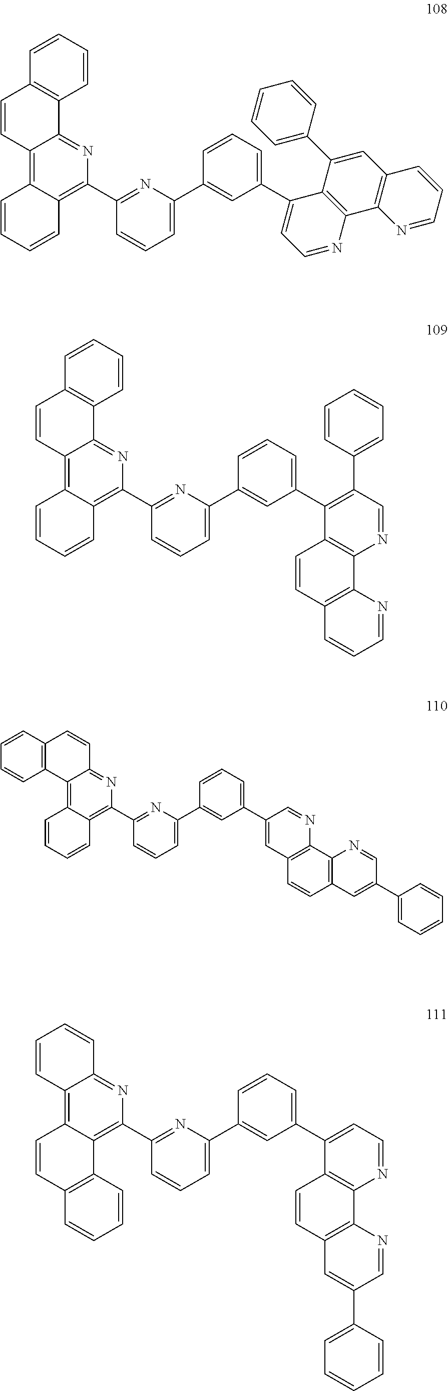 [SMILES: c1ccc(-c2cc3cccnc3c3nccc(-c4cccc(-c5cccc(-c6nc7c8ccccc8ccc7c7ccccc67)n5)c4)c23)cc1 ; c1ccc(-c2cnc3c(ccc4cccnc43)c2-c2cccc(-c3cccc(-c4nc5c6ccccc6ccc5c5ccccc45)n3)c2)cc1 ; c1ccc(-c2cnc3c(ccc4cc(-c5cccc(-c6cccc(-c7nc8ccc9ccccc9c8c8ccccc78)n6)c5)cnc43)c2)cc1 ; c1ccc(-c2cnc3c(ccc4c(-c5cccc(-c6cccc(-c7nc8ccccc8c8ccc9ccccc9c78)n6)c5)ccnc43)c2)cc1]

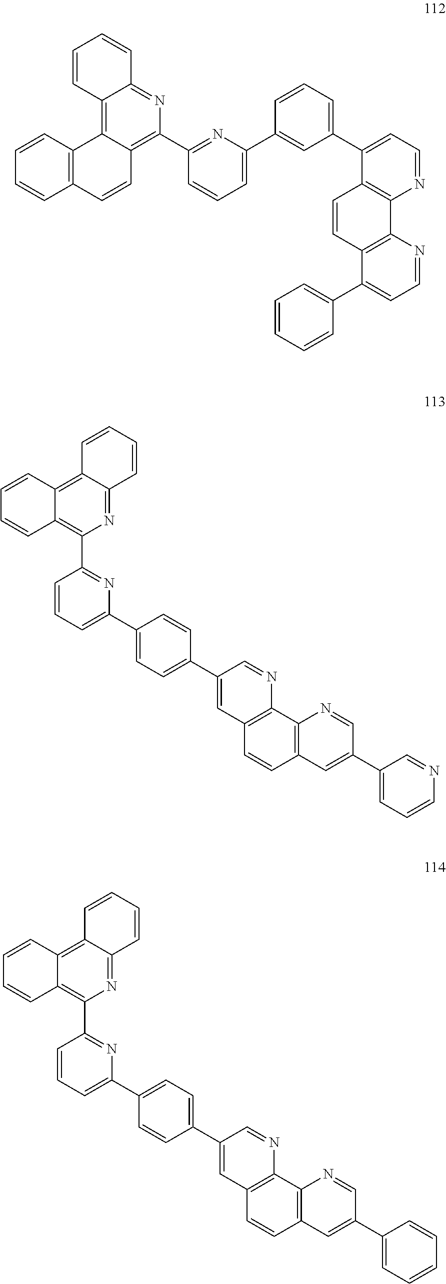 [SMILES: c1ccc(-c2ccnc3c2ccc2c(-c4cccc(-c5cccc(-c6nc7ccccc7c7c6ccc6ccccc67)n5)c4)ccnc23)cc1 ; c1cncc(-c2cnc3c(ccc4cc(-c5ccc(-c6cccc(-c7nc8ccccc8c8ccccc78)n6)cc5)cnc43)c2)c1 ; c1ccc(-c2cnc3c(ccc4cc(-c5ccc(-c6cccc(-c7nc8ccccc8c8ccccc78)n6)cc5)cnc43)c2)cc1]

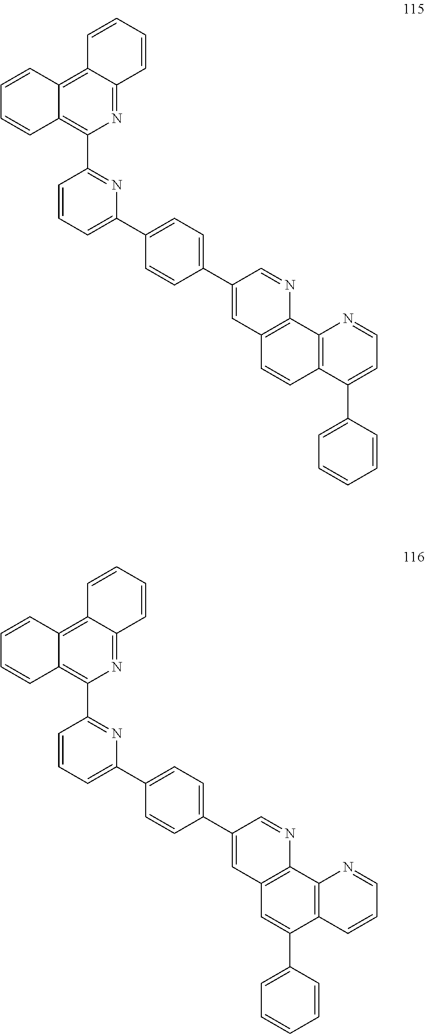 [SMILES: c1ccc(-c2ccnc3c2ccc2cc(-c4ccc(-c5cccc(-c6nc7ccccc7c7ccccc67)n5)cc4)cnc23)cc1 ; c1ccc(-c2cc3cc(-c4ccc(-c5cccc(-c6nc7ccccc7c7ccccc67)n5)cc4)cnc3c3ncccc23)cc1]

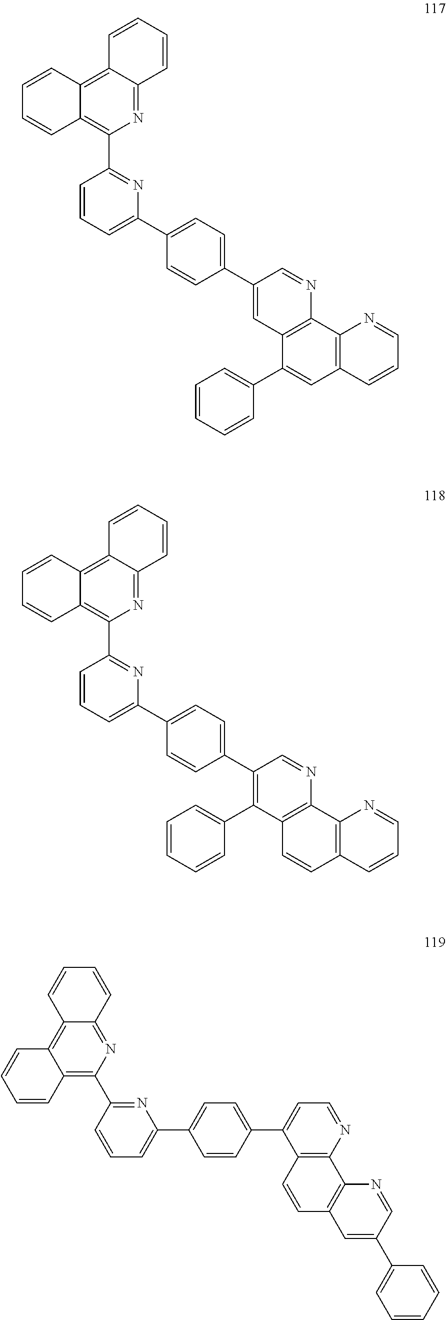 [SMILES: c1ccc(-c2cc3cccnc3c3ncc(-c4ccc(-c5cccc(-c6nc7ccccc7c7ccccc67)n5)cc4)cc23)cc1 ; c1ccc(-c2c(-c3ccc(-c4cccc(-c5nc6ccccc6c6ccccc56)n4)cc3)cnc3c2ccc2cccnc23)cc1 ; c1ccc(-c2cnc3c(ccc4c(-c5ccc(-c6cccc(-c7nc8ccccc8c8ccccc78)n6)cc5)ccnc43)c2)cc1]

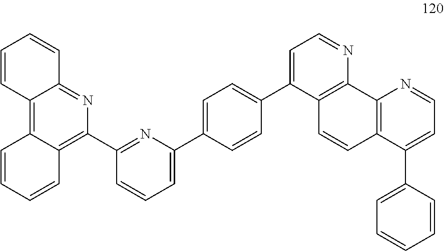 [SMILES: c1ccc(-c2ccnc3c2ccc2c(-c4ccc(-c5cccc(-c6nc7ccccc7c7ccccc67)n5)cc4)ccnc23)cc1]

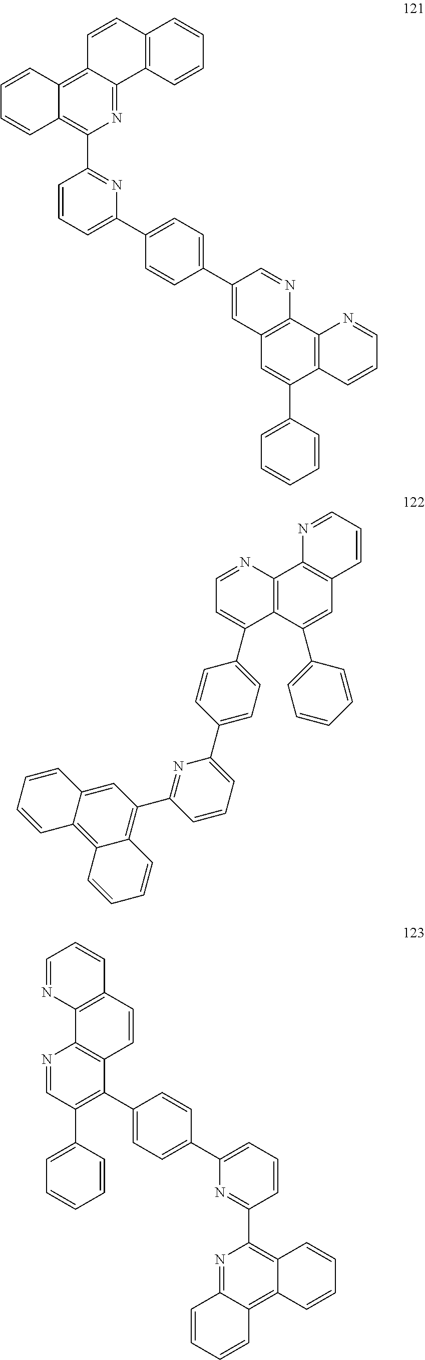

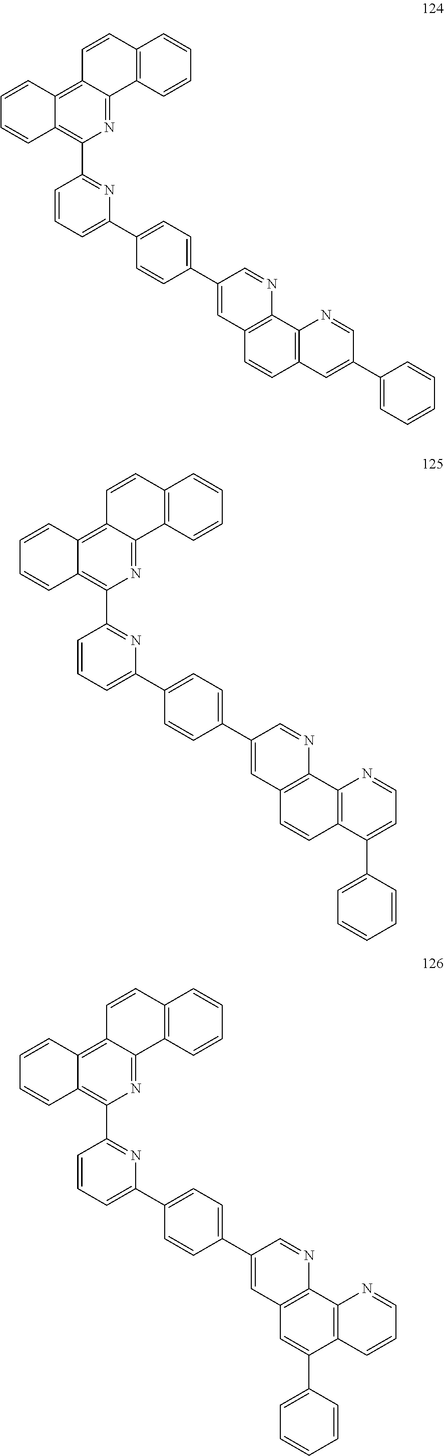 [SMILES: c1ccc(-c2cnc3c(ccc4cc(-c5ccc(-c6cccc(-c7nc8c9ccccc9ccc8c8ccccc78)n6)cc5)cnc43)c2)cc1 ; c1ccc(-c2ccnc3c2ccc2cc(-c4ccc(-c5cccc(-c6nc7c8ccccc8ccc7c7ccccc67)n5)cc4)cnc23)cc1 ; c1ccc(-c2cc3cc(-c4ccc(-c5cccc(-c6nc7c8ccccc8ccc7c7ccccc67)n5)cc4)cnc3c3ncccc23)cc1]

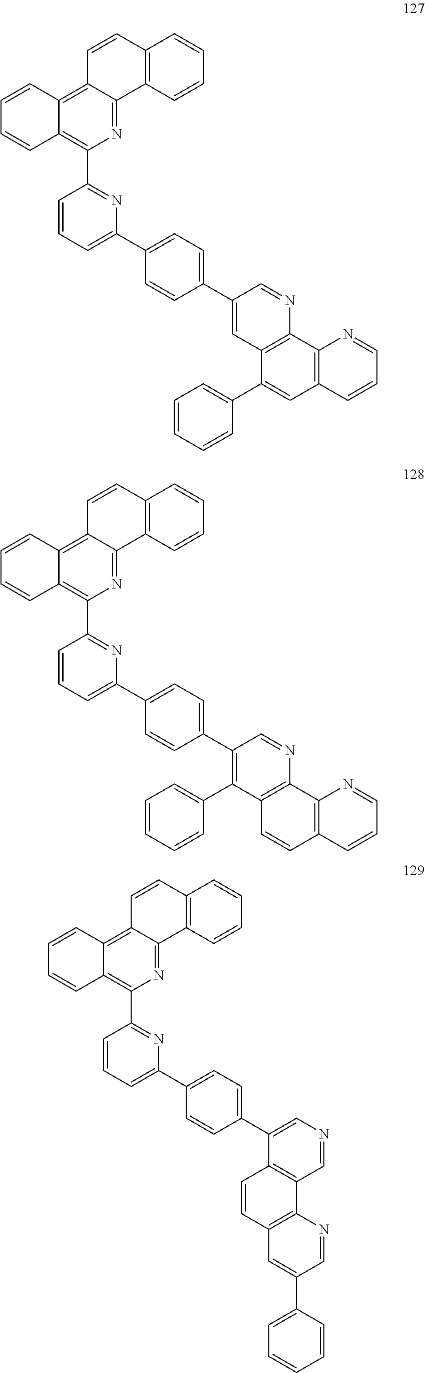

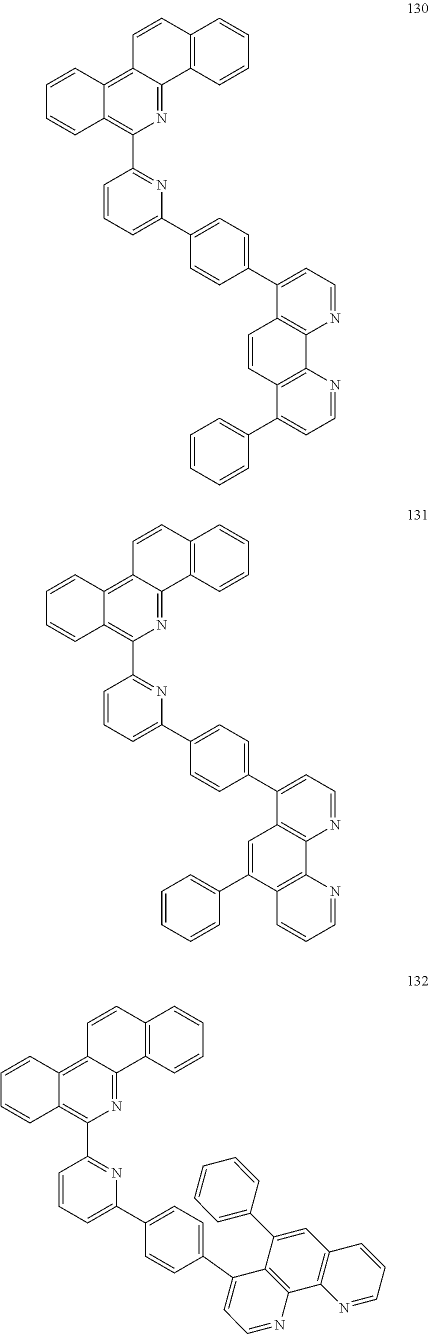

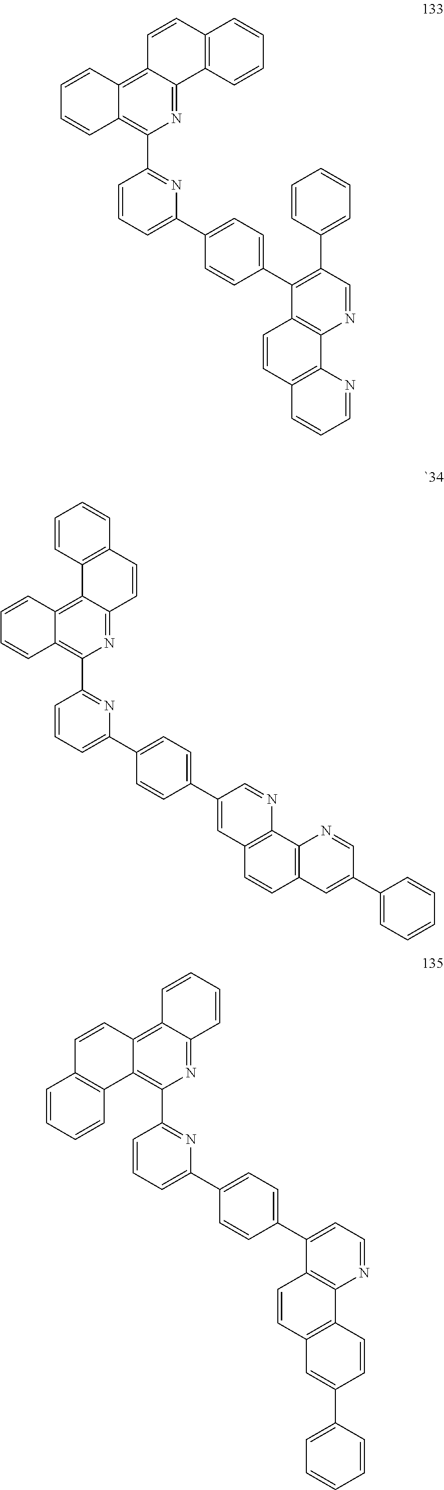 [SMILES: c1ccc(-c2cnc3c(ccc4cccnc43)c2-c2ccc(-c3cccc(-c4nc5c6ccccc6ccc5c5ccccc45)n3)cc2)cc1 ; c1ccc(-c2cnc3c(ccc4cc(-c5ccc(-c6cccc(-c7nc8ccc9ccccc9c8c8ccccc78)n6)cc5)cnc43)c2)cc1 ; c1ccc(-c2ccc3c(ccc4c(-c5ccc(-c6cccc(-c7nc8ccccc8c8ccc9ccccc9c78)n6)cc5)ccnc43)c2)cc1]

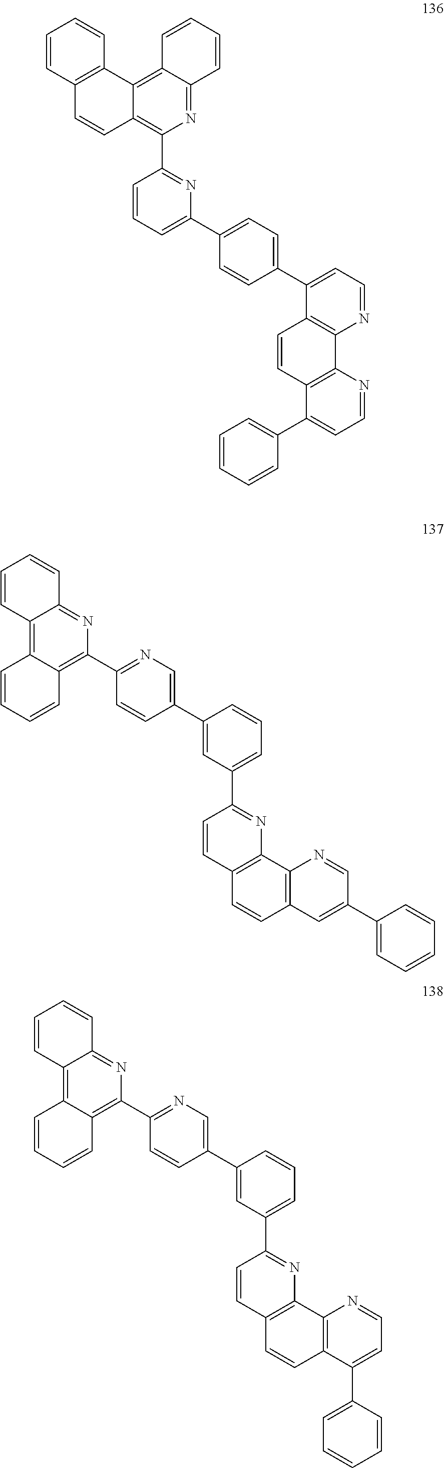 [SMILES: c1ccc(-c2ccnc3c2ccc2c(-c4ccc(-c5cccc(-c6nc7ccccc7c7c6ccc6ccccc67)n5)cc4)ccnc23)cc1 ; c1ccc(-c2cnc3c(ccc4ccc(-c5cccc(-c6ccc(-c7nc8ccccc8c8ccccc78)nc6)c5)nc43)c2)cc1 ; c1ccc(-c2ccnc3c2ccc2ccc(-c4cccc(-c5ccc(-c6nc7ccccc7c7ccccc67)nc5)c4)nc23)cc1]

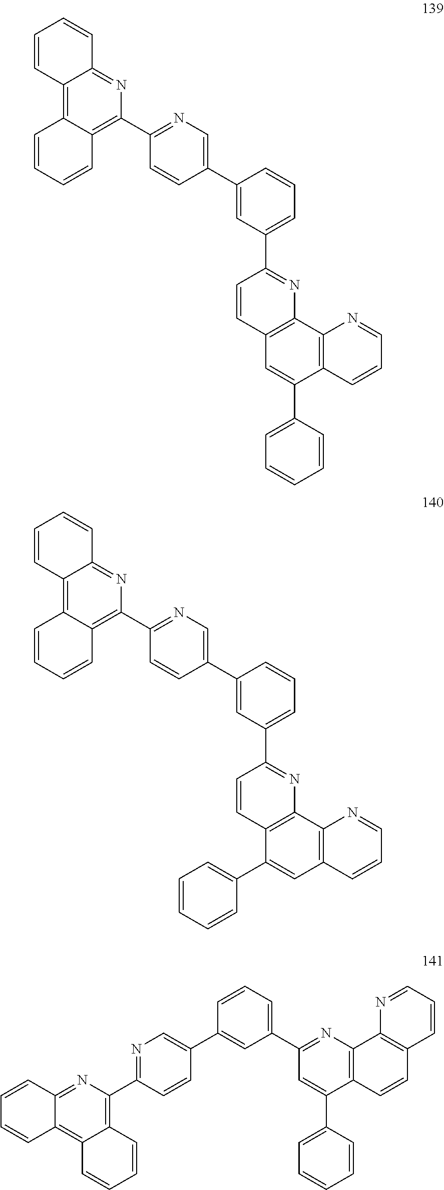 [SMILES: c1ccc(-c2cc3ccc(-c4cccc(-c5ccc(-c6nc7ccccc7c7ccccc67)nc5)c4)nc3c3ncccc23)cc1 ; c1ccc(-c2cc3cccnc3c3nc(-c4cccc(-c5ccc(-c6nc7ccccc7c7ccccc67)nc5)c4)ccc23)cc1 ; c1ccc(-c2cc(-c3cccc(-c4ccc(-c5nc6ccccc6c6ccccc56)nc4)c3)nc3c2ccc2cccnc23)cc1]

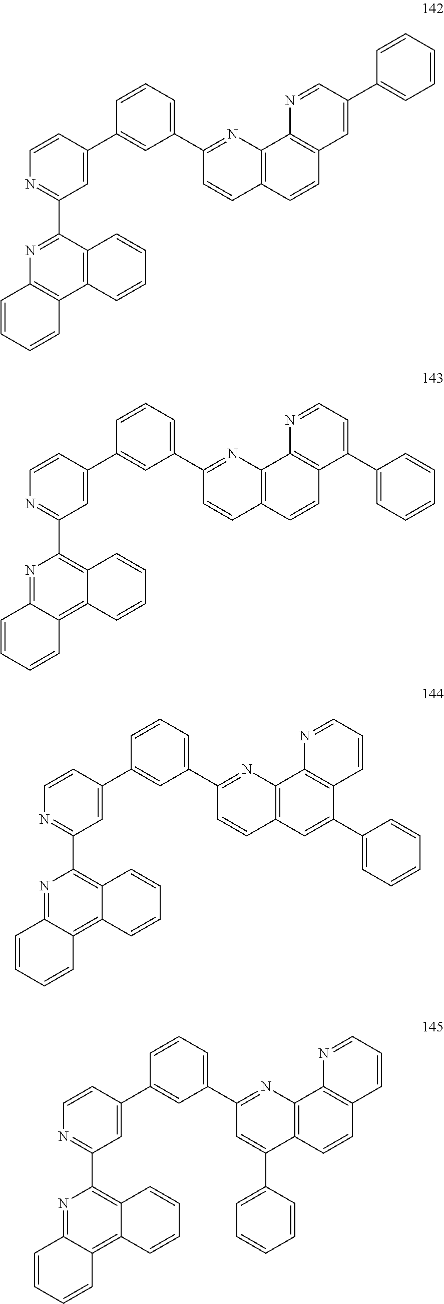 [SMILES: c1ccc(-c2cnc3c(ccc4ccc(-c5cccc(-c6ccnc(-c7nc8ccccc8c8ccccc78)c6)c5)nc43)c2)cc1 ; c1ccc(-c2ccnc3c2ccc2ccc(-c4cccc(-c5ccnc(-c6nc7ccccc7c7ccccc67)c5)c4)nc23)cc1 ; c1ccc(-c2cc3ccc(-c4cccc(-c5ccnc(-c6nc7ccccc7c7ccccc67)c5)c4)nc3c3ncccc23)cc1 ; c1ccc(-c2cc(-c3cccc(-c4ccnc(-c5nc6ccccc6c6ccccc56)c4)c3)nc3c2ccc2cccnc23)cc1]

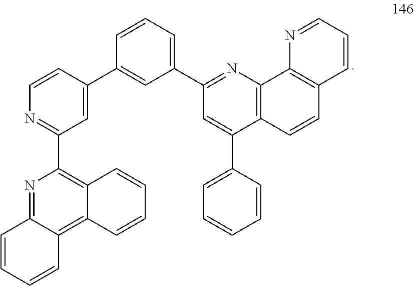 [SMILES: c1ccc(-c2cc(-c3cccc(-c4ccnc(-c5nc6ccccc6c6ccccc56)c4)c3)nc3c2ccc2cccnc23)cc1]

7. An organic electroluminescence device comprising:
an anode; a cathode; one or more organic layer interposed between the anode and the cathode,
wherein at least one of the one or more organic layers comprises the compound represented by the following Chemical Formula 1 according to claim 1: 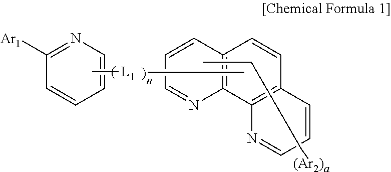
wherein,
Ar₁ is a C9-C40 heteroaryl group containing one nitrogen (N) atom,
n is an integer in a range of 1 to 3,
L₁ is a C6-C40 arylene group,
a is an integer in a range of 0 to 7,
Ar₂ is selected from the group consisting of: hydrogen, deuterium (D), halogen group, cyano group, nitro group, amino group, C1-C40 alkyl group, C2-C40 alkenyl group, C2-C40 alkynyl group, C3-C40 cycloalkyl group, heterocycloalkyl group having 3 to 40 nuclear atoms, C6-C60 aryl group, heteroaryl group having 5 to 60 nuclear atoms, C1-C40 alkyloxy group, C6-C60 aryloxy group, C1-C40 alkylsilyl group, C6-C60 arylsilyl group, C1-C40 alkylboron group, C6-C60 arylboron group, C6-C60 arylphosphine group, C6-C60 arylphosphine oxide group, C6-C60 arylamine group, (C6-C60 aryl) (heteroaryl having 5 to 60 nuclear atoms) amine group, and heteroarylamine group having 5 to 60 nuclear atoms, or fused with an adjacent group to form a fused ring;
the heteroaryl group of Ar₁, the arylene group of L₁, and the alkyl group, alkenyl group, alkynyl group, cycloalkyl group, heterocycloalkyl group, aryl group, heteroaryl group, alkyloxy group, aryloxy group, alkylsilyl group, arylsilyl group, alkylboron group, arylboron group, arylphosphine group, arylphosphine oxide, arylamine group, (aryl) (heteroaryl)amine, and heteroarylamine group of Ar₂ are each independently substituted or unsubstituted with one or more substituents selected from the group consisting of: deuterium (D), halogen group, cyano group, nitro group, amino group, C1-C40 alkyl group, C2-C40 alkenyl group, C2-C40 alkynyl group, C3-C40 cycloalkyl group, heterocycloalkyl group having 3 to 40 nuclear atoms, C6-C60 aryl group, heteroaryl group having 5 to 60 nuclear atoms, C1-C40 alkyloxy group, C6-C60 aryloxy group, C1-C40 alkylsilyl group, C6-C60 arylsilyl group, C1-C40 alkylboron group, C6-C60 arylboron group, C6-C60 arylphosphine group, C6-C60 arylphosphine oxide group, C6-C60 arylamine group, (C6-C60 aryl) (heteroaryl having 5 to 60 nuclear atoms) amine group, and heteroarylamine group having 5 to 60 nuclear atoms, and when a plurality of the substituents are present, they are the same as or different from each other.

8. The organic electroluminescence device of claim 7,
wherein the organic layer comprising the organic compound is an electron transport layer.

9. An organic electroluminescence device comprising:
an anode and a cathode that are arranged at a distance;
a plurality of light-emitting units interposed between the anode and the cathode; and
an N-type charge generation layer and a P-type charge generation layer, interposed between the adjacent light-emitting units,
wherein each of the light-emitting units comprises a hole transport layer, a light-emitting layer, and an electron transport layer, and
the N-type charge generation layer comprises the organic compound represented by the following Chemical Formula 1 according to claim 1: 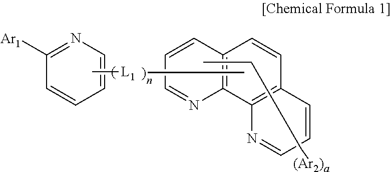
wherein,
Ar₁ is a C9-C40 heteroaryl group containing one nitrogen (N) atom,
n is an integer in a range of 1 to 3,
L₁ is a C6-C40 arylene group,
a is an integer in a range of 0 to 7,
Ar₂ is selected from the group consisting of: hydrogen, deuterium (D), halogen group, cyano group, nitro group, amino group, C1-C40 alkyl group, C2-C40 alkenyl group, C2-C40 alkynyl group, C3-C40 cycloalkyl group, heterocycloalkyl group having 3 to 40 nuclear atoms, C6-C60 aryl group, heteroaryl group having 5 to 60 nuclear atoms, C1-C40 alkyloxy group, C6-C60 aryloxy group, C1-C40 alkylsilyl group, C6-C60 arylsilyl group, C1-C40 alkylboron group, C6-C60 arylboron group, C6-C60 arylphosphine group, C6-C60 arylphosphine oxide group, C6-C60 arylamine group, (C6-C60 aryl) (heteroaryl having 5 to 60 nuclear atoms) amine group, and heteroarylamine group having 5 to 60 nuclear atoms, or fused with an adjacent group to form a fused ring;
the heteroaryl group of Ar₁, the arylene group of L₁, and the alkyl group, alkenyl group, alkynyl group, cycloalkyl group, heterocycloalkyl group, aryl group, heteroaryl group, alkyloxy group, aryloxy group, alkylsilyl group, arylsilyl group, alkylboron group, arylboron group, arylphosphine group, arylphosphine oxide, arylamine group, (aryl) (heteroaryl)amine, and heteroarylamine group of Ar₂ are each independently substituted or unsubstituted with one or more substituents selected from the group consisting of: deuterium (D), halogen group, cyano group, nitro group, amino group, C1-C40 alkyl group, C2-C40 alkenyl group, C2-C40 alkynyl group, C3-C40 cycloalkyl group, heterocycloalkyl group having 3 to 40 nuclear atoms, C6-C60 aryl group, heteroaryl group having 5 to 60 nuclear atoms, C1-C40 alkyloxy group, C6-C60 aryloxy group, C1-C40 alkylsilyl group, C6-C60 arylsilyl group, C1-C40 alkylboron group, C6-C60 arylboron group, C6-C60 arylphosphine group, C6-C60 arylphosphine oxide group, C6-C60 arylamine group, (C6-C60 aryl) (heteroaryl having 5 to 60 nuclear atoms) amine group, and heteroarylamine group having 5 to 60 nuclear atoms, and when a plurality of the substituents are present, they are the same as or different from each other.

10. The organic electroluminescence device of claim 9,
wherein the N-type charge generation layer comprises one host exhibiting an electron transport property, and the host is the organic compound represented by the Chemical Formula 1.

11. The organic electroluminescence device of claim 10,
wherein the N-type charge generation layer further comprises an N-type dopant.

12. The organic electroluminescence device of claim 7,
wherein Ar₁ is a substituent represented by the following Chemical Formula S1-1 or S1-2: 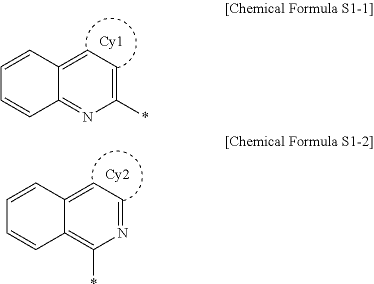
wherein in the above Chemical Formulas S1-1 and S1-2,
Cy1 and Cy2 are each independently a C6-C30 fused aromatic ring,
the fused aromatic ring of Cy1 and Cy2 are each independently substituted or unsubstituted with one or more substituents selected from the group consisting of: deuterium (D), halogen group, cyano group, nitro group, amino group, C1-C40 alkyl group, C2-C40 alkenyl group, C2-C40 alkynyl group, C3-C40 cycloalkyl group, heterocycloalkyl group having 3 to 40 nuclear atoms, C6-C60 aryl group, heteroaryl group having 5 to 60 nuclear atoms, C1-C40 alkyloxy group, C6-C60 aryloxy group, C1-C40 alkylsilyl group, C6-C60 arylsilyl group, C1-C40 alkylboron group, C6-C60 arylboron group, C6-C60 arylphosphine group, C6-C60 arylphosphine oxide group, C6-C60 arylamine group, (C6-C60 aryl) (heteroaryl having 5 to 60 nuclear atoms) amine group, and heteroarylamine group having 5 to 60 nuclear atoms, and when a plurality of the substituents are present, they are the same as or different from each other.

13. The organic electroluminescence device of claim 7,
wherein Ar₁ is selected from the group consisting of substituents represented by the following Chemical Formulas S2-1 to S2-9:

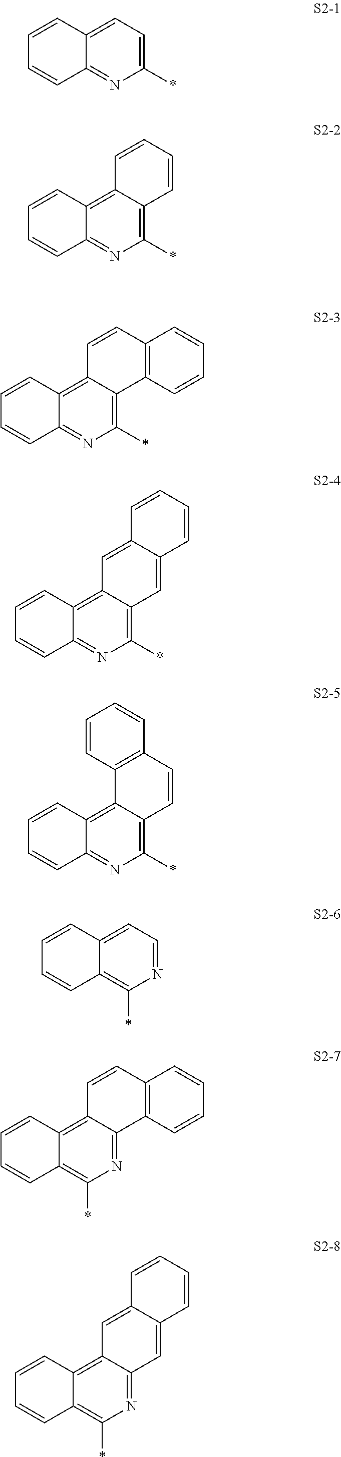

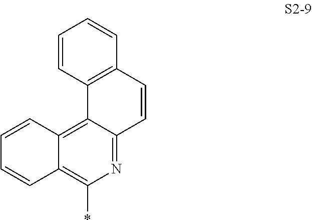 [SMILES: *c1nc2ccc3ccccc3c2c2ccccc12]

14. The organic electroluminescence device of claim 7,
wherein the compound represented by the above Chemical Formula 1 is represented by any one of Chemical Formulas 2 to 7: 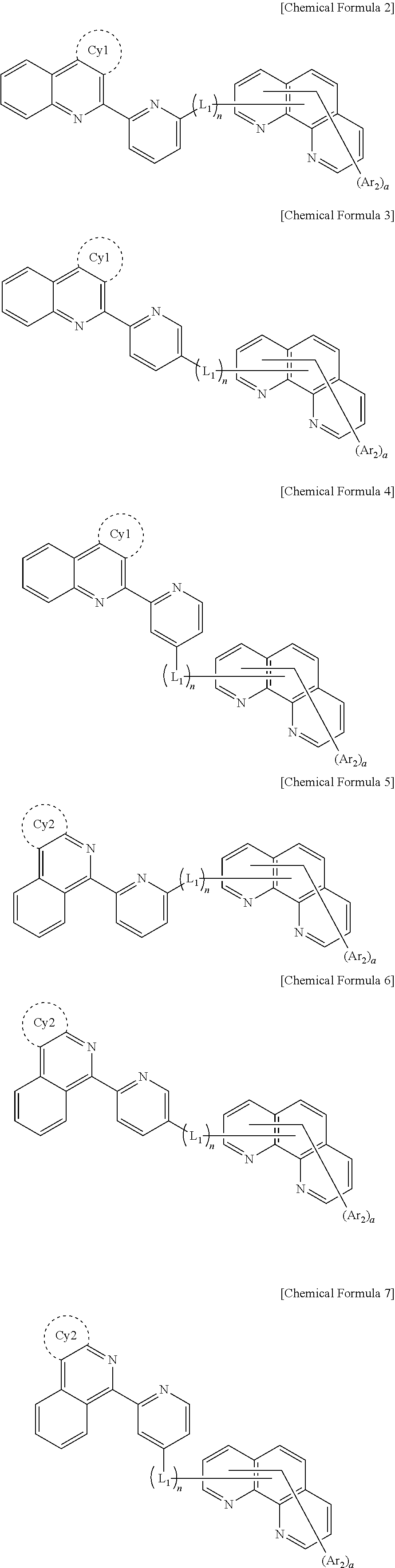
wherein in the above Chemical Formulas 2 to 7,
n, L₁, a, and Ar₂ are the same as defined in claim 7, respectively,
Cy1 and Cy2 are each independently a C6-C30 fused aromatic ring,
the fused aromatic ring of Cy1 and Cy2 are each independently substituted or unsubstituted with one or more substituents selected from the group consisting of: deuterium (D), halogen group, cyano group, nitro group, amino group, C1-C40 alkyl group, C2-C40 alkenyl group, C2-C40 alkynyl group, C3-C40 cycloalkyl group, heterocycloalkyl group having 3 to 40 nuclear atoms, C6-C60 aryl group, heteroaryl group having 5 to 60 nuclear atoms, C1-C40 alkyloxy group, C6-C60 aryloxy group, C1-C40 alkylsilyl group, C6-C60 arylsilyl group, C1-C40 alkylboron group, C6-C60 arylboron group, C6-C60 arylphosphine group, C6-C60 arylphosphine oxide group, C6-C60 arylamine group, (C6-C60 aryl) (heteroaryl having 5 to 60 nuclear atoms) amine group, and heteroarylamine group having 5 to 60 nuclear atoms, and when a plurality of the substituents are present, they are the same as or different from each other.

15. The organic electroluminescence device of claim 7,
wherein the compound represented by the above Chemical Formula 1 is selected from the group consisting of the following Compounds 001 to 146:

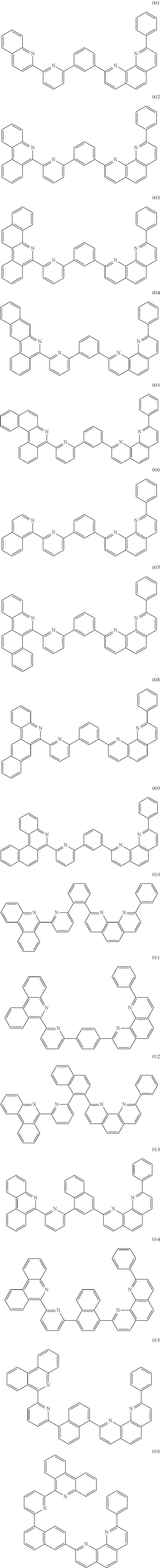

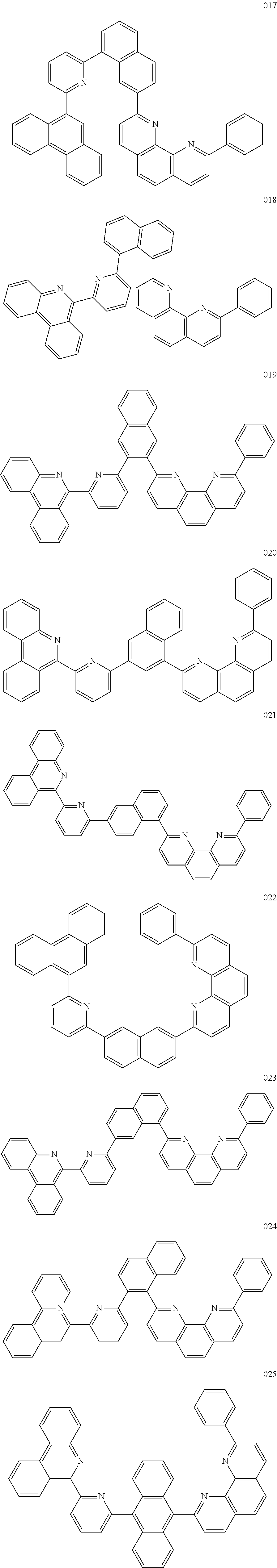

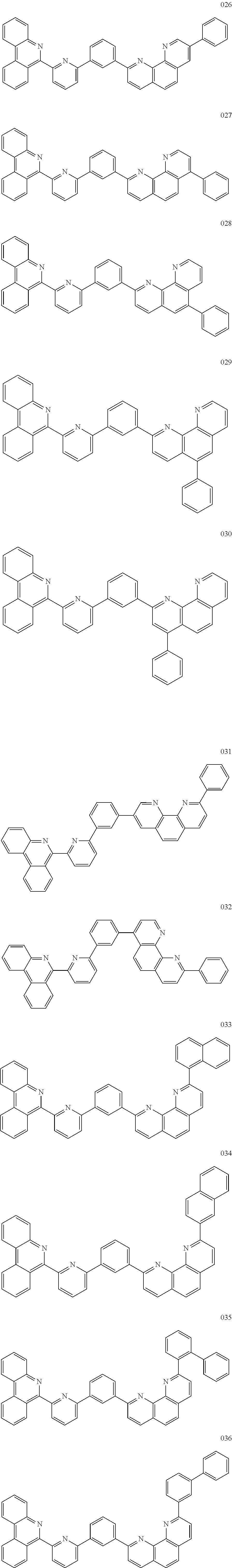

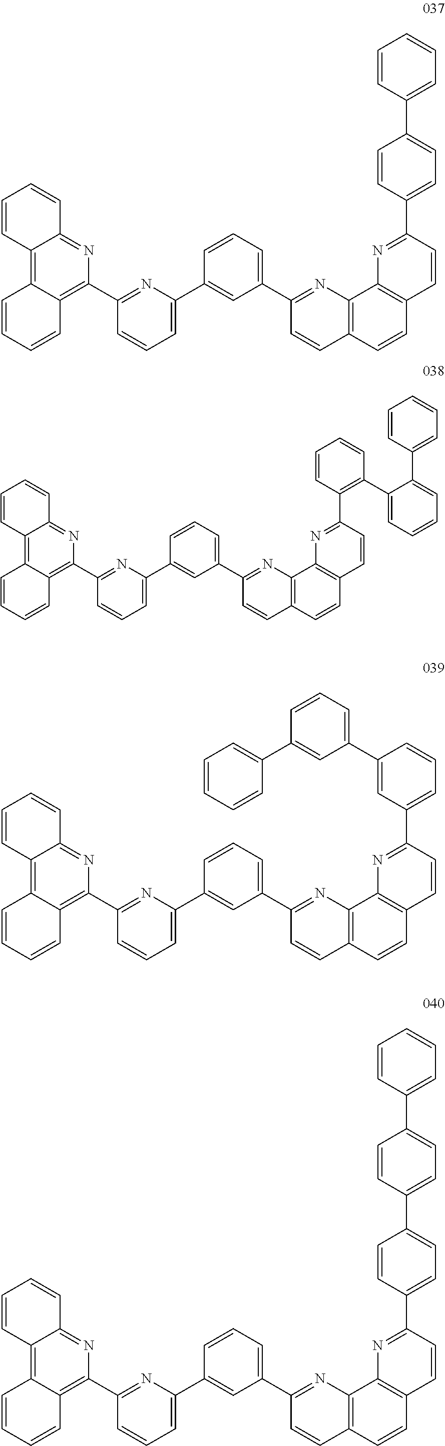 [SMILES: c1ccc(-c2ccc(-c3ccc4ccc5ccc(-c6cccc(-c7cccc(-c8nc9ccccc9c9ccccc89)n7)c6)nc5c4n3)cc2)cc1 ; c1ccc(-c2ccccc2-c2ccccc2-c2ccc3ccc4ccc(-c5cccc(-c6cccc(-c7nc8ccccc8c8ccccc78)n6)c5)nc4c3n2)cc1 ; c1ccc(-c2cccc(-c3cccc(-c4ccc5ccc6ccc(-c7cccc(-c8cccc(-c9nc%10ccccc%10c%10ccccc9%10)n8)c7)nc6c5n4)c3)c2)cc1 ; c1ccc(-c2ccc(-c3ccc(-c4ccc5ccc6ccc(-c7cccc(-c8cccc(-c9nc%10ccccc%10c%10ccccc9%10)n8)c7)nc6c5n4)cc3)cc2)cc1]

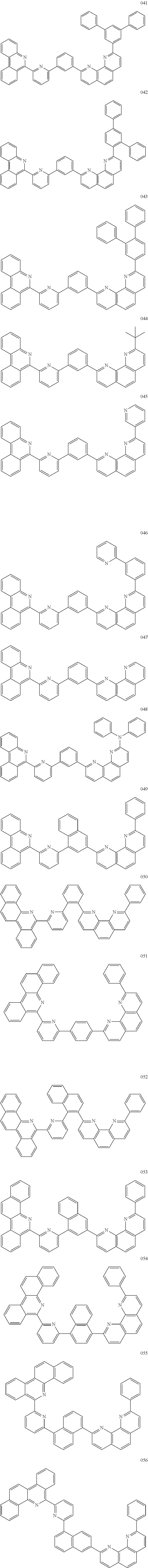

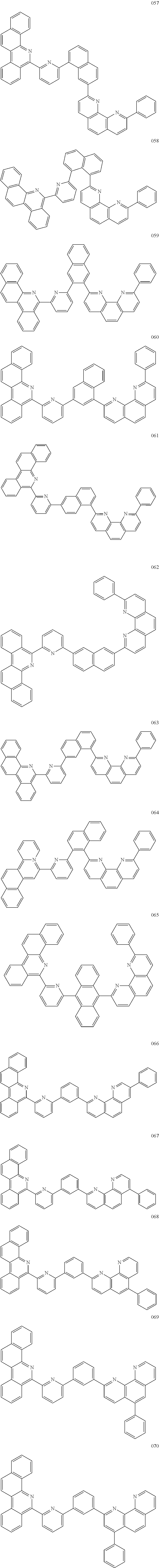

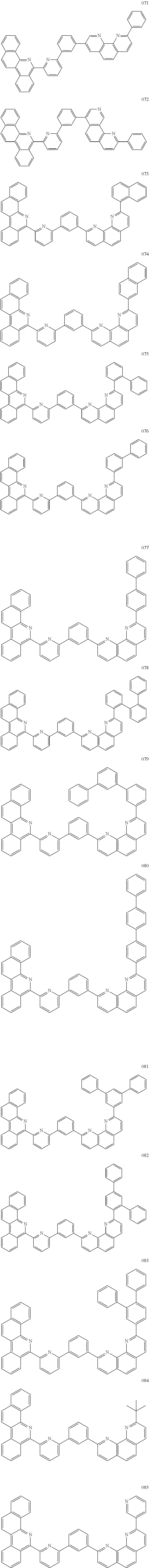

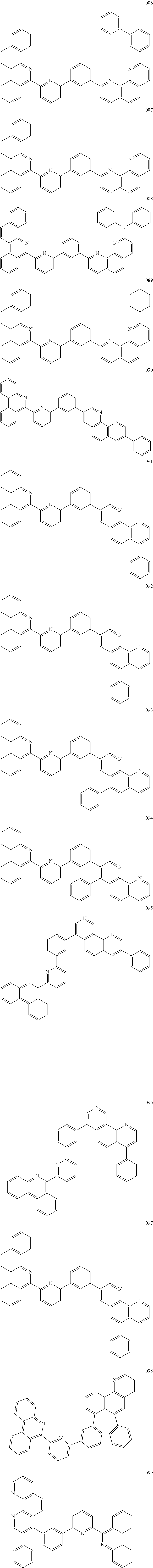

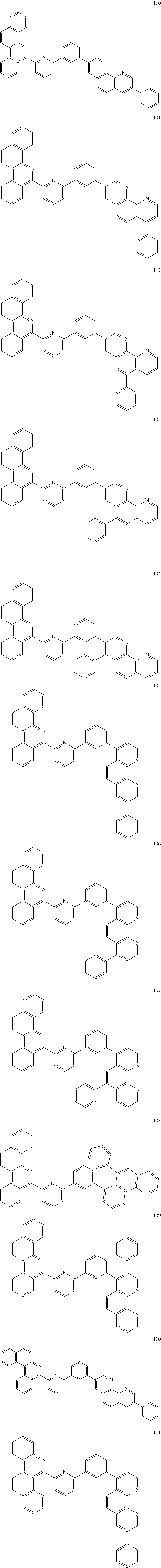

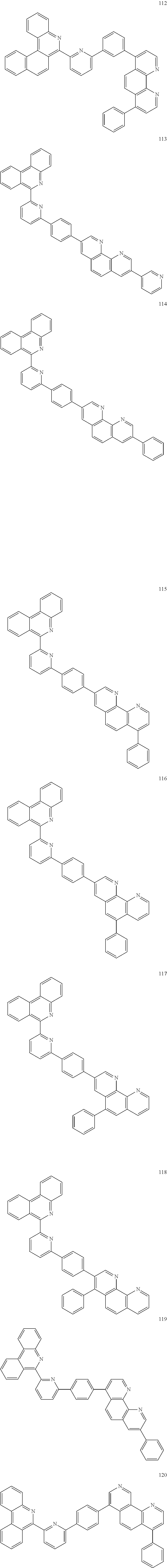

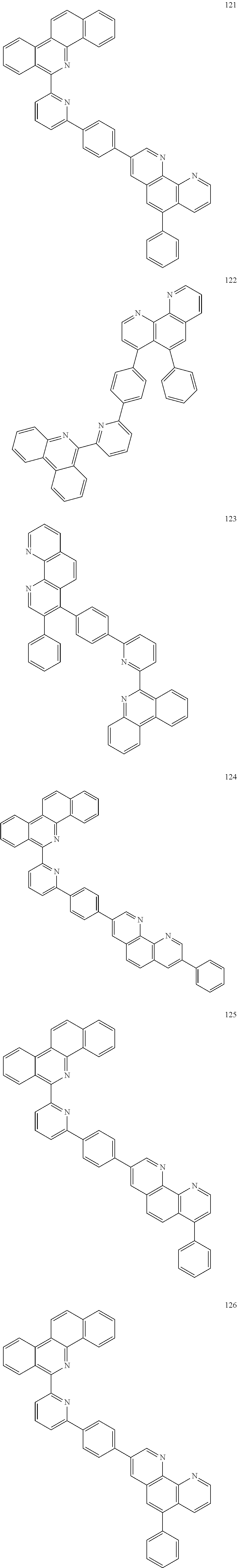 [SMILES: c1ccc(-c2cc3cc(-c4ccc(-c5cccc(-c6nc7c8ccccc8ccc7c7ccccc67)n5)cc4)cnc3c3ncccc23)cc1 ; c1ccc(-c2cc3cccnc3c3nccc(-c4ccc(-c5cccc(-c6nc7ccccc7c7ccccc67)n5)cc4)c23)cc1 ; c1ccc(-c2cnc3c(ccc4cccnc43)c2-c2ccc(-c3cccc(-c4nc5ccccc5c5ccccc45)n3)cc2)cc1 ; c1ccc(-c2cnc3c(ccc4cc(-c5ccc(-c6cccc(-c7nc8c9ccccc9ccc8c8ccccc78)n6)cc5)cnc43)c2)cc1 ; c1ccc(-c2ccnc3c2ccc2cc(-c4ccc(-c5cccc(-c6nc7c8ccccc8ccc7c7ccccc67)n5)cc4)cnc23)cc1 ; c1ccc(-c2cc3cc(-c4ccc(-c5cccc(-c6nc7c8ccccc8ccc7c7ccccc67)n5)cc4)cnc3c3ncccc23)cc1]

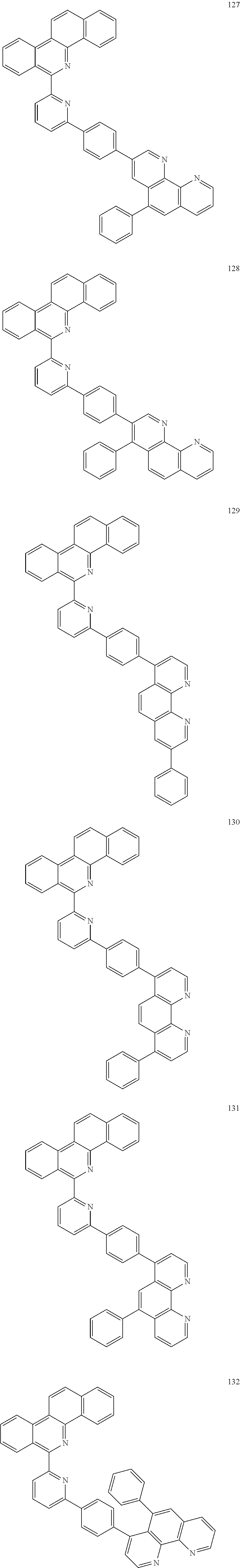

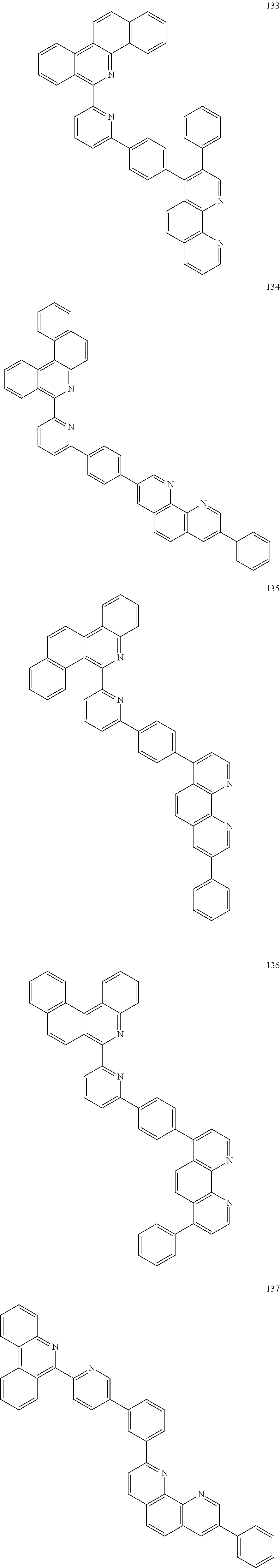 [SMILES: c1ccc(-c2cnc3c(ccc4cccnc43)c2-c2ccc(-c3cccc(-c4nc5c6ccccc6ccc5c5ccccc45)n3)cc2)cc1 ; c1ccc(-c2cnc3c(ccc4cc(-c5ccc(-c6cccc(-c7nc8ccc9ccccc9c8c8ccccc78)n6)cc5)cnc43)c2)cc1 ; c1ccc(-c2cnc3c(ccc4c(-c5ccc(-c6cccc(-c7nc8ccccc8c8ccc9ccccc9c78)n6)cc5)ccnc43)c2)cc1 ; c1ccc(-c2ccnc3c2ccc2c(-c4ccc(-c5cccc(-c6nc7ccccc7c7c6ccc6ccccc67)n5)cc4)ccnc23)cc1 ; c1ccc(-c2cnc3c(ccc4ccc(-c5cccc(-c6ccc(-c7nc8ccccc8c8ccccc78)nc6)c5)nc43)c2)cc1]

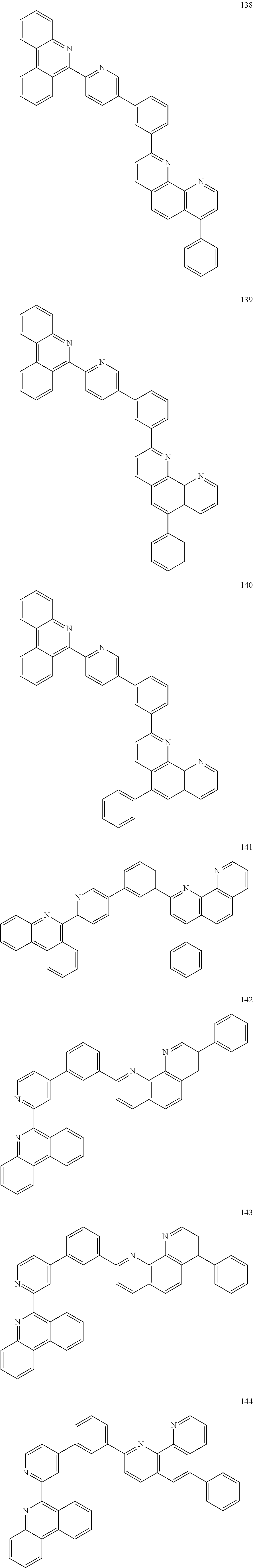

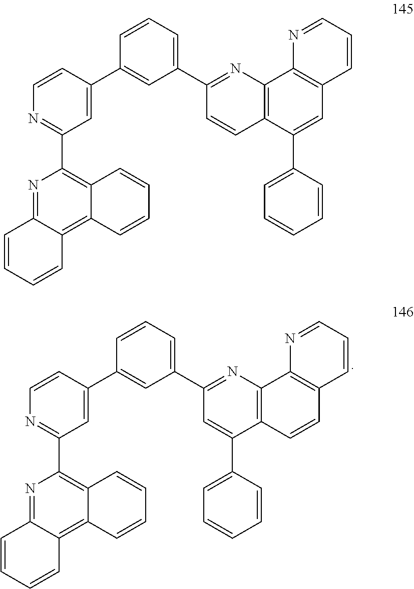 [SMILES: c1ccc(-c2cc3cccnc3c3nc(-c4cccc(-c5ccnc(-c6nc7ccccc7c7ccccc67)c5)c4)ccc23)cc1 ; c1ccc(-c2cc(-c3cccc(-c4ccnc(-c5nc6ccccc6c6ccccc56)c4)c3)nc3c2ccc2cccnc23)cc1]

16. The organic electroluminescence device of claim 9,
wherein Ar₁ is a substituent represented by the following Chemical Formula S1-1 or S1-2: 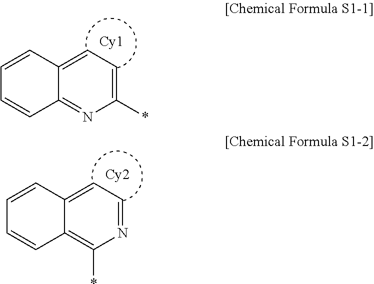
wherein in the above Chemical Formulas S1-1 and S1-2,
Cy1 and Cy2 are each independently a C6-C30 fused aromatic ring,
the fused aromatic ring of Cy1 and Cy2 are each independently substituted or unsubstituted with one or more substituents selected from the group consisting of: deuterium (D), halogen group, cyano group, nitro group, amino group, C1-C40 alkyl group, C2-C40 alkenyl group, C2-C40 alkynyl group, C3-C40 cycloalkyl group, heterocycloalkyl group having 3 to 40 nuclear atoms, C6-C60 aryl group, heteroaryl group having 5 to 60 nuclear atoms, C1-C40 alkyloxy group, C6-C60 aryloxy group, C1-C40 alkylsilyl group, C6-C60 arylsilyl group, C1-C40 alkylboron group, C6-C60 arylboron group, C6-C60 arylphosphine group, C6-C60 arylphosphine oxide group, C6-C60 arylamine group, (C6-C60 aryl) (heteroaryl having 5 to 60 nuclear atoms) amine group, and heteroarylamine group having 5 to 60 nuclear atoms, and when a plurality of the substituents are present, they are the same as or different from each other.

17. The organic electroluminescence device of claim 9,
wherein Ar₁ is selected from the group consisting of substituents represented by the following Chemical Formulas S2-1 to S2-9:

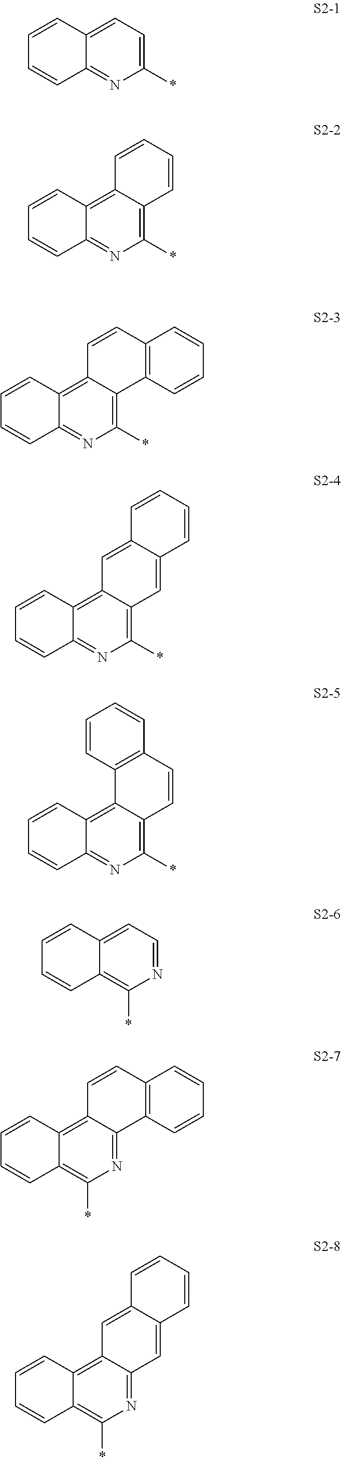

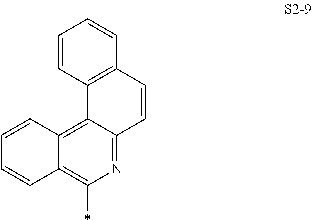 [SMILES: *c1nc2ccc3ccccc3c2c2ccccc12]

18. The organic electroluminescence device of claim 9,
wherein the compound represented by the above Chemical Formula 1 is represented by any one of Chemical Formulas 2 to 7: 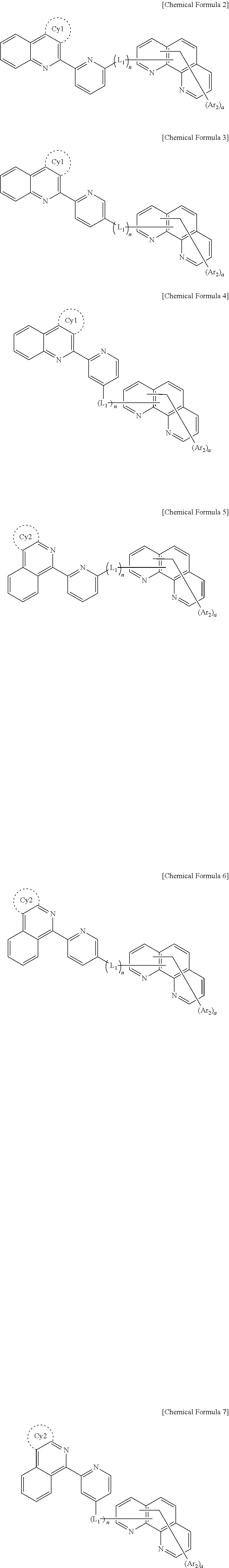
wherein in the above Chemical Formulas 2 to 7,
n, L₁, a, and Ar₂ are the same as defined in claim 9, respectively,
Cy1 and Cy2 are each independently a C6-C30 fused aromatic ring,
the fused aromatic ring of Cy1 and Cy2 are each independently substituted or unsubstituted with one or more substituents selected from the group consisting of: deuterium (D), halogen group, cyano group, nitro group, amino group, C1-C40 alkyl group, C2-C40 alkenyl group, C2-C40 alkynyl group, C3-C40 cycloalkyl group, heterocycloalkyl group having 3 to 40 nuclear atoms, C6-C60 aryl group, heteroaryl group having 5 to 60 nuclear atoms, C1-C40 C40 alkylboron group, C6-C60 arylboron group, C6-C60 arylphosphine group, C6-C60 arylphosphine oxide group, C6-C60 arylamine group, (C6-C60 aryl) (heteroaryl having 5 to 60 nuclear atoms) amine group, and heteroarylamine group having 5 to 60 nuclear atoms, and when a plurality of the substituents are present, they are the same as or different from each other.

19. The organic electroluminescence device of claim 9,
wherein the compound represented by the above Chemical Formula 1 is selected from the group consisting of the following Compounds 001 to 146: 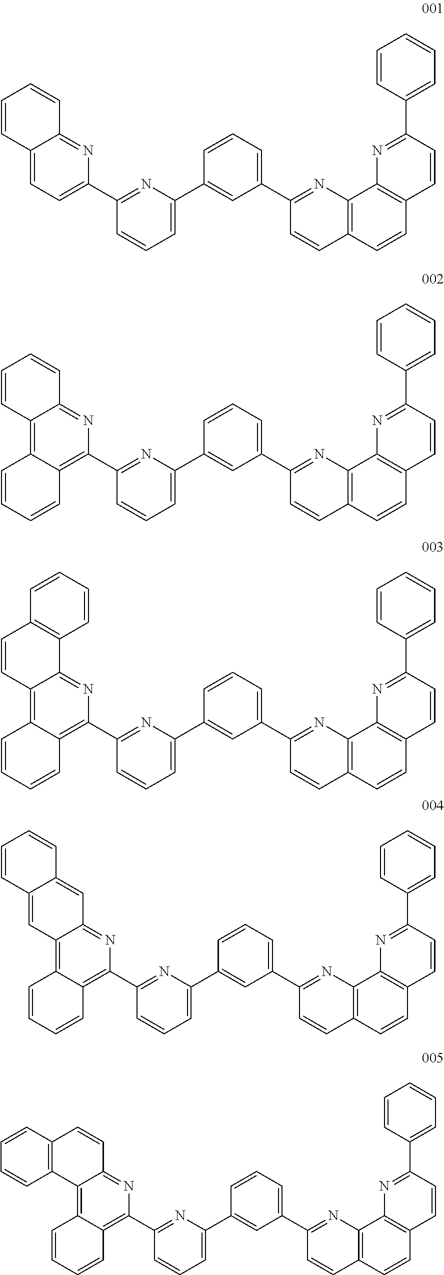 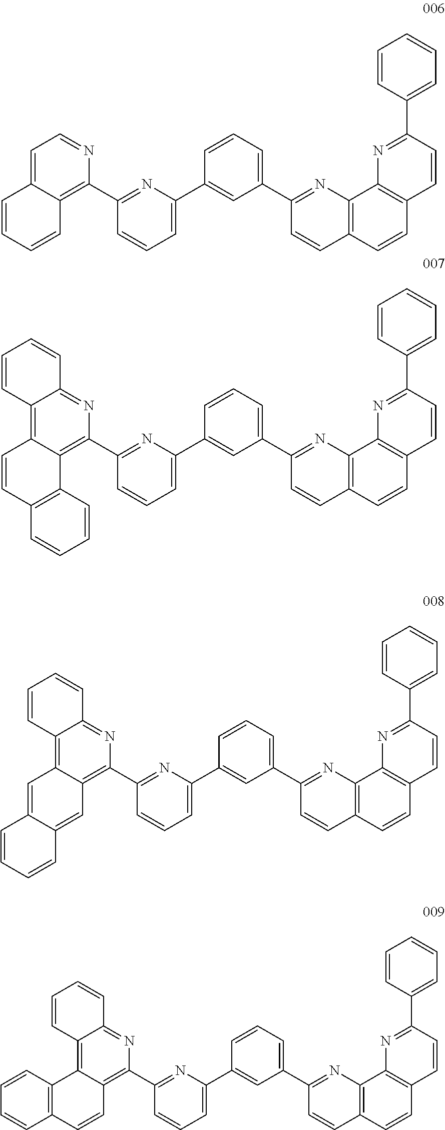 [SMILES: c1ccc(-c2ccc3ccc4ccc(-c5cccc(-c6cccc(-c7nccc8ccccc78)n6)c5)nc4c3n2)cc1 ; c1ccc(-c2ccc3ccc4ccc(-c5cccc(-c6cccc(-c7nc8ccccc8c8ccc9ccccc9c78)n6)c5)nc4c3n2)cc1 ; c1ccc(-c2ccc3ccc4ccc(-c5cccc(-c6cccc(-c7nc8ccccc8c8cc9ccccc9cc78)n6)c5)nc4c3n2)cc1 ; c1ccc(-c2ccc3ccc4ccc(-c5cccc(-c6cccc(-c7nc8ccccc8c8c7ccc7ccccc78)n6)c5)nc4c3n2)cc1] 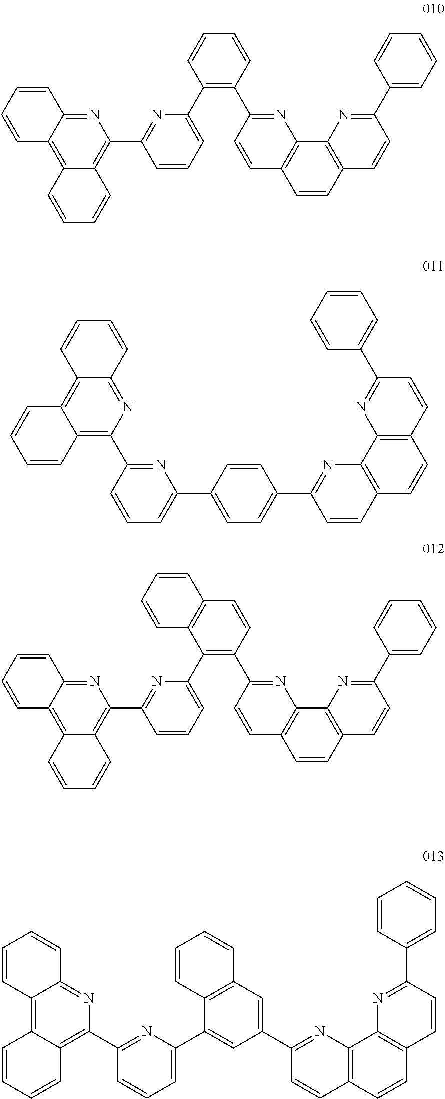 [SMILES: c1ccc(-c2ccc3ccc4ccc(-c5ccccc5-c5cccc(-c6nc7ccccc7c7ccccc67)n5)nc4c3n2)cc1 ; c1ccc(-c2ccc3ccc4ccc(-c5ccc(-c6cccc(-c7nc8ccccc8c8ccccc78)n6)cc5)nc4c3n2)cc1 ; c1ccc(-c2ccc3ccc4ccc(-c5ccc6ccccc6c5-c5cccc(-c6nc7ccccc7c7ccccc67)n5)nc4c3n2)cc1 ; c1ccc(-c2ccc3ccc4ccc(-c5cc(-c6cccc(-c7nc8ccccc8c8ccccc78)n6)c6ccccc6c5)nc4c3n2)cc1] 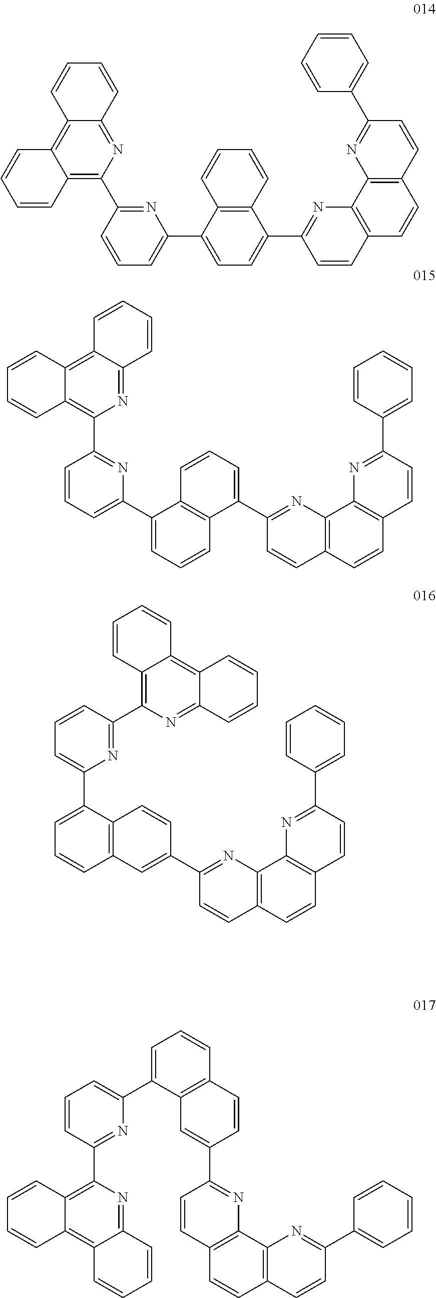 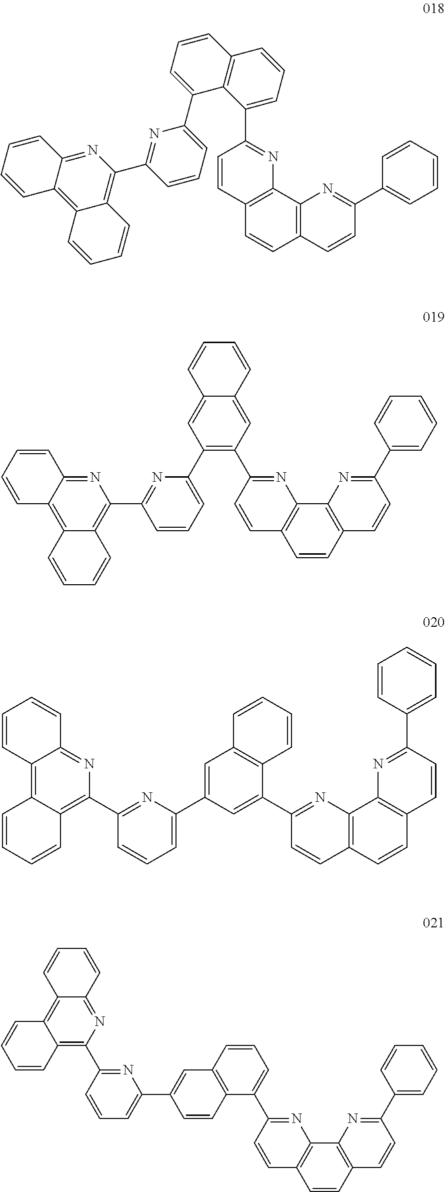 [SMILES: c1ccc(-c2ccc3ccc4ccc(-c5cccc6cccc(-c7cccc(-c8nc9ccccc9c9ccccc89)n7)c56)nc4c3n2)cc1 ; c1ccc(-c2ccc3ccc4ccc(-c5cc6ccccc6cc5-c5cccc(-c6nc7ccccc7c7ccccc67)n5)nc4c3n2)cc1 ; c1ccc(-c2ccc3ccc4ccc(-c5cc(-c6cccc(-c7nc8ccccc8c8ccccc78)n6)cc6ccccc56)nc4c3n2)cc1 ; c1ccc(-c2ccc3ccc4ccc(-c5cccc6cc(-c7cccc(-c8nc9ccccc9c9ccccc89)n7)ccc56)nc4c3n2)cc1] 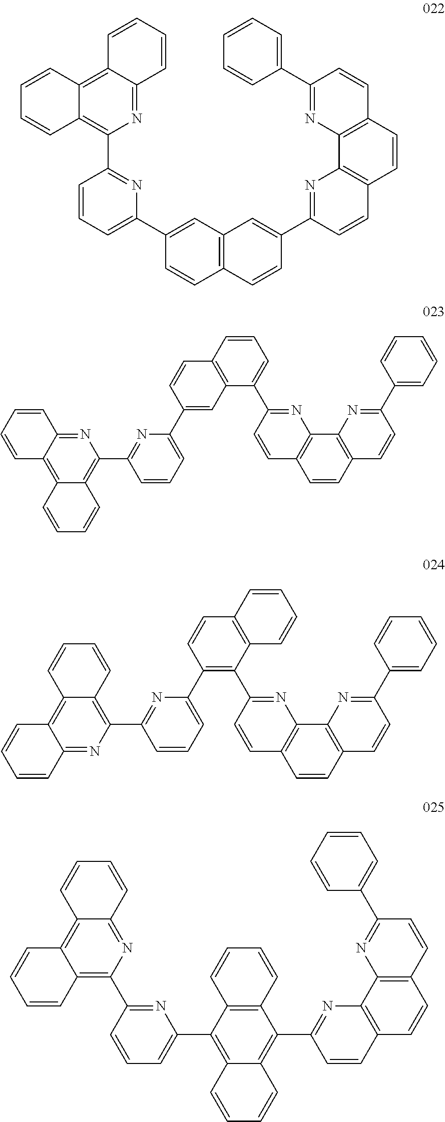 [SMILES: c1ccc(-c2ccc3ccc4ccc(-c5ccc6ccc(-c7cccc(-c8nc9ccccc9c9ccccc89)n7)cc6c5)nc4c3n2)cc1 ; c1ccc(-c2ccc3ccc4ccc(-c5cccc6ccc(-c7cccc(-c8nc9ccccc9c9ccccc89)n7)cc56)nc4c3n2)cc1 ; c1ccc(-c2ccc3ccc4ccc(-c5c(-c6cccc(-c7nc8ccccc8c8ccccc78)n6)ccc6ccccc56)nc4c3n2)cc1 ; c1ccc(-c2ccc3ccc4ccc(-c5c6ccccc6c(-c6cccc(-c7nc8ccccc8c8ccccc78)n6)c6ccccc56)nc4c3n2)cc1] 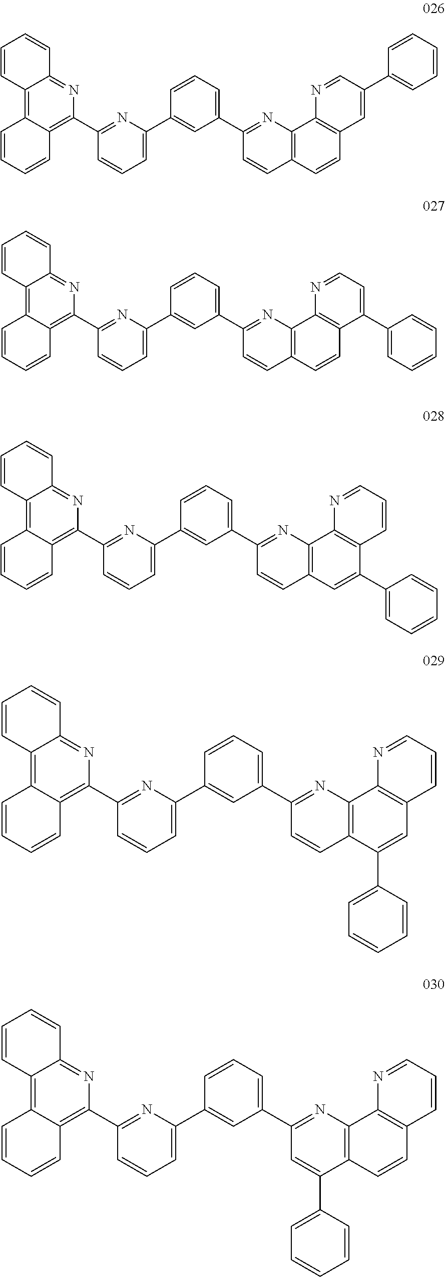 [SMILES: c1ccc(-c2cnc3c(ccc4ccc(-c5cccc(-c6cccc(-c7nc8ccccc8c8ccccc78)n6)c5)nc43)c2)cc1 ; c1ccc(-c2ccnc3c2ccc2ccc(-c4cccc(-c5cccc(-c6nc7ccccc7c7ccccc67)n5)c4)nc23)cc1 ; c1ccc(-c2cc3ccc(-c4cccc(-c5cccc(-c6nc7ccccc7c7ccccc67)n5)c4)nc3c3ncccc23)cc1 ; c1ccc(-c2cc3cccnc3c3nc(-c4cccc(-c5cccc(-c6nc7ccccc7c7ccccc67)n5)c4)ccc23)cc1 ; c1ccc(-c2cc(-c3cccc(-c4cccc(-c5nc6ccccc6c6ccccc56)n4)c3)nc3c2ccc2cccnc23)cc1] 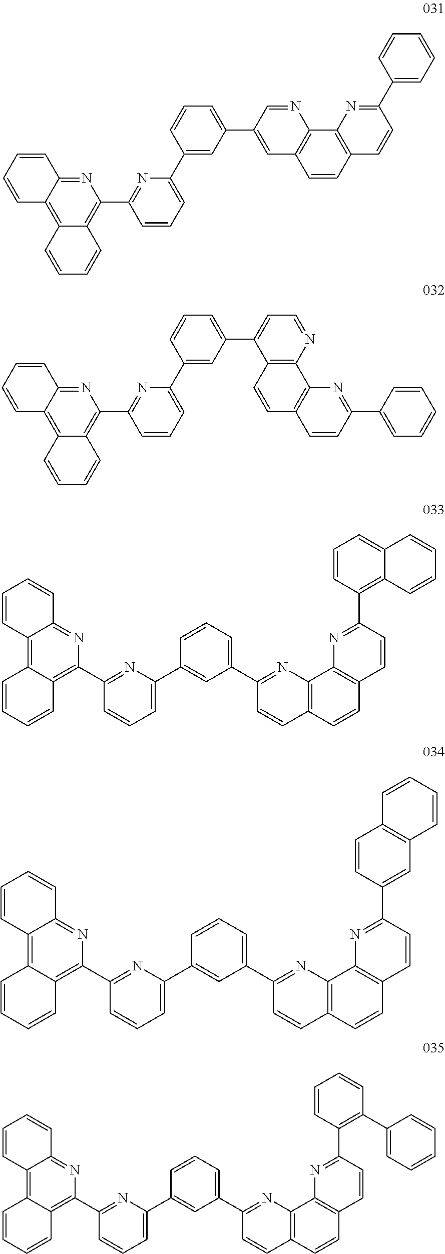 [SMILES: c1ccc(-c2ccc3ccc4cc(-c5cccc(-c6cccc(-c7nc8ccccc8c8ccccc78)n6)c5)cnc4c3n2)cc1 ; c1ccc(-c2ccc3ccc4c(-c5cccc(-c6cccc(-c7nc8ccccc8c8ccccc78)n6)c5)ccnc4c3n2)cc1 ; c1cc(-c2cccc(-c3nc4ccccc4c4ccccc34)n2)cc(-c2ccc3ccc4ccc(-c5cccc6ccccc56)nc4c3n2)c1 ; c1cc(-c2cccc(-c3nc4ccccc4c4ccccc34)n2)cc(-c2ccc3ccc4ccc(-c5ccc6ccccc6c5)nc4c3n2)c1 ; c1ccc(-c2ccccc2-c2ccc3ccc4ccc(-c5cccc(-c6cccc(-c7nc8ccccc8c8ccccc78)n6)c5)nc4c3n2)cc1] 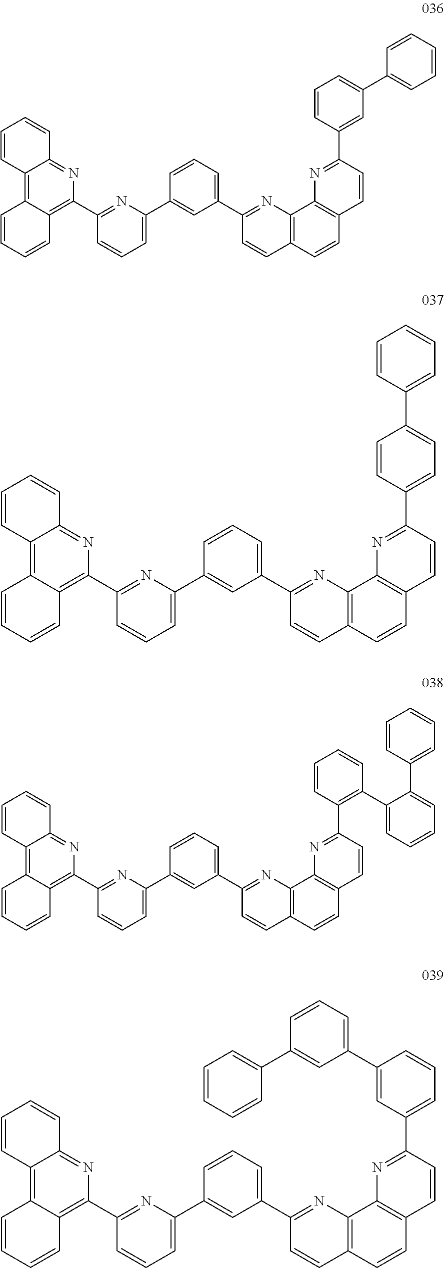 [SMILES: c1ccc(-c2cccc(-c3ccc4ccc5ccc(-c6cccc(-c7cccc(-c8nc9ccccc9c9ccccc89)n7)c6)nc5c4n3)c2)cc1 ; c1ccc(-c2ccc(-c3ccc4ccc5ccc(-c6cccc(-c7cccc(-c8nc9ccccc9c9ccccc89)n7)c6)nc5c4n3)cc2)cc1 ; c1ccc(-c2ccccc2-c2ccccc2-c2ccc3ccc4ccc(-c5cccc(-c6cccc(-c7nc8ccccc8c8ccccc78)n6)c5)nc4c3n2)cc1 ; c1ccc(-c2cccc(-c3cccc(-c4ccc5ccc6ccc(-c7cccc(-c8cccc(-c9nc%10ccccc%10c%10ccccc9%10)n8)c7)nc6c5n4)c3)c2)cc1] 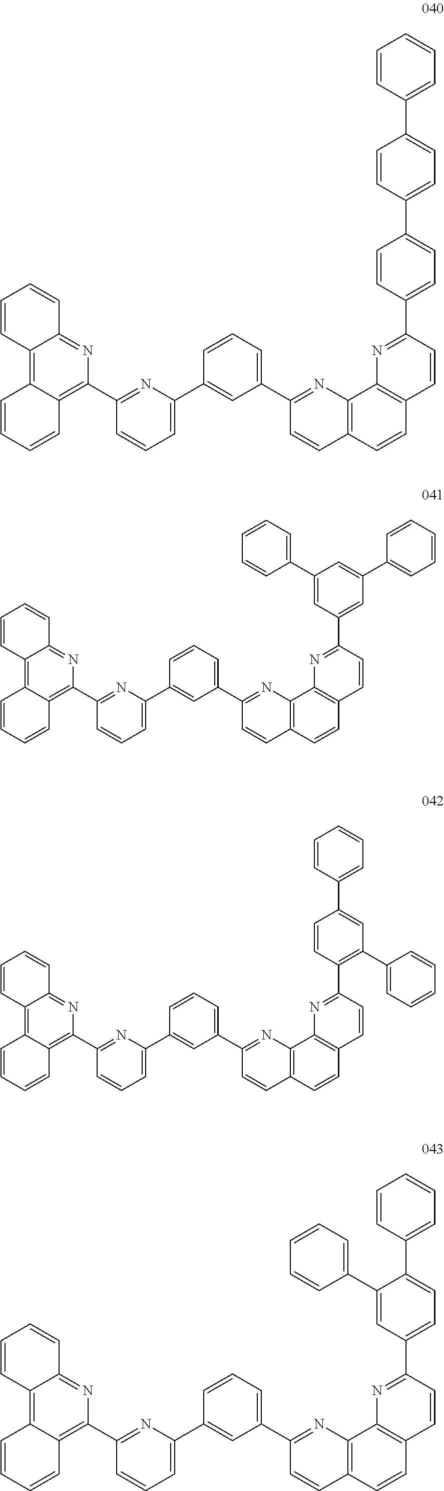 [SMILES: c1ccc(-c2ccc(-c3ccc(-c4ccc5ccc6ccc(-c7cccc(-c8cccc(-c9nc%10ccccc%10c%10ccccc9%10)n8)c7)nc6c5n4)cc3)cc2)cc1 ; c1ccc(-c2cc(-c3ccccc3)cc(-c3ccc4ccc5ccc(-c6cccc(-c7cccc(-c8nc9ccccc9c9ccccc89)n7)c6)nc5c4n3)c2)cc1 ; c1ccc(-c2ccc(-c3ccc4ccc5ccc(-c6cccc(-c7cccc(-c8nc9ccccc9c9ccccc89)n7)c6)nc5c4n3)c(-c3ccccc3)c2)cc1 ; c1ccc(-c2ccc(-c3ccc4ccc5ccc(-c6cccc(-c7cccc(-c8nc9ccccc9c9ccccc89)n7)c6)nc5c4n3)cc2-c2ccccc2)cc1] 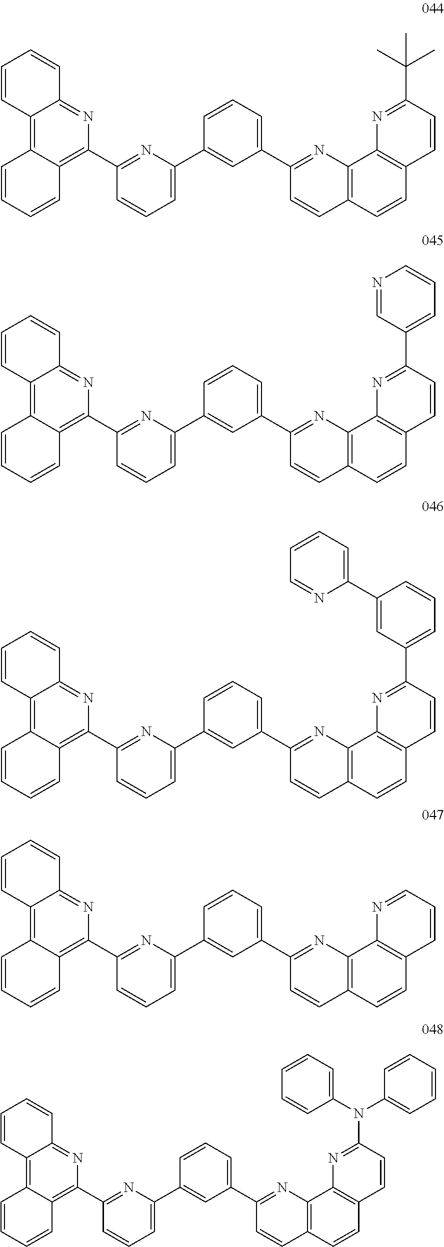 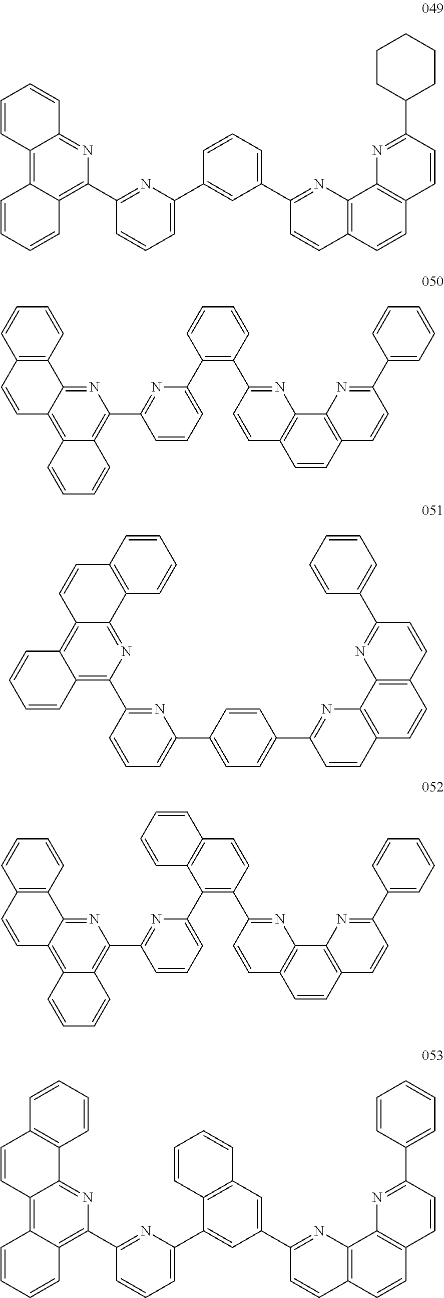 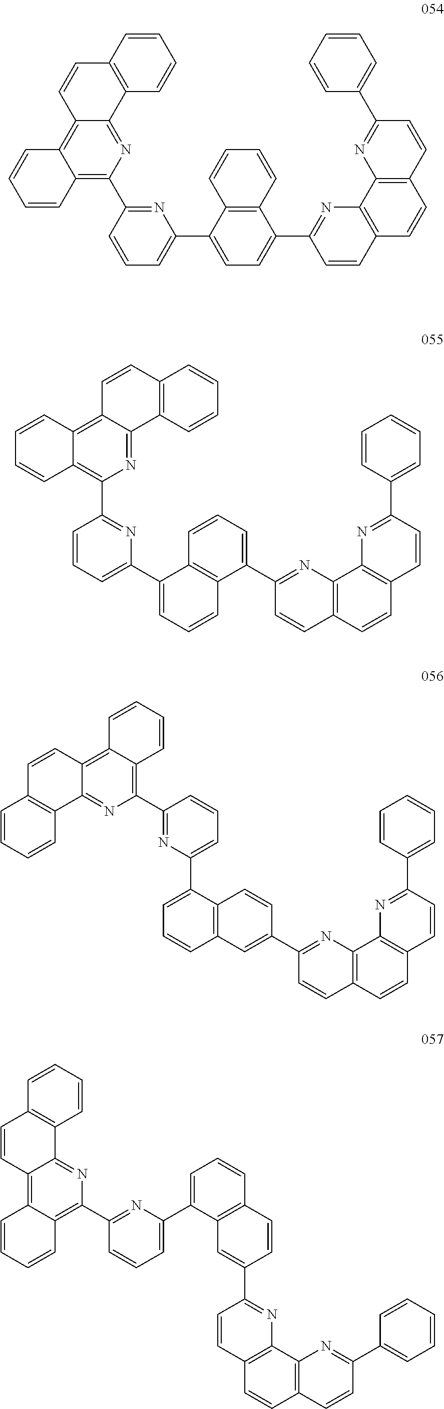 [SMILES: c1ccc(-c2ccc3ccc4ccc(-c5ccc(-c6cccc(-c7nc8c9ccccc9ccc8c8ccccc78)n6)c6ccccc56)nc4c3n2)cc1 ; c1ccc(-c2ccc3ccc4ccc(-c5cccc6c(-c7cccc(-c8nc9c%10ccccc%10ccc9c9ccccc89)n7)cccc56)nc4c3n2)cc1 ; c1ccc(-c2ccc3ccc4ccc(-c5ccc6c(-c7cccc(-c8nc9c%10ccccc%10ccc9c9ccccc89)n7)cccc6c5)nc4c3n2)cc1 ; c1ccc(-c2ccc3ccc4ccc(-c5ccc6cccc(-c7cccc(-c8nc9c%10ccccc%10ccc9c9ccccc89)n7)c6c5)nc4c3n2)cc1] 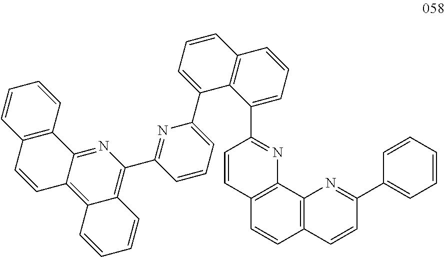 [SMILES: c1ccc(-c2ccc3ccc4ccc(-c5cccc6cccc(-c7cccc(-c8nc9c%10ccccc%10ccc9c9ccccc89)n7)c56)nc4c3n2)cc1] 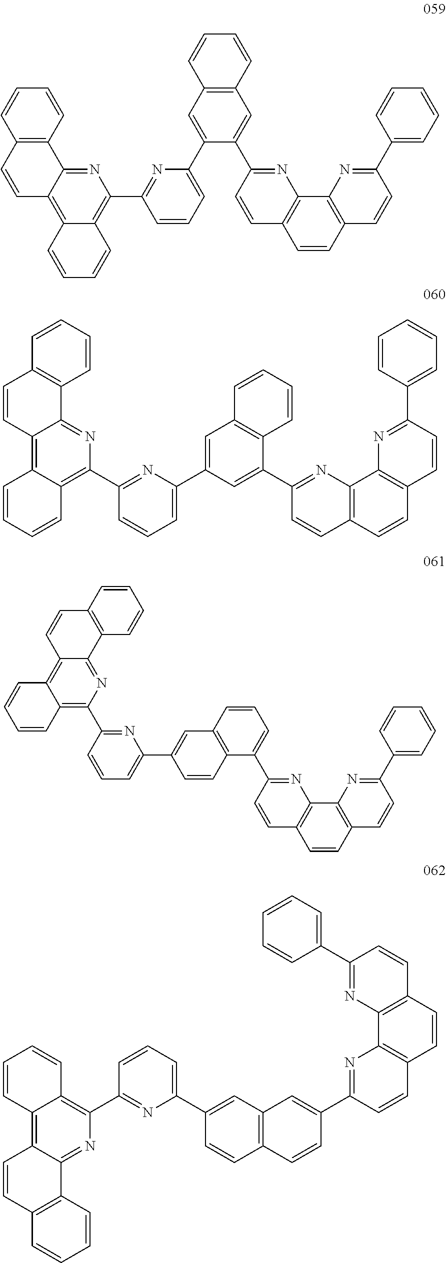 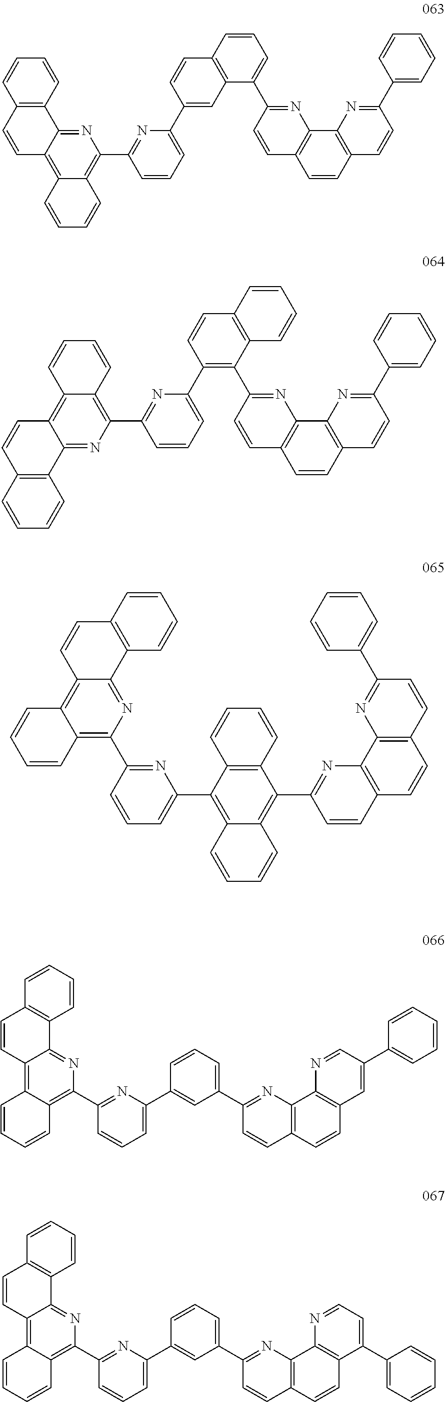 [SMILES: c1ccc(-c2ccc3ccc4ccc(-c5cccc6ccc(-c7cccc(-c8nc9c%10ccccc%10ccc9c9ccccc89)n7)cc56)nc4c3n2)cc1 ; c1ccc(-c2ccc3ccc4ccc(-c5c(-c6cccc(-c7nc8c9ccccc9ccc8c8ccccc78)n6)ccc6ccccc56)nc4c3n2)cc1 ; c1ccc(-c2ccc3ccc4ccc(-c5c6ccccc6c(-c6cccc(-c7nc8c9ccccc9ccc8c8ccccc78)n6)c6ccccc56)nc4c3n2)cc1 ; c1ccc(-c2cnc3c(ccc4ccc(-c5cccc(-c6cccc(-c7nc8c9ccccc9ccc8c8ccccc78)n6)c5)nc43)c2)cc1 ; c1ccc(-c2ccnc3c2ccc2ccc(-c4cccc(-c5cccc(-c6nc7c8ccccc8ccc7c7ccccc67)n5)c4)nc23)cc1] 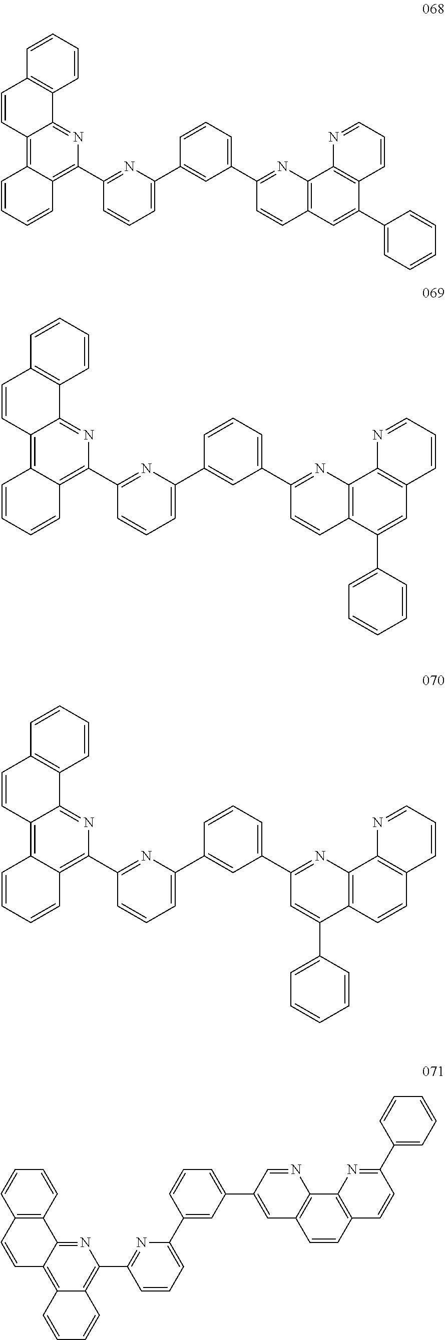 [SMILES: c1ccc(-c2cc3ccc(-c4cccc(-c5cccc(-c6nc7c8ccccc8ccc7c7ccccc67)n5)c4)nc3c3ncccc23)cc1 ; c1ccc(-c2cc3cccnc3c3nc(-c4cccc(-c5cccc(-c6nc7c8ccccc8ccc7c7ccccc67)n5)c4)ccc23)cc1 ; c1ccc(-c2cc(-c3cccc(-c4cccc(-c5nc6c7ccccc7ccc6c6ccccc56)n4)c3)nc3c2ccc2cccnc23)cc1 ; c1ccc(-c2ccc3ccc4cc(-c5cccc(-c6cccc(-c7nc8c9ccccc9ccc8c8ccccc78)n6)c5)cnc4c3n2)cc1] 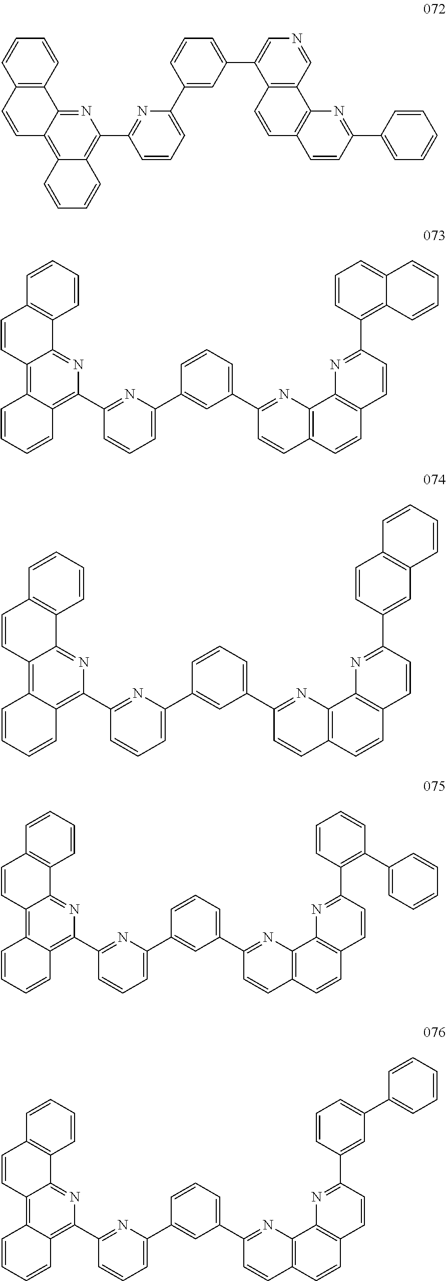 [SMILES: c1ccc(-c2ccc3ccc4c(-c5cccc(-c6cccc(-c7nc8c9ccccc9ccc8c8ccccc78)n6)c5)cncc4c3n2)cc1 ; c1cc(-c2cccc(-c3nc4c5ccccc5ccc4c4ccccc34)n2)cc(-c2ccc3ccc4ccc(-c5cccc6ccccc56)nc4c3n2)c1 ; c1cc(-c2cccc(-c3nc4c5ccccc5ccc4c4ccccc34)n2)cc(-c2ccc3ccc4ccc(-c5ccc6ccccc6c5)nc4c3n2)c1 ; c1ccc(-c2ccccc2-c2ccc3ccc4ccc(-c5cccc(-c6cccc(-c7nc8c9ccccc9ccc8c8ccccc78)n6)c5)nc4c3n2)cc1 ; c1ccc(-c2cccc(-c3ccc4ccc5ccc(-c6cccc(-c7cccc(-c8nc9c%10ccccc%10ccc9c9ccccc89)n7)c6)nc5c4n3)c2)cc1] 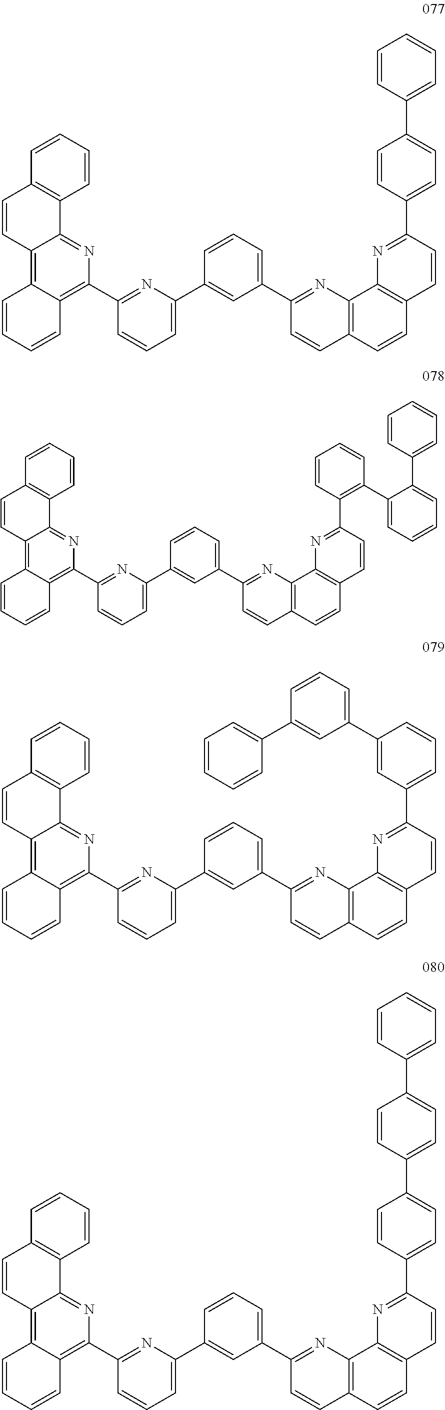 [SMILES: c1ccc(-c2ccc(-c3ccc4ccc5ccc(-c6cccc(-c7cccc(-c8nc9c%10ccccc%10ccc9c9ccccc89)n7)c6)nc5c4n3)cc2)cc1 ; c1ccc(-c2ccccc2-c2ccccc2-c2ccc3ccc4ccc(-c5cccc(-c6cccc(-c7nc8c9ccccc9ccc8c8ccccc78)n6)c5)nc4c3n2)cc1 ; c1ccc(-c2cccc(-c3cccc(-c4ccc5ccc6ccc(-c7cccc(-c8cccc(-c9nc%10c%11ccccc%11ccc%10c%10ccccc9%10)n8)c7)nc6c5n4)c3)c2)cc1 ; c1ccc(-c2ccc(-c3ccc(-c4ccc5ccc6ccc(-c7cccc(-c8cccc(-c9nc%10c%11ccccc%11ccc%10c%10ccccc9%10)n8)c7)nc6c5n4)cc3)cc2)cc1] 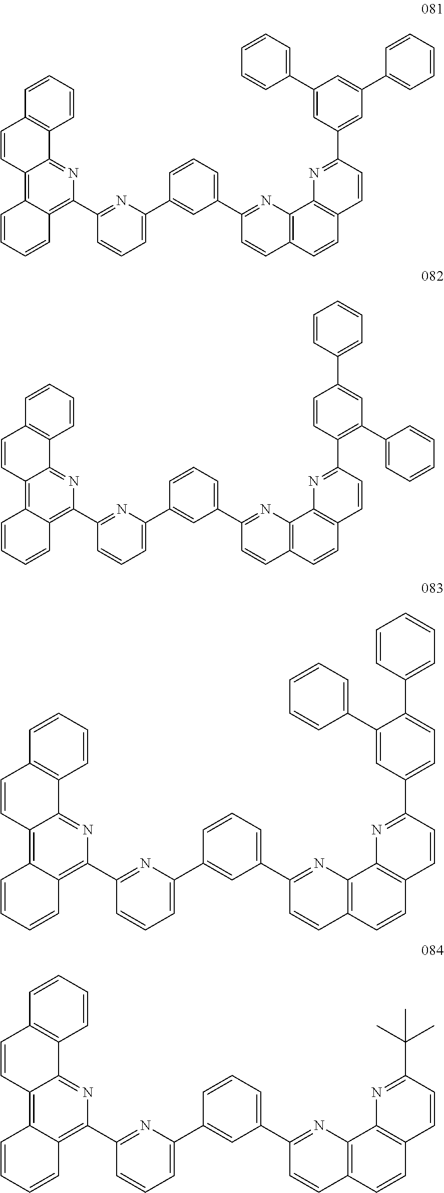 [SMILES: c1ccc(-c2cc(-c3ccccc3)cc(-c3ccc4ccc5ccc(-c6cccc(-c7cccc(-c8nc9c%10ccccc%10ccc9c9ccccc89)n7)c6)nc5c4n3)c2)cc1 ; c1ccc(-c2ccc(-c3ccc4ccc5ccc(-c6cccc(-c7cccc(-c8nc9c%10ccccc%10ccc9c9ccccc89)n7)c6)nc5c4n3)c(-c3ccccc3)c2)cc1 ; c1ccc(-c2ccc(-c3ccc4ccc5ccc(-c6cccc(-c7cccc(-c8nc9c%10ccccc%10ccc9c9ccccc89)n7)c6)nc5c4n3)cc2-c2ccccc2)cc1 ; CC(C)(C)c1ccc2ccc3ccc(-c4cccc(-c5cccc(-c6nc7c8ccccc8ccc7c7ccccc67)n5)c4)nc3c2n1] 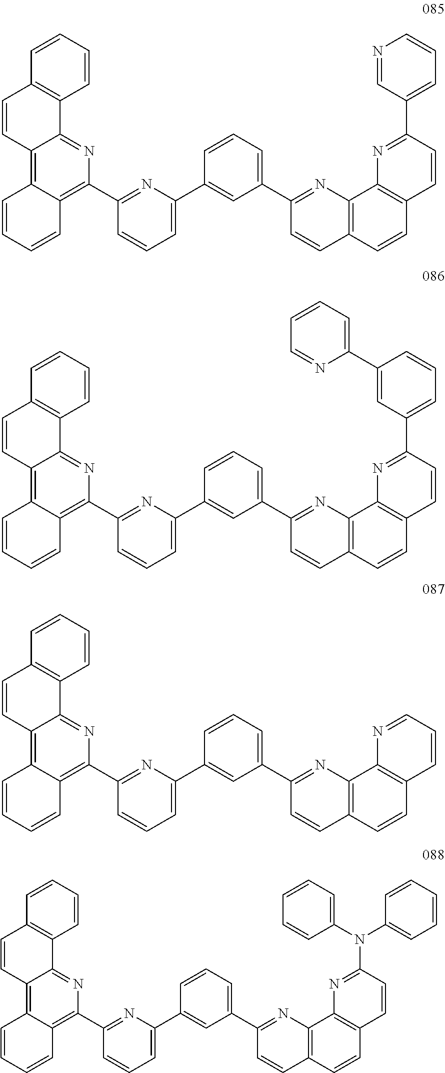 [SMILES: c1cncc(-c2ccc3ccc4ccc(-c5cccc(-c6cccc(-c7nc8c9ccccc9ccc8c8ccccc78)n6)c5)nc4c3n2)c1 ; c1ccc(-c2cccc(-c3ccc4ccc5ccc(-c6cccc(-c7cccc(-c8nc9c%10ccccc%10ccc9c9ccccc89)n7)c6)nc5c4n3)c2)nc1 ; c1cc(-c2cccc(-c3nc4c5ccccc5ccc4c4ccccc34)n2)cc(-c2ccc3ccc4cccnc4c3n2)c1 ; c1ccc(N(c2ccccc2)c2ccc3ccc4ccc(-c5cccc(-c6cccc(-c7nc8c9ccccc9ccc8c8ccccc78)n6)c5)nc4c3n2)cc1] 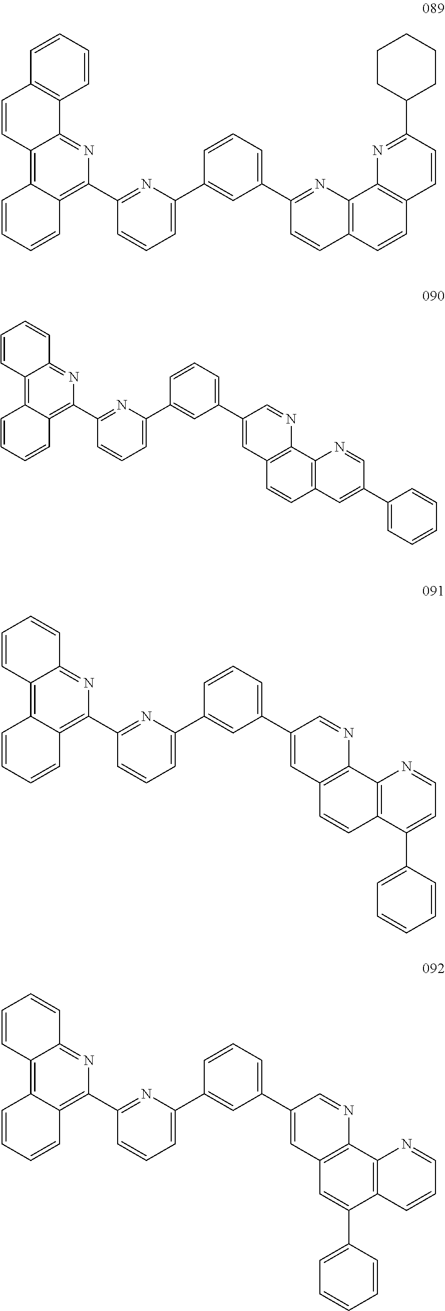 [SMILES: c1cc(-c2cccc(-c3nc4c5ccccc5ccc4c4ccccc34)n2)cc(-c2ccc3ccc4ccc(C5CCCCC5)nc4c3n2)c1 ; c1ccc(-c2cnc3c(ccc4cc(-c5cccc(-c6cccc(-c7nc8ccccc8c8ccccc78)n6)c5)cnc43)c2)cc1 ; c1ccc(-c2ccnc3c2ccc2cc(-c4cccc(-c5cccc(-c6nc7ccccc7c7ccccc67)n5)c4)cnc23)cc1 ; c1ccc(-c2cc3cc(-c4cccc(-c5cccc(-c6nc7ccccc7c7ccccc67)n5)c4)cnc3c3ncccc23)cc1] 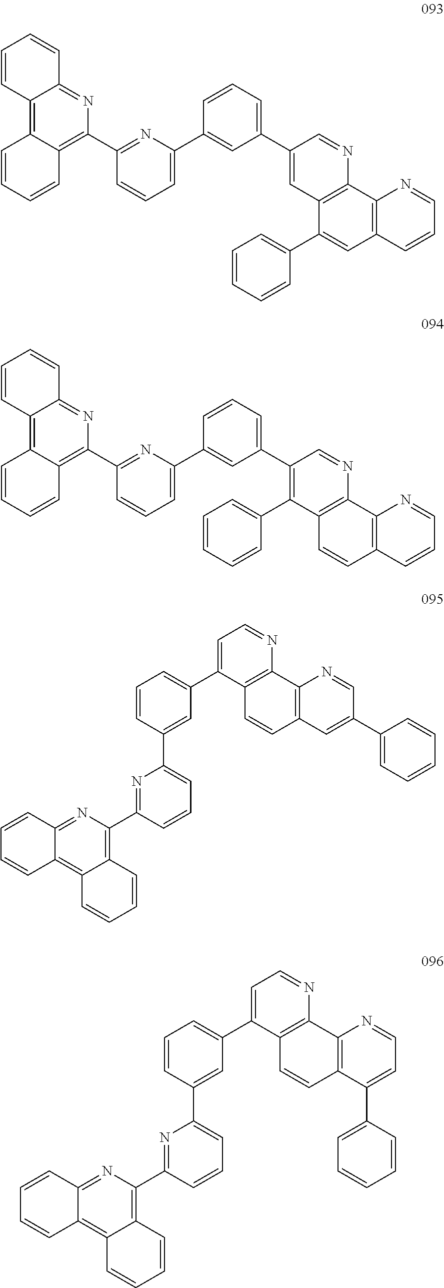 [SMILES: c1ccc(-c2cc3cccnc3c3ncc(-c4cccc(-c5cccc(-c6nc7ccccc7c7ccccc67)n5)c4)cc23)cc1 ; c1ccc(-c2c(-c3cccc(-c4cccc(-c5nc6ccccc6c6ccccc56)n4)c3)cnc3c2ccc2cccnc23)cc1 ; c1ccc(-c2cnc3c(ccc4c(-c5cccc(-c6cccc(-c7nc8ccccc8c8ccccc78)n6)c5)ccnc43)c2)cc1 ; c1ccc(-c2ccnc3c2ccc2c(-c4cccc(-c5cccc(-c6nc7ccccc7c7ccccc67)n5)c4)ccnc23)cc1] 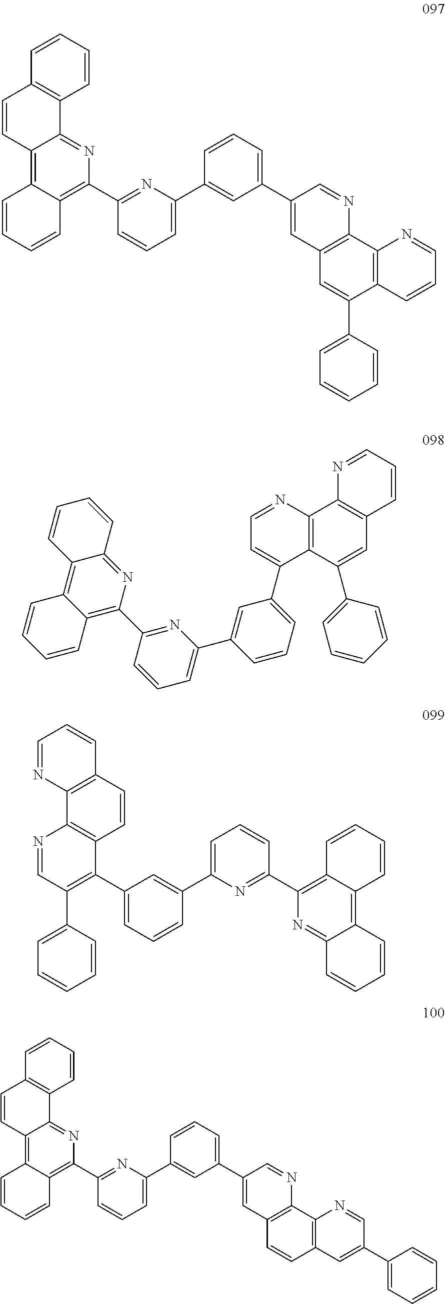 [SMILES: c1ccc(-c2cc3cc(-c4cccc(-c5cccc(-c6nc7c8ccccc8ccc7c7ccccc67)n5)c4)cnc3c3ncccc23)cc1 ; c1ccc(-c2cc3cccnc3c3nccc(-c4cccc(-c5cccc(-c6nc7ccccc7c7ccccc67)n5)c4)c23)cc1 ; c1ccc(-c2cnc3c(ccc4cccnc43)c2-c2cccc(-c3cccc(-c4nc5ccccc5c5ccccc45)n3)c2)cc1 ; c1ccc(-c2cnc3c(ccc4cc(-c5cccc(-c6cccc(-c7nc8c9ccccc9ccc8c8ccccc78)n6)c5)cnc43)c2)cc1] 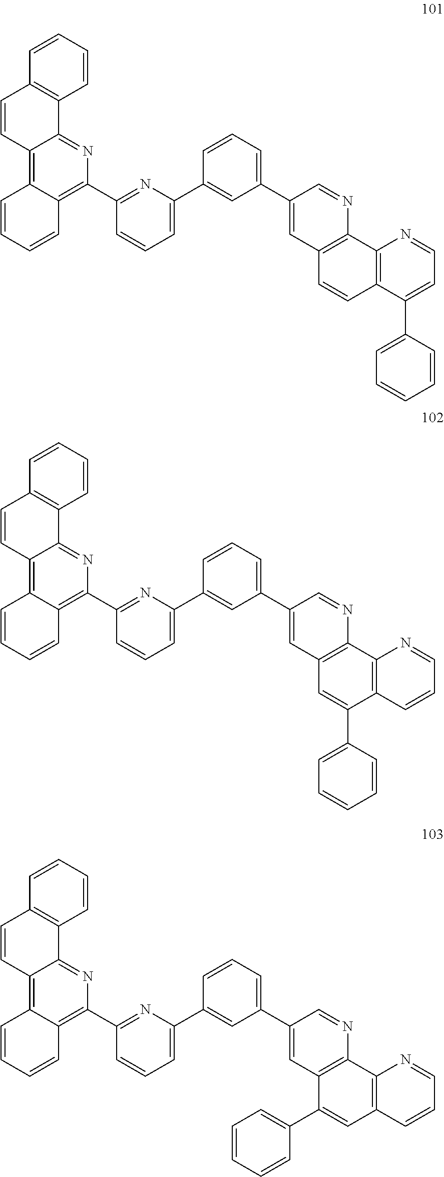 [SMILES: c1ccc(-c2ccnc3c2ccc2cc(-c4cccc(-c5cccc(-c6nc7c8ccccc8ccc7c7ccccc67)n5)c4)cnc23)cc1 ; c1ccc(-c2cc3cc(-c4cccc(-c5cccc(-c6nc7c8ccccc8ccc7c7ccccc67)n5)c4)cnc3c3ncccc23)cc1 ; c1ccc(-c2cc3cccnc3c3ncc(-c4cccc(-c5cccc(-c6nc7c8ccccc8ccc7c7ccccc67)n5)c4)cc23)cc1] 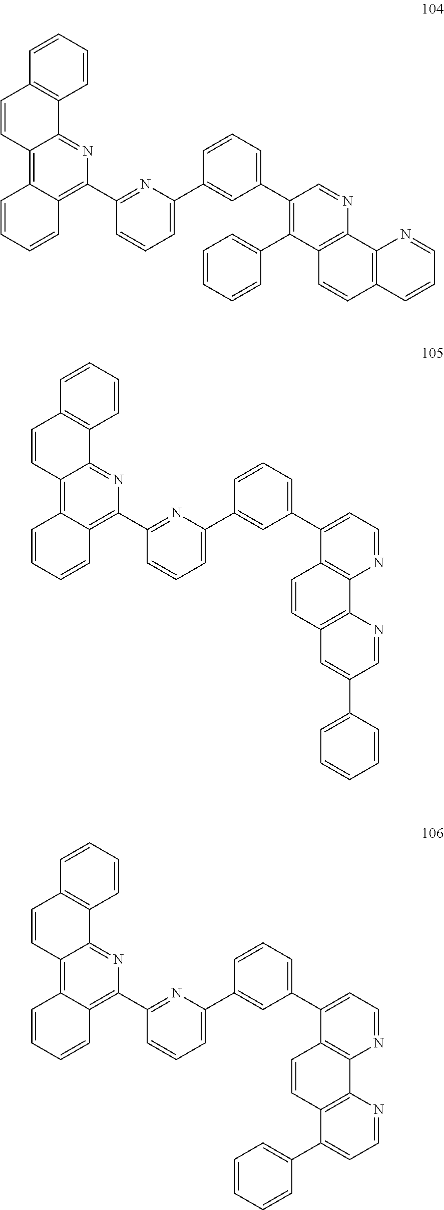 [SMILES: c1ccc(-c2c(-c3cccc(-c4cccc(-c5nc6c7ccccc7ccc6c6ccccc56)n4)c3)cnc3c2ccc2cccnc23)cc1 ; c1ccc(-c2cnc3c(ccc4c(-c5cccc(-c6cccc(-c7nc8c9ccccc9ccc8c8ccccc78)n6)c5)ccnc43)c2)cc1 ; c1ccc(-c2ccnc3c2ccc2c(-c4cccc(-c5cccc(-c6nc7c8ccccc8ccc7c7ccccc67)n5)c4)ccnc23)cc1] 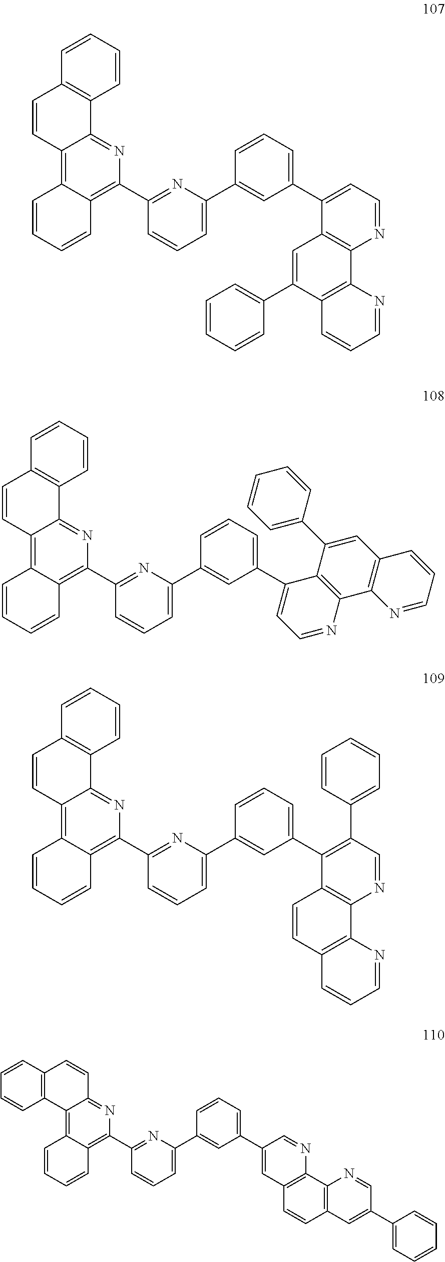 [SMILES: c1ccc(-c2cc3c(-c4cccc(-c5cccc(-c6nc7c8ccccc8ccc7c7ccccc67)n5)c4)ccnc3c3ncccc23)cc1 ; c1ccc(-c2cc3cccnc3c3nccc(-c4cccc(-c5cccc(-c6nc7c8ccccc8ccc7c7ccccc67)n5)c4)c23)cc1 ; c1ccc(-c2cnc3c(ccc4cccnc43)c2-c2cccc(-c3cccc(-c4nc5c6ccccc6ccc5c5ccccc45)n3)c2)cc1 ; c1ccc(-c2cnc3c(ccc4cc(-c5cccc(-c6cccc(-c7nc8ccc9ccccc9c8c8ccccc78)n6)c5)cnc43)c2)cc1] 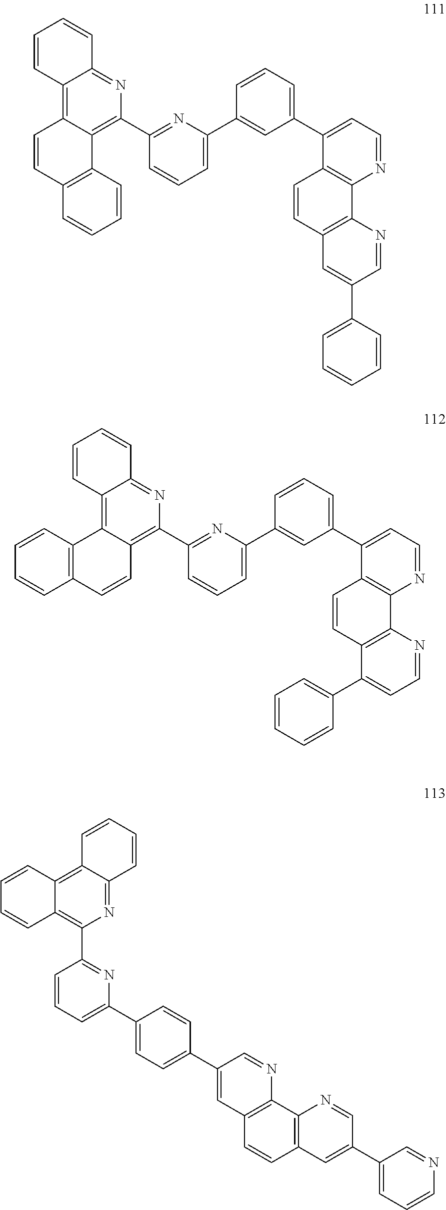 [SMILES: c1ccc(-c2cnc3c(ccc4c(-c5cccc(-c6cccc(-c7nc8ccccc8c8ccc9ccccc9c78)n6)c5)ccnc43)c2)cc1 ; c1ccc(-c2ccnc3c2ccc2c(-c4cccc(-c5cccc(-c6nc7ccccc7c7c6ccc6ccccc67)n5)c4)ccnc23)cc1 ; c1cncc(-c2cnc3c(ccc4cc(-c5ccc(-c6cccc(-c7nc8ccccc8c8ccccc78)n6)cc5)cnc43)c2)c1] 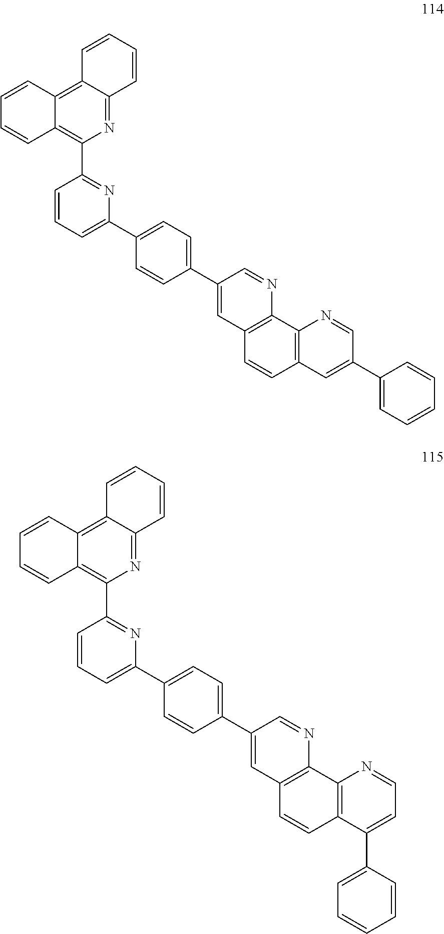 [SMILES: c1ccc(-c2cnc3c(ccc4cc(-c5ccc(-c6cccc(-c7nc8ccccc8c8ccccc78)n6)cc5)cnc43)c2)cc1 ; c1ccc(-c2ccnc3c2ccc2cc(-c4ccc(-c5cccc(-c6nc7ccccc7c7ccccc67)n5)cc4)cnc23)cc1] 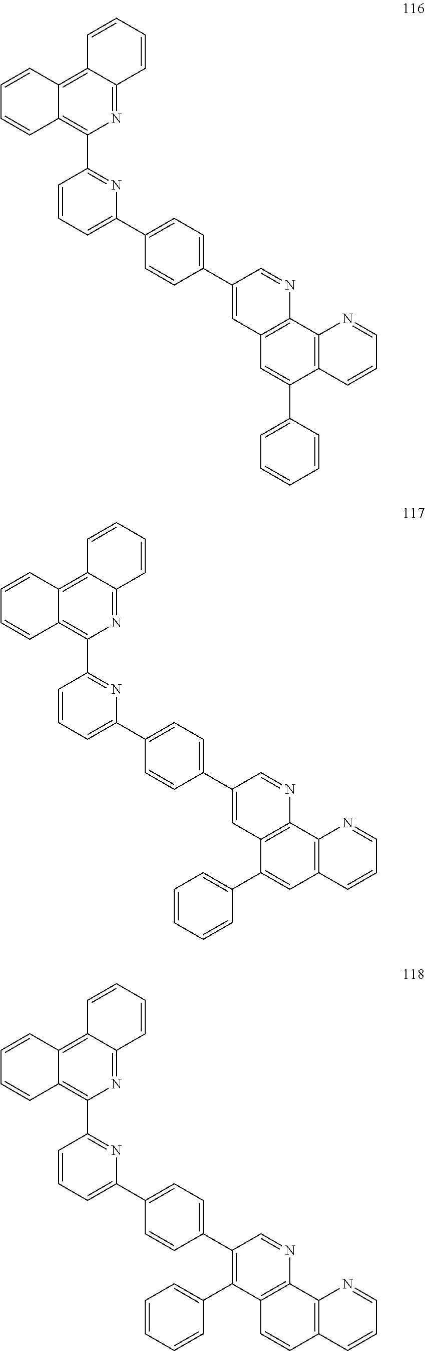 [SMILES: c1ccc(-c2cc3cc(-c4ccc(-c5cccc(-c6nc7ccccc7c7ccccc67)n5)cc4)cnc3c3ncccc23)cc1 ; c1ccc(-c2cc3cccnc3c3ncc(-c4ccc(-c5cccc(-c6nc7ccccc7c7ccccc67)n5)cc4)cc23)cc1 ; c1ccc(-c2c(-c3ccc(-c4cccc(-c5nc6ccccc6c6ccccc56)n4)cc3)cnc3c2ccc2cccnc23)cc1] 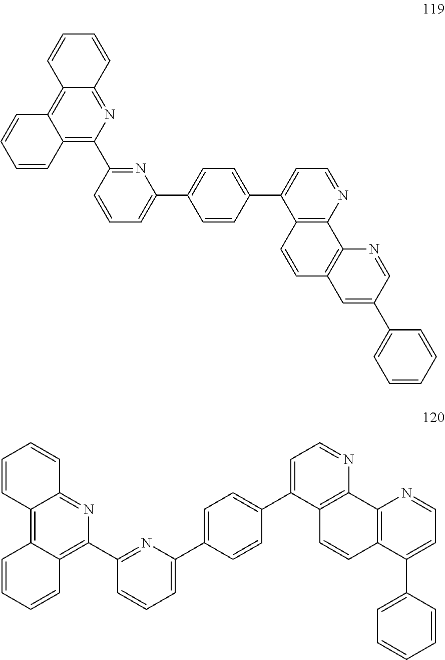 [SMILES: c1ccc(-c2cnc3c(ccc4c(-c5ccc(-c6cccc(-c7nc8ccccc8c8ccccc78)n6)cc5)ccnc43)c2)cc1 ; c1ccc(-c2ccnc3c2ccc2c(-c4ccc(-c5cccc(-c6nc7ccccc7c7ccccc67)n5)cc4)ccnc23)cc1] 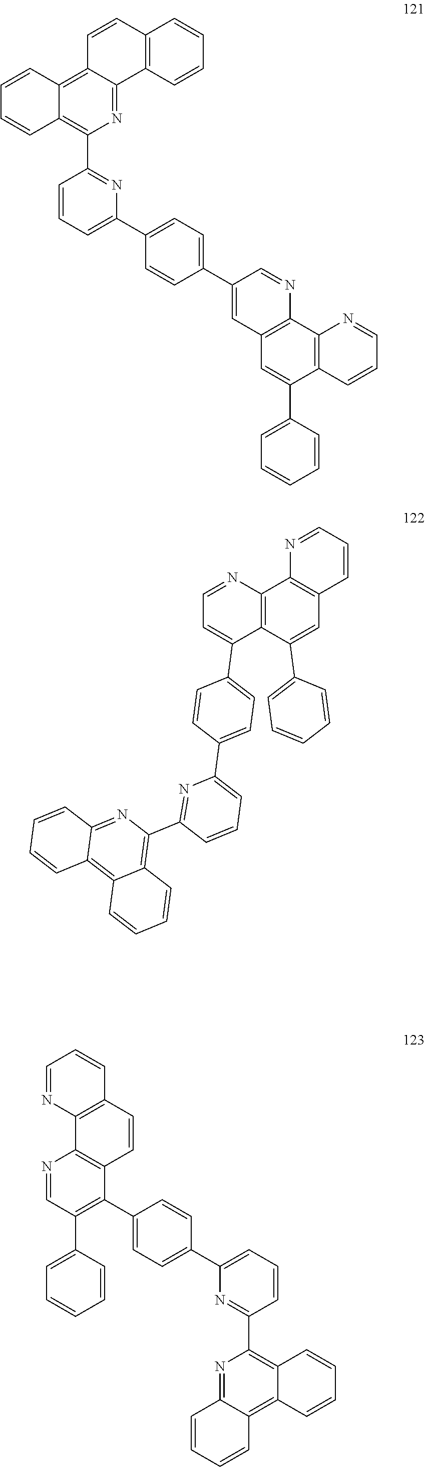 [SMILES: c1ccc(-c2cc3cc(-c4ccc(-c5cccc(-c6nc7c8ccccc8ccc7c7ccccc67)n5)cc4)cnc3c3ncccc23)cc1 ; c1ccc(-c2cc3cccnc3c3nccc(-c4ccc(-c5cccc(-c6nc7ccccc7c7ccccc67)n5)cc4)c23)cc1 ; c1ccc(-c2cnc3c(ccc4cccnc43)c2-c2ccc(-c3cccc(-c4nc5ccccc5c5ccccc45)n3)cc2)cc1] 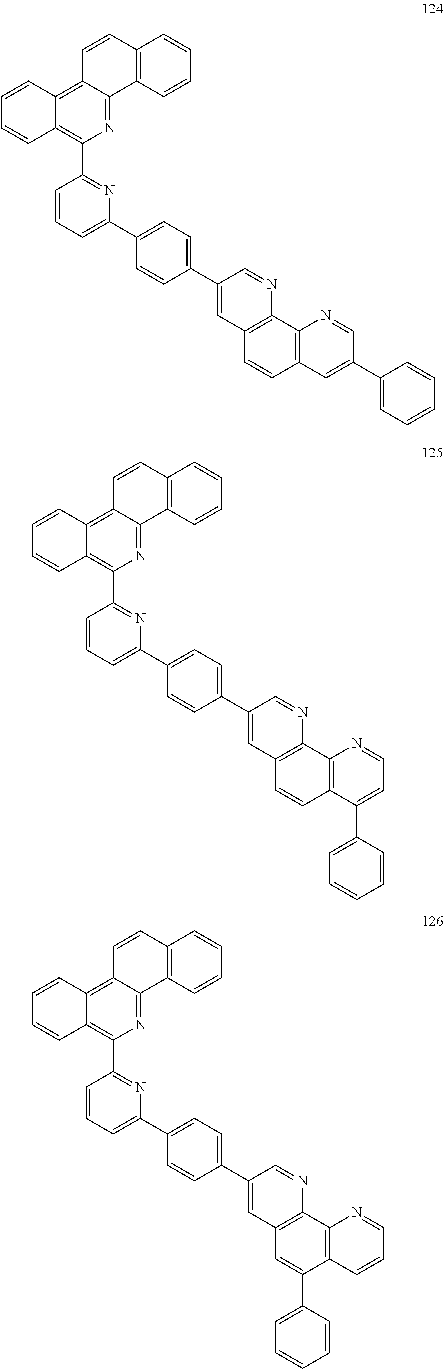 [SMILES: c1ccc(-c2cnc3c(ccc4cc(-c5ccc(-c6cccc(-c7nc8c9ccccc9ccc8c8ccccc78)n6)cc5)cnc43)c2)cc1 ; c1ccc(-c2ccnc3c2ccc2cc(-c4ccc(-c5cccc(-c6nc7c8ccccc8ccc7c7ccccc67)n5)cc4)cnc23)cc1 ; c1ccc(-c2cc3cc(-c4ccc(-c5cccc(-c6nc7c8ccccc8ccc7c7ccccc67)n5)cc4)cnc3c3ncccc23)cc1] 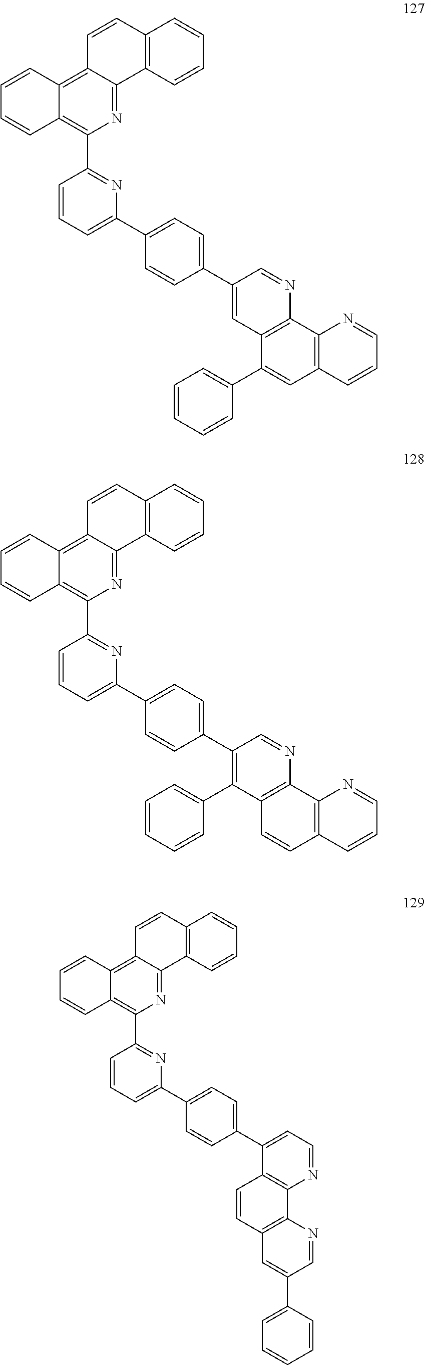 [SMILES: c1ccc(-c2cc3cccnc3c3ncc(-c4ccc(-c5cccc(-c6nc7c8ccccc8ccc7c7ccccc67)n5)cc4)cc23)cc1 ; c1ccc(-c2c(-c3ccc(-c4cccc(-c5nc6c7ccccc7ccc6c6ccccc56)n4)cc3)cnc3c2ccc2cccnc23)cc1 ; c1ccc(-c2cnc3c(ccc4c(-c5ccc(-c6cccc(-c7nc8c9ccccc9ccc8c8ccccc78)n6)cc5)ccnc43)c2)cc1] 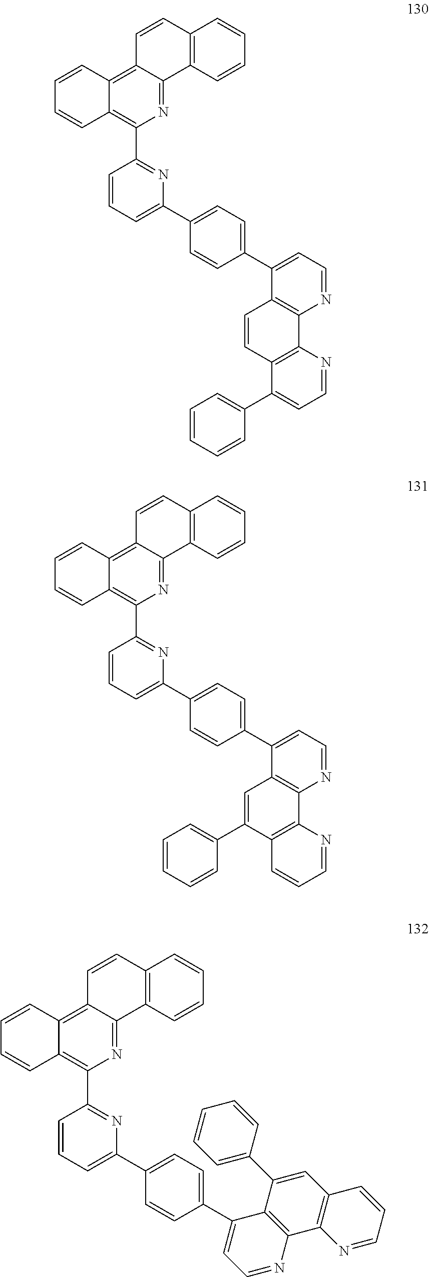 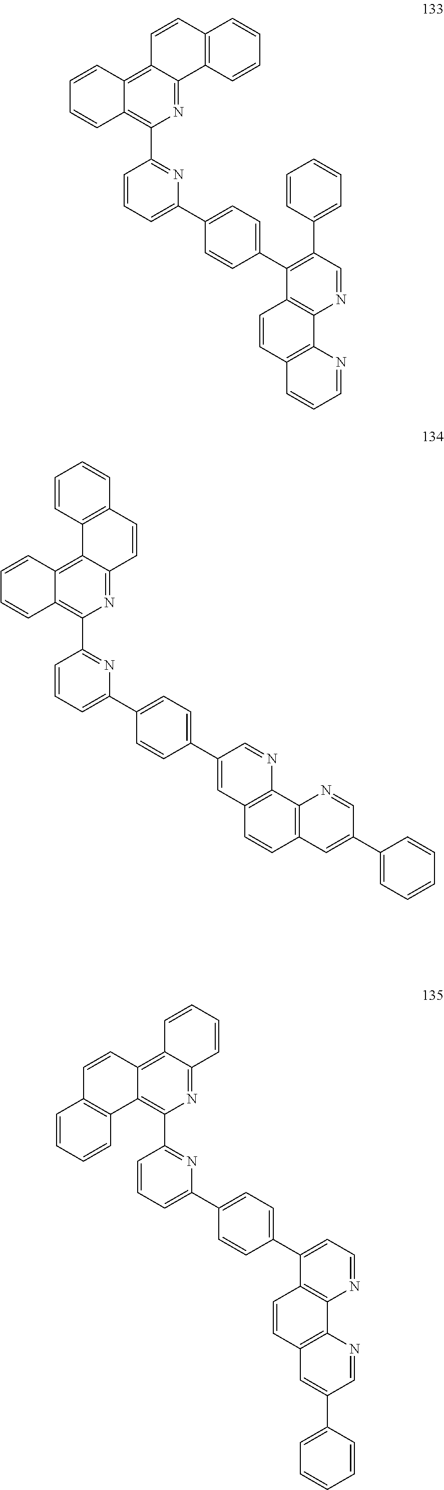 [SMILES: c1ccc(-c2cnc3c(ccc4cccnc43)c2-c2ccc(-c3cccc(-c4nc5c6ccccc6ccc5c5ccccc45)n3)cc2)cc1 ; c1ccc(-c2cnc3c(ccc4cc(-c5ccc(-c6cccc(-c7nc8ccc9ccccc9c8c8ccccc78)n6)cc5)cnc43)c2)cc1 ; c1ccc(-c2cnc3c(ccc4c(-c5ccc(-c6cccc(-c7nc8ccccc8c8ccc9ccccc9c78)n6)cc5)ccnc43)c2)cc1] 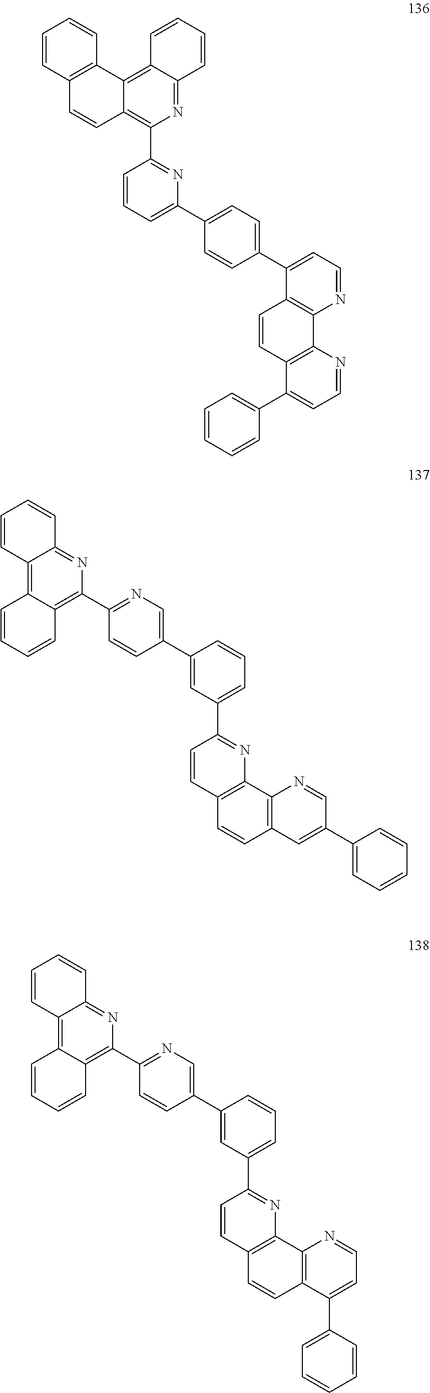 [SMILES: c1ccc(-c2ccnc3c2ccc2c(-c4ccc(-c5cccc(-c6nc7ccccc7c7c6ccc6ccccc67)n5)cc4)ccnc23)cc1 ; c1ccc(-c2cnc3c(ccc4ccc(-c5cccc(-c6ccc(-c7nc8ccccc8c8ccccc78)nc6)c5)nc43)c2)cc1 ; c1ccc(-c2ccnc3c2ccc2ccc(-c4cccc(-c5ccc(-c6nc7ccccc7c7ccccc67)nc5)c4)nc23)cc1] 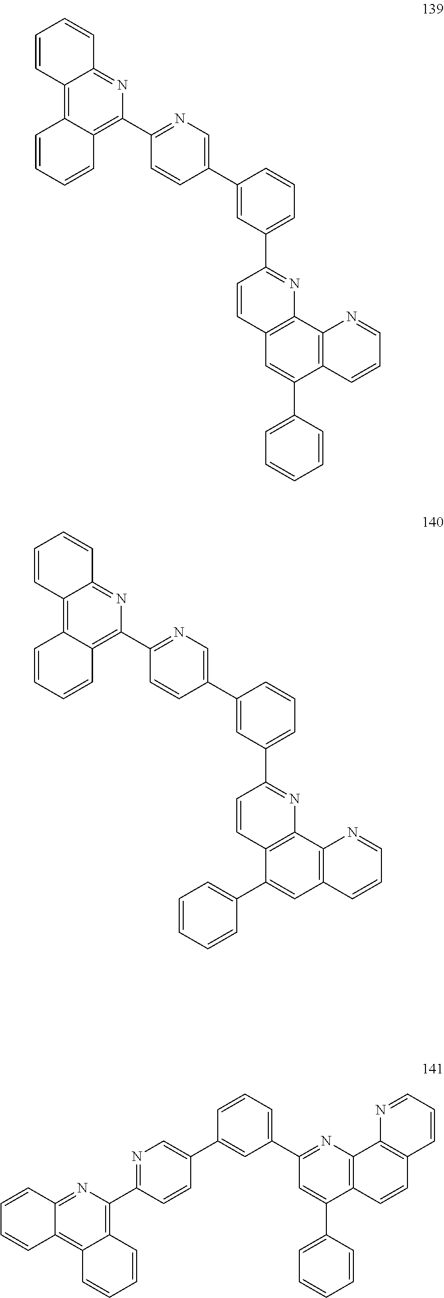 [SMILES: c1ccc(-c2cc3ccc(-c4cccc(-c5ccc(-c6nc7ccccc7c7ccccc67)nc5)c4)nc3c3ncccc23)cc1 ; c1ccc(-c2cc3cccnc3c3nc(-c4cccc(-c5ccc(-c6nc7ccccc7c7ccccc67)nc5)c4)ccc23)cc1 ; c1ccc(-c2cc(-c3cccc(-c4ccc(-c5nc6ccccc6c6ccccc56)nc4)c3)nc3c2ccc2cccnc23)cc1] 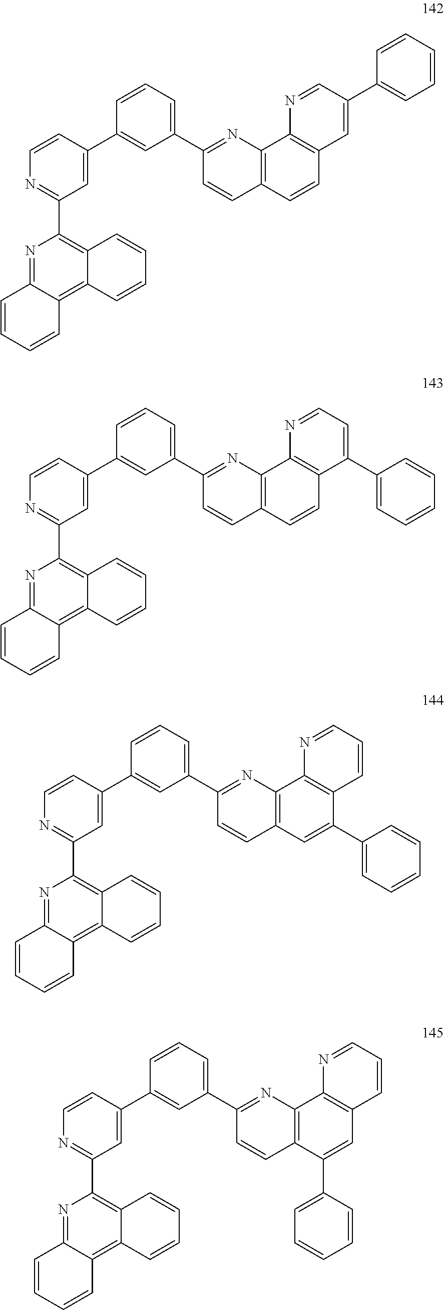 [SMILES: c1ccc(-c2cnc3c(ccc4ccc(-c5cccc(-c6ccnc(-c7nc8ccccc8c8ccccc78)c6)c5)nc43)c2)cc1 ; c1ccc(-c2ccnc3c2ccc2ccc(-c4cccc(-c5ccnc(-c6nc7ccccc7c7ccccc67)c5)c4)nc23)cc1 ; c1ccc(-c2cc3ccc(-c4cccc(-c5ccnc(-c6nc7ccccc7c7ccccc67)c5)c4)nc3c3ncccc23)cc1 ; c1ccc(-c2cc3cccnc3c3nc(-c4cccc(-c5ccnc(-c6nc7ccccc7c7ccccc67)c5)c4)ccc23)cc1] 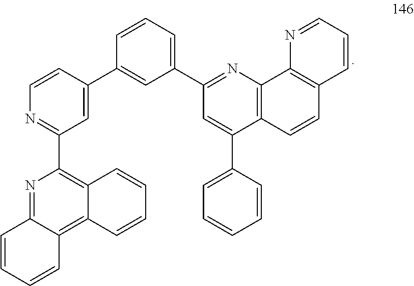 [SMILES: c1ccc(-c2cc(-c3cccc(-c4ccnc(-c5nc6ccccc6c6ccccc56)c4)c3)nc3c2ccc2cccnc23)cc1]
